SEQUENCE LISTING (290 sequences)

SEQ ID NO: 1 - protein, 5 aa, Artificial Sequence
  Description of Artificial Sequence Synthetic peptide
SYWIG

SEQ ID NO: 2 - protein, 17 aa, Artificial Sequence
  Description of Artificial Sequence Synthetic peptide
IIYPGDSDTRYSPSFQG

SEQ ID NO: 3 - protein, 14 aa, Artificial Sequence
  Description of Artificial Sequence Synthetic peptide
HGRGYNGYEGAFDI

SEQ ID NO: 4 - protein, 11 aa, Artificial Sequence
  Description of Artificial Sequence Synthetic peptide
RASQGISSALA

SEQ ID NO: 5 - protein, 7 aa, Artificial Sequence
  Description of Artificial Sequence Synthetic peptide
DASSLES

SEQ ID NO: 6 - protein, 10 aa, Artificial Sequence
  Description of Artificial Sequence Synthetic peptide
CQQFNSYPLT

SEQ ID NO: 7 - protein, 120 aa, Artificial Sequence
  Description of Artificial Sequence Synthetic polypeptide
EVQLVESGGGLVQPGGSLRLSCAASGFTFSDYAMSWVRQAPGKGLEWVAVISENGSDTYYADSVKGRFTISRDDSKNTLYLQMNSLRAEDTAVYYCARDRGGAVSYFDVWGQGTLVTVSS

SEQ ID NO: 8 - protein, 109 aa, Artificial Sequence
  Description of Artificial Sequence Synthetic polypeptide
DIQMTQSPSSLSASVGDRVTITCRASQDVSSYLAWYQQKPGKAPKLLIYAASSLESGVPSRFSGSGSGTDFTLTISSLQPEDFATYYCQQYNSLPYTFGQGTKVEIKRT

SEQ ID NO: 9 - protein, 121 aa, Artificial Sequence
  Description of Artificial Sequence Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSS

SEQ ID NO: 10 - protein, 107 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIK

SEQ ID NO: 11 - protein, 9 aa, Artificial Sequence
  Description of Artificial Sequence Synthetic peptide
FTFSDADMD

SEQ ID NO: 12 - protein, 19 aa, Artificial Sequence
  Description of Artificial Sequence Synthetic peptide
RTRNKAGSYTTEYAASVKG

SEQ ID NO: 13 - protein, 12 aa, Artificial Sequence
  Description of Artificial Sequence Synthetic peptide
AREPKYWIDFDL

SEQ ID NO: 14 - protein, 11 aa, Artificial Sequence
  Description of Artificial Sequence Synthetic peptide
RASQSISSYLN

SEQ ID NO: 15 - protein, 7 aa, Artificial Sequence
  Description of Artificial Sequence Synthetic peptide
AASSLQS

SEQ ID NO: 16 - protein, 9 aa, Artificial Sequence
  Description of Artificial Sequence Synthetic peptide
QQSYIAPYT

SEQ ID NO: 17 - DNA, 363 bp, Artificial Sequence
  Description of Artificial Sequence Synthetic polynucleotide
gaggtgcagctggtggagtctgggggaggcttggtccagcctggagggtccctgagactctcctgtgcagcctctggattcaccttcagtgacgccgacatggactgggtccgccaggctccagggaaggggctggagtgggttggccgtactagaaacaaagcaggaagttacaccacagaatacgccgcgtctgtgaaaggcagattcaccatctcaagagatgattcaaagaactcactgtatctgcaaatgaacagcctgaaaaccgaggacacggcggtgtactactgcgccagagagcctaaatactggatcgacttcgacctatgggggagaggtaccttggtcaccgtctcctca

SEQ ID NO: 18 - DNA, 321 bp, Artificial Sequence
  Description of Artificial Sequence Synthetic polynucleotide
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaagctacatcgccccttacacttttggcggagggaccaaggttgagatcaaa

SEQ ID NO: 19 - protein, 119 aa, Artificial Sequence
  Description of Artificial Sequence Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSS

SEQ ID NO: 20 - protein, 106 aa, Artificial Sequence
  Description of Artificial Sequence Synthetic polypeptide
DIQMTQSPSSLSASVGDRVTITCRASQSINSYLNWYQQKPGKAPKLLIYAASSLQSGVPSRFSGSGSGTDFTLTISSLQPEDFATYYCQQGVSDITFGGGTKVEIK

SEQ ID NO: 21 - protein, 9 aa, Artificial Sequence
  Description of Artificial Sequence Synthetic peptide
GTFRIYAIS

SEQ ID NO: 22 - protein, 17 aa, Artificial Sequence
  Description of Artificial Sequence Synthetic peptide
GIIPDFGVANYAQKFQG

SEQ ID NO: 23 - protein, 12 aa, Artificial Sequence
  Description of Artificial Sequence Synthetic peptide
ARGGLDTDEFDL

SEQ ID NO: 24 - protein, 11 aa, Artificial Sequence
  Description of Artificial Sequence Synthetic peptide
RASQSINSYLN

SEQ ID NO: 25 - protein, 7 aa, Artificial Sequence
  Description of Artificial Sequence Synthetic peptide
AASSLQS

SEQ ID NO: 26 - protein, 8 aa, Artificial Sequence
  Description of Artificial Sequence Synthetic peptide
QQGVSDIT

SEQ ID NO: 27 - DNA, 357 bp, Artificial Sequence
  Description of Artificial Sequence Synthetic polynucleotide
caggtgcagctggtgcagtctggggctgaggtgaagaagcctgggtcctcggtgaaggtctcctgcaaggcttctggaggcaccttccgaatctatgctatcagctgggtgcgacaggcccctggacaagggcttgagtggatgggagggatcatccctgacttcggtgtagcaaactacgcacagaagttccagggcagagtcacgattaccgcggacgaatccacgagcacagcctacatggagctgagcagcctgagatctgaggacacggcggtgtactactgcgccagaggtggattggacacagacgagttcgacctatgggggagaggtaccttggtcaccgtctcctca

SEQ ID NO: 28 - DNA, 318 bp, Artificial Sequence
  Description of Artificial Sequence Synthetic polynucleotide
gacatccagatgacccagtctccatcctccctgtctgcatctgtaggagacagagtcaccatcacttgccgggcaagtcagagcattaacagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaggagtcagtgacatcacttttggcggagggaccaaggttgagatcaaa

SEQ ID NO: 29 - protein, 119 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGGLDTDEFDLWGRGTLVTVSS

SEQ ID NO: 30 - protein, 106 aa, Artificial Sequence; identical to SEQ ID NO: 20
  Description of Artificial Sequence Synthetic polypeptide

SEQ ID NO: 31 - protein, 9 aa, Artificial Sequence
  Description of Artificial Sequence Synthetic peptide
GTFSSYAIS

SEQ ID NO: 32 - protein, 17 aa, Artificial Sequence
  Description of Artificial Sequence Synthetic peptide
GIIPIFGTANYAQKFQG

SEQ ID NO: 33 - protein, 12 aa, Artificial Sequence
  Description of Artificial Sequence Synthetic peptide
ARGGLDTDEFDL

SEQ ID NO: 34 - protein, 11 aa, Artificial Sequence
  Description of Artificial Sequence Synthetic peptide
RASQSINSYLN

SEQ ID NO: 35 - protein, 7 aa, Artificial Sequence
  Description of Artificial Sequence Synthetic peptide
AASSLQS

SEQ ID NO: 36 - protein, 8 aa, Artificial Sequence
  Description of Artificial Sequence Synthetic peptide
QQGVSDIT

SEQ ID NO: 37 - DNA, 357 bp, Artificial Sequence
  Description of Artificial Sequence Synthetic polynucleotide
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggcggtgtactactgcgccagaggtggattggacacagacgagttcgacctatgggggagaggtaccttggtcaccgtctcctca

SEQ ID NO: 38 - DNA, 318 bp, Artificial Sequence; identical to SEQ ID NO: 28
  Description of Artificial Sequence Synthetic polynucleotide

SEQ ID NO: 39 - protein, 119 aa, Artificial Sequence
  Description of Artificial Sequence Synthetic polypeptide
QVQLVQSGAEVKKPGSSVKVSCKASGGTFSLYAISWVRQAPGQGLEWMGGIIPAFGTANYAQKFQGRVTITADESTSTAYMELSSLRSEDTAVYYCARGGLDTDEFDLWGRGTLVTVSS

SEQ ID NO: 40 - protein, 106 aa, Artificial Sequence; identical to SEQ ID NO: 20
  Description of Artificial Sequence Synthetic polypeptide

SEQ ID NO: 41 - protein, 9 aa, Artificial Sequence
  Description of Artificial Sequence Synthetic peptide
GTFSLYAIS

SEQ ID NO: 42 - protein, 17 aa, Artificial Sequence
  Description of Artificial Sequence Synthetic peptide
GIIPAFGTANYAQKFQG

SEQ ID NO: 43 - protein, 12 aa, Artificial Sequence
  Description of Artificial Sequence Synthetic peptide
ARGGLDTDEFDL

SEQ ID NO: 44 - protein, 11 aa, Artificial Sequence
  Description of Artificial Sequence Synthetic peptide
RASQSINSYLN

SEQ ID NO: 45 - protein, 7 aa, Artificial Sequence
  Description of Artificial Sequence Synthetic peptide
AASSLQS

SEQ ID NO: 46 - protein, 8 aa, Artificial Sequence
  Description of Artificial Sequence Synthetic peptide
QQGVSDIT

SEQ ID NO: 47 - DNA, 357 bp, Artificial Sequence
  Description of Artificial Sequence Synthetic polynucleotide
caggtgcagctggtgcagtctggggctgaggtgaagaagcctgggtcctcggtgaaggtctcctgcaaggcttctggaggcaccttcagcctctatgctatctcctgggtgcgacaggcccctggacaagggcttgagtggatgggagggatcatccctgccttcggtaccgcaaactacgcacagaagttccagggcagagtcacgattaccgcggacgaatccacgagcacagcctacatggagctgagcagcctgagatctgaggacacggcggtgtactactgcgccagaggtggattggacacagacgagttcgacctatgggggagaggtaccttggtcaccgtctcctca

SEQ ID NO: 48 - DNA, 318 bp, Artificial Sequence; identical to SEQ ID NO: 28
  Description of Artificial Sequence Synthetic polynucleotide

SEQ ID NO: 49 - protein, 119 aa, Artificial Sequence
  Description of Artificial Sequence Synthetic polypeptide
QVQLVQSGAEVKKPGSSVKVSCKASGGTFSLYAISWVRQAPGQGLEWMGGIIPHFGLANYAQKFQGRVTITADESTSTAYMELSSLRSEDTAVYYCARGGLDTDEFDLWGRGTLVTVSS

SEQ ID NO: 50 - protein, 106 aa, Artificial Sequence; identical to SEQ ID NO: 20
  Description of Artificial Sequence Synthetic polypeptide

SEQ ID NO: 51 - protein, 9 aa, Artificial Sequence
  Description of Artificial Sequence Synthetic peptide
GTFSLYAIS

SEQ ID NO: 52 - protein, 17 aa, Artificial Sequence
  Description of Artificial Sequence Synthetic peptide
GIIPHFGLANYAQKFQG

SEQ ID NO: 53 - protein, 12 aa, Artificial Sequence
  Description of Artificial Sequence Synthetic peptide
ARGGLDTDEFDL

SEQ ID NO: 54 - protein, 11 aa, Artificial Sequence
  Description of Artificial Sequence Synthetic peptide
RASQSINSYLN

SEQ ID NO: 55 - protein, 7 aa, Artificial Sequence
  Description of Artificial Sequence Synthetic peptide
AASSLQS

SEQ ID NO: 56 - protein, 8 aa, Artificial Sequence
  Description of Artificial Sequence Synthetic peptide
QQGVSDIT

SEQ ID NO: 57 - DNA, 357 bp, Artificial Sequence
  Description of Artificial Sequence Synthetic polynucleotide
caggtgcagctggtgcagtctggggctgaggtgaagaagcctgggtcctcggtgaaggtctcctgcaaggcttctggaggcaccttctccctctatgctatcagctgggtgcgacaggcccctggacaagggcttgagtggatgggagggatcatccctcacttcggtctcgcaaactacgcacagaagttccagggcagagtcacgattaccgcggacgaatccacgagcacagcctacatggagctgagcagcctgagatctgaggacacggcggtgtactactgcgccagaggtggattggacacagacgagttcgacctatgggggagaggtaccttggtcaccgtctcctca

SEQ ID NO: 58 - DNA, 318 bp, Artificial Sequence; identical to SEQ ID NO: 28
  Description of Artificial Sequence Synthetic polynucleotide

SEQ ID NO: 59 - protein, 123 aa, Artificial Sequence
  Description of Artificial Sequence Synthetic polypeptide
EVQLLESGGGLVQPGGSLRLSCAASGFTFSNYAMSWVRQAPGKGLEWVSAISGSGGSTYYADSVKGRFTISRDNSKNTLYLQMNSLRAEDTAVYYCAKGPPTYHTNYYYMDVWGKGTTVTVSS

SEQ ID NO: 60 - protein, 107 aa, Artificial Sequence
  Description of Artificial Sequence Synthetic polypeptide
DIQMTQSPSSVSASVGDRVTITCRASQGISSWLAWYQQKPGKAPKLLIYAASSLQSGVPSRFSGSGSGTDFTLTISSLQPEDFATYYCQQTNSFPYTFGGGTKVEIK

SEQ ID NO: 61 - protein, 9 aa, Artificial Sequence
  Description of Artificial Sequence Synthetic peptide
FTFSNYAMS

SEQ ID NO: 62 - protein, 17 aa, Artificial Sequence
  Description of Artificial Sequence Synthetic peptide
AISGSGGSTYYADSVKG

SEQ ID NO: 63 - protein, 16 aa, Artificial Sequence
  Description of Artificial Sequence Synthetic peptide
AKGPPTYHTNYYYMDV

SEQ ID NO: 64 - protein, 11 aa, Artificial Sequence
  Description of Artificial Sequence Synthetic peptide
RASQGISSWLA

SEQ ID NO: 65 - protein, 7 aa, Artificial Sequence
  Description of Artificial Sequence Synthetic peptide
AASSLQS

SEQ ID NO: 66 - protein, 9 aa, Artificial Sequence
  Description of Artificial Sequence Synthetic peptide
QQTNSFPYT

SEQ ID NO: 67 - DNA, 369 bp, Artificial Sequence
  Description of Artificial Sequence Synthetic polynucleotide
gaggtgcagctgttggagtctgggggaggcttggtacagcctggggggtccctgagactctcctgtgcagcctctggattcacctttagcaattatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggcggtgtactactgcgccaagggccctcctacataccacacaaactactactacatggacgtatggggcaagggtacaactgtcaccgtctcctca

SEQ ID NO: 68 - DNA, 321 bp, Artificial Sequence
  Description of Artificial Sequence Synthetic polynucleotide
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttattactgtcagcaaacaaatagtttcccttacacttttggcggagggaccaaggttgagatcaaa

SEQ ID NO: 69 - protein, 123 aa, Artificial Sequence
  Description of Artificial Sequence Synthetic polypeptide
EVQLLESGGGLVQPGGSLRLSCAASGFTFSSYVMIWVRQAPGKGLEWVSSISGDSVTTYYADSVKGRFTISRDNSKNTLYLQMNSLRAEDTAVYYCAKGPPTYHTNYYYMDVWGKGTTVTVSS

SEQ ID NO: 70 - protein, 107 aa, Artificial Sequence; identical to SEQ ID NO: 60
  Description of Artificial Sequence Synthetic polypeptide

SEQ ID NO: 71 - protein, 9 aa, Artificial Sequence
  Description of Artificial Sequence Synthetic peptide
FTFSSYVMI

SEQ ID NO: 72 - protein, 17 aa, Artificial Sequence
  Description of Artificial Sequence Synthetic peptide
SISGDSVTTYYADSVKG

SEQ ID NO: 73 - protein, 16 aa, Artificial Sequence
  Description of Artificial Sequence Synthetic peptide
AKGPPTYHTNYYYMDV

SEQ ID NO: 74 - protein, 11 aa, Artificial Sequence
  Description of Artificial Sequence Synthetic peptide
RASQGISSWLA

SEQ ID NO: 75 - protein, 7 aa, Artificial Sequence
  Description of Artificial Sequence Synthetic peptide
AASSLQS

SEQ ID NO: 76 - protein, 9 aa, Artificial Sequence
  Description of Artificial Sequence Synthetic peptide
QQTNSFPYT

SEQ ID NO: 77 - DNA, 369 bp, Artificial Sequence
  Description of Artificial Sequence Synthetic polynucleotide
gaggtgcagctgttggagtctgggggaggcttggtacagcctggggggtccctgagactctcctgtgcagcctctggattcacctttagcagctatgtcatgatctgggtccgccaggctccagggaaggggctggagtgggtctcaagcattagtggtgacagcgtaacaacatactacgcagactccgtgaagggccggttcaccatctccagagacaattccaagaacacgctgtatctgcaaatgaacagcctgagagccgaggacacggcggtgtactactgcgccaagggccctcctacataccacacaaactactactacatggacgtatggggcaagggtacaactgtcaccgtctcctca

SEQ ID NO: 78 - DNA, 321 bp, Artificial Sequence; identical to SEQ ID NO: 68
  Description of Artificial Sequence Synthetic polynucleotide

SEQ ID NO: 79 - protein, 121 aa, Artificial Sequence
  Description of Artificial Sequence Synthetic polypeptide
EVQLVESGGGLVQPGGSLRLSCAASGFTFSDHYMDWVRQAPGKGLEWVGRTRNKASSYTTEYAASVKGRFTISRDDSKNSLYLQMNSLKTEDTAVYYCAREPKYWIDFDLWGRGTLVTVSS

SEQ ID NO: 80 - protein, 107 aa, Artificial Sequence; identical to SEQ ID NO: 10
  Description of Artificial Sequence Synthetic polypeptide

SEQ ID NO: 81 - protein, 9 aa, Artificial Sequence
  Description of Artificial Sequence Synthetic peptide
FTFSDHYMD

SEQ ID NO: 82 - protein, 19 aa, Artificial Sequence
  Description of Artificial Sequence Synthetic peptide
RTRNKASSYTTEYAASVKG

SEQ ID NO: 83 - protein, 12 aa, Artificial Sequence
  Description of Artificial Sequence Synthetic peptide
AREPKYWIDFDL

SEQ ID NO: 84 - protein, 11 aa, Artificial Sequence
  Description of Artificial Sequence Synthetic peptide
RASQSISSYLN

SEQ ID NO: 85 - protein, 7 aa, Artificial Sequence
  Description of Artificial Sequence Synthetic peptide
AASSLQS

SEQ ID NO: 86 - protein, 9 aa, Artificial Sequence
  Description of Artificial Sequence Synthetic peptide
QQSYIAPYT

SEQ ID NO: 87 - DNA, 363 bp, Artificial Sequence
  Description of Artificial Sequence Synthetic polynucleotide
gaggtgcagctggtggagtctgggggaggcttggtccagcctggagggtccctgagactctcctgtgcagcctctggattcaccttcagtgaccactacatggactgggtccgccaggctccagggaaggggctggagtgggttggccgtactagaaacaaagctagtagttacaccacagaatacgccgcgtctgtgaaaggcagattcaccatctcaagagatgattcaaagaactcactgtatctgcaaatgaacagcctgaaaaccgaggacacggcggtgtactactgcgccagagagcctaaatactggatcgacttcgacctatgggggagaggtaccttggtcaccgtctcctca

SEQ ID NO: 88 - DNA, 321 bp, Artificial Sequence; identical to SEQ ID NO: 18
  Description of Artificial Sequence Synthetic polynucleotide

SEQ ID NO: 89 - protein, 121 aa, Artificial Sequence
  Description of Artificial Sequence Synthetic polypeptide
EVQLVESGGGLVQPGRSLRLSCTASGFTFSDHDMNWVRQAPGKGLEWVGRTRNAAGSYTTEYAASVKGRFTISRDDSKNSLYLQMNSLKTEDTAVYYCAREPKYWIDFDLWGRGTLVTVSS

SEQ ID NO: 90 - protein, 107 aa, Artificial Sequence; identical to SEQ ID NO: 10
  Description of Artificial Sequence Synthetic polypeptide

SEQ ID NO: 91 - protein, 9 aa, Artificial Sequence
  Description of Artificial Sequence Synthetic peptide
FTFSDHDMN

SEQ ID NO: 92 - protein, 19 aa, Artificial Sequence
  Description of Artificial Sequence Synthetic peptide
RTRNAAGSYTTEYAASVKG

SEQ ID NO: 93 - protein, 12 aa, Artificial Sequence
  Description of Artificial Sequence Synthetic peptide
AREPKYWIDFDL

SEQ ID NO: 94 - protein, 11 aa, Artificial Sequence
  Description of Artificial Sequence Synthetic peptide
RASQSISSYLN

SEQ ID NO: 95 - protein, 7 aa, Artificial Sequence
  Description of Artificial Sequence Synthetic peptide
AASSLQS

SEQ ID NO: 96 - protein, 9 aa, Artificial Sequence
  Description of Artificial Sequence Synthetic peptide
QQSYIAPYT

SEQ ID NO: 97 - DNA, 363 bp, Artificial Sequence
  Description of Artificial Sequence Synthetic polynucleotide
gaggtgcagctggtggagtctgggggaggcttggtacagccagggcggtccctgagactctcctgtacagcttctggattcaccttcagtgaccacgacatgaactgggtccgccaggctccagggaaggggctggagtgggttggccgtactagaaacgccgctggaagttacaccacagaatacgccgcgtctgtgaaaggcagattcaccatctcaagagatgattcaaagaactcactgtatctgcaaatgaacagcctgaaaaccgaggacacggcggtgtactactgcgccagagagcctaaatactggatcgacttcgacctatgggggagaggtaccttggtcaccgtctcctca

SEQ ID NO: 98 - DNA, 321 bp, Artificial Sequence; identical to SEQ ID NO: 18
  Description of Artificial Sequence Synthetic polynucleotide

SEQ ID NO: 99 - protein, 121 aa, Artificial Sequence
  Description of Artificial Sequence Synthetic polypeptide
EVQLVESGGGLVQPGGSLRLSCAASGFTFVDHDMDWVRQAPGKGLEWVGRTRNKLGSYTTEYAASVKGRFTISRDDSKNSLYLQMNSLKTEDTAVYYCAREPKYWIDFDLWGRGTLVTVSS

SEQ ID NO: 100 - protein, 107 aa, Artificial Sequence; identical to SEQ ID NO: 10
  Description of Artificial Sequence Synthetic polypeptide

SEQ ID NO: 101 - protein, 9 aa, Artificial Sequence
  Description of Artificial Sequence Synthetic peptide
FTFVDHDMD

SEQ ID NO: 102 - protein, 19 aa, Artificial Sequence
  Description of Artificial Sequence Synthetic peptide
RTRNKLGSYTTEYAASVKG

SEQ ID NO: 103 - protein, 12 aa, Artificial Sequence
  Description of Artificial Sequence Synthetic peptide
AREPKYWIDFDL

SEQ ID NO: 104 - protein, 11 aa, Artificial Sequence
  Description of Artificial Sequence Synthetic peptide
RASQSISSYLN

SEQ ID NO: 105 - protein, 7 aa, Artificial Sequence
  Description of Artificial Sequence Synthetic peptide
AASSLQS

SEQ ID NO: 106 - protein, 9 aa, Artificial Sequence
  Description of Artificial Sequence Synthetic peptide
QQSYIAPYT

SEQ ID NO: 107 - DNA, 363 bp, Artificial Sequence
  Description of Artificial Sequence Synthetic polynucleotide
gaggtgcagctggtggagtctgggggaggcttggtccagcctggagggtccctgagactctcctgtgcagcctctggattcaccttcgtagaccacgacatggactgggtccgccaggctccagggaaggggctggagtgggttggccgtactagaaacaaactaggaagttacaccacagaatacgccgcgtctgtgaaaggcagattcaccatctcaagagatgattcaaagaactcactgtatctgcaaatgaacagcctgaaaaccgaggacacggcggtgtactactgcgccagagagcctaaatactggatcgacttcgacctatgggggagaggtaccttggtcaccgtctcctca

SEQ ID NO: 108 - DNA, 321 bp, Artificial Sequence; identical to SEQ ID NO: 18
  Description of Artificial Sequence Synthetic polynucleotide

SEQ ID NO: 109 - protein, 214 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 451 aa, Artificial Sequence
  Description of Artificial Sequence Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 451 aa, Artificial Sequence
  Description of Artificial Sequence Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 451 aa, Artificial Sequence
  Description of Artificial Sequence Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 451 aa, Artificial Sequence
  Description of Artificial Sequence Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 114 - protein, 451 aa, Artificial Sequence
  Description of Artificial Sequence Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 115 - protein, 213 aa, Artificial Sequence
  Description of Artificial Sequence Synthetic polypeptide
DIQMTQSPSSLSASVGDRVTITCRASQSINSYLNWYQQKPGKAPKLLIYAASSLQSGVPSRFSGSGSGTDFTLTISSLQPEDFATYYCQQGVSDITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 449 aa, Artificial Sequence
  Description of Artificial Sequence Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 449 aa, Artificial Sequence
  Description of Artificial Sequence Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 449 aa, Artificial Sequence
  Description of Artificial Sequence Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 449 aa, Artificial Sequence
  Description of Artificial Sequence Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 120 - protein, 449 aa, Artificial Sequence
  Description of Artificial Sequence Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 121 - protein, 107 aa, Artificial Sequence [sequence omitted]

SEQ ID NO: 122 - protein, 330 aa, Homo sapiens [sequence omitted]

SEQ ID NO: 123 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 126 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 127 - protein, 9 aa, Artificial Sequence
  Description of Artificial Sequence Synthetic peptide
  MOD_RES (4)..(4): Ser or Arg
  MOD_RES (5)..(5): Ser, Ile or Leu
GTFXXYAIS

SEQ ID NO: 128 - protein, 17 aa, Artificial Sequence
  Description of Artificial Sequence Synthetic peptide
  MOD_RES (5)..(5): Ile, Asp, Ala or His
  MOD_RES (8)..(8): Thr, Val or Leu
GIIPXFGXANYAQKFQG

SEQ ID NO: 129 - protein, 12 aa, Artificial Sequence
  Description of Artificial Sequence Synthetic peptide
ARGGLDTDEFDL

SEQ ID NO: 130 - protein, 11 aa, Artificial Sequence
  Description of Artificial Sequence Synthetic peptide
RASQSINSYLN

SEQ ID NO: 131 - protein, 7 aa, Artificial Sequence
  Description of Artificial Sequence Synthetic peptide
AASSLQS

SEQ ID NO: 132 - protein, 8 aa, Artificial Sequence
  Description of Artificial Sequence Synthetic peptide
QQGVSDIT

SEQ ID NO: 133 - protein, 9 aa, Artificial Sequence
  Description of Artificial Sequence Synthetic peptide
  MOD_RES (5)..(5): Asn or Ser
  MOD_RES (7)..(7): Ala or Val
  MOD_RES (9)..(9): Ser or Ile
FTFSXYXMX

SEQ ID NO: 134 - protein, 17 aa, Artificial Sequence
  Description of Artificial Sequence Synthetic peptide
  MOD_RES (1)..(1): Ala or Ser
  MOD_RES (5)..(5): Ser or Asp
  MOD_RES (6)..(6): Gly or Ser
  MOD_RES (7)..(7): Gly or Val
  MOD_RES (8)..(8): Ser or Thr
XISGXXXXTYYADSVKG

SEQ ID NO: 135 - protein, 16 aa, Artificial Sequence
  Description of Artificial Sequence Synthetic peptide
AKGPPTYHTNYYYMDV

SEQ ID NO: 136 - protein, 11 aa, Artificial Sequence
  Description of Artificial Sequence Synthetic peptide
RASQGISSWLA

SEQ ID NO: 137 - protein, 7 aa, Artificial Sequence
  Description of Artificial Sequence Synthetic peptide
AASSLQS

SEQ ID NO: 138 - protein, 9 aa, Artificial Sequence
  Description of Artificial Sequence Synthetic peptide
QQTNSFPYT

SEQ ID NO: 139 - protein, 9 aa, Artificial Sequence
  Description of Artificial Sequence Synthetic peptide
  MOD_RES (4)..(4): Ser or Val
  MOD_RES (6)..(6): His or Ala
  MOD_RES (7)..(7): Tyr or Asp
  MOD_RES (9)..(9): Asp or Asn
FTFXDXXMX

SEQ ID NO: 140 - protein, 19 aa, Artificial Sequence
  Description of Artificial Sequence Synthetic peptide
  MOD_RES (5)..(5): Lys or Ala
  MOD_RES (6)..(6): Ala or Leu
  MOD_RES (7)..(7): Ser or Gly
RTRNXXXSYTTEYAASVKG

SEQ ID NO: 141 - protein, 12 aa, Artificial Sequence
  Description of Artificial Sequence Synthetic peptide
AREPKYWIDFDL

SEQ ID NO: 142 - protein, 11 aa, Artificial Sequence
  Description of Artificial Sequence Synthetic peptide
RASQSISSYLN

SEQ ID NO: 143 - protein, 7 aa, Artificial Sequence
  Description of Artificial Sequence Synthetic peptide
AASSLQS

SEQ ID NO: 144 - protein, 9 aa, Artificial Sequence
  Description of Artificial Sequence Synthetic peptide
QQSYIAPYT

SEQ ID NO: 145 - protein, 976 aa, Homo sapiens [sequence omitted]

SEQ ID NO: 146 - protein, 972 aa, Homo sapiens
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 147 - protein, 123 aa, Artificial Sequence
  Description of Artificial Sequence Synthetic polypeptide
QVQLVQSGAAVKKPGESLKISCKGSGYRFTTYWIGWVRQMPGKGLEWMGIIYPGDSDTRYSPSFQGQVTISAGKSISTAYLQWSSLKASDTAMYYCARHGRGYNGYEGAFDIWGQGTMVTVSS

SEQ ID NO: 148 - protein, 107 aa, Artificial Sequence
  Description of Artificial Sequence Synthetic polypeptide
AIQLTQSPSSLSASVGDRVTITCRASQGVSSALAWYQQKPGKAPKLLIYDASSLESGVPSRFSGSGSGTDFTLTISSLQPEDFATYYCQQFNSYPLTFGGGTKVEIK

SEQ ID NO: 149 - protein, 107 aa, Artificial Sequence
  Description of Artificial Sequence Synthetic polypeptide
DIQLTQSPSSLSASVGDRVTITCRASQGIRTDLGWYQQKPGKAPKLLIYDASSLESGVPSRFSGSGSGTDFTLTISSLQPEDFATYYCQQFNSYPLTFGGGTKVEIK

SEQ ID NO: 150 - protein, 107 aa, Artificial Sequence
  Description of Artificial Sequence Synthetic polypeptide
AIRMTQSPSSLSASVGDRVTITCRASQGIRNDLAWYQQKPGKTPKLLIYDASSLESGVPSRFSGSGSGTDFTLTISSLQPEDFATYYCQQFNSYPLTFGGGTKVEIK

SEQ ID NO: 151 - protein, 107 aa, Artificial Sequence
  Description of Artificial Sequence Synthetic polypeptide
AIQMTQSPSSLSASVGDRVTITCRASQGIRNDLGWYQQKPGKAPKLLIYDASSLESGVPSRFSGSGSGTDFTLTISSLQPEDFATYYCQQFNSYPLTFGGGTKVDIK

SEQ ID NO: 152 - protein, 107 aa, Artificial Sequence
  Description of Artificial Sequence Synthetic polypeptide
NIQMTQSPSSLSASVGDRVTITCRASQAISDYLAWFQQKPGKAPKLLIYDASNLETGVPSRFSGSGSGTDFTLTISSLQPEDFATYYCQQLNSYPLTFGGGTKVEIK

SEQ ID NO: 153 - protein, 107 aa, Artificial Sequence
  Description of Artificial Sequence Synthetic polypeptide
AIRMTQSPSSLSASVGDRVIIACRASQGIGGALAWYQQKPGNAPKVLVYDASTLESGVPSRFSGGGSGTDFTLTISSLQPEDFATYYCQQFNSYPLTFGGGTKLEIK

SEQ ID NO: 154 - protein, 107 aa, Artificial Sequence
  Description of Artificial Sequence Synthetic polypeptide
DIAMTQSPPSLSAFVGDRVTITCRASQGIISSLAWYQQKPGKAPKLLIYDASSLESGVPSRFSGSGSGTDFTLTIRSLQPEDFATYYCQQFNSYPLTFGGGTKLEIK

SEQ ID NO: 155 - protein, 107 aa, Artificial Sequence
  Description of Artificial Sequence Synthetic polypeptide
DIQMTQSPSSLSASVGDRVTITCRASQGISSALAWYQQKAGKAPKVLISDASSLESGVPSRFSGSGSGTDFTLSISSLQPEDFATYYCQQFNGYPLTFGGGTKVDIK

SEQ ID NO: 156 - protein, 107 aa, Artificial Sequence
  Description of Artificial Sequence Synthetic polypeptide
AIRMTQSPSSLSASVGDRVTITCQASQGIRNDLGWYQQKPGKAPKLLIYDASNLETGVPSRFSGSGSGTDFTFTISSLQPEDIATYYCQQFNSYPLTFGGGTKLEIK

SEQ ID NO: 157 - protein, 107 aa, Artificial Sequence
  Description of Artificial Sequence Synthetic polypeptide
NIQMTQSPSSLSTSVGDRVTITCRASQGIGTSLAWYQQKPGKPPKLLIYDASSLESGVPSRLSGSGSGTDFTLTISSLQPEDFATYYCQQSNSYPITFGQGTRLEIK

SEQ ID NO: 158 - protein, 107 aa, Artificial Sequence
  Description of Artificial Sequence Synthetic polypeptide
AIQLTQSPSSLSASVGDRVTITCRASQSIGDYLTWYQQKPGKAPKVLIYGASSLQSGVPPRFSGSGSGTDFTLTVSSLQPEDFATYYCQQLNSYPLTFGGGTKLEIK

SEQ ID NO: 159 - protein, 107 aa, Artificial Sequence
  Description of Artificial Sequence Synthetic polypeptide
DIQLTQSPSSLSASVGDRVTITCRASQGVRSTLAWYQQKPGKAPKLLIYDASILESGVPSRFSGSGSGTDFTLTISSLQPEDFATYYCQQFNGYPLTFGQGTRLEIK

SEQ ID NO: 160 - protein, 107 aa, Artificial Sequence
  Description of Artificial Sequence Synthetic polypeptide
DIVMTQSPSSLSASVGDRVTITCRASQGIRNDLGWYQQKPGKAPKLLIYDASSLESGVPSRFSGSGSGTDFTLTISSLQPEDFATYYCQQFNSYPLTFGGGTKLEIK

SEQ ID NO: 161 - protein, 107 aa, Artificial Sequence
  Description of Artificial Sequence Synthetic polypeptide
DIQLTQSPSSLSASVGDRVTITCRASQGISSFLAWYQQKPGKAPKLLIYDASTLQSGVPSRFSGSASGTDFTLTISSLQPEDFATYYCQQLNGYPLTFGGGTKVEIK

SEQ ID NO: 162 - protein, 107 aa, Artificial Sequence
  Description of Artificial Sequence Synthetic polypeptide
AIQLTQSPSSLSASVGDRVTITCRASQGIGSALAWYQQKPGIGPKLLIYDASTLESGVPARFSGSGSRTDFTLTITSLQPEDFATYYCQQFNGYPLTFGGGTKLEIK

SEQ ID NO: 163 - protein, 107 aa, Artificial Sequence
  Description of Artificial Sequence Synthetic polypeptide
AIQLTQSPSSLSASVGDRVTITCRASQGITSALAWYQEKPGKAPNLLIYDASSLESGVPSRFSGSGYGTDFTLTISSLQPEDFATYYCQQLNSYPLTFGGGTKVDIK

SEQ ID NO: 164 - protein, 121 aa, Artificial Sequence
  Description of Artificial Sequence Synthetic polypeptide
QIQLVQSGPELRKPGESVKISCKASGYTFTDYAMYWVKQAPGKGLKWMGWINTYTGKPTYADDFKGRFVFSLEASANTANLQISNLKNEDTATYFCARARGLVDDYVMDAWGQGTSVTVSS

SEQ ID NO: 165 - protein, 108 aa, Artificial Sequence
  Description of Artificial Sequence Synthetic polypeptide
SYELIQPPSASVTLGNTVSLTCVGDELSKRYAQWYQQKPDKTIVSVIYKDSERPSGISDRFSGSSSGTTATLTIHGTLAEDEADYYCLSTYSDDNLPVFGGGTKLTVL

SEQ ID NO: 166 - protein, 117 aa, Artificial Sequence
  Description of Artificial Sequence Synthetic polypeptide
EVQLQQYGAELGKPGTSVRLSCKVSGYNIRNTYIHWVNQRPGEGLEWIGRIDPTNGNTISAEKFKTKATLTADTSSHTAYLQFSQLKSDDTAIYFCALNYEGYADYWGQGVMVTGSS

SEQ ID NO: 167 - protein, 106 aa, Artificial Sequence
  Description of Artificial Sequence Synthetic polypeptide
DIQMTQSPSFLSASVGDRVTINCKASQNINKYLNWYQQKVGEAPKRLIFKTNSLQTGIPSRFSGSGSGTDYTLTISSLQTEDVATYFCFQYNIGYTFGAGTKVELK

SEQ ID NO: 168 - protein, 124 aa, Artificial Sequence
  Description of Artificial Sequence Synthetic polypeptide
EVQLQESGPGLVKPSQSLSLTCSVTGYSISSNYRWNWIRKFPGNKVEWMGYINSAGSTNYNPSLKSRISMTRDTSKNQFFLQVNSVTTEDTATYYCARSLRGYITDYSGFFDYWGQGVMVTVSS

SEQ ID NO: 169 - protein, 107 aa, Artificial Sequence
  Description of Artificial Sequence Synthetic polypeptide
DIRMTQSPASLSASLGETVNIECLASEDIFSDLAWYQQKPGKSPQLLIYNANSLQNGVPSRFSGSGSGTRYSLKINSLQSEDVATYFCQQYKNYPLTFGSGTKLEIK

SEQ ID NO: 170 - protein, 117 aa, Artificial Sequence
  Description of Artificial Sequence Synthetic polypeptide
EVQLQQYGAELGKPGTSVRLSCKLSGYKIRNTYIHWVNQRPGKGLEWIGRIDPANGNTIYAEKFKSKVTLTADTSSNTAYMQLSQLKSDDTALYFCAMNYEGYEDYWGQGVMVTVSS

SEQ ID NO: 171 - protein, 106 aa, Artificial Sequence
  Description of Artificial Sequence Synthetic polypeptide
DIQMTQSPSFLSASVGDSVTINCKASQNINKYLNWYQQKLGEAPKRLIHKTDSLQTGIPSRFSGSGSGTDYTLTISSLQPEDVATYFCFQYKSGFMFGAGTKLELK

SEQ ID NO: 172 - protein, 120 aa, Artificial Sequence
  Description of Artificial Sequence Synthetic polypeptide
QIQLVQSGPELKKPGESVKISCKASGYTFTDYAVYWVIQAPGKGLKWMGWINTYTGKPTYADDFKGRFVFSLETSASTANLQISNLKNEDTATYFCARGAGMTKDYVMDAWGRGVLVTVS

SEQ ID NO: 173 - protein, 108 aa, Artificial Sequence
  Description of Artificial Sequence Synthetic polypeptide
SYELIQPPSASVTLGNTVSLTCVGDELSKRYAQWYQQKPDKTIVSVIYKDSERPSDISDRFSGSSSGTTATLTIHGTLAEDEADYYCLSTYSDDNLPVFGGGTKLTVL

SEQ ID NO: 174 - protein, 116 aa, Artificial Sequence
  Description of Artificial Sequence Synthetic polypeptide
QVQLKESGPGLVQPSQTLSLTCTVSGFSLTSYLVHWVRQPPGKTLEWVGLMWNDGDTSYNSALKSRLSISRDTSKSQVFLKMHSLQAEDTATYYCARESNLGFTYWGHGTLVTVSS

SEQ ID NO: 175 - protein, 107 aa, Artificial Sequence
  Description of Artificial Sequence Synthetic polypeptide
DIQMTQSPASLSASLEEIVTITCKASQGIDDDLSWYQQKPGKSPQLLIYDVTRLADGVPSRFSGSRSGTQYSLKISRPQVADSGIYYCLQSYSTPYTFGAGTKLELK

SEQ ID NO: 176 - protein, 117 aa, Artificial Sequence
  Description of Artificial Sequence Synthetic polypeptide
EVQLQQYGAELGKPGTSVRLSCKVSGYNIRNTYIHWVHQRPGEGLEWIGRIDPTNGNTISAEKFKSKATLTADTSSNTAYMQFSQLKSDDTAIYFCAMNYEGYADYWGQGVMVTVSS

SEQ ID NO: 177 - protein, 106 aa, Artificial Sequence
  Description of Artificial Sequence Synthetic polypeptide
DIQMTQSPSFLSASVGDRLTINCKASQNINKYLNWYQQKLGEAPKRLIFKTNSLQTGIPSRFSGSGSGTDYTLTISSLQPEDVATYFCFQYNIGFTFGAGTKLELK

SEQ ID NO: 178 - protein, 124 aa, Artificial Sequence
  Description of Artificial Sequence Synthetic polypeptide
EVQLVESGGGLVQSGRSLKLSCAASGFTVSDYYMAWVRQAPTKGLEWVATINYDGSTTYHRDSVKGRFTISRDNAKSTLYLQMDSLRSEDTATYYCARHGDYGYHYGAYYFDYWGQGVMVTVSS

SEQ ID NO: 179 - protein, 109 aa, Artificial Sequence
  Description of Artificial Sequence Synthetic polypeptide
DIVLTQSPALAVSLGQRATISCRASQTVSLSGYNLIHWYQQRTGQQPKLLIYRASNLAPGIPARFSGSGSGTDFTLTISPVQSDDIATYYCQQSRESWTFGGGTNLEMK

SEQ ID NO: 180 - protein, 116 aa, Artificial Sequence
  Description of Artificial Sequence Synthetic polypeptide
QIQLVQSGPELKKPGESVKISCKASGYTFTDYAIHWVKQAPGQGLRWMAWINTETGKPTYADDFKGRFVFSLEASASTAHLQISNLKNEDTATFFCAGGSHWFAYWGQGTLVTVSS

SEQ ID NO: 181 - protein, 108 aa, Artificial Sequence
  Description of Artificial Sequence Synthetic polypeptide
SYELIQPPSASVTLENTVSITCSGDELSNKYAHWYQQKPDKTILEVIYNDSERPSGISDRFSGSSSGTTAILTIRDAQAEDEADYYCLSTFSDDDLPIFGGGTKLTVL

SEQ ID NO: 182 - protein, 108 aa, Artificial Sequence
  Description of Artificial Sequence Synthetic polypeptide
SYELIQPPSTSVTLGNTVSLTCVGNELPKRYAYWFQQKPDQSIVRLIYDDDRRPSGISDRFSGSSSGTTATLTIRDAQAEDEAYYYCHSTYTDDKVPIFGGGTKLTVL

SEQ ID NO: 183 - protein, 123 aa, Artificial Sequence
  Description of Artificial Sequence Synthetic polypeptide
EVQLVESGGGLVQPGRSMKLSCKASGFTFSNYDMAWVRQAPTRGLEWVASISYDGITAYYRDSVKGRFTISRENAKSTLYLQLVSLRSEDTATYYCTTEGGYVYSGPHYFDYWGQGVMVTVSS

SEQ ID NO: 184 - protein, 107 aa, Artificial Sequence
  Description of Artificial Sequence Synthetic polypeptide
DIQMTQSPSSMSVSLGDTVTITCRASQDVGIFVNWFQQKPGRSPRRMIYRATNLADGVPSRFSGSRSGSDYSLTISSLESEDVADYHCLQYDEFPRTFGGGTKLELK

SEQ ID NO: 185 - protein, 117 aa, Artificial Sequence
  Description of Artificial Sequence Synthetic polypeptide
EVQLQQYGAELGKPGTSVRLSCKVSGYKIRNTYIHWVNQRPGKGLEWIGRIDPANGNTIYAEKFKSKVTLTADTSSNTAYMQLSQLKSDDTALYFCAMNYEGYEDYWGQGVMVTVSS

SEQ ID NO: 186 - protein, 106 aa, Artificial Sequence
  Description of Artificial Sequence Synthetic polypeptide
DIQMTQSPSFLSASVGDSVTINCKASQNINKYLNWYQQKLGEAPKRLIHKTNSLQPGFPSRFSGSGSGTDYTLTISSLQPEDVAAYFCFQYNSGFTFGAGTKLELK

SEQ ID NO: 187 - protein, 125 aa, Artificial Sequence
  Description of Artificial Sequence Synthetic polypeptide
QVQLVQSGAEVKKPGASVKVSCKASGYTFTDYYIHWVRQAPGQGLEWMGWMNPHSGDTGYAQKFQGRVTMTRDTSTSTVYMELSSLRSEDTAVYYCARHGRGYNGYEGAFDIWGQGTLVTVSSAS

SEQ ID NO: 188 - protein, 107 aa, Artificial Sequence
  Description of Artificial Sequence Synthetic polypeptide
DIQMTQSPSSLSASVGDRVTITCRASQGIGNELGWYQQKPGKAPKLLIYAASNLQSGVPSRFSGSGSGTDFTLTISSLQPEDFATYYCQQYDNLPLTFGQGTKVEIK

SEQ ID NO: 189 - protein, 125 aa, Artificial Sequence
  Description of Artificial Sequence Synthetic polypeptide
QVQLVQSGAEVKKPGASVKVSCKASGYTFTGYYLHWVRQAPGQGLEWMGWINPNSGDTNYAQNFQGRVTMTRDTSTSTVYMELSSLRSEDTAVYYCARHGRGYNGYEGAFDIWGQGTLVTVSSAS

SEQ ID NO: 190 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASSLESGVPSRFSGSGSGTDFTLTISSLQPEDFATYYCQQLNGYPLTFGGGTKVEIK

SEQ ID NO: 191 - protein, 125 aa, Artificial Sequence
  Description of Artificial Sequence Synthetic polypeptide
QVQLVQSGAEVKKPGASVKVSCKASGYTFTGYYLHWVRQAPGQGLEWMGWINPNSGGTNYAQKFQGRVTMTRDTSTSTVYMELSSLRSEDTAVYYCARHGRGYEGYEGAFDIWGQGTLVTVSSAS

SEQ ID NO: 192 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASELETGVPSRFSGSGSGTDFTLTISSLQPEDFATYYCQQLNGYPITFGQGTKVEIK

SEQ ID NO: 193 - protein, 125 aa, Artificial Sequence
  Description of Artificial Sequence Synthetic polypeptide
QVQLVQSGAEVKKPGASVKVSCKASGYTFTSYYIHWVRQAPGQGLEWMGWLNPSGGGTSYAQKFQGRVTMTRDTSTSTVYMELSSLRSEDTAVYYCARHGRGYDGYEGAFDIWGQGTLVTVSSAS

SEQ ID NO: 194 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASNLETGVPSRFSGSGSGTDFTLTISSLQPEDFATYYCQQLNGYPLTFGGGTKVEIK

SEQ ID NO: 195 - protein, 125 aa, Artificial Sequence
  Description of Artificial Sequence Synthetic polypeptide
QVQLVQSGAEVKKPGASVKVSCKASGYTFSTYYMHWVRQAPGQGLEWMGIINPSGGSTSYAQKFQGRVTMTRDTSTSTVYMKLSSLRSEDTAVYYCARHGRGYEGYEGAFDIWGQGTLVTVSSAS

SEQ ID NO: 196 - protein, 107 aa, Artificial Sequence
  Description of Artificial Sequence Synthetic polypeptide
DIQMTQSPSSLSASVGDRVTITCRASQGIRDDLGWYQQKPGKAPKLLIYDASNLETGVPSRFSGSGSGTDFTLTISSLQPEDFATYYCQQANGFPLTFGGGTKVEIK

SEQ ID NO: 197 - protein, 125 aa, Artificial Sequence
  Description of Artificial Sequence Synthetic polypeptide
QVQLVQSGAEVKKPGASVKVSCKASGYTFTGYYIHWVRQAPGQGLEWMGIINPSGGNTNYAQNFQGRVTMTRDTSTSTVYMELSSLRSEDTAVYYCARHGRGYNAYEGAFDIWGQGTLVTVSSAS

SEQ ID NO: 198 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASNLETGVPSRFSGSGSGTDFTLTISSLQPEDFATYYCQQVNGYPLTFGGGTKVEIK

SEQ ID NO: 199 - protein, 125 aa, Artificial Sequence
  Description of Artificial Sequence Synthetic polypeptide
QVQLVQSGAEVKKPGASVKVSCKASGGTFSSYAISWVRQAPGQGLEWMGVINPTVGGANYAQKFQGRVTMTRDTSTSTVYMELSSLRSEDTAVYYCARHGRGYNEYEGAFDIWGQGTLVTVSSAS

SEQ ID NO: 200 - protein, 107 aa, Artificial Sequence
  Description of Artificial Sequence Synthetic polypeptide
DIQMTQSPSSLSASVGDRVTITCQASQDISDYLNWYQQKPGKAPKLLIYDASNLETGVPSRFSGSGSGTDFTLTISSLQPEDFATYYCQQGNSFPLTFGGGTKLEIK

SEQ ID NO: 201 - protein, 125 aa, Artificial Sequence
  Description of Artificial Sequence Synthetic polypeptide
QVQLVQSGAEVKKLGASVKVSCKASGYTFSSYYMHWVRQAPGQGLEWMGVINPNGAGTNFAQKFQGRVTMTRDTSTSTVYMELSSLRSEDTAVYYCARHGRGYEGYEGAFDIWGQGTLVTVSSAS

SEQ ID NO: 202 - protein, 125 aa, Artificial Sequence
  Description of Artificial Sequence Synthetic polypeptide
QVQLVQSGAEVKKPGASVKVSCKASGYTFTTYYMHWVRQAPGQGLEWMGWINPTGGGTNYAQNFQGRVTMTRDTSTSTVYMELSSLRSEDTAVYYCARHGRGYEGYEGAFDIWGQGTLVTVSSAS

SEQ ID NO: 203 - protein, 107 aa, Artificial Sequence
  Description of Artificial Sequence Synthetic polypeptide
DIQMTQSPSSLSASVGDRVTITCRASQGIRNDVSWYQQKPGKAPKLLIYDASNLETGVPSRFSGSGSGTDFTLTISSLQPEDFATYYCQQLSGYPITFGQGTKLEIK

SEQ ID NO: 204 - protein, 125 aa, Artificial Sequence
  Description of Artificial Sequence Synthetic polypeptide
QVQLVQSGAEVKKPGASVKVSCKASGYTFTSYYIHWVRQAPGQGLEWMGMINPSGGSTNYAQKFQGRVTMTRDTSTSTVYMELSSLRSEDTAVYYCARHGRGYNDYEGAFDIWGQGTLVTVSSAS

SEQ ID NO: 205 - protein, 107 aa, Artificial Sequence
  Description of Artificial Sequence Synthetic polypeptide
DIQMTQSPSSLSASVGDRVTITCRASQSISDWLAWYQQKPGKAPKLLIYEASNLEGGVPSRFSGSGSGTDFTLTISSLQPEDFATYYCQQANSFPYTFGQGTKVEIK

SEQ ID NO: 206 - protein, 125 aa, Artificial Sequence
  Description of Artificial Sequence Synthetic polypeptide
QVQLVQSGAEVKKPGASVKVSCKASGYIFSAYYIHWVRQAPGQGLEWMGIINPSGGSTRYAQKFQGRVTMTRDTSTSTVYMELSSLRSEDTAVYYCARHGRGYGGYEGAFDIWDQGTLVTVSSAS

SEQ ID NO: 207 - protein, 107 aa, Artificial Sequence
  Description of Artificial Sequence Synthetic polypeptide
DIQMTQSPSSLSASVGDRVTITCRASQGIGDYVAWYQQKPGKAPKLLIYDASNLETGVPSRFSGSGSGTDFTLTISSLQPEDFATYYCQQLNGYPITFGQGTRLEIK

SEQ ID NO: 208 - protein, 125 aa, Artificial Sequence
  Description of Artificial Sequence Synthetic polypeptide
EVQLVQSGAEVKKPGESLKISCKGSGYRFTSYWIGWVRQMPGKGLEWMGIIYPDDSDTRYSPSFQGQVTISVDKSNSTAYLQWSSLKASDTAMYYCARHGRGYNGYEGAFDIWGQGTLVTVSSAS

SEQ ID NO: 209 - protein, 107 aa, Artificial Sequence
  Description of Artificial Sequence Synthetic polypeptide
DIQMTQSPSSLSASVGDRVTITCRASQGISSYLAWYQQKPGKAPKLLIYDASNLETGVPSRFSGSGSGTYFTLTISSLQPEDFATYYCQQGASFPITFGQGTKVEIK

SEQ ID NO: 210 - protein, 125 aa, Artificial Sequence
  Description of Artificial Sequence Synthetic polypeptide
EVQLVQSGAEVKKPGESLKISCKGSGSSFPNSWIAWVRQMPGKGLEWMGIIYPSDSDTRYSPSFQGQVTISADKSISTAYLQWSSLEASDTAMYYCARHGRGYNGYEGAFDIWGQGTLVTVSSAS

SEQ ID NO: 211 - protein, 107 aa, Artificial Sequence
  Description of Artificial Sequence Synthetic polypeptide
DIQMTQSPSSLSASVGDRVTITCRASQGIRNYLAWYQQKPGKAPKLLIYDASSLQSGVPSRFSGSGSGTDFTLTISSLQPEDFATYYCQQLNSYPLTFGGGTKVEIK

SEQ ID NO: 212 - protein, 125 aa, Artificial Sequence
  Description of Artificial Sequence Synthetic polypeptide
EVQLVQSGAEVKKPGESLKISCKGSGYSFDSYWIGWVRQMPGKGLEWMGIMYPGDSDTRYSPSFQGQVTISADKSISTAYLQWSSLKASDTAMYYCARHGRGYNAYEGAFDIWGQGTLVTVSSAS

SEQ ID NO: 213 - protein, 107 aa, Artificial Sequence
  Description of Artificial Sequence Synthetic polypeptide
DIQMTQSPSSLSASVGDRVTITCRASQSINNWLAWYQQKPGKAPKLLIYDAFILQSGVPSRFSGSGSGTDFTLTISSLQPEDFATYYCLQLNSYPLTFGPGTKVDIK

SEQ ID NO: 214 - protein, 124 aa, Artificial Sequence
  Description of Artificial Sequence Synthetic polypeptide
EVQLVQSGAEVKKPGESLKISCKGSGYSFTNWIAWVRQMPGKGLEWMGIIYPGDSETRYSPSFQGQVTISADKSISTAYLQWSSLKASDTAMYYCARHGRGYYGYEGAFDIWGQGTLVTVSSAS

SEQ ID NO: 215 - protein, 107 aa, Artificial Sequence
  Description of Artificial Sequence Synthetic polypeptide
DIQMTQSPSSLSASVGDRVTITCRASQGISDNLNWYQQKPGKAPKLLIYDASNLETGVPSRFSGSGSGTDFTLTISSLQPEDFATYYCQQAISFPLTFGQGTKVEIK

SEQ ID NO: 216 - protein, 125 aa, Artificial Sequence
  Description of Artificial Sequence Synthetic polypeptide
EVQLVQSGAEVKKPGESLKISCKGSGYNFTSYWIGWVRQMPGKGLEWMGVIYPDDSETRYSPSFQGQVTISADKSISTAYLQWSSLKASDTAMYYCARHGRGYNGYEGAFDIWGQGTLVTVSSAS

SEQ ID NO: 217 - protein, 107 aa, Artificial Sequence
  Description of Artificial Sequence Synthetic polypeptide
DIQMTQSPSSLSASVGDRVTITCRASRDIRDDLGWYQQKPGKAPKLLIYDASNLETGVPSRFSGSGSGTDFTLTISSLQPEDFATYYCQQANSFPLTFGGGTKVEIK

SEQ ID NO: 218 - protein, 124 aa, Artificial Sequence
  Description of Artificial Sequence Synthetic polypeptide
EVQLVQSGAEVKKPGESLKISCKGSGYTFNTYIGWVRQMPGKGLEWMGIIYPGDSGTRYSPSFQGQVTISADKAISTAYLQWSSLKASDTAMYYCARHSRGYNGYEGAFDIWGQGTLVTVSSAS

SEQ ID NO: 219 - protein, 107 aa, Artificial Sequence
  Description of Artificial Sequence Synthetic polypeptide
DIQMTQSPSSLSASVGDRVTITCRASQGISNYLAWYQQKPGKAPKLLIYDASNLETGVPSRFSGSGSGTDFTLTISSLQPEDFATYYCQQANSFPVTFGQGTKVEIK

SEQ ID NO: 220 - protein, 125 aa, Artificial Sequence
  Description of Artificial Sequence Synthetic polypeptide
EVQLVQSGAEVKKPGESLKISCKGSGYNFTTYWIGWVRQMPGKGLEWMGIIHPADSDTRYNPSFQGQVTISADKSISTAYLQWSSLKASDTAMYYCARHGRGYNGYEGAFDIWGQGTLVTVSSAS

SEQ ID NO: 221 - protein, 107 aa, Artificial Sequence
  Description of Artificial Sequence Synthetic polypeptide
DIQMTQSPSSLSASVGDRVTITCRVSQGISSYLAWYQQKPGKAPKLLIYDASNLETGVPSRFSGSGSGTDFTLTISSLQPEDFATYYCQQANSFPLTFGGGTKVEIK

SEQ ID NO: 222 - protein, 125 aa, Artificial Sequence
  Description of Artificial Sequence Synthetic polypeptide
EVQLVQSGAEVKKPGESLKISCKGSGYRFSNYWIAWVRQMPGKGLEWMGIIYPDNSDTRYSPSFQGQVTISADKSISTAYLQWSSLKASDTAMYYCARHGRGYDGYEGAFDIWGQGTLVTVSSAS

SEQ ID NO: 223 - protein, 107 aa, Artificial Sequence
  Description of Artificial Sequence Synthetic polypeptide
DIQMTQSPSSLSASVGDRVTITCRASQGIRSDLAWYQQKPGKAPKLLIYGASSLQSGVPSRFSGSGSGTDFTLTISSLQPEDFATYYCQQANSFPLSFGQGTKVEIK

SEQ ID NO: 224 - protein, 124 aa, Artificial Sequence
  Description of Artificial Sequence Synthetic polypeptide
EVQLVQSGAEVKKPGESLKISCKGSGYRFASYWIGWVRQMPGKGLEWMGITYPGDSETRYNPSQGQVTISADKSISTAYLQWSSLKASDTAMYYCARHGRGYGGYEGAFDIWGQGTLVTVSSAS

SEQ ID NO: 225 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASNLETGVPSRFSGSGSGTDFTLTISSLQPEDFATYYCQQANSFPLTFGGGTKVEIK

SEQ ID NO: 226 - protein, 125 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHGRGYNGYEGAFDIWGQGTLVTVSSAS

SEQ ID NO: 227 - protein, 107 aa, Artificial Sequence
  Description of Artificial Sequence Synthetic polypeptide
DIQMTQSPSSLSASVGDRVTITCRASQSISNWLAWYQQKPGKAPKLLIYDASNLETGVPSRFSGSGSGTDFTLTISSLQPEDFATYYCQQTNSFPLTFGQGTRLEIK

SEQ ID NO: 228 - protein, 107 aa, Artificial Sequence
  Description of Artificial Sequence Synthetic polypeptide
DIQLTQSPSSLSASVGDRVTITCRASQGVISALAWYQQKPGKAPKLLIYDASSLESGVPSRFSGSGSGTDFTLTISSLQPEDFATYYCQQFNSYPLTFGGGTKVEIK

SEQ ID NO: 229 - protein, 107 aa, Artificial Sequence
  Description of Artificial Sequence Synthetic polypeptide
DIQLTQSPSSLSASVGDRVTITCRASQGIRSALAWYQQKPGKAPKLLIYDASSLESGVPSRFSGSGSGTDFTLTISSLQPEDFATYYCQQFNSYPLTFGGGTKVEIK

SEQ ID NO: 230 - protein, 107 aa, Artificial Sequence
  Description of Artificial Sequence Synthetic polypeptide
DIQLTQSPSSLSASVGDRVTITCRASQGVGSALAWYQQKPGKAPKLLIYDASSLESGVPSRFSGSGSGTDFTLTISSLQPEDFATYYCQQFNSYPLTFGGGTKVEIK

SEQ ID NO: 231 - protein, 107 aa, Artificial Sequence
  Description of Artificial Sequence Synthetic polypeptide
DIQLTQSPSSLSASVGDRVTITCRASQGVISALAWYQQKPGKAPKLLIYDASILESGVPSRFSGSGSGTDFTLTISSLQPEDFATYYCQQFNSYPLTFGGGTKVEIK

SEQ ID NO: 232 - protein, 107 aa, Artificial Sequence
  Description of Artificial Sequence Synthetic polypeptide
DIQLTQSPSSLSASVGDRVTITCRASQGIRSALAWYQQKPGKAPKLLIYDASILESGVPSRFSGSGSGTDFTLTISSLQPEDFATYYCQQFNSYPLTFGGGTKVEIK

SEQ ID NO: 233 - protein, 107 aa, Artificial Sequence
  Description of Artificial Sequence Synthetic polypeptide
DIQLTQSPSSLSASVGDRVTITCRASQGVGSALAWYQQKPGKAPKLLIYDASILESGVPSRFSGSGSGTDFTLTISSLQPEDFATYYCQQFNSYPLTFGGGTKVEIK

SEQ ID NO: 234 - protein, 107 aa, Artificial Sequence
  Description of Artificial Sequence Synthetic polypeptide
DIQLTQSPSSLSASVGDRVTITCRASQGISSALAWYQQKPGKAPKLLIYDASILESGVPSRFSGSGSGTDFTLTISSLQPEDFATYYCQQFNSYPLTFGGGTKVEIK

SEQ ID NO: 235 - protein, 107 aa, Artificial Sequence
  Description of Artificial Sequence Synthetic polypeptide
DIQLTQSPSSLSASVGDRVTITCRASQGVISALAWYQQKPGKAPKLLIYDASTLESGVPSRFSGSGSGTDFTLTISSLQPEDFATYYCQQFNSYPLTFGGGTKVEIK

SEQ ID NO: 236 - protein, 107 aa, Artificial Sequence
  Description of Artificial Sequence Synthetic polypeptide
DIQLTQSPSSLSASVGDRVTITCRASQGIRSALAWYQQKPGKAPKLLIYDASTLESGVPSRFSGSGSGTDFTLTISSLQPEDFATYYCQQFNSYPLTFGGGTKVEIK

SEQ ID NO: 237 - protein, 107 aa, Artificial Sequence
  Description of Artificial Sequence Synthetic polypeptide
DIQLTQSPSSLSASVGDRVTITCRASQGVGSALAWYQQKPGKAPKLLIYDASTLESGVPSRFSGSGSGTDFTLTISSLQPEDFATYYCQQFNSYPLTFGGGTKVEIK

SEQ ID NO: 238 - protein, 123 aa, Artificial Sequence
  Description of Artificial Sequence Synthetic polypeptide
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSS

SEQ ID NO: 239 - protein, 107 aa, Artificial Sequence
  Description of Artificial Sequence Synthetic polypeptide
DIQMTQSPSSLSASVGDRVTITCRASQGIGSALAWYQQKPGKAPKLLIYDASTLESGVPSRFSGSGSGTDFTLTISSLQPEDFATYYCQQFNGYPLTFGQGTRLEIK

SEQ ID NO: 240 - protein, 107 aa, Artificial Sequence
  Description of Artificial Sequence Synthetic polypeptide
DIQMTQSPSSLSASVGDRVTITCRASRGISDYLAWYQQKPGKAPKLLIYDASNLETGVPSRFSGSGSGTDFTLTISSLQPEDFATYYCQQANSFPITFGQGTRLEIK

SEQ ID NO: 241 - protein, 107 aa, Artificial Sequence
  Description of Artificial Sequence Synthetic polypeptide
DIQMTQSPSSLSASVGDRVTITCRASQGIGSALAWYQQKPGKAPKLLIYDASTLESGVPSRFSGSGSGTDFTLTISSLQPEDFATYYCQQLNGYPLTFGQGTRLEIK

SEQ ID NO: 242 - protein, 107 aa, Artificial Sequence
  Description of Artificial Sequence Synthetic polypeptide
DIQMTQSPSSLSASVGDRVTITCRASQGIGSALAWYQQKPGKAPKLLIYDASNLETGVPSRFSGSGSGTDFTLTISSLQPEDFATYYCQQLNGYPLTFGQGTRLEIK

SEQ ID NO: 243 - protein, 123 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSS

SEQ ID NO: 244 - protein, 107 aa, Artificial Sequence
  Description of Artificial Sequence Synthetic polypeptide
DIQMTQSPSSLSASVGDRVTITCRSSQGIRSDLGWYQQKPGKAPKLLIYDASNLETGVPSRFSGSGSGTDFTLTISSLQPEDFATYYCQQANGFPLTFGGGTKVEIK

SEQ ID NO: 245 - protein, 5 aa, Artificial Sequence
  Description of Artificial Sequence Synthetic peptide
NYWIG

SEQ ID NO: 246 - protein, 17 aa, Artificial Sequence
  Description of Artificial Sequence Synthetic peptide
IINPRDSDTRYRPSFQG

SEQ ID NO: 247 - protein, 14 aa, Artificial Sequence
  Description of Artificial Sequence Synthetic peptide
HGRGYEGYEGAFDI

SEQ ID NO: 248 - protein, 11 aa, Artificial Sequence
  Description of Artificial Sequence Synthetic peptide
RSSQGIRSDLG

SEQ ID NO: 249 - protein, 7 aa, Artificial Sequence
  Description of Artificial Sequence Synthetic peptide
DASNLET

SEQ ID NO: 250 - protein, 9 aa, Artificial Sequence
  Description of Artificial Sequence Synthetic peptide
QQANGFPLT

SEQ ID NO: 251 - protein, 123 aa, Artificial Sequence
  Description of Artificial Sequence Synthetic polypeptide
EVQLVQSGAEVKKPGESLKISCKGSGYSFTNYWIGWVRQMPGKGLEWMGIIYPGDSDIRYSPSLQGQVTISVDTSTSTAYLQWNSLKPSDTAMYYCARHGRGYNGYEGAFDIWGQGTLVTVSS

SEQ ID NO: 252 - protein, 107 aa, Artificial Sequence
  Description of Artificial Sequence Synthetic polypeptide
DIQMTQSPSSLSASVGDRVTITCRASQGIGDSLAWYQQKPGKAPKLLIYDASNLETGVPSRFSGSGSGTDFTLTISSLQPEDFATYYCQQLNGYPITFGQGTKVEIK

SEQ ID NO: 253 - protein, 17 aa, Artificial Sequence
  Description of Artificial Sequence Synthetic peptide
IIYPGDSDIRYSPSLQG

SEQ ID NO: 254 - protein, 11 aa, Artificial Sequence
  Description of Artificial Sequence Synthetic peptide
RASQGIGDSLA

SEQ ID NO: 255 - protein, 9 aa, Artificial Sequence
  Description of Artificial Sequence Synthetic peptide
QQLNGYPIT

SEQ ID NO: 256 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASSLESGVPSRFSGSGSGTDFTLTISSLQPEDFATYYCQQLNGYPITFGQGTKVEIK

SEQ ID NO: 257 - protein, 11 aa, Artificial Sequence
  Description of Artificial Sequence Synthetic peptide
RASQGIRNDLG

SEQ ID NO: 258 - protein, 123 aa, Artificial Sequence
  Description of Artificial Sequence Synthetic polypeptide
EVQLVQSGAEVKKPGESLKISCKGSGYSFTNYWIGWVRQMPGKGLEWMGIIYPGDSLTRYSPSFQGQVTISADKSISTAYLQWSSLKASDTAMYYCARHGRGYNGYEGAFDIWGQGTLVTVSS

SEQ ID NO: 259 - protein, 17 aa, Artificial Sequence
  Description of Artificial Sequence Synthetic peptide
IIYPGDSLTRYSPSFQG

SEQ ID NO: 260 - protein, 123 aa, Artificial Sequence
  Description of Artificial Sequence Synthetic polypeptide
EVQLVQSGAEVKKPGESLKISCKGSGYSFTNYWIGWVRQMPGKGLEWMGIIYPGDSDTRYSPSFQGQVTISADKSISTAYLQWSSLKASDTAMYYCARHGRGYNGYEGAFDIWGQGTLVTVSS

SEQ ID NO: 261 - protein, 123 aa, Artificial Sequence
  Description of Artificial Sequence Synthetic polypeptide
QVQLVQSGAAVKKPGESLKISCKGSGYRFTSYWIGWVRQMPGKGLEWMGIIYPGDSDTRYSPSFQGQVTISAGKSISTAYLQWSSLKASDTAMYYCARHGRGYNGYEGAFDIWGQGTMVTVSS

SEQ ID NO: 262 - protein, 107 aa, Artificial Sequence [sequence omitted]

SEQ ID NO: 263 - protein, 5 aa, Artificial Sequence
  Description of Artificial Sequence Synthetic peptide
TYWIG

SEQ ID NO: 264 - protein, 11 aa, Artificial Sequence
  Description of Artificial Sequence Synthetic peptide
RASQGVISALA

SEQ ID NO: 265 - protein, 7 aa, Artificial Sequence
  Description of Artificial Sequence Synthetic peptide
DASILES

SEQ ID NO: 266 - protein, 9 aa, Artificial Sequence
  Description of Artificial Sequence Synthetic peptide
QQFNSYPLT

SEQ ID NO: 267 - protein, 11 aa, Artificial Sequence
  Description of Artificial Sequence Synthetic peptide
RASQGVGSALA

SEQ ID NO: 268 - protein, 7 aa, Artificial Sequence
  Description of Artificial Sequence Synthetic peptide
DASTLES

SEQ ID NO: 269 - protein, 330 aa, Homo sapiens [sequence omitted]

SEQ ID NO: 270 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 271 - protein, 330 aa, Artificial Sequence; identical to SEQ ID NO: 123
  Description of Artificial Sequence Synthetic polypeptide

SEQ ID NO: 272 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 273 - protein, 330 aa, Artificial Sequence; identical to SEQ ID NO: 124
  Description of Artificial Sequence Synthetic polypeptide

SEQ ID NO: 274 - protein, 330 aa, Artificial Sequence; identical to SEQ ID NO: 126
  Description of Artificial Sequence Synthetic polypeptide

SEQ ID NO: 275 - protein, 453 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 276 - protein, 453 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 277 - protein, 453 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 278 - protein, 453 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 279 - protein, 453 aa, Artificial Sequence
  Description of Artificial Sequence Synthetic polypeptide
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 280 - protein, 453 aa, Artificial Sequence
  Description of Artificial Sequence Synthetic polypeptide
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 281 - protein, 453 aa, Artificial Sequence
  Description of Artificial Sequence Synthetic polypeptide
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 282 - protein, 453 aa, Artificial Sequence
  Description of Artificial Sequence Synthetic polypeptide
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 283 - protein, 107 aa, Artificial Sequence [sequence omitted]

SEQ ID NO: 284 - protein, 214 aa, Artificial Sequence
  Description of Artificial Sequence Synthetic polypeptide
DIQMTQSPSSLSASVGDRVTITCRSSQGIRSDLGWYQQKPGKAPKLLIYDASNLETGVPSRFSGSGSGTDFTLTISSLQPEDFATYYCQQANGFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 285 - protein, 214 aa, Artificial Sequence
  Description of Artificial Sequence Synthetic polypeptide
DIQMTQSPSSLSASVGDRVTITCRASQGIGSALAWYQQKPGKAPKLLIYDASNLETGVPSRFSGSGSGTDFTLTISSLQPEDFATYYCQQLNGYPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 286 - protein, 5 aa, Artificial Sequence
  Description of Artificial Sequence Synthetic peptide
TSWIG

SEQ ID NO: 287 - protein, 14 aa, Artificial Sequence
  Description of Artificial Sequence Synthetic peptide
HGLGYNGYEGAFDI

SEQ ID NO: 288 - protein, 11 aa, Artificial Sequence
  Description of Artificial Sequence Synthetic peptide
RASQGIGSALA

SEQ ID NO: 289 - protein, 10 aa, Artificial Sequence
  Description of Artificial Sequence Synthetic peptide
CQQLNGYPLT

SEQ ID NO: 290 - protein, 501 aa, Homo sapiens
QPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTHHHHHH

(19) United States
(12) Patent Application Publication
(10) Pub. No.: US 2022/0175944 A1
(43) Pub. Date: Jun. 9, 2022

(54) ANTI-CD117 ANTIBODY-DRUG CONJUGATES AND USES THEREOF

(71) Applicant: Magenta Therapeutics, Inc., Cambridge, MA (US)

(72) Inventors: Charlotte F. McDonagh, Winchester, MA (US); Anthony Boitano, Newton, MA (US); Michael Cooke, Boston, MA (US); Ganapathy N. Sarma, Belmont, MA (US); Rajiv Panwar, Acton, MA (US)

(21) Appl. No.: 17/507,618

(22) Filed: Oct. 21, 2021

Related U.S. Application Data
(63) Continuation of application No. PCT/US2020/029657, filed on Apr. 23, 2020.
(60) Provisional application No. 62/838,293, filed on Apr. 24, 2019.

(51) Int. Cl. A61K 47/68; C07K 16/28; A61K 47/65; A61K 35/28

(52) U.S. Cl. CPC A61K 47/6803; A61K 47/6849; A61K 35/28; A61K 47/65; C07K 16/2803

(57) ABSTRACT

Anti-CD117 antibody-drug conjugates (ADCs) comprising pyrrolobenzodiazepine are provided, as well as compositions and methods of using the same. The compositions and methods provided herein can also be used to prepare a patient for hematopoietic stem cell transplant therapy and to improve the engraftment of hematopoietic stem cell transplants by selectively depleting endogenous hematopoietic stem cells prior to the transplant procedure. Methods and compositions for the treatment of various hematopoietic diseases, metabolic disorders, cancers, and autoimmune diseases, are provided.

### RELATED APPLICATIONS

This application is a continuation of PCT Appln. No. PCT/US2020/029657, filed on Apr. 23, 2020, which claims priority to U.S. Provisional Application No. 62/838,293, filed on Apr. 24, 2019. The contents of the aforementioned applications are incorporated by reference herein in their entirety.

### SEQUENCE LISTING

The instant application contains a Sequence Listing which has been submitted electronically in ASCII format and is hereby incorporated by reference in its entirety. Said ASCII copy, created on Oct. 20, 2021, is named M103034_2090US_C1_SL.txt and is 317,372 bytes in size.

### FIELD

The present disclosure relates to anti-CD117 Antibody-Drug Conjugates (ADCs) and methods for using the same for therapeutic purposes.

### BACKGROUND

Monoclonal antibodies (mAb) can be conjugated to a therapeutic agent to form an antibody drug conjugate (ADC). ADCs can exhibit increased efficacy, as compared to an unconjugated antibody. The linkage of the antibody to the drug (e.g., a cytotoxic drug) can be direct, or indirect via a linker. An important aspect of successful therapeutic ADCs is that the ADC be not only effective, but also well-tolerated. Often the cytotoxin impacts both efficacy and tolerability.

ADCs have been proposed as a therapeutic regimen for preparing patients for transplant and stem cell therapy. By conditioning a patient with a cell-specific ADC, stem cells or immune cells can be selectively depleted while leaving the patient's remaining immune system largely intact. For example, Palchaudhuri et al. (2016) Nat. Biotechnol. 34, 738-745 describes the use of a single dose of an anti-CD45 ADC, where an anti-CD45 antibody was conjugated to saporin (SAP), and its ability to enable engraftment of donor cells for treatment in a sickle-cell anemia model. Unlike irradiation, the CD45-SAP ADC was reported to have avoided neutropenia and anemia, and provided for rapid recovery of T and B cells with minimal overall toxicity. However, there remains a need for a combination of effective cell targets and toxins that can be used for non-genotoxic, targeted ADC-conditioning.

### SUMMARY

The present disclosure provides anti-CD117 antibody drug conjugates (ADCs) for delivery of to a target cell.

In one aspect, the present disclosure provides an antibody drug conjugate (ADC) comprising an anti-CD117 antibody, or antigen binding portion thereof (Ab), conjugated to a cytotoxin (Cy) via a linker (L), wherein the cytotoxin comprises a pyrrolobenzodiazepine (PBD).

In some embodiments, the cytotoxin is a PBD dimer.

In some embodiments, the PBD dimer is represented by Formula (I):

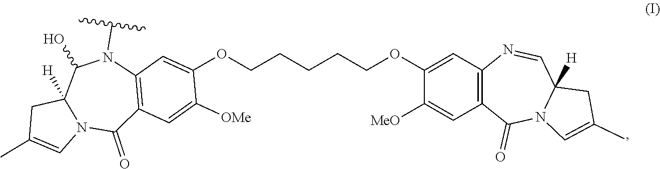

wherein the wavy line indicates the point of covalent attachment to the linker of the ADC.

In some embodiments, the linker comprises one or more of a peptide, oligosaccharide, —(CH₂)ₚ—, —(CH₂CH₂O)_{q}—, —(C═O)(CH₂)ᵣ—, —(C═O)(CH₂CH₂O)ₜ—, —(NHCH₂CH₂)ᵤ—, -PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB, wherein each of p, q, r, t, and u are integers from 1-12, selected independently for each occurrence.

In some embodiments, the linker has the structure of formula (II):

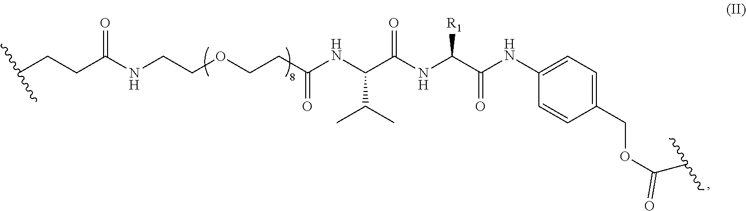

wherein R₁ is CH₃ (Ala) or (CH₂)₃NH(CO)NH₂ (Cit).

In some embodiments, the linker, prior to conjugation to the antibody and including the reactive substituent Z′, taken together as L-Z′, has the structure:

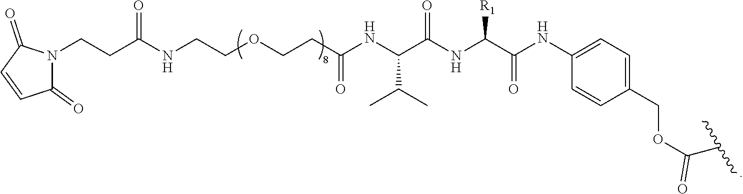

In certain embodiments, R₁ is CH₃.

In some embodiments, the cytotoxin-linker conjugate, prior to conjugation to the antibody and including the reactive substituent Z′, taken together as Cy-L-Z′, is tesirine, having the structure of formula (IV):

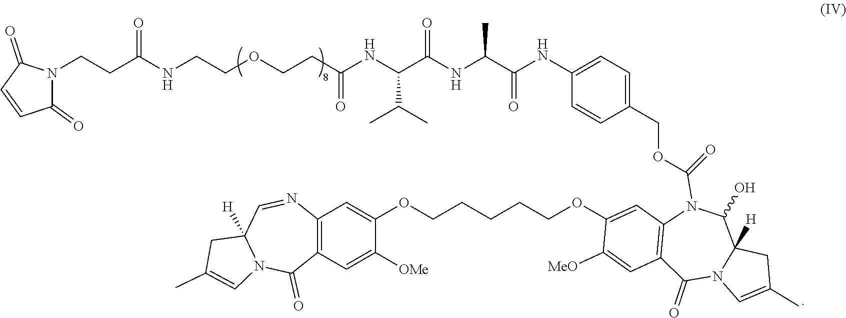

In some embodiments, the ADC has the structure of formula (V):

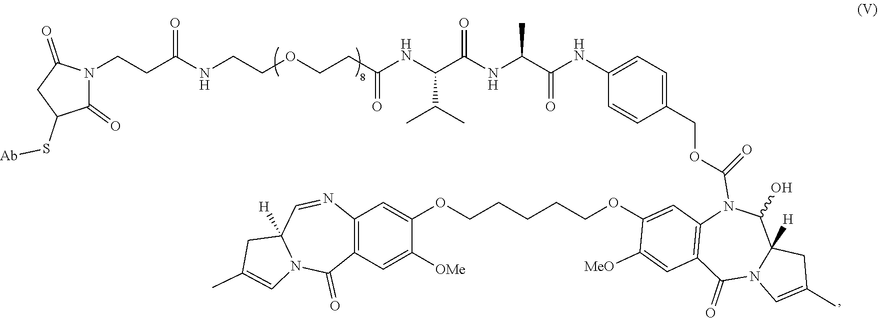

wherein Ab is the anti-CD117 antibody or antigen binding fragment thereof, and S represents a sulfur atom present in or introduced into the antibody or antigen binding fragment thereof.

In some embodiments, the antibody, or antigen binding portion thereof, comprises an Fc domain, and wherein the antibody, or antigen binding portion thereof, is conjugated to the PBD by way of a cysteine residue in the Fc domain. In certain embodiments, the cysteine residue is introduced by way of an amino acid substitution in the Fc domain. In some embodiments, the amino acid substitution is D265C and/or V205C (EU numbering).

In some embodiments, the ADC has a drug to antibody ratio (DAR) of about 1, about 2, about 3, about 4, about 5, about 6, about 7, or about 8. In some embodiments, the ADC has a drug to antibody ratio (DAR) of 1-5, 2-4, 1-4, 2-3, or 1-3.

In some embodiments, the antibody, or antigen binding portion thereof, is a human antibody or antigen binding portion thereof, or a humanized antibody or antigen binding portion thereof.

In some embodiments, the antibody, or antigen binding portion thereof, is an IgG.

In some embodiments, the antibody, or antigen binding portion thereof, is an IgG1 or an IgG4.

In some embodiments, the antibody is an intact antibody.

In some embodiments, the antibody, or the antigen binding fragment thereof, comprises a heavy chain comprising an HC-CDR1, an HC-CDR2, and an HC-CDR3 or a variable region from the heavy chain variable region amino acid sequence of Ab55, Ab54, Ab56, Ab57, Ab58, Ab61, Ab66, Ab67, Ab68, Ab69, Ab85, Ab86, Ab87, Ab88, Ab89, Ab77, Ab79, Ab81, Ab85, or Ab249, and a light chain comprising a LC-CDR1, a LC-CDR2, and a LC-CDR3 or a variable region from the light chain variable region amino acid sequence of Ab55, Ab54, Ab56, Ab57, Ab58, Ab61, Ab66, Ab67, Ab68, Ab69, Ab85, Ab86, Ab87, Ab88, Ab89, Ab77, Ab79, Ab81, Ab85, or Ab249, a heavy chain comprising an HC-CDR1, an HC-CDR2, and an HC-CDR3 or a variable region from the heavy chain variable region amino acid sequence of SEQ ID NO: 147, 164, 166, 168, 170, 172, 174, 176, 178, 180, 183, 185, 187, 189, 191, 193, 195, 197, 199, 201, 202, 204, 206, 208, 210, 212, 214, 216, 218, 220, 222, 224, 226, 238, or 243, and a light chain comprising an LC-CDR1, an LC-CDR2, and an LC-CDR3 or a variable region from the light chain variable region amino acid sequence of SEQ ID NO: 148, 149, 150, 151, 152, 153, 154, 155, 156, 157, 158, 159, 160, 161, 162, 163, 165, 167, 169, 171, 173, 175, 177, 179, 181, 182, 184, 186, 188, 190, 192, 194, 196, 198, 200, 203, 205, 207, 209, 211, 213, 215, 217, 219, 221, 223, 225, 227, 228, 229, 230, 231, 232, 233, 234, 235, 236, 237, 239, 240, 241, 242, or 244; a heavy chain comprising a variable region comprising an amino acid sequence as set forth in SEQ ID NO: 9 and a light chain comprising a variable region comprising an amino acid sequence as set forth in SEQ ID NO: 10; or a heavy chain comprising a variable region comprising an amino acid sequence as set forth in SEQ ID NO: 243, and a light chain comprising a variable region comprising an amino acid sequence as set forth in SEQ ID NO: 244.

In some embodiments, the antibody, or the antigen binding fragment thereof comprises a heavy chain comprising a heavy chain (HC)-CDR1, HC-CDR2, and HC-CDR3 comprising an amino acid sequence as set forth in SEQ ID NO: 11, 12, and 13, respectively, and a light chain comprising a light chain (LC)-CDR1, LC-CDR2, and LC-CDR3 comprising an amino acid sequence as set forth in SEQ ID NOs: 14, 15, and 16, respectively; or a heavy chain comprising a heavy chain (HC)-CDR1, HC-CDR2, and HC-CDR3 comprising an amino acid sequence as set forth in SEQ ID NOs: 245, 246, and 247, respectively, and a light chain comprising a light chain (LC)-CDR1, LC-CDR2, and LC-CDR3 comprising an amino acid sequence as set forth in SEQ ID NOs: 248, 249, and 250, respectively.

In some embodiments, the antibody, or the antigen binding fragment thereof, comprises an Fc region comprising at least one mutation selected from the group consisting of D265C, H435A, L234A, or L235A (according to EU index).

In some embodiments, the antibody or antigen binding fragment thereof comprises an Fc region comprising D265C, H435A, L234A, or L235A (according to EU index) mutations. In some embodiments, the antibody or antigen binding fragment thereof comprises an Fc region comprising S239C.

In another aspect, the present disclosure provides a pharmaceutical composition comprising any ADCs described herein, and a pharmaceutically acceptable carrier.

In another aspect, the present disclosure provides a method of depleting a population of hematopoietic stem cells (HSC) in a human patient, the method comprising administering to the patient an effective amount of any ADC described herein, or a pharmaceutical composition described herein.

In some embodiments, the method further comprises administering to the patient a transplant comprising hematopoietic stem cells. In some embodiments, the transplant is allogeneic. In some embodiments, the transplant is autologous.

In another aspect, the present disclosure provides a method comprising administering to a human patient a transplant comprising hematopoietic stem cells, wherein the patient has previously been administered any ADC described herein, or a pharmaceutical composition described herein, in an amount sufficient to deplete a population of hematopoietic stem cells from the patient.

In some embodiments, the patient has a blood disease, a metabolic disorder, a cancer, an autoimmune disease, or severe combined immunodeficiency disease (SCID).

In some embodiments, the patient has a hematological cancer. In certain embodiments, the hematological cancer is leukemia or lymphoma.

In some embodiments, the autoimmune disease is multiple sclerosis. In certain embodiments, the autoimmune disease is scleroderma.

In another aspect, provided herein is a method of depleting a population of CD117+ cells in a human patient in need of a hematopoietic stem cell transplant, the method comprising administering to the human patient an effective amount of an antibody drug conjugate (ADC) comprising a pyrrolobenzodiazepine (PBD) conjugated to an antibody or antigen binding portion thereof capable of specifically binding human CD117, wherein the antibody or antigen binding portion thereof comprises an Fc domain and is internalized by a CD117+ cell, and wherein the antibody comprises

a heavy chain comprising an HC-CDR1, an HC-CDR2, and an HC-CDR3 or a variable region from the heavy chain variable region amino acid sequence of Ab55, Ab54, Ab56, Ab57, Ab58, Ab61, Ab66, Ab67, Ab68, Ab69, Ab85, Ab86, Ab87, Ab88, Ab89, Ab77, Ab79, Ab81, Ab85, or Ab249, and a light chain comprising an LC-CDR1, an LC-CDR2, and an LC-CDR3 or a variable region from the light chain variable region amino acid sequence of Ab55, Ab54, Ab56, Ab57, Ab58, Ab61, Ab66, Ab67, Ab68, Ab69, Ab85, Ab86, Ab87, Ab88, Ab89, Ab77, Ab79, Ab81, Ab85, or Ab249,

a heavy chain comprising an HC-CDR1, an HC-CDR2, and an HC-CDR3 or a variable region from the heavy chain variable region amino acid sequence of SEQ ID NO: 147, 164, 166, 168, 170, 172, 174, 176, 178, 180, 183, 185, 187, 189, 191, 193, 195, 197, 199, 201, 202, 204, 206, 208, 210, 212, 214, 216, 218, 220, 222, 224, 226, 238, or 243, and a light chain comprising an LC-CDR1, an LC-CDR2, and an LC-CDR3 or a variable region from the light chain variable region amino acid sequence of SEQ ID NO: 148, 149, 150, 151, 152, 153, 154, 155, 156, 157, 158, 159, 160, 161, 162, 163, 165, 167, 169, 171, 173, 175, 177, 179, 181, 182, 184, 186, 188, 190, 192, 194, 196, 198, 200, 203, 205, 207, 209, 211, 213, 215, 217, 219, 221, 223, 225, 227, 228, 229, 230, 231, 232, 233, 234, 235, 236, 237, 239, 240, 241, 242, or 244;

a heavy chain comprising a variable region comprising an amino acid sequence as set forth in SEQ ID NO: 9 and a light chain comprising a variable region comprising an amino acid sequence as set forth in SEQ ID NO: 10; or

a heavy chain comprising a variable region comprising an amino acid sequence as set forth in SEQ ID NO: 243, and a light chain comprising a variable region comprising an amino acid sequence as set forth in SEQ ID NO: 244

In some embodiments, the patient has a hematological cancer. In certain embodiments, the hematological cancer is leukemia or lymphoma.

In another aspect, the present disclosure provides a method of conditioning a human patient for receiving a hematopoietic stem cell (HSC) transplant, the method comprising administering to the human patient an effective amount of an antibody drug conjugate (ADC) comprising a pyrrolobenzodiazepine (PBD) conjugated to an antibody or antigen binding portion thereof capable of specifically binding human CD117, wherein the antibody or antigen binding portion thereof comprises an Fc domain and is internalized by a CD117+ cell, and wherein the human patient has a stem cell disorder, and wherein the antibody comprises

a heavy chain comprising an HC-CDR1, an HC-CDR2, and an HC-CDR3 or a variable region from the heavy chain variable region amino acid sequence of Ab55, Ab54, Ab56, Ab57, Ab58, Ab61, Ab66, Ab67, Ab68, Ab69, Ab85, Ab86, Ab87, Ab88, Ab89, Ab77, Ab79, Ab81, Ab85, or Ab249, and a light chain comprising an LC-CDR1, an LC-CDR2, and an LC-CDR3 or a variable region from the light chain variable region amino acid sequence of Ab55, Ab54, Ab56, Ab57, Ab58, Ab61, Ab66, Ab67, Ab68, Ab69, Ab85, Ab86, Ab87, Ab88, Ab89, Ab77, Ab79, Ab81, Ab85, or Ab249,

a heavy chain comprising an HC-CDR1, an HC-CDR2, and an HC-CDR3 or a variable region from the heavy chain variable region amino acid sequence of SEQ ID NO: 147, 164, 166, 168, 170, 172, 174, 176, 178, 180, 183, 185, 187, 189, 191, 193, 195, 197, 199, 201, 202, 204, 206, 208, 210, 212, 214, 216, 218, 220, 222, 224, 226, 238, or 243, and a light chain comprising an LC-CDR1, an LC-CDR2, and an LC-CDR3 or a variable region from the light chain variable region amino acid sequence of SEQ ID NO: 148, 149, 150, 151, 152, 153, 154, 155, 156, 157, 158, 159, 160, 161, 162, 163, 165, 167, 169, 171, 173, 175, 177, 179, 181, 182, 184, 186, 188, 190, 192, 194, 196, 198, 200, 203, 205, 207, 209, 211, 213, 215, 217, 219, 221, 223, 225, 227, 228, 229, 230, 231, 232, 233, 234, 235, 236, 237, 239, 240, 241, 242, or 244;

a heavy chain comprising a variable region comprising an amino acid sequence as set forth in SEQ ID NO: 9 and a light chain comprising a variable region comprising an amino acid sequence as set forth in SEQ ID NO: 10; or

a heavy chain comprising a variable region comprising an amino acid sequence as set forth in SEQ ID NO: 243, and a light chain comprising a variable region comprising an amino acid sequence as set forth in SEQ ID NO: 244.

In some embodiments, the stem cell disorder is a hematological cancer or an autoimmune disease.

In some embodiments, the method further comprises administering a hematopoietic stem cell transplant to the subject.

In some embodiments, the transplant is administered to the human patient after the ADC has substantially cleared from the blood of the human patient.

In some embodiments, the hematopoietic stem cell transplant comprises allogeneic cells.

In some embodiments, the hematopoietic stem cell transplant comprises autologous cells.

### BRIEF DESCRIPTION OF FIGURES

FIGS. 1A-1C graphically depict the results of an in vivo cell depletion assay to assess killing of CD34+ bone marrow cells in baboon by an anti-CD117 antibody conjugated to PBD or calicheamicin. FIG. 1A depicts an example of the flow cytometry gating strategy used to analyze the killing of bone marrow cells isolated from baboon treated with the indicated anti-CD117 ADC. The absolute number of live cells (FIG. 1B) and CD34+ CD90+ cells (FIG. 1C) isolated from the bone marrow of baboons dosed with the indicated ADC are shown as a function of ADC concentration.

FIGS. 2A-2C graphically depict the results of in vitro cell killing assays in both Kasumi-1 cells (FIGS. 2A and 2B) or primary human stem cells (FIG. 2C) for an anti-CD117-PBD, an anti-CD117-PNU, an anti-CD117-DM (duocarmycin), and an anti-CD117-calicheamicin (D4). FIGS. 2A and 2B graphically depict the results of in vitro cell killing assays that show Kasumi-1 cell viability (FIG. 2A) or CD117(−) Kasumi-1 cell viability (FIG. 2B) as measured in luminescence (RLU) by Celltiter Glo as a function of the indicated ADC concentration. FIG. 2C graphically depicts the results of in vitro cell killing assays that show the dose-dependent effect of each indicated ADC on the viability of human CD34+ bone marrow cells based on viable CD34+CD90+ cell counts (y-axis).

FIG. 3A graphically depicts the results of an in vivo HSC depletion assay in hNSG mice with an anti-CD117 antibody conjugated to PNU, PBD, D4 (calicheamicin), or DM1 (duocarmycin). FIG. 3A graphically depicts the percentage of hCD33 cells normalized to baseline in mice treated with the indicated anti-CD117 ADC and dosage 7 days, 14 days, or 21 days post-administration. FIG. 3B graphically depicts the percentage of hCD34+ cells and FIG. 3C graphically depicts the hCD34+ count per femur in mice treated with the indicated anti-CD117-ADC and dosage 21 days post-administration.

FIG. 4 graphically depicts the results of an in vivo HSC depletion assay in hNSG mice treated with high-doses of an anti-CD117 antibody conjugated to PBD or D4 (calicheamicin), showing the hCD34+ count per femur in mice treated with the indicated ADC and dosage 21 days post-administration.

FIG. 5 graphically depicts the percent change in body weight overtime in C57BL/6 mice treated with an anti-CD117-PBD or anti-CD117-calicheamicin ADC.

### DETAILED DESCRIPTION

For clarity of disclosure, and not by way of limitation, the detailed description of the present disclosure is divided into the subsections which follow.

### Definitions

The term “acyl” as used herein refers to —C(═O)R, wherein R is hydrogen (“aldehyde”), C₁-C₁₂ alkyl, C₂-C₁₂ alkenyl, C₂-C₁₂ alkynyl, C₃-C₇ carbocyclyl, C₆-C₂₀ aryl, 5-10 membered heteroaryl, or 5-10 membered heterocyclyl, as defined herein. Non-limiting examples include formyl, acetyl, propanoyl, benzoyl, and acryloyl.

The term “C₁-C₁₂ alkyl” as used herein refers to a straight chain or branched, saturated hydrocarbon having from 1 to 12 carbon atoms. Representative C₁-C₁₂ alkyl groups include, but are not limited to, -methyl, -ethyl, -n-propyl, -n-butyl, -n-pentyl, and -n-hexyl; while branched C₁-C₁₂ alkyls include, but are not limited to, -isopropyl, -sec-butyl, -isobutyl, -tert-butyl, -isopentyl, and 2-methylbutyl. A C₁-C₁₂ alkyl group can be unsubstituted or substituted.

The term “alkenyl” as used herein refers to C₂-C₁₂ hydrocarbon containing normal, secondary, or tertiary carbon atoms with at least one site of unsaturation, i.e., a carbon-carbon, sp² double bond. Examples include, but are not limited to: ethylene or vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, and the like. An alkenyl group can be unsubstituted or substituted.

“Alkynyl” as used herein refers to a C₂-C₁₂ hydrocarbon containing normal, secondary, or tertiary carbon atoms with at least one site of unsaturation, i.e., a carbon-carbon, sp triple bond. Examples include, but are not limited to acetylenic and propargyl. An alkynyl group can be unsubstituted or substituted.

“Aryl” as used herein refers to a C₆-C₂₀ carbocyclic aromatic group. Examples of aryl groups include, but are not limited to, phenyl, naphthyl and anthracenyl. An aryl group can be unsubstituted or substituted.

“Arylalkyl” as used herein refers to an acyclic alkyl radical in which one of the hydrogen atoms bonded to a carbon atom, typically a terminal or spa carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g. the alkyl moiety, including alkanyl, alkenyl or alkynyl groups, of the arylalkyl group is 1 to 6 carbon atoms and the aryl moiety is 5 to 14 carbon atoms. An alkaryl group can be unsubstituted or substituted.

“Cycloalkyl” as used herein refers to a saturated carbocyclic radical, which may be mono- or bicyclic. Cycloalkyl groups include a ring having 3 to 7 carbon atoms as a monocycle or 7 to 12 carbon atoms as a bicycle. Examples of monocyclic cycloalkyl groups include cyclopropyl, cyclobutyl, cyclopentyl, cyclohexyl, cycloheptyl, and cyclooctyl. A cycloalkyl group can be unsubstituted or substituted.

“Cycloalkenyl” as used herein refers to an unsaturated carbocyclic radical, which may be mono- or bicyclic. Cycloalkenyl groups include a ring having 3 to 6 carbon atoms as a monocycle or 7 to 12 carbon atoms as a bicycle. Examples of monocyclic cycloalkenyl groups include 1-cyclopent-1-enyl, 1-cyclopent-2-enyl, 1-cyclopent-3-enyl, 1-cyclohex-1-enyl, 1-cyclohex-2-enyl, and 1-cyclohex-3-enyl. A cycloalkenyl group can be unsubstituted or substituted.

“Heteroaralkyl” as used herein refers to an acyclic alkyl radical in which one of the hydrogen atoms bonded to a carbon atom, typically a terminal or spa carbon atom, is replaced with a heteroaryl radical. Typical heteroarylalkyl groups include, but are not limited to, 2-benzimidazolylmethyl, 2-furylethyl, and the like. The heteroarylalkyl group comprises 6 to 20 carbon atoms, e.g. the alkyl moiety, including alkanyl, alkenyl or alkynyl groups, of the heteroarylalkyl group is 1 to 6 carbon atoms and the heteroaryl moiety is 5 to 14 carbon atoms and 1 to 3 heteroatoms selected from N, O, P, and S. The heteroaryl moiety of the heteroarylalkyl group may be a monocycle having 3 to 7 ring members (2 to 6 carbon atoms or a bicycle having 7 to 10 ring members (4 to 9 carbon atoms and 1 to 3 heteroatoms selected from N, O, P, and S), for example: a bicyclo[4,5], [5,5], [5,6], or [6,6] system.

“Heteroaryl” and “heterocycloalkyl” as used herein refer to an aromatic or non-aromatic ring system, respectively, in which one or more ring atoms is a heteroatom, e.g. nitrogen, oxygen, and sulfur. The heteroaryl or heterocycloalkyl radical comprises 2 to 20 carbon atoms and 1 to 3 heteroatoms selected from N, O, P, and S. A heteroaryl or heterocycloalkyl may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo[4,5], [5,5], [5,6], or [6,6] system. Heteroaryl and heterocycloalkyl can be unsubstituted or substituted.

Heteroaryl and heterocycloalkyl groups are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566.

Examples of heteroaryl groups include by way of example and not limitation pyridyl, thiazolyl, tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, benzotriazolyl, benzisoxazolyl, and isatinoyl.

Examples of heterocycloalkyls include by way of example and not limitation dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl.

By way of example and not limitation, carbon bonded heteroaryls and heterocycloalkyls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heteroaryls and heterocycloalkyls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or beta-carboline. Still more typically, nitrogen bonded heterocycles include 1-aziridyl, 1-azetedyl, 1-pyrrolyl, 1-imidazolyl, 1-pyrazolyl, and 1-piperidinyl.

“Substituted” as used herein and as applied to any of the above alkyl, alkenyl, alkynyl, aryl, arylalkyl, cycloalkyl, heteroaryl, heterocyclyl, and the like, means that one or more hydrogen atoms are each independently replaced with a substituent. Unless otherwise constrained by the definition of the individual substituent, the foregoing chemical moieties, such as “alkyl”, “alkylene”, “heteroalkyl”, “heteroalkylene”, “alkenyl”, “alkenylene”, “heteroalkenyl”, “heteroalkenylene”, “alkynyl”, “alkynylene”, “heteroalkynyl”, “heteroalkynylene”, “cycloalkyl”, “cycloalkylene”, “heterocyclolalkyl”, heterocycloalkylene”, “aryl,” “arylene”, “heteroaryl”, and “heteroarylene” groups can optionally be substituted. Typical substituents include, but are not limited to, —X, —R, —OH, —OR, —SH, —SR, NH₂, —NHR, —N(R)₂, —N⁺(R)₃, —CX₃, —CN, —OCN, —SCN, —NCO, —NCS, —NO, —NO₂, —N₃, —NC(═O)H, —NC(═O)R, —C(═O)H, —C(═O)R, —C(═O)NH₂, —C(═O)N(R)₂, —SO₃—, —SO₃H, —S(═O)₂R, —OS(═O)₂OR, —S(═O)₂NH₂, —S(═O)₂N(R)₂, —S(═O)R, —OP(═O)(OH)₂, —OP(═O)(OR)₂, —P(═O)(OR)₂, —PO₃, —PO₃H₂, —C(═O)X, —C(═S)R, —CO₂H, —CO₂R, —CO₂—, —C(═S)OR, —C(═O)SR, —C(═S)SR, —C(═O)NH₂, —C(═O)N(R)₂, —C(═S)NH₂, —C(═S)N(R)₂, —C(═NH)NH₂, and —C(═NR)N(R)₂; wherein each X is independently selected for each occasion from F, Cl, Br, and I; and each R is independently selected for each occasion from C₁-C₁₂ alkyl, C₆-C₂₀ aryl, C₃-C₁₄ heterocycloalkyl or heteroaryl, protecting group and prodrug moiety. Wherever a group is described as “optionally substituted,” that group can be substituted with one or more of the above substituents, independently for each occasion.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene,” “alkenylene,” “arylene,” “heterocycloalkylene,” and the like.

Wherever a substituent is depicted as a di-radical (i.e., has two points of attachment to the rest of the molecule), it is to be understood that the substituent can be attached in any directional configuration unless otherwise indicated.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers,” or sometimes “optical isomers.”

A carbon atom bonded to four non-identical substituents is termed a “chiral center.” “Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116). A mixture containing equal amounts of individual enantiomeric forms of opposite chirality is termed a “racemic mixture.”

The compounds disclosed in this description and in the claims may comprise one or more asymmetric centers, and different diastereomers and/or enantiomers of each of the compounds may exist. The description of any compound in this description and in the claims is meant to include all enantiomers, diastereomers, and mixtures thereof, unless stated otherwise. In addition, the description of any compound in this description and in the claims is meant to include both the individual enantiomers, as well as any mixture, racemic or otherwise, of the enantiomers, unless stated otherwise. When the structure of a compound is depicted as a specific enantiomer, it is to be understood that the disclosure of the present application is not limited to that specific enantiomer. Accordingly, enantiomers, optical isomers, and diastereomers of each of the structural formulae of the present disclosure are contemplated herein. In the present specification, the structural formula of the compound represents a certain isomer for convenience in some cases, but the present disclosure includes all isomers, such as geometrical isomers, optical isomers based on an asymmetrical carbon, stereoisomers, tautomers, and the like, it being understood that not all isomers may have the same level of activity. The compounds may occur in different tautomeric forms. The compounds according to the disclosure are meant to include all tautomeric forms, unless stated otherwise. When the structure of a compound is depicted as a specific tautomer, it is to be understood that the disclosure of the present application is not limited to that specific tautomer.

The compounds of any formula described herein include the compounds themselves, as well as their salts, and their solvates, if applicable. A salt, for example, can be formed between an anion and a positively charged group (e.g., amino) on a compound of the disclosure. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt. Likewise, a salt can also be formed between a cation and a negatively charged group (e.g., carboxylate) on a compound of the disclosure. Suitable cations include sodium ion, potassium ion, magnesium ion, calcium ion, and an ammonium cation such as tetramethylammonium ion. Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. The compounds of the disclosure also include those salts containing quaternary nitrogen atoms.

Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Additionally, the compounds of the present disclosure, for example, the salts of the compounds, can exist in either hydrated or unhydrated (the anhydrous) form or as solvates with other solvent molecules. Non-limiting examples of hydrates include monohydrates, dihydrates, etc. Non-limiting examples of solvates include ethanol solvates, acetone solvates, etc. “Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O. A hydrate refers to, for example, a mono-hydrate, a di-hydrate, a tri-hydrate, etc.

In addition, a crystal polymorphism may be present for the compounds or salts thereof represented by the formulae disclosed herein. It is noted that any crystal form, crystal form mixture, or anhydride or hydrate thereof, is included in the scope of the present disclosure.

As used herein, the term “about” refers to a value that is within 10% above or below the value being described. For example, the term “about 5 nM” indicates a range of from 4.5 nM to 5.5 nM.

As used herein, the term “antibody” refers to an immunoglobulin molecule that specifically binds to, or is immunologically reactive with, a particular antigen. An antibody includes, but is not limited to, monoclonal antibodies, polyclonal antibodies, multispecific antibodies (e.g., bispecific antibodies), genetically engineered, and otherwise modified forms of antibodies, including but not limited to de-immunized antibodies, chimeric antibodies, humanized antibodies, heteroconjugate antibodies (e.g., bi- tri- and quad-specific antibodies, diabodies, triabodies, and tetrabodies), and antibody fragments (i.e., antigen binding fragments of antibodies), including, for example, Fab′, F(ab′)₂, Fab, Fv, rlgG, and scFv fragments, so long as they exhibit the desired antigen-binding activity.

The term “monoclonal antibody” (mAb) as used herein refers to an antibody that is derived from a single clone, including any eukaryotic, prokaryotic, or phage clone, by any means available or known in the art, and is not limited to antibodies produced through hybridoma technology. Monoclonal antibodies useful with the present disclosure can be prepared using a wide variety of techniques known in the art including the use of hybridoma, recombinant, and phage display technologies, or a combination thereof. The term “monoclonal antibody” is meant to include both intact molecules, as well as antibody fragments (including, for example, Fab and F(ab′)₂ fragments) that are capable of specifically binding to a target protein. As used herein, the Fab and F(ab′)₂ fragments refer to antibody fragments that lack the Fc fragment of an intact antibody. Examples of these antibody fragments are described herein.

The antibodies of the present disclosure are generally isolated or recombinant. “Isolated,” when used herein refers to a polypeptide, e.g., an antibody, that has been separated and/or recovered from a cell or cell culture from which it was expressed. Ordinarily, an isolated antibody will be prepared by at least one purification step. Thus, an “isolated antibody,” refers to an antibody which is substantially free of other antibodies having different antigenic specificities. For instance, an isolated antibody that specifically binds to CD117 is substantially free of antibodies that specifically bind antigens other than CD117.

The term “antigen-binding fragment,” as used herein, refers to one or more portions of an antibody that retain the ability to specifically bind to a target antigen. The antigen-binding function of an antibody can be performed by fragments of a full-length antibody. The antibody fragments can be, for example, a Fab, F(ab′)₂, scFv, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment containing two Fab fragments linked by a disulfide bridge at the hinge region; (iii) a Fd fragment consisting of the VH and C_{H}1 domains; (iv) a Fv fragment consisting of the V_{L} and VH domains of a single arm of an antibody, (v) a dAb including V_{H} and V_{L} domains; (vi) a dAb fragment that consists of a V_{H} domain (see, e.g., Ward et al., Nature 341:544-546, 1989); (vii) a dAb which consists of a V_{H} or a V_{L} domain; (viii) an isolated complementarity determining region (CDR); and (ix) a combination of two or more (e.g., two, three, four, five, or six) isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv); see, for example, Bird et al., Science 242:423-426, 1988 and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

As used herein, the term “anti-CD117 antibody” or “an antibody that binds to CD117” refers to an antibody that is capable of binding CD117, e.g., human CD117, with sufficient affinity such that the antibody is useful as a diagnostic and/or therapeutic agent in targeting CD117. The amino acid sequences of the two main isoforms of human CD117 are provided in SEQ ID NO: 145 (isoform 1) and SEQ ID NO: 146 (isoform 2).

As used herein, the term “bispecific antibody” refers to an antibody, for example, a monoclonal, a human or humanized antibody, that is capable of binding at least two different epitopes that can be on the same or different antigens. For instance, one of the binding specificities can be directed towards an epitope on a hematopoietic stem cell surface antigen, such as CD117 (e.g., GNNK+ CD117), and the other can specifically bind an epitope on a different hematopoietic stem cell surface antigen or another cell surface protein, such as a receptor or receptor subunit involved in a signal transduction pathway that potentiates cell growth, among others. In some embodiments, the binding specificities can be directed towards unique, non-overlapping epitopes on the same target antigen (i.e., a biparatopic antibody).

As used herein, the term “complementarity determining region” (CDR) refers to a hypervariable region found both in the light chain and the heavy chain variable domains of an antibody. The more highly conserved portions of variable domains are referred to as framework regions (FRs). The amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies described herein may contain modifications in these hybrid hypervariable positions. The variable domains of native heavy and light chains each contain four framework regions that primarily adopt a β-sheet configuration, connected by three CDRs, which form loops that connect, and in some cases form part of, the β-sheet structure. The CDRs in each chain are held together in close proximity by the framework regions in the order FR1-CDR1-FR2-CDR2-FR3-CDR3-FR4 and, with the CDRs from the other antibody chains, contribute to the formation of the target binding site of antibodies (see Kabat et al., Sequences of Proteins of Immunological Interest, National Institute of Health, Bethesda, Md., 1987). In certain embodiments, numbering of immunoglobulin amino acid residues is performed according to the immunoglobulin amino acid residue numbering system of Kabat et al., unless otherwise indicated (although any antibody numbering scheme, including, but not limited to IMGT and Chothia, can be utilized).

The term “de-immunized” or “de-immunization”, as used herein, relates to modification of an original wild type construct (or parent antibody) by rendering said wild type construct non-immunogenic or less immunogenic in humans. De-immunized antibodies contain part(s), e.g., a framework region(s) and/or a CDR(s), of non-human origin. As used herein, the term “deimmunized antibody” refers to an antibody that is de-immunized by mutation not to activate the immune system of a subject (for example, Nanus et al., J. Urology 170: S84-S89, 2003; WO98/52976; WO00/34317).

As used herein, the terms “condition” and “conditioning” refer to processes by which a patient is prepared for receipt of a transplant, e.g., a transplant containing hematopoietic stem cells. Such procedures promote the engraftment of a hematopoietic stem cell transplant (for instance, as inferred from a sustained increase in the quantity of viable hematopoietic stem cells within a blood sample isolated from a patient following a conditioning procedure and subsequent hematopoietic stem cell transplantation). According to the methods described herein, a patient may be conditioned for hematopoietic stem cell transplant therapy by administration to the patient of an ADC, antibody or antigen-binding fragment thereof capable of binding an antigen expressed by hematopoietic stem cells, such as CD117 (e.g., GNNK+ CD117). As described herein, the antibody may be covalently conjugated to a cytotoxin so as to form an antibody drug conjugate (ADC). Administration of an ADC, antibody, or antigen-binding fragment thereof, capable of binding one or more of the foregoing antigens to a patient in need of hematopoietic stem cell transplant therapy can promote the engraftment of a hematopoietic stem cell graft, for example, by selectively depleting endogenous hematopoietic stem cells, thereby creating a vacancy filled by an exogenous hematopoietic stem cell transplant.

As used herein, the term “conjugate” or “antibody drug conjugate” or “ADC” refers to an antibody which is linked to a cytotoxin. An ADC is formed by the chemical bonding of a reactive functional group of one molecule, such as an antibody or antigen-binding fragment thereof, with an appropriately reactive functional group of another molecule, such as a cytotoxin described herein. Conjugates may include a linker between the two molecules bound to one another, e.g., between an antibody and a cytotoxin. Examples of linkers that can be used for the formation of a conjugate include peptide-containing linkers, such as those that contain naturally occurring or non-naturally occurring amino acids, such as D-amino acids. Linkers can be prepared using a variety of strategies described herein and known in the art. Depending on the reactive components therein, a linker may be cleaved, for example, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012). Notably, the term “conjugate” (when referring to a compound) is also referred to interchangeably herein as a “drug conjugate”, “antibody drug conjugate” or “ADC”.

As used herein, the term “coupling reaction” refers to a chemical reaction in which two or more substituents suitable for reaction with one another react so as to form a chemical moiety that joins (e.g., covalently) the molecular fragments bound to each substituent. Coupling reactions include those in which a reactive substituent bound to a fragment that is a cytotoxin, such as a cytotoxin known in the art or described herein, reacts with a suitably reactive substituent bound to a fragment that is an antibody, or antigen-binding fragment thereof, such as an antibody, antigen-binding fragment thereof, or specific anti-CD117 antibody that binds CD117 (such as GNNK+CD117) known in the art or described herein. Examples of suitably reactive substituents include a nucleophile/electrophile pair (e.g., a thiol/haloalkyl pair, an amine/carbonyl pair, or a thiol/α,β-unsaturated carbonyl pair, among others), a diene/dienophile pair (e.g., an azide/alkyne pair, among others), and the like. Coupling reactions include, without limitation, thiol alkylation, hydroxyl alkylation, amine alkylation, amine condensation, amidation, esterification, disulfide formation, cycloaddition (e.g., [4+2] Diels-Alder cycloaddition, [3+2] Huisgen cycloaddition, among others), nucleophilic aromatic substitution, electrophilic aromatic substitution, and other reactive modalities known in the art or described herein.

As used herein, “CRU (competitive repopulating unit)” refers to a unit of measure of long-term engrafting stem cells, which can be detected after in-vivo transplantation.

As used herein, the term “donor” refers to a human or animal from which one or more cells are isolated prior to administration of the cells, or progeny thereof, into a recipient. The one or more cells may be, for example, a population of hematopoietic stem cells.

As used herein, the term “diabody” refers to a bivalent antibody containing two polypeptide chains, in which each polypeptide chain includes V_{H} and V_{L} domains joined by a linker that is too short (e.g., a linker composed of five amino acids) to allow for intramolecular association of V_{H} and V_{L} domains on the same peptide chain. This configuration forces each domain to pair with a complementary domain on another polypeptide chain so as to form a homodimeric structure. Accordingly, the term “triabody” refers to trivalent antibodies containing three peptide chains, each of which contains one V_{H} domain and one V_{L} domain joined by a linker that is exceedingly short (e.g., a linker composed of 1-2 amino acids) to permit intramolecular association of V_{H} and V_{L} domains within the same peptide chain. In order to fold into their native structures, peptides configured in this way typically trimerize so as to position the V_{H} and V_{L} domains of neighboring peptide chains spatially proximal to one another (see, for example, Holliger et al., Proc. Natl. Acad. Sci. USA 90:6444-48, 1993).

As used herein, “drug-to-antibody ratio” or “DAR” refers to the number of drugs, e.g., pyrrolobenzodiazepine, attached to the antibody of a conjugate. The DAR of an ADC can range from 1 to 8, although higher loads are also possible depending on the number of linkage sites on an antibody. In certain embodiments, the conjugate has a DAR of about 1, about 2, about 3, about 4, about 5, about 6, about 7, or about 8.

As used herein, the term “endogenous” describes a substance, such as a molecule, cell, tissue, or organ (e.g., a hematopoietic stem cell or a cell of hematopoietic lineage, such as a megakaryocyte, thrombocyte, platelet, erythrocyte, mast cell, myeloblast, basophil, neutrophil, eosinophil, microglial cell, granulocyte, monocyte, osteoclast, antigen-presenting cell, macrophage, dendritic cell, natural killer cell, T-lymphocyte, or B-lymphocyte) that is found naturally in a particular organism, such as a human patient.

As used herein, the term “engraftment potential” is used to refer to the ability of hematopoietic stem and progenitor cells to repopulate a tissue, whether such cells are naturally circulating or are provided by transplantation. The term encompasses all events surrounding or leading up to engraftment, such as tissue homing of cells and colonization of cells within the tissue of interest. The engraftment efficiency or rate of engraftment can be evaluated or quantified using any clinically acceptable parameter as known to those of skill in the art and can include, for example, assessment of competitive repopulating units (CRU); incorporation or expression of a marker in tissue(s) into which stem cells have homed, colonized, or become engrafted; or by evaluation of the progress of a subject through disease progression, survival of hematopoietic stem and progenitor cells, or survival of a recipient. Engraftment can also be determined by measuring white blood cell counts in peripheral blood during a post-transplant period. Engraftment can also be assessed by measuring recovery of marrow cells by donor cells in a bone marrow aspirate sample.

As used herein, the term “exogenous” describes a substance, such as a molecule, cell, tissue, or organ (e.g., a hematopoietic stem cell or a cell of hematopoietic lineage, such as a megakaryocyte, thrombocyte, platelet, erythrocyte, mast cell, myeloblast, basophil, neutrophil, eosinophil, microglial cell, granulocyte, monocyte, osteoclast, antigen-presenting cell, macrophage, dendritic cell, natural killer cell, T-lymphocyte, or B-lymphocyte) that is not found naturally in a particular organism, such as a human patient. A substance that is exogenous to a recipient organism, e.g., a recipient patient, may be naturally present in a donor organism, e.g., a donor subject, from which the substance is derived. For example, an allogeneic cell transplant contains cells that are exogenous to the recipient, but native to the donor. Exogenous substances include those that are provided from an external source to an organism or to cultured matter extracted therefrom.

The terms “Fc”, “Fc region,” and “Fc domain,” as used herein refer to the portion of an immunoglobulin, e.g., an IgG molecule, that correlates to a crystallizable fragment obtained by papain digestion of an IgG molecule. The Fc region comprises the C-terminal half of two heavy chains of an IgG molecule that are linked by disulfide bonds. It has no antigen binding activity but contains the carbohydrate moiety and binding sites for complement and Fc receptors, including the FcRn receptor (see below). For example, an Fc region contains the second constant domain CH2 (e.g., residues at EU positions 231-340 of IgG1) and the third constant domain CH3 (e.g., residues at EU positions 341-447 of human IgG1). As used herein, the Fc domain includes the “lower hinge region” (e.g., residues at EU positions 233-239 of IgG1).

Fc can refer to this region in isolation, or this region in the context of an antibody, antibody fragment, or Fc fusion protein. Polymorphisms have been observed at a number of positions in Fc domains, including but not limited to EU positions 270, 272, 312, 315, 356, and 358, and thus slight differences between the sequences presented in the instant application and sequences known in the art can exist. Thus, a “wild type IgG Fc domain” or “WT IgG Fc domain” refers to any naturally occurring IgG Fc region (i.e., any allele). The sequences of the heavy chains of human IgG1, IgG2, IgG3 and IgG4 can be found in a number of sequence databases, for example, at the Uniprot database (www.uniprot.org) under accession numbers P01857 (IGHG1_HUMAN), P01859 (IGHG2 HUMAN), P01860 (IGHG3 HUMAN), and P01861 (IGHG1_HUMAN), respectively. An example of a “WT” Fc region is provided in SEQ ID NO: 122 (which provides a heavy chain constant region containing an Fc region).

The terms “modified Fc region” or “variant Fc region” as used herein refers to an IgG Fc domain comprising one or more amino acid substitutions, deletions, insertions or modifications introduced at any position within the Fc region. In certain aspects a variant IgG Fc domain comprises one or more amino acid substitutions resulting in decreased or ablated binding affinity for an Fc gamma R and/or C1q as compared to the wild type Fc domain not comprising the one or more amino acid substitutions. Further, Fc binding interactions are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a variant Fc domain (e.g., an antibody, fusion protein or conjugate) can exhibit altered binding affinity for at least one or more Fc ligands (e.g., Fc gamma Rs) relative to a corresponding antibody otherwise having the same amino acid sequence but not comprising the one or more amino acid substitution, deletion, insertion or modifications such as, for example, an unmodified Fc region containing naturally occurring amino acid residues at the corresponding position in the Fc region.

Variant Fc domains are defined according to the amino acid modifications that compose them. For all amino acid substitutions discussed herein in regard to the Fc region, numbering is always according to the EU index as in Kabat. Thus, for example, D265C is an Fc variant with the aspartic acid (D) at EU position 265 substituted with cysteine (C) relative to the parent Fc domain. It is noted that the order in which substitutions are provided is arbitrary. Likewise, e.g., D265C/L234A/L235A defines a variant Fc variant with substitutions at EU positions 265 (D to C), 234 (L to A), and 235 (L to A) relative to the parent Fc domain. A variant can also be designated according to its final amino acid composition in the mutated EU amino acid positions. For example, the L234A/L235A mutant can be referred to as “LALA”. As a further example, the E233P.L234V.L235A.delG236 (deletion of 236) mutant can be referred to as “EPLVLAdelG”. As yet another example, the I253A.H310A.H435A mutant can be referred to as “IHH”. It is noted that the order in which substitutions are provided is arbitrary.

The terms “Fc gamma receptor” or “Fc gamma R” as used herein refer to any member of the family of proteins that bind the IgG antibody Fc region and are encoded by the Fc gamma R genes. In humans this family includes but is not limited to Fcg amma RI (CD64), including isoforms Fc gamma RIa, Fc gamma RIb, and Fc gamma RIc; Fc gamma RII (CD32), including isoforms Fc gamma RIIa (including allotypes H131 and R131), Fc gamma RIIb (including Fc gamma RIIb-1 and Fc gamma RIIb-2), and Fc gamma RIIc; and Fc gamma RIII (CD16), including isoforms Fc gamma RIIIa (including allotypes V158 and F158) and Fc gamma RIIIb (including allotypes Fc gamma RIIIb-NA1 and Fc gamma RIIIb-NA2), as well as any undiscovered human Fc gamma Rs or Fc gamma R isoforms or allotypes. An Fc gamma R can be from any organism, including but not limited to humans, mice, rats, rabbits, and monkeys. Mouse Fc gamma Rs include but are not limited to Fc gamma RI (CD64), Fc gamma RII (CD32), Fc gamma RIII (CD16), and Fc gamma RIII-2 (CD16-2), as well as any undiscovered mouse Fc gamma Rs or Fc gamma R isoforms or allotypes.

The term “effector function” as used herein refers to a biochemical event that results from the interaction of an Fc domain with an Fc receptor. Effector functions include but are not limited to ADCC, ADCP, and CDC. By “effector cell” as used herein is meant a cell of the immune system that expresses or one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and gamma delta T cells, and can be from any organism included but not limited to humans, mice, rats, rabbits, and monkeys.

The term “silent”, “silenced”, or “silencing” as used herein refers to an antibody having a modified Fc region described herein that has decreased binding to an Fc gamma receptor (FcγR) relative to binding of an identical antibody comprising an unmodified Fc region to the FcγR (e.g., a decrease in binding to a FcγR by at least 70%, at least 80%, at least 90%, at least 95%, at least 98%, at least 99%, or 100% relative to binding of the identical antibody comprising an unmodified Fc region to the FcγR as measured by, e.g., BLI). In some embodiments, the Fc silenced antibody has no detectable binding to an FcγR. Binding of an antibody having a modified Fc region to an FcγR can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE™ analysis or Octet™ analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

As used herein, the term “identical antibody comprising an unmodified Fc region” refers to an antibody that lacks the recited amino acid substitutions (e.g., D265C, H435A, L234A, and/or L235A), but otherwise has the same amino acid sequence as the Fc modified antibody to which it is being compared.

The terms “antibody-dependent cell-mediated cytotoxicity” or “ADCC” refer to a form of cytotoxicity in which a polypeptide comprising an Fc domain, e.g., an antibody, bound onto Fc receptors (FcRs) present on certain cytotoxic cells (e.g., primarily NK cells, neutrophils, and macrophages) and enables these cytotoxic effector cells to bind specifically to an antigen-bearing “target cell” and subsequently kill the target cell with cytotoxins. (Hogarth et al., Nature review Drug Discovery 2012, 11:313) It is contemplated that, in addition to antibodies and fragments thereof, other polypeptides comprising Fc domains, e.g., Fc fusion proteins and Fc conjugate proteins, having the capacity to bind specifically to an antigen-bearing target cell will be able to effect cell-mediated cytotoxicity.

For simplicity, the cell-mediated cytotoxicity resulting from the activity of a polypeptide comprising an Fc domain is also referred to herein as ADCC activity. The ability of any particular polypeptide of the present disclosure to mediate lysis of the target cell by ADCC can be assayed. To assess ADCC activity, a polypeptide of interest (e.g., an antibody) is added to target cells in combination with immune effector cells, resulting in cytolysis of the target cell. Cytolysis is generally detected by the release of label (e.g., radioactive substrates, fluorescent dyes or natural intracellular proteins) from the lysed cells. Useful effector cells for such assays include peripheral blood mononuclear cells (PBMC) and Natural Killer (NK) cells. Specific examples of in vitro ADCC assays are described in Bruggemann et al., J. Exp. Med. 166:1351 (1987); Wilkinson et al., J. Immunol. Methods 258:183 (2001); Patel et al., J. Immunol. Methods 184:29 (1995). Alternatively, or additionally, ADCC activity of the antibody of interest can be assessed in vivo, e.g., in an animal model such as that disclosed in Clynes et al., Proc. Natl. Acad. Sci. USA 95:652 (1998).

The terms “full length antibody” or “intact antibody” are used herein interchangeably to refer to an antibody in its substantially intact form, and not an antibody fragment as defined herein. Thus, for an IgG antibody, an intact antibody comprises two heavy chains each comprising a variable region, a constant region and an Fc region, and two light chains each comprising a variable region and a constant region. More specifically, an intact IgG comprises two light chains each comprising a light chain variable region (VL) and a light chain constant region (CL), and comprises two heavy chains each comprising a heavy chain variable region (VH) and three heavy chain constant regions (CH1, CH2, and CH3). CH2 and CH3 represent the Fc region of the heavy chain.

As used herein, the term “framework region” or “FW region” includes amino acid residues that are adjacent to the CDRs of an antibody or antigen-binding fragment thereof. FW region residues may be present in, for example, human antibodies, humanized antibodies, monoclonal antibodies, antibody fragments, Fab fragments, single chain antibody fragments, scFv fragments, antibody domains, and bispecific antibodies, among others.

Also provided are “conservative sequence modifications” of the sequences set forth in SEQ ID NOs described herein, i.e., nucleotide and amino acid sequence modifications which do not abrogate the binding of the antibody encoded by the nucleotide sequence or containing the amino acid sequence, to the antigen. Such conservative sequence modifications include conservative nucleotide and amino acid substitutions, as well as, nucleotide and amino acid additions and deletions. For example, modifications can be introduced into SEQ ID NOs described herein by standard techniques known in the art, such as site-directed mutagenesis and PCR-mediated mutagenesis. Conservative sequence modifications include conservative amino acid substitutions, in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an anti-CD117 antibody is preferably replaced with another amino acid residue from the same side chain family. Methods of identifying nucleotide and amino acid conservative substitutions that do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)).

As used herein, the term “half-life” refers to the time it takes for the plasma concentration of the antibody drug in the body to be reduced by one half or 50% in a subject, e.g., a human subject. This 50% reduction in serum concentration reflects the amount of drug circulating.

As used herein, the term “hematopoietic stem cells” (“HSCs”) refers to immature blood cells having the capacity to self-renew and to differentiate into mature blood cells containing diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Such cells may include CD34⁺ cells. CD34⁺ cells are immature cells that express the CD34 cell surface marker. In humans, CD34+ cells are believed to include a subpopulation of cells with the stem cell properties defined above, whereas in mice, HSCs are CD34−. In addition, HSCs also refer to long term repopulating HSCs (LT-HSC) and short term repopulating HSCs (ST-HSC). LT-HSCs and ST-HSCs are differentiated, based on functional potential and on cell surface marker expression. For example, human HSCs are CD34+, CD38−, CD45RA−, CD90+, CD49F+, and lin− (negative for mature lineage markers including CD2, CD3, CD4, CD7, CD8, CD10, CD11B, CD19, CD20, CD56, CD235A). In mice, bone marrow LT-HSCs are CD34−, SCA−1+, C-kit+, CD135−, Slamfl/CD150+, CD48−, and lin− (negative for mature lineage markers including Ter119, CD11b, Gr1, CD3, CD4, CD8, B220, IL7ra), whereas ST-HSCs are CD34+, SCA-1+, C-kit+, CD135−, Slamfl/CD150+, and lin− (negative for mature lineage markers including Ter119, CD11b, Gr1, CD3, CD4, CD8, B220, IL7ra). In addition, ST-HSCs are less quiescent and more proliferative than LT-HSCs under homeostatic conditions. However, LT-HSC have greater self-renewal potential (i.e., they survive throughout adulthood, and can be serially transplanted through successive recipients), whereas ST-HSCs have limited self-renewal (i.e., they survive for only a limited period of time, and do not possess serial transplantation potential). Any of these HSCs can be used in the methods described herein. ST-HSCs are particularly useful because they are highly proliferative and thus, can more quickly give rise to differentiated progeny.

As used herein, the term “hematopoietic stem cell functional potential” refers to the functional properties of hematopoietic stem cells which include 1) multi-potency (which refers to the ability to differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal (which refers to the ability of hematopoietic stem cells to give rise to daughter cells that have equivalent potential as the mother cell, and further that this ability can repeatedly occur throughout the lifetime of an individual without exhaustion), and 3) the ability of hematopoietic stem cells or progeny thereof to be reintroduced into a transplant recipient whereupon they home to the hematopoietic stem cell niche and re-establish productive and sustained hematopoiesis.

As used herein, the term “human antibody” is intended to include antibodies having variable and constant regions derived from human germline immunoglobulin sequences. A human antibody may include amino acid residues not encoded by human germline immunoglobulin sequences (e.g., mutations introduced by random or site-specific mutagenesis in vitro or during gene rearrangement or by somatic mutation in vivo). However, the term “human antibody”, as used herein, is not intended to include antibodies in which CDR sequences derived from the germline of another mammalian species, such as a mouse, have been grafted onto human framework sequences. A human antibody can be produced in a human cell (for example, by recombinant expression) or by a non-human animal or a prokaryotic or eukaryotic cell that is capable of expressing functionally rearranged human immunoglobulin (such as heavy chain and/or light chain) genes. When a human antibody is a single chain antibody, it can include a linker peptide that is not found in native human antibodies. For example, an Fv can contain a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. Human antibodies can also be produced using transgenic mice that are incapable of expressing functional endogenous immunoglobulins, but which can express human immunoglobulin genes (see, for example, PCT Publication Nos. WO 1998/24893; WO 1992/01047; WO 1996/34096; WO 1996/33735; U.S. Pat. Nos. 5,413,923; 5,625,126; 5,633,425; 5,569,825; 5,661,016; 5,545,806; 5,814,318; 5,885,793; 5,916,771; and 5,939,598).

“Humanized” forms of non-human (e.g., murine or rat) antibodies are immunoglobulins that contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence A humanized antibody can also comprise at least a portion of an immunoglobulin constant region (Fc), typically that of a human immunoglobulin consensus sequence. Methods of antibody humanization are known in the art and have been described, for example, in Riechmann et al., Nature 332:323-7, 1988; U.S. Pat. Nos. 5,530,101; 5,585,089; 5,693,761; 5,693,762; and U.S. Pat. No. 6,180,370 to Queen et al.; EP239400; PCT publication WO 91/09967; U.S. Pat. No. 5,225,539; EP592106; EP519596; Padlan, 1991, Mol. Immunol., 28:489-498; Studnicka et al., 1994, Prot. Eng. 7:805-814; Roguska et al., 1994, Proc. Natl. Acad. Sci. 91:969-973; and U.S. Pat. No. 5,565,33.

As used herein, patients that are “in need of” a hematopoietic stem cell transplant include patients that exhibit a defect or deficiency in one or more blood cell types, as well as patients having a stem cell disorder, autoimmune disease, cancer, or other pathology described herein. Hematopoietic stem cells generally exhibit 1) multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal, and can thus give rise to daughter cells that have equivalent potential as the mother cell, and 3) the ability to be reintroduced into a transplant recipient whereupon they home to the hematopoietic stem cell niche and re-establish productive and sustained hematopoiesis. Hematopoietic stem cells can thus be administered to a patient defective or deficient in one or more cell types of the hematopoietic lineage in order to re-constitute the defective or deficient population of cells in vivo. For example, the patient may be suffering from cancer, and the deficiency may be caused by administration of a chemotherapeutic agent or other medicament that depletes, either selectively or non-specifically, the cancerous cell population. Additionally or alternatively, the patient may be suffering from a hemoglobinopathy (e.g., a non-malignant hemoglobinopathy), such as sickle cell anemia, thalassemia, Fanconi anemia, aplastic anemia, and Wiskott-Aldrich syndrome. The subject may be one that is suffering from adenosine deaminase severe combined immunodeficiency (ADA SCID), HIV/AIDS, metachromatic leukodystrophy, Diamond-Blackfan anemia, and Schwachman-Diamond syndrome. The subject may have or be affected by an inherited blood disorder (e.g., sickle cell anemia) or an autoimmune disorder. Additionally or alternatively, the subject may have or be affected by a malignancy, such as neuroblastoma or a hematologic cancer. For instance, the subject may have a leukemia, lymphoma, or myeloma. In some embodiments, the subject has acute myeloid leukemia, acute lymphoid leukemia, chronic myeloid leukemia, chronic lymphoid leukemia, multiple myeloma, diffuse large B-cell lymphoma, or non-Hodgkin's lymphoma. In some embodiments, the subject has myelodysplastic syndrome. In some embodiments, the subject has an autoimmune disease, such as scleroderma, multiple sclerosis, ulcerative colitis, Crohn's disease, Type 1 diabetes, or another autoimmune pathology described herein. In some embodiments, the subject is in need of chimeric antigen receptor T-cell (CART) therapy. In some embodiments, the subject has or is otherwise affected by a metabolic storage disorder. The subject may suffer or otherwise be affected by a metabolic disorder selected from the group consisting of glycogen storage diseases, mucopolysaccharidoses, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy. Additionally or alternatively, a patient “in need of” a hematopoietic stem cell transplant may one that is or is not suffering from one of the foregoing pathologies, but nonetheless exhibits a reduced level (e.g., as compared to that of an otherwise healthy subject) of one or more endogenous cell types within the hematopoietic lineage, such as megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes. One of skill in the art can readily determine whether one's level of one or more of the foregoing cell types, or other blood cell type, is reduced with respect to an otherwise healthy subject, for instance, by way of flow cytometry and fluorescence activated cell sorting (FACS) methods, among other procedures, known in the art.

As used herein a “neutral antibody” refers to an antibody, or an antigen binding fragment thereof, that is not capable of significantly neutralizing, blocking, inhibiting, abrogating, reducing or interfering with the activities of a particular or specified target (e.g., CD117), including the binding of receptors to ligands or the interactions of enzymes with substrates. In one embodiment, a neutral anti-CD117 antibody, or fragment thereof, is an anti-CD117 antibody that does not substantially inhibit SCF-dependent cell proliferation and does not cross block SCF binding to CD117. An example of a neutral antibody is Ab67 (or an antibody having the binding regions of Ab67). In contrast, an “antagonist” anti-CD117 antibody inhibits SCF-dependent proliferation and is able to cross block SCF binding to CD117. An example of an antagonist antibody is Ab55 (or an antibody having the binding regions of Ab55).

As used herein, the term “recipient” refers to a patient that receives a transplant, such as a transplant containing a population of hematopoietic stem cells. The transplanted cells administered to a recipient may be, e.g., autologous, syngeneic, or allogeneic cells.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) taken from a subject.

As used herein, the term “scFv” refers to a single chain Fv antibody in which the variable domains of the heavy chain and the light chain from an antibody have been joined to form one chain. scFv fragments contain a single polypeptide chain that includes the variable region of an antibody light chain (V_{L}) (e.g., CDR-L1, CDR-L2, and/or CDR-L3) and the variable region of an antibody heavy chain (V_{H}) (e.g., CDR-H1, CDR-H2, and/or CDR-H3) separated by a linker. The linker that joins the V_{L} and V_{H} regions of a scFv fragment can be a peptide linker composed of proteinogenic amino acids. Alternative linkers can be used to so as to increase the resistance of the scFv fragment to proteolytic degradation (for example, linkers containing D-amino acids), in order to enhance the solubility of the scFv fragment (for example, hydrophilic linkers such as polyethylene glycol-containing linkers or polypeptides containing repeating glycine and serine residues), to improve the biophysical stability of the molecule (for example, a linker containing cysteine residues that form intramolecular or intermolecular disulfide bonds), or to attenuate the immunogenicity of the scFv fragment (for example, linkers containing glycosylation sites). It will also be understood by one of ordinary skill in the art that the variable regions of the scFv molecules described herein can be modified such that they vary in amino acid sequence from the antibody molecule from which they were derived. For example, nucleotide or amino acid substitutions leading to conservative substitutions or changes at amino acid residues can be made (e.g., in CDR and/or framework residues) so as to preserve or enhance the ability of the scFv to bind to the antigen recognized by the corresponding antibody.

The terms “specific binding” or “specifically binds”, as used herein, refers to the ability of an antibody (or ADC) to recognize and bind to a specific protein structure (epitope) rather than to proteins generally. If an antibody is specific for epitope “A”, the presence of a molecule containing epitope A (or free, unlabeled A), in a reaction containing labeled “A” and the antibody, will reduce the amount of labeled A bound to the antibody. By way of example, an antibody “binds specifically” to a target if the antibody, when labeled, can be competed away from its target by the corresponding non-labeled antibody. In one embodiment, an antibody specifically binds to a target, e.g., CD117, if the antibody has a K_{D} for the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, or less (less meaning a number that is less than 10⁻¹², e.g. 10⁻¹³). In one embodiment, the term “specific binding to CD117” or “specifically binds to CD117,” as used herein, refers to an antibody (or ADC) that binds to CD117 and has a dissociation constant (K_{D}) of 1.0×10⁻⁷ M or less, as determined by surface plasmon resonance. In one embodiment, K_{D} (M) is determined according to standard bio-layer interferometery (BLI). In one embodiment, K_{off} (1/s) is determined according to standard bio-layer interferometery (BLI). It shall be understood, however, that the antibody may be capable of specifically binding to two or more antigens which are related in sequence. For example, in one embodiment, an antibody can specifically bind to both human and a non-human (e.g., mouse or non-human primate) orthologs of CD117.

As used herein, the terms “subject” and “patient” refer to an organism, such as a human, that receives treatment for a particular disease or condition as described herein. For instance, a patient, such as a human patient, may receive treatment prior to hematopoietic stem cell transplant therapy in order to promote the engraftment of exogenous hematopoietic stem cells.

As used herein, the phrase “substantially cleared from the blood” refers to a point in time following administration of a therapeutic agent, e.g., an ADC comprising a pyrrolobenzodiazepine, to a patient when the concentration of the therapeutic agent in a blood sample isolated from the patient is such that the therapeutic agent is not detectable by conventional means (for instance, such that the therapeutic agent is not detectable above the noise threshold of the device or assay used to detect the therapeutic agent). A variety of techniques known in the art can be used to detect antibodies, or antibody fragments, such as ELISA-based detection assays known in the art or described herein. Additional assays that can be used to detect antibodies, or antibody fragments, include immunoprecipitation techniques and immunoblot assays, among others known in the art.

As used herein, the phrase “stem cell disorder” broadly refers to any disease, disorder, or condition that may be treated or cured by conditioning a subject's target tissues, and/or by ablating an endogenous stem cell population in a target tissue (e.g., ablating an endogenous hematopoietic stem or progenitor cell population from a subject's bone marrow tissue) and/or by engrafting or transplanting stem cells in a subject's target tissues. For example, Type I diabetes has been shown to be cured by hematopoietic stem cell transplant and may benefit from conditioning in accordance with the compositions and methods described herein. Additional disorders that can be treated using the compositions and methods described herein include, without limitation, sickle cell anemia, thalassemias, Fanconi anemia, aplastic anemia, Wiskott-Aldrich syndrome, ADA SCID, HIV/AIDS, metachromatic leukodystrophy, Diamond-Blackfan anemia, and Schwachman-Diamond syndrome. Additional diseases that may be treated using the patient conditioning and/or hematopoietic stem cell transplant methods described herein include inherited blood disorders (e.g., sickle cell anemia) and autoimmune disorders, such as scleroderma, multiple sclerosis, ulcerative colitis, and Crohn's disease. Additional diseases that may be treated using the conditioning and/or transplantation methods described herein include a malignancy, such as a neuroblastoma or a hematologic cancer, such as leukemia, lymphoma, and myeloma. For instance, the cancer may be acute myeloid leukemia, acute lymphoid leukemia, chronic myeloid leukemia, chronic lymphoid leukemia, multiple myeloma, diffuse large B-cell lymphoma, or non-Hodgkin's lymphoma. Additional diseases treatable using the conditioning and/or transplantation methods described herein include myelodysplastic syndrome. In some embodiments, the subject has or is otherwise affected by a metabolic storage disorder. For example, the subject may suffer or otherwise be affected by a metabolic disorder selected from the group consisting of glycogen storage diseases, mucopolysaccharidoses, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy.

As used herein, the term “transfection” refers to any of a wide variety of techniques commonly used for the introduction of exogenous DNA into a prokaryotic or eukaryotic host cell, such as electroporation, lipofection, calcium-phosphate precipitation, DEAE-dextran transfection and the like.

As used herein, the terms “treat” or “treatment” refers to reducing the severity and/or frequency of disease symptoms, eliminating disease symptoms and/or the underlying cause of said symptoms, reducing the frequency or likelihood of disease symptoms and/or their underlying cause, and improving or remediating damage caused, directly or indirectly, by disease, any improvement of any consequence of disease, such as prolonged survival, less morbidity, and/or a lessening of side effects which are the byproducts of an alternative therapeutic modality; as is readily appreciated in the art, full eradication of disease is a preferred but albeit not a requirement for a treatment act. Beneficial or desired clinical results include, but are not limited to, promoting the engraftment of exogenous hematopoietic cells in a patient following antibody conditioning therapy as described herein and subsequent hematopoietic stem cell transplant therapy. Additional beneficial results include an increase in the cell count or relative concentration of hematopoietic stem cells in a patient in need of a hematopoietic stem cell transplant following conditioning therapy and subsequent administration of an exogenous hematopoietic stem cell graft to the patient. Beneficial results of therapy described herein may also include an increase in the cell count or relative concentration of one or more cells of hematopoietic lineage, such as a megakaryocyte, thrombocyte, platelet, erythrocyte, mast cell, myeloblast, basophil, neutrophil, eosinophil, microglial cell, granulocyte, monocyte, osteoclast, antigen-presenting cell, macrophage, dendritic cell, natural killer cell, T-lymphocyte, or B-lymphocyte, following conditioning therapy and subsequent hematopoietic stem cell transplant therapy. Additional beneficial results may include the reduction in quantity of a disease-causing cell population, such as a population of cancer cells (e.g., CD117+ leukemic cells) or autoimmune cells (e.g., CD117+ autoimmune lymphocytes, such as a CD117+ T-cell that expresses a T-cell receptor that cross-reacts with a self-antigen). Insofar as the methods of the present disclosure are directed to preventing disorders, it is understood that the term “prevent” does not require that the disease state be completely thwarted. Rather, as used herein, the term preventing refers to the ability of the skilled artisan to identify a population that is susceptible to disorders, such that administration of the compounds of the present disclosure may occur prior to onset of a disease. The term does not imply that the disease state is completely avoided.

The term “effective amount” refers to the amount or dose of a therapeutic agent, e.g., an anti-CD117 antibody or an anti-CD117 ADC, which is sufficient to result in the desired outcome.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material.

As used herein, the term “vector” includes a nucleic acid vector, such as a plasmid, a DNA vector, a plasmid, a RNA vector, virus, or other suitable replicon. Expression vectors described herein may contain a polynucleotide sequence as well as, for example, additional sequence elements used for the expression of proteins and/or the integration of these polynucleotide sequences into the genome of a mammalian cell. Certain vectors that can be used for the expression of antibodies and antibody fragments of the present disclosure include plasmids that contain regulatory sequences, such as promoter and enhancer regions, which direct gene transcription. Other useful vectors for expression of antibodies and antibody fragments contain polynucleotide sequences that enhance the rate of translation of these genes or improve the stability or nuclear export of the mRNA that results from gene transcription. These sequence elements may include, for example, 5′ and 3′ untranslated regions and a polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, and nourseothricin.

### Antibody-Drug Conjugates (ADCs)

Antibodies, and antigen-binding fragments thereof that bind CD117 as described herein can be conjugated (linked) to a cytotoxic molecule (i.e., a cytotoxin such as a pyrrolobenzodiazepine (PBD)), thus forming an antibody-drug conjugate (ADC). As used herein, the terms “cytotoxin”, “cytotoxic moiety”, and “drug” are used interchangeably.

In particular, the ADCs as disclosed herein include an antibody that binds CD117 (including an antigen-binding fragment thereof) conjugated (i.e., covalently attached by a linker) to a cytotoxic moiety (e.g., a PBD), wherein the cytotoxic moiety, when not conjugated to an antibody moiety, has a cytotoxic or cytostatic effect. In various embodiments, the cytotoxic moiety exhibits reduced or no cytotoxicity when bound in a conjugate, but resumes cytotoxicity after cleavage from the linker. In various embodiments, the cytotoxic moiety maintains cytotoxicity without cleavage from the linker. In some embodiments, the cytotoxic molecule is conjugated to a cell internalizing antibody, or antigen-binding fragment thereof as disclosed herein, such that following the cellular uptake of the antibody, or fragment thereof, the cytotoxin may access its intracellular target and, e.g., mediate hematopoietic cell death. ADCs of the present disclosure therefore may be of the general formula

Ab-(Z-L-Cy)ₙ,

wherein an antibody or antigen-binding fragment thereof (Ab) is conjugated (covalently linked) to linker (L), through a chemical moiety (Z), to a cytotoxic moiety (Cy).

Accordingly, the antibody or antigen-binding fragment thereof may be conjugated to a number of drug moieties as indicated by integer n, which represents the average number of cytotoxins per antibody, which may range, e.g., from about 1 to about 20. In some embodiments, n is from 1 to 4. In some embodiments, n is 2. In some embodiments, n is 1. The average number of drug moieties per antibody in preparations of ADC from conjugation reactions may be characterized by conventional means such as mass spectroscopy, ELISA assay, and HPLC. The quantitative distribution of ADC in terms of n may also be determined. In some instances, separation, purification, and characterization of homogeneous ADC where n is a certain value from ADC with other drug loadings may be achieved by means such as reverse phase HPLC or electrophoresis.

For some antibody-drug conjugates, n may be limited by the number of attachment sites on the antibody. For example, where the attachment is a cysteine thiol, an antibody may have only one or several cysteine thiol groups, or may have only one or several sufficiently reactive thiol groups through which a linker may be attached. Generally, antibodies do not contain many free and reactive cysteine thiol groups which may be linked to a drug moiety; primarily, cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups.

In certain embodiments, fewer than the theoretical maximum of drug moieties are conjugated to an antibody during a conjugation reaction. An antibody may contain, for example, lysine residues that do not react with the drug-linker intermediate or linker reagent, as discussed below. Only the most reactive lysine groups may react with an amine-reactive linker reagent. In certain embodiments, an antibody is subjected to denaturing conditions to reveal reactive nucleophilic groups such as lysine or cysteine.

The loading (drug/antibody ratio) of an ADC may be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments.

### Cytotoxins

Anti-CD117 antibodies, and antigen-binding fragments thereof, as described herein can be conjugated (linked) to a cytotoxin. In some embodiments, the cytotoxin is a pyrrolobenzodiazepine (PBD).

### Pyrrolobenzodiazepines (PBDs)

In some embodiments, the antibodies, or antigen-binding fragments thereof, that bind CD117 as described herein can be conjugated to a cytotoxin that is a pyrrolobenzodiazepine (PBD) or a cytotoxin that comprises a PBD. PBDs are natural products produced by certain actinomycetes and have been shown to be sequence selective DNA alkylating compounds. PBD cytotoxins include, but are not limited to, anthramycin, dimeric PBDs, and those disclosed in, for example, Hartley, J A (2011) The development of pyrrolobenzodiazepines as antitumor agents. Expert Opin Inv Drug, 20(6), 733-744 and Antonow D, Thurston D E (2011) Synthesis of DNA-interactive pyrrolo[2,1-c][1,4]benzodiazepines (PBDs). Chem Rev 111: 2815-2864.

PBDs are of the general structure:

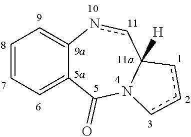

They differ in the number, type and position of substituents, in both their aromatic (“A”) rings and pyrrolo (“C”) rings, and in the degree of saturation of the C ring. In the diazepine B-ring there is either an imine (N═C), a carbinolamine (NH—CH(OH)), or a carbinolamine methyl ether (NH—CH(OMe)) at the N10-C11 position. This position is the electrophilic moiety responsible for DNA alkylation. All of the known natural product PDBs have an (S)-configuration at the chiral C11a position which provides them with a right-handed twist when viewed from the C ring towards the A ring. This provides the appropriate three-dimensional shape for isohelicity with the minor groove of B-form DNA, leading to a tight fit at the binding site (Kohn, In Antibiotics III. Springer-Verlag, New York, pp. 3-11 (1975); Hurley and Needham-VanDevanter, Acc. Chem. Res., 19, 230-237 (1986)). The ability of PDBs to form an adduct in the minor groove enables them to interfere with DNA processing, resulting in anti-tumor activity.

It has been previously disclosed that the biological activity of these molecules can be potentiated by joining two PBD units together through their C₈-hydroxyl functionalities via a flexible alkylene linker (Bose, D. S., et al., J. Am. Chem. Soc., 114, 4939-4941 (1992); Thurston, D. E., et al., J. Org. Chem., 61, 8141-8147 (1996)). The PBD dimers are thought to form sequence-selective DNA lesions, such as the palindromic 5′-Pu-GATC-Py-3′ inter-strand cross-link (Smellie, M., et al., Biochemistry, 42, 8232-8239 (2003); Martin, C., et al., Biochemistry, 44, 4135-4147) which is thought to be mainly responsible for their biological activity. An advantageous dimeric pyrrolobenzodiazepine compound has been described by Gregson et al. (Chem. Commun. 1999, 797-798; “compound 1”, and by Gregson et al. (J. Med. Chem. 2001, 44, 1161-1174; “compound 4a”). This compound, also known as SG2000, is of the structural formula:

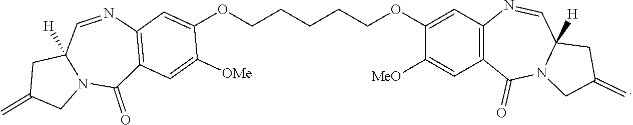

Generally, modifications to the pyrrolidine alkene moiety provide the handle with which to covalently bond the linking moiety and, hence the antibodies or antigen-binding fragments thereof (-L-Z′ and -L-Z-Ab, respectively, as described herein). Alternatively, a linker may be attached at position N10.

In some embodiments, the cytotoxin is a pyrrolobenzodiazepine dimer represented by the structural formula:

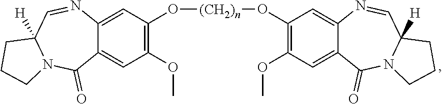

wherein n is an integer from 2 to 5. The compound of this formula wherein n is 3 is known as DSB-120 (Bose et al., J. Am. Chem. Soc. 1992, 114, 4939-4941).

In some embodiments, the cytotoxin is a pyrrolobenzodiazepine dimer represented by the structural formula:

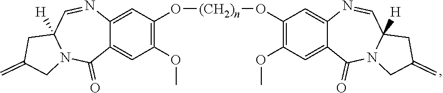

wherein n is an integer from 2 to 5. The compound of this formula wherein n is 3 is known as SJG-136 (Gregson et al., J. Med. Chem. 2001, 44, 737-748). The compound of this formula wherein n is 5 is known as DRG-16 (Gregson et al., Med. Chem. 2004; 47:1161-1174).

In some embodiments, the cytotoxin is a pyrrolobenzodiazepine dimer represented by the structural formula:

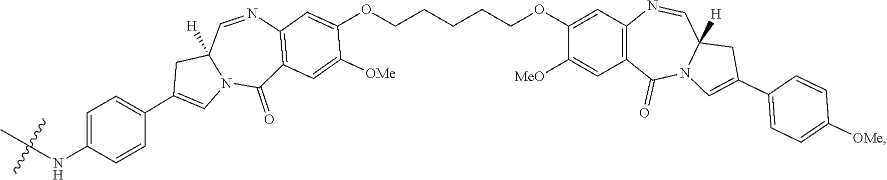 [SMILES: *Nc1ccc(C2=CN3C(=O)c4cc(OC)c(OCCCCCOc5cc6c(cc5OC)C(=O)N5C=C(c7ccc(OC)cc7)C[C@H]5C=N6)cc4N=C[C@@H]3C2)cc1]

wherein the wavy line indicates the point of covalent attachment to the linker of the ADC as described herein. ADCs based on this PBD are disclosed in, for example, Sutherland et al., Blood 2013 122:1455-1463, which is incorporated by reference herein in its entirety.

In some embodiments, the cytotoxin is a PBD dimer represented by the structural formula:

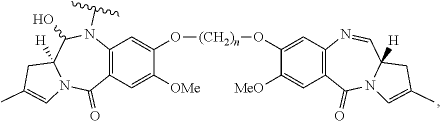

wherein n is 3 or 5, and wherein the wavy line indicates the point of covalent attachment to the linker of the ADC as described herein.

In some embodiments, the cytotoxin is a PBD dimer represented by the structural formula (I):

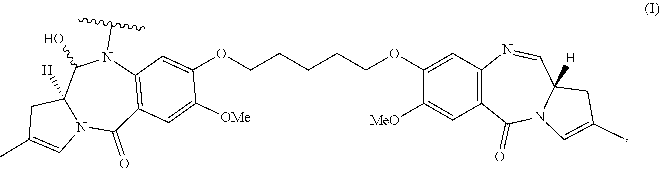

wherein the wavy line indicates the point of covalent attachment to the linker of the ADC as described herein.

In some embodiments, the cytotoxin is an indolinobenzodiazepine pseudodimer having the structure of formula:

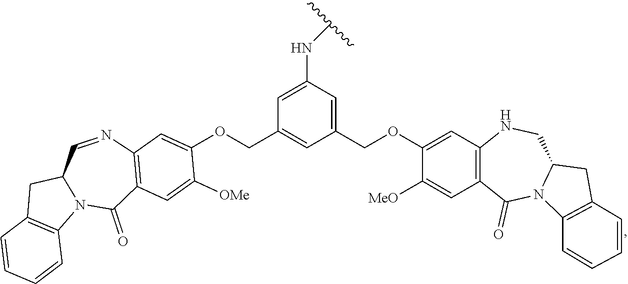 [SMILES: *Nc1cc(COc2cc3c(cc2OC)C(=O)N2c4ccccc4C[C@H]2C=N3)cc(COc2cc3c(cc2OC)C(=O)N2c4ccccc4C[C@H]2CN3)c1]

wherein the wavy line indicates the attachment point of the linker.

### Linkers

The term “Linker” as used herein means a divalent chemical moiety comprising a covalent bond or a chain of atoms that covalently attaches an anti-CD117 antibody or fragment thereof (Ab) to a cytotoxin (e.g., a PDB) to form an antibody-drug conjugate (ADC).

Covalent attachment of the antibody and the drug moiety requires the linker to have two reactive functional groups, i.e. bivalency in a reactive sense. Bivalent linker reagents which are useful to attach two or more functional or biologically active moieties, such as peptides, nucleic acids, drugs, toxins, antibodies, haptens, and reporter groups are known, and methods have been described their resulting conjugates (Hermanson, G. T. (1996) Bioconjugate Techniques; Academic Press: New York, p. 234-242).

Accordingly, present linkers have two reactive termini, one for conjugation to an antibody and the other for conjugation to a cytotoxin. The antibody conjugation reactive terminus of the linker (reactive moiety, defined herein as Z′) is typically a chemical moiety that is capable of conjugation to the antibody through, e.g., a cysteine thiol or lysine amine group on the antibody, and so is typically a thiol-reactive group such as a Michael acceptor (as in maleimide), a leaving group, such as a chloro, bromo, iodo, or an R-sulfanyl group, or an amine-reactive group such as a carboxyl group. Conjugation of the linker to the antibody is described more fully herein below.

The cytotoxin conjugation reactive terminus of the linker is typically a chemical moiety that is capable of conjugation to the cytotoxin through formation of a bond with a reactive substituent within the cytotoxin molecule. Non-limiting examples include, for example, formation of an amide bond with a basic amine or carboxyl group on the cytotoxin, via a carboxyl or basic amine group on the linker, respectively, or formation of an ether, amide, or the like, via alkylation of an OH or NH group, respectively, on the cytotoxin.

When the term “linker” is used in describing the linker in conjugated form, one or both of the reactive termini will be absent (such as reactive moiety Z′, having been converted to chemical moiety Z, as described herein below) or incomplete (such as being only the carbonyl of the carboxylic acid) because of the formation of the bonds between the linker and/or the cytotoxin, and between the linker and/or the antibody or antigen-binding fragment thereof. Such conjugation reactions are described further herein below.

A variety of linkers can be used to conjugate the antibodies, antigen-binding fragments, and ligands described to a cytotoxic molecule. Generally, linkers suitable for the present disclosure may be substantially stable in circulation, but allow for release of pyrrolobenzodiazepine within or in close proximity to the target cells. In some embodiments, certain linkers suitable for the present disclosure may be categorized as “cleavable” or “non-cleavable”. Generally, cleavable linkers contain one or more functional groups that is cleaved in response to a physiological environment. For example, a cleavable linker may contain an enzymatic substrate (e.g., valine-alanine) that degrades in the presence of an intracellular enzyme (e.g., cathepsin B), an acid-cleavable group (e.g., a hydrozone) that degrades in the acidic environment of a cellular compartment, or a reducible group (e.g., a disulfide) that degrades in an intracellular reducing environment. By contast, generally, non-cleavable linkers are released from the ADC during degradation (e.g., lysosomal degradation) of the antibody moiety of the ADC inside the target cell.

### Non-Cleavable Linkers

Non-cleavable linkers suitable for use herein further may include one or more groups selected from a bond, —(C═O)—, C₁-C₁₂ alkylene, C₁-C₁₂ heteroalkylene, C₂-C₁₂ alkenylene, C₂-C₁₂ heteroalkenylene, C₂-C₁₂ alkynylene, C₂-C₁₂ heteroalkynylene, C₃-C₁₂ cycloalkylene, heterocycloalkylene, arylene, heteroarylene, and combinations thereof, each of which may be optionally substituted, and/or may include one or more heteroatoms (e.g., S, N, or O) in place of one or more carbon atoms. Non-limiting examples of such groups include alkylene (CH₂)ₚ, (C═O)(CH₂)ᵣ, and polyethyleneglycol (PEG; (CH₂CH₂O)_{q}), units, —(NHCH₂CH₂)ᵤ, wherein each of p, q, r, t, and u are integers from 1-12, selected independently for each occurrence.

In some embodiments, the linker L comprises one or more of a bond, —(C═O)—, a —C(O)NH— group, an —OC(O)NH— group, C₁-C₁₂ alkylene, C₁-C₁₂ heteroalkylene, C₂-C₁₂ alkenylene, C₂-C₁₂ heteroalkenylene, C₂-C₁₂ alkynylene, C₂-C₁₂ heteroalkynylene, C₃-C₁₂ cycloalkylene, heterocycloalkylene, arylene, heteroarylene, or a —(CH₂CH₂O)_{q}— group where q is an integer from 1-12;

wherein each C₁-C₁₂ alkylene, C₁-C₁₂ heteroalkylene, C₂-C₁₂ alkenylene, C₂-C₁₂ heteroalkenylene, C₂-C₁₂ alkynylene, C₂-C₁₂ heteroalkynylene, C₃-C₁₂ cycloalkylene, heterocycloalkylene, arylene, or heteroarylene may optionally be substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro;

In some embodiments, each C₁-C₁₂ alkylene, C₁-C₁₂ heteroalkylene, C₂-C₁₂ alkenylene, C₂-C₁₂ heteroalkenylene, C₂-C₁₂ alkynylene, C₂-C₁₂ heteroalkynylene, C₃-C₁₂ cycloalkylene, heterocycloalkylene, arylene, or heteroarylene may optionally be interrupted by one or more heteroatoms selected from O, S and N.

In some embodiments, each C₁-C₆ alkylene, C₁-C₁₂ heteroalkylene, C₂-C₁₂ alkenylene, C₂-C₁₂ heteroalkenylene, C₂-C₁₂ alkynylene, C₂-C₁₂ heteroalkynylene, C₃-C₁₂ cycloalkylene, heterocycloalkylene, arylene, or heteroarylene may optionally be interrupted by one or more heteroatoms selected from O, S and N and may be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

### Cleavable Linkers

In some embodiments, the linker conjugating the anti-CD117 antibody or antigen binding fragment thereof and the cytotoxin (e.g., a PBD) is cleavable under intracellular conditions, such that cleavage of the linker releases the cytotoxin unit from the antibody in the intracellular environment. Cleavable linkers are designed to exploit the differences in local environments, e.g., extracellular and intracellular environments, including, for example, pH, reduction potential or enzyme concentration, to trigger the release of the cytotoxin in the target cell. Generally, cleavable linkers are relatively stable in circulation, but are particularly susceptible to cleavage in the intracellular environment through one or more mechanisms (e.g., including, but not limited to, activity of proteases, peptidases, and glucuronidases). Cleavable linkers used herein are stable in circulating plasma and/or outside the target cell and may be cleaved at some efficacious rate inside the target cell or in close proximity to the target cell.

Suitable cleavable linkers include those that may be cleaved, for instance, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012, the disclosure of which is incorporated herein by reference as it pertains to linkers suitable for covalent conjugation). Suitable cleavable linkers may include, for example, chemical moieties such as a hydrazine, a disulfide, a thioether or a dipeptide.

Linkers hydrolyzable under acidic conditions include, for example, hydrazones, semicarbazones, thiosemicarbazones, cis-aconitic amides, orthoesters, acetals, ketals, or the like. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661, the disclosure of each of which is incorporated herein by reference in its entirety as it pertains to linkers suitable for covalent conjugation. Such linkers are relatively stable under neutral pH conditions, such as those in the blood, but are unstable at below pH 5.5 or 5.0, the approximate pH of the lysosome.

Linkers cleavable under reducing conditions include, for example, a disulfide. A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935, the disclosure of each of which is incorporated herein by reference in its entirety as it pertains to linkers suitable for covalent conjugation.

Linkers susceptible to enzymatic hydrolysis can be, e.g., a peptide-containing linker that is cleaved by an intracellular peptidase or protease enzyme, including, but not limited to, a lysosomal or endosomal protease. One advantage of using intracellular proteolytic release of the therapeutic agent is that the agent is typically attenuated when conjugated and the serum stabilities of the conjugates are typically high. In some embodiments, the peptidyl linker is at least two amino acids long or at least three amino acids long. Exemplary amino acid linkers include a dipeptide, a tripeptide, a tetrapeptide or a pentapeptide. Examples of suitable peptides include those containing amino acids such as Valine, Alanine, Citrulline (Cit), Phenylalanine, Lysine, Leucine, and Glycine. Amino acid residues which comprise an amino acid linker component include those occurring naturally, as well as minor amino acids and non-naturally occurring amino acid analogs, such as citrulline. Exemplary dipeptides include valine-citrulline (vc or val-cit) and alanine-phenylalanine (af or ala-phe). Exemplary tripeptides include glycine-valine-citrulline (gly-val-cit) and glycine-glycine-glycine (gly-gly-gly). In some embodiments, the linker includes a dipeptide such as Val-Cit, Ala-Val, or Phe-Lys, Val-Lys, Ala-Lys, Phe-Cit, Leu-Cit, Ile-Cit, Phe-Arg, or Trp-Cit. Linkers containing dipeptides such as Val-Cit or Phe-Lys are disclosed in, for example, U.S. Pat. No. 6,214,345, the disclosure of which is incorporated herein by reference in its entirety as it pertains to linkers suitable for covalent conjugation. In some embodiments, the linker comprises a dipeptide selected from Val-Ala and Val-Cit.

Linkers suitable for conjugating the CD117 antibodies and antigen-binding fragments as described herein to a cytotoxic molecule include those capable of releasing a cytotoxin by a 1,6-elimination process. Chemical moieties capable of this elimination process include the p-aminobenzyl (PAB) group, 6-maleimidohexanoic acid, pH-sensitive carbonates, and other reagents as described in Jain et al., Pharm. Res. 32:3526-3540, 2015, the disclosure of which is incorporated herein by reference in its entirety as it pertains to linkers suitable for covalent conjugation.

In some embodiments, the linker includes a “self-immolative” group such as the afore-mentioned PAB or PABC (para-aminobenzyloxycarbonyl), which are disclosed in, for example, Carl et al., J. Med. Chem. (1981) 24:479-480; Chakravarty et al., (1983) J. Med. Chem. 26:638-644; U.S. Pat. Nos. 6,214,345; 6,218,519; 6,835,807; 6,268,488; 6,759,509; 6,677,435; 5,621,002; US Patent Application Publication Nos. US20030130189; US20030096743; US20040052793; US20040018194; US20040052793; US20040121940; and International Patent Application Publication Nos. WO98/13059 and WO2004/032828). Other such chemical moieties capable of this process (“self-immolative linkers”) include methylene carbamates and heteroaryl groups such as aminothiazoles, aminoimidazoles, aminopyrimidines, and the like. Linkers containing such heterocyclic self-immolative groups are disclosed in, for example, U.S. Patent Publication Nos. 20160303254 and 20150079114, and U.S. Pat. No. 7,754,681; Hay et al. (1999) Bioorg. Med. Chem. Lett. 9:2237; US 2005/0256030; de Groot et al (2001) J. Org. Chem. 66:8815-8830; and U.S. Pat. No. 7,223,837. In some embodiments, a dipeptide is used in combination with a self-immolative linker.

In some embodiments, the linker L comprises one or more of a hydrazine, a disulfide, a thioether, an amino acid, a peptide consisting of up to 10 amino acids, a p-aminobenzyl (PAB) group, a heterocyclic self-immolative group, C₁-C₁₂ alkyl, C₁-C₁₂ heteroalkyl, C₂-C₁₂ alkenyl, C₂-C₁₂ heteroalkenyl, C₂-C₁₂ alkynyl, C₂-C₁₂ heteroalkynyl, C₃-C₁₂ cycloalkyl, heterocycloalkyl, aryl, heteroaryl, a —(C═O)— group, a —C(O)NH— group, an —OC(O)NH— group, a —(CH₂CH₂O)_{q}— group where p is an integer from 1-12, or a solubility enhancing group;

wherein each C₁-C₁₂ alkyl, C₁-C₁₂ heteroalkyl, C₂-C₁₂ alkenyl, C₂-C₁₂ heteroalkenyl, C₂-C₁₂ alkynyl, C₂-C₁₂ heteroalkynyl, C₃-C₁₂ cycloalkyl, heterocycloalkyl, aryl, or heteroaryl group may be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

In some embodiments, each C₁-C₁₂ alkyl, C₁-C₁₂ heteroalkyl, C₂-C₁₂ alkenyl, C₂-C₁₂ heteroalkenyl, C₂-C₁₂ alkynyl, C₂-C₁₂ heteroalkynyl, C₃-C₁₂ cycloalkyl, heterocycloalkyl, aryl, or heteroaryl group may optionally be interrupted by one or more heteroatoms selected from O, S and N.

In some embodiments, each C₁-C₁₂ alkyl, C₁-C₁₂ heteroalkyl, C₂-C₁₂ alkenyl, C₂-C₁₂ heteroalkenyl, C₂-C₁₂ alkynyl, C₂-C₁₂ heteroalkynyl, C₃-C₁₂ cycloalkyl, heterocycloalkyl, aryl, or heteroaryl group may optionally be interrupted by one or more heteroatoms selected from O, S and N and may be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

One of skill in the art will recognize that one or more of the groups listed may be present in the form of a bivalent (diradical) species, e.g., C₁-C₁₂ alkylene and the like.

In some embodiments, the linker L comprises the moiety *-L₁L₂-**, wherein:

L₁ is absent or is —(CH₂)ₘNR¹C(═O)—, —(CH₂)ₘNR¹—, —(CH₂)ₘX₃(CH₂)ₘ—,

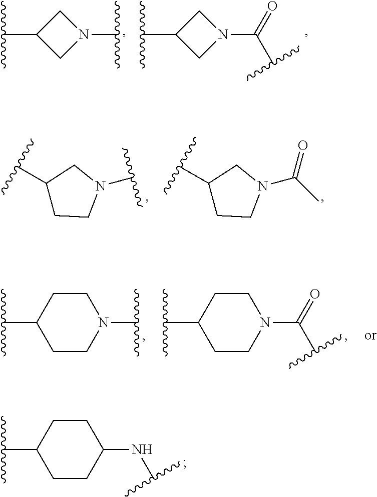

L₂ is absent or is —(CH₂)ₘ—, —NR¹(CH₂)ₘ—, —(CH₂)ₘNR¹C(═O)(CH₂)ₘ—, —X₄, —(CH₂)ₘNR¹C(═O)X₄, (CH₂)ₘNR¹C(═O)—, —((CH₂)ₘO)ₙ(CH₂)ₘ—, ((CH₂)ₘO)ₙ(CH₂)ₘX₃(CH₂)ₘ—, —NR¹((CH₂)ₘO)ₙX₃(CH₂)ₘ—, —NR¹((CH₂)ₘO)ₙ(CH₂)ₘX₃(CH₂)ₘ—, —X₁X₂C(═O)(CH₂)ₘ—, (CH₂)ₘ(O(CH₂)ₘ)ₙ—, —(CH₂)ₘNR¹(CH₂)ₘ—, (CH₂)ₘNR¹C(═O)(CH₂)ₘX₃(CH₂)ₘ—, —(CH₂)ₘC(═O)NR¹(CH₂)ₘNR¹C(═O)(CH₂)ₘ—, —(CH₂)ₘC(═O)—, —(CH₂)ₘNR¹(CH₂)ₘC(═O)X₂X₁C(═O)—, —(CH₂)ₘX₃(CH₂)ₘC(═O)X₂X₁C(═O)—, —(CH₂)ₘC(═O)NR¹(CH₂)ₘ—, —(CH₂)ₘC(═O)NR¹(CH₂)ₘX₃(CH₂)ₘ—, (CH₂)ₘX₃(CH₂)ₘNR¹C(═O)(CH₂)ₘ—, —(CH₂)ₘX₃(CH₂)ₘC(═O)NR¹(CH₂)ₘ—, (CH₂)ₘO)ₙ(CH₂)ₘNR¹C(═O)(CH₂)ₘ—, —(CH₂)ₘC(═O)NR¹(CH₂)ₘ(O(CH₂)ₘ)ₙ—, (CH₂)ₘ(O(CH₂)ₘ)ₙC(═O)—, —(CH₂)ₘNR¹(CH₂)ₘC(═O)—, —(CH₂)ₘC(═O)NR¹(CH₂)ₘNR¹C(═O)—, —(CH₂)ₘ(O(CH₂)ₘ)ₙX₃(CH₂)ₘ—, —(CH₂)ₘX₃((CH₂)ₘO)ₙ(CH₂)ₘ—, —(CH₂)ₘX₃(CH₂)ₘC(═O)—, —(CH₂)ₘC(═O)NR¹(CH₂)ₘO)ₙ(CH₂)ₘX₃(CH₂)ₘ—, (CH₂)ₘX₃(CH₂)ₘ(O(CH₂)ₘ)ₙNR¹C(═O)(CH₂)ₘ—, —(CH₂)ₘX₃(CH₂)ₘ(O(CH₂)ₘ)ₙC(═O)—, (CH₂)ₘX₃(CH₂)ₘ(O(CH₂)ₘ)ₙ—, —(CH₂)ₘC(═O)NR¹(CH₂)ₘC(═O)—, (CH₂)ₘC(═O)NR¹(CH₂)ₘ(O(CH₂)ₘ)ₙC(═O)—, —((CH₂)ₘO)ₙ(CH₂)ₘNR¹C(═O)(CH₂)ₘ—, (CH₂)ₘC(═O)NR¹(CH₂)ₘC(═O)NR¹(CH₂)ₘ—, —(CH₂)ₘNR¹C(═O)(CH₂)ₘNR¹C(═O)(CH₂) —(CH₂)ₘX₃(CH₂)ₘC(═O)NR¹—, —(CH₂)ₘC(═O)NR¹—, —(CH₂)ₘX₃—, —C(R¹)₂(CH₂)ₘ—, (CH₂)ₘC(R¹)₂NR¹—, —(CH₂)ₘC(═O)NR¹(CH₂)ₘNR¹—, (CH₂)ₘC(═O)NR¹(CH₂)ₘNR¹C(═O)NR¹—, —(CH₂)ₘC(═O)X₂X₁C(═O)—, C(R¹)₂(CH₂)ₘNR¹C(═O)(CH₂)ₘ—, —(CH₂)ₘC(═O)NR¹(CH₂)ₘC(R¹)₂NR¹—, C(R¹)₂(CH₂)ₘX₃(CH₂)ₘ—, —(CH₂)ₘX₃(CH₂)ₘC(R¹)₂NR¹—, —C(R¹)₂(CH₂)ₘO C(═O)NR¹(CH₂)ₘ—, —(CH₂)ₘNR¹C(═O)O(CH₂)ₘC(R¹)₂NR¹—, —(CH₂)ₘX₃(CH₂)ₘNR¹—, (CH₂)ₘX₃(CH₂)ₘ(O(CH₂)ₘ)ₙNR¹—, —(CH₂)ₘNR¹—, —(CH₂)ₘC(═O)NR¹(CH₂)ₘ(O(CH₂)ₘ)ₙNR¹—, —(CH₂)ₘ(O(CH₂)ₘ)ₙNR¹—, —(CH₂CH₂O)ₙ(CH₂)ₘ—, —(CH₂)ₘ(OCH₂CH₂)ₙ; —(CH₂)ₘO(CH₂)ₘ—, —(CH₂)ₘS(═O)₂—, —(CH₂)ₘC(═O)NR¹(CH₂)ₘS(═O)₂—, —(CH₂)ₘX₃(CH₂)ₘS(═O)₂—, —(CH₂)ₘX₂X₁C(═O)—, —(CH₂)ₘ(O(CH₂)ₘ)ₙC(═O)X₂X₁C(═O)—, —(CH₂)ₘ(O(CH₂)ₘ)ₙX₂X₁C(═O)—, —(CH₂)ₘX₃(CH₂)ₘX₂X₁C(═O)—, —(CH₂)ₘX₃(CH₂)ₘ(O(CH₂)ₘ)ₙX₂X₁C(═O)—, —(CH₂)ₘX₃(CH₂)ₘC(═O)NR¹(CH₂)ₘNR¹C(═O)—, —(CH₂)ₘX₃(CH₂)ₘC(═O)NR¹(CH₂)ₘC(═O)—, —(CH₂)ₘX₃(CH₂)ₘC(═O)NR¹(CH₂)ₘ(O(CH₂)ₘ)ₙC(═O)—, —(CH₂)ₘC(═O)X₂X₁C(═O)NR¹(CH₂)ₘ—, —(CH₂)ₘX₃(O(CH₂)ₘ)ₙC(═O)—, —(CH₂)ₘNR¹C(═O)((CH₂)ₘO)ₙ(CH₂)ₘ—, (CH₂)ₘ(O(CH₂)ₘ)ₙC(═O)NR¹(CH₂)ₘ—, —(CH₂)ₘNR¹C(═O)NR¹(CH₂)ₘ— or (CH₂)ₘX₃(CH₂)ₘNR¹C(═O)—;

wherein

X₁ is

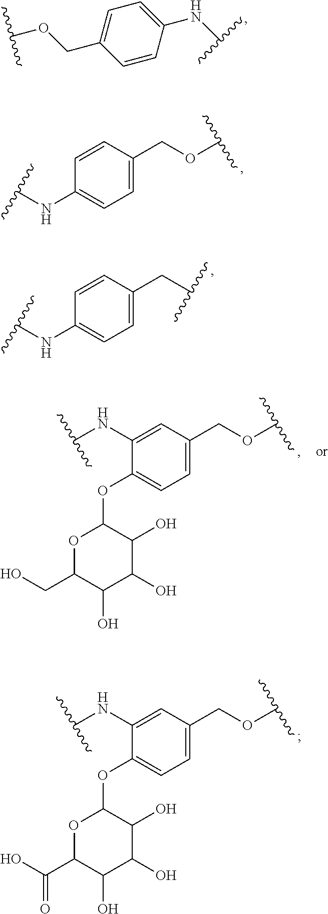 [SMILES: *Nc1ccc(CO*)cc1 ; *Nc1ccc(CO*)cc1 ; *Cc1ccc(N*)cc1 ; *Nc1cc(CO*)ccc1OC1OC(CO)C(O)C(O)C1O ; *Nc1cc(CO*)ccc1OC1OC(C(=O)O)C(O)C(O)C1O]

X₂ is

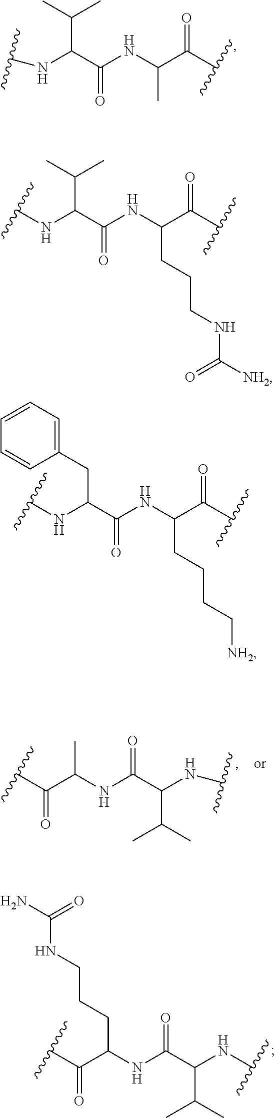 [SMILES: *NC(C(=O)NC(C)C(*)=O)C(C)C ; *NC(C(=O)NC(CCCNC(N)=O)C(*)=O)C(C)C ; *NC(Cc1ccccc1)C(=O)NC(CCCCN)C(*)=O ; *NC(C(=O)NC(C)C(*)=O)C(C)C ; *NC(C(=O)NC(CCCNC(N)=O)C(*)=O)C(C)C]

X₃ is

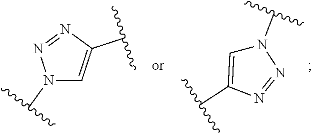 [SMILES: *c1cn(*)nn1 ; *c1cn(*)nn1]

and

X₄ is

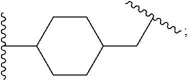

wherein

R¹ is independently selected for each occasion from H and C₁-C₆ alkyl;

m is independently selected for each occasion from 1, 2, 3, 4, 5, 6, 7, 8, 9 and 10;

n is independently selected for each occasion from 1, 2, 3, 4, 5, 6, 7, 8, 9, 10, 11, 12, 13 and 14; and

wherein the single asterisk (*) indicates the attachment point to the cytotoxin (e.g., a PBD), and the double asterisk (**) indicates the attachment point to the reactive substituent Z′ or chemical moiety Z, with the proviso that L₁ and L₂ are not both absent.

In some embodiments, the linker includes a p-aminobenzyl group (PAB). In one embodiment, the p-aminobenzyl group is disposed between the cytotoxic drug and a protease cleavage site in the linker. In one embodiment, the p-aminobenzyl group is part of a p-aminobenzyloxycarbonyl unit. In one embodiment, the p-aminobenzyl group is part of a p-aminobenzylamido unit.

In some embodiments, the linker comprises a dipeptide selected from the group consisting of Phe-Lys, Val-Lys, Phe-Ala, Phe-Cit, Val-Ala, Val-Cit, and Val-Arg. In some embodiments, the linker comprises one or more of PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

In some embodiments, the linker comprises a combination of one or more of a peptide, oligosaccharide, —(CH₂)ₚ—, —(CH₂CH₂O)_{q}—, —(C═O)(CH₂)ᵣ—, —(C═O)(CH₂CH₂O)ₜ—, —(NHCH₂CH₂)ᵤ—, -PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB, wherein each of p, q, r, t, and u are integers from 1-12, selected independently for each occurrence.

In some embodiments, the linker comprises

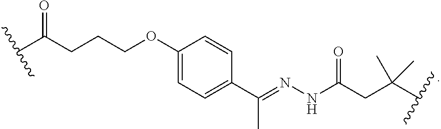 [SMILES: *C(=O)CCCOc1ccc(/C(C)=N/NC(=O)CC(*)(C)C)cc1]

In some embodiments, the linker comprises MCC (4-[N-maleimidomethyl]cyclohexane 1-carboxylate).

In some embodiments, the linker comprises PAB-Ala-Val- or PAB-Cit-Val-, a —(C═O)(CH₂)ᵣ— unit, a —(C═O)(CH₂CH₂O)ₜ— unit, and a —(NHCH₂CH₂)ᵤ— unit, wherein r=2, t=8, and u=1. In particular embodiments, the linker may be represented by formula (II):

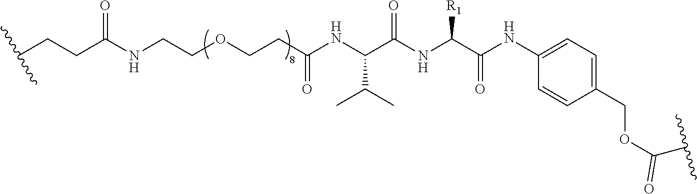

where R₁ is CH₃ (Ala) or (CH₂)₃NH(CO)NH₂ (Cit).

It will be recognized by one of skill in the art that any one or more of the chemical groups, moieties, and features disclosed herein may be combined in multiple ways to form linkers useful for conjugation of the antibodies and cytotoxins as disclosed herein.

### Linker-Cytotoxin and Linker-Antibody Conjugation

In certain embodiments, the linker is reacted with the cytotoxin under appropriate conditions to form a linker-cytotoxin conjugate. In certain embodiments, reactive groups are used on the cytotoxin or linker to form a covalent attachment.

In some embodiments, the cytotoxin is a PBD or derivative thereof according to formula (I). The cytotoxin-linker conjugate is subsequently reacted with the antibody, derivatized antibody, or antigen-binding fragment thereof that binds CD117, under appropriate conditions to form the ADC. Alternatively, the linker may first be reacted with the antibody, derivatized antibody or antigen-binding fragment thereof that binds CD117, to form a linker-antibody conjugate, and then reacted with the cytotoxin to form the ADC. Such conjugation reactions will now be described more fully.

A number of different reactions are available for covalent attachment of linkers or cytotoxin-linker conjugates to the antibody or antigen-binding fragment thereof. Suitable attachment points on the antibody molecule include, but are not limited to, the amine groups of lysine, the free carboxylic acid groups of glutamic acid and aspartic acid, the sulfhydryl groups of cysteine, and the various moieties of aromatic amino acids. For instance, non-specific covalent attachment may be undertaken using a carbodiimide reaction to link a carboxy (or amino) group on a linker to an amino (or carboxy) group on an antibody moiety. Additionally, bifunctional agents such as dialdehydes or imidoesters may also be used to link the amino group on a linker to an amino group on an antibody moiety. Also available for attachment of cytotoxins to antibody moieties is the Schiff base reaction. This method involves the periodate oxidation of a glycol or hydroxy group on either the antibody or linker, thus forming an aldehyde which is then reacted with the linker or antibody, respectively. Covalent bond formation occurs via formation of a Schiff base between the aldehyde and an amino group. Isothiocyanates may also be used as coupling agents for covalently attaching cytotoxins or antibody moieties to linkers. Other techniques are known to the skilled artisan and within the scope of the present disclosure.

Linkers useful in for conjugation to the antibodies or antigen-binding fragments as described herein include, without limitation, linkers containing a chemical moiety Z formed by a coupling reaction between the antibody and a reactive chemical moiety (referred to herein as a reactive substituent, Z′) on the linker as depicted in Table 1, below. Curved lines designate points of attachment to the antibody or antigen-binding fragment, and the cytotoxic molecule, respectively.

TABLE 1
Exemplary chemical moieties Z formed by coupling reactions in the formation of
antibody-drug conjugates.

| Exemplary Coupling | |
|---|---|
| Reactions | Chemical Moiety Z Formed by Coupling Reactions |
| [3 + 2] Cycloaddition | 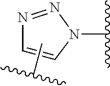 |
| [3 + 2] Cycloaddition | 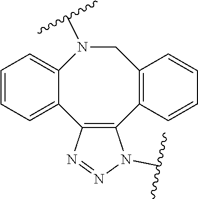 [SMILES: *N1Cc2ccccc2-c2c(nnn2*)-c2ccccc21] |
| [3 + 2] Cycloaddition, Esterification | 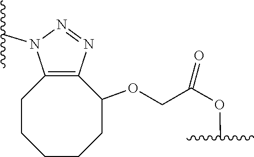 [SMILES: *OC(=O)COC1CCCCCc2c1nnn2*] |
| [3 + 2] Cycloaddition, Esterification | 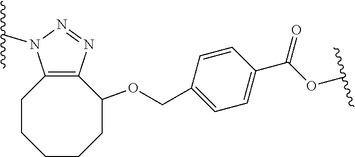 [SMILES: *OC(=O)c1ccc(COC2CCCCCc3c2nnn3*)cc1] |
| [3 + 2] Cycloaddition, Esterification | 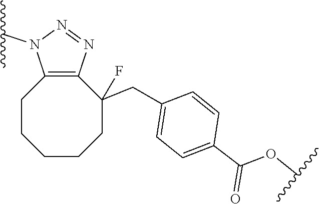 [SMILES: *OC(=O)c1ccc(CC2(F)CCCCCc3c2nnn3*)cc1] |
| [3 + 2] Cycloaddition, Esterification | 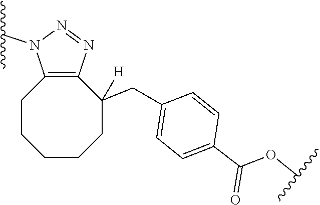 [SMILES: *OC(=O)c1ccc(CC2CCCCCc3c2nnn3*)cc1] |
| [3 + 2] Cycloaddition, Esterification | 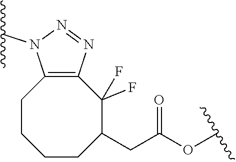 [SMILES: *OC(=O)CC1CCCCc2c(nnn2*)C1(F)F] |
| [3 + 2] Cycloaddition, Esterification | 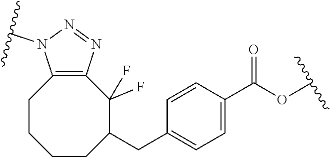 [SMILES: *OC(=O)c1ccc(CC2CCCCc3c(nnn3*)C2(F)F)cc1] |
| [3 + 2] Cycloaddition, Esterification | 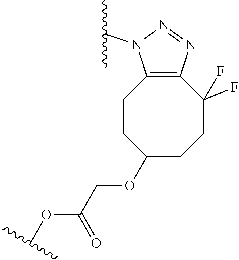 [SMILES: *OC(=O)COC1CCc2c(nnn2*)C(F)(F)CC1] |
| [3 + 2] Cycloaddition, Esterification | 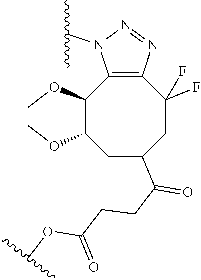 [SMILES: *OC(=O)CCC(=O)C1C[C@H](OC)[C@@H](OC)c2c(nnn2*)C(F)(F)C1] |
| [3 + 2] Cycloaddition, Esterification | 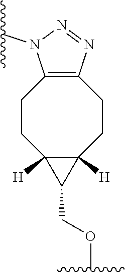 [SMILES: *OC[C@@H]1[C@@H]2CCc3c(nnn3*)CC[C@@H]21] |
| [3 + 2] Cycloaddition, Esterification | 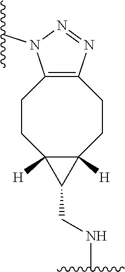 [SMILES: *NC[C@@H]1[C@@H]2CCc3c(nnn3*)CC[C@@H]21] |
| [3 + 2] Cycloaddition, Esterification | 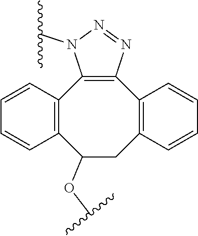 [SMILES: *OC1Cc2ccccc2-c2nnn(*)c2-c2ccccc21] |
| [3 + 2] Cycloaddition, Etherification | 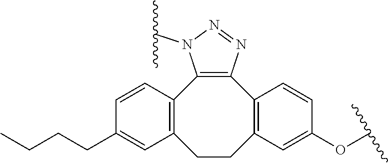 [SMILES: *Oc1ccc2c(c1)CCc1cc(CCCC)ccc1-c1c-2nnn1*] |
| [3 + 2] Cycloaddition | 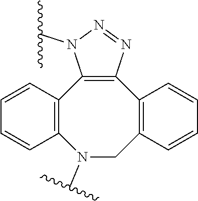 [SMILES: *N1Cc2ccccc2-c2nnn(*)c2-c2ccccc21] |
| Michael addition | 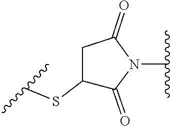 [SMILES: *SC1CC(=O)N(*)C1=O] |
| Michael addition | 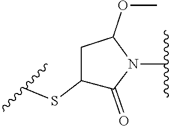 [SMILES: *SC1CC(OC)N(*)C1=O] |
| Imine condensation, Amidation | 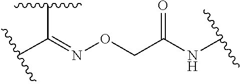 [SMILES: *NC(=O)CON=C(*)*] |
| Imine condensation | 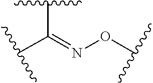 [SMILES: *ON=C(*)*] |
| Disulfide formation | 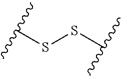 [SMILES: *SS*] |
| Thiol alkylation | 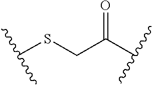 [SMILES: *SCC(*)=O] |
| Condensation, Michael addition | 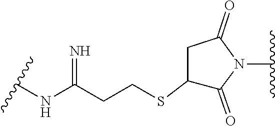 [SMILES: *NC(=N)CCSC1CC(=O)N(*)C1=O] |

One of skill in the art will recognize that a reactive substituent Z′ attached to the linker and a reactive substituent on the antibody or antigen-binding fragment thereof, are engaged in the covalent coupling reaction to produce the chemical moiety Z, and will recognize the reactive substituent Z′. Therefore, antibody-drug conjugates useful in conjunction with the methods described herein may be formed by the reaction of an antibody, or antigen-binding fragment thereof, with a linker or cytotoxin-linker conjugate, as described herein, the linker or cytotoxin-linker conjugate including a reactive substituent Z′, suitable for reaction with a reactive substituent on the antibody, or antigen-binding fragment thereof, to form the chemical moiety Z.

In some embodiments, Z′ is —NR¹C(═O)CH═CH₂, —N₃, —SH, —S(═O)₂(CH═CH₂), —(CH₂)₂S(═O)₂(CH═CH₂), —NR¹S(═O)₂(CH═CH₂), —NR¹C(═O)CH₂R², —NR¹C(═O)CH₂Br, —NR¹C(═O)CH₂I, —NHC(═O)CH₂Br, —NHC(═O)CH₂I, —ONH₂, —C(O)NHNH₂, —CO₂H, —NH₂, —NH(C═O), —NC(═S),

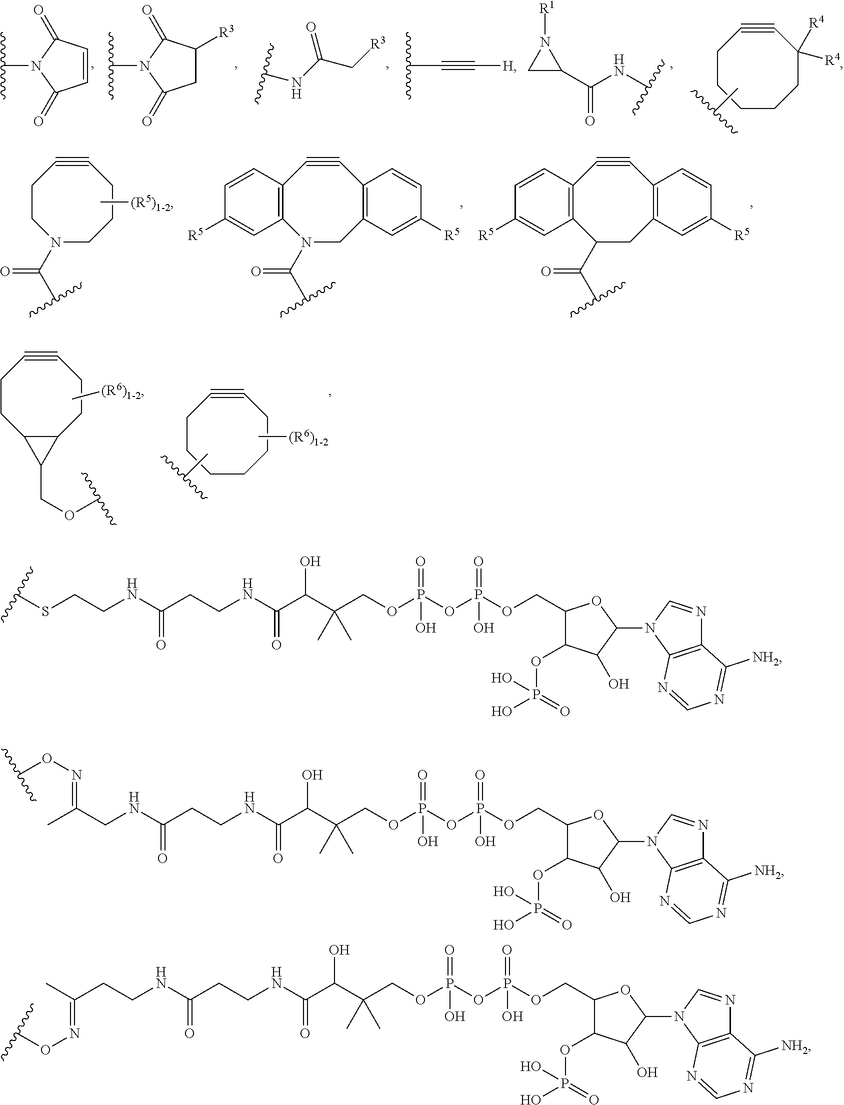

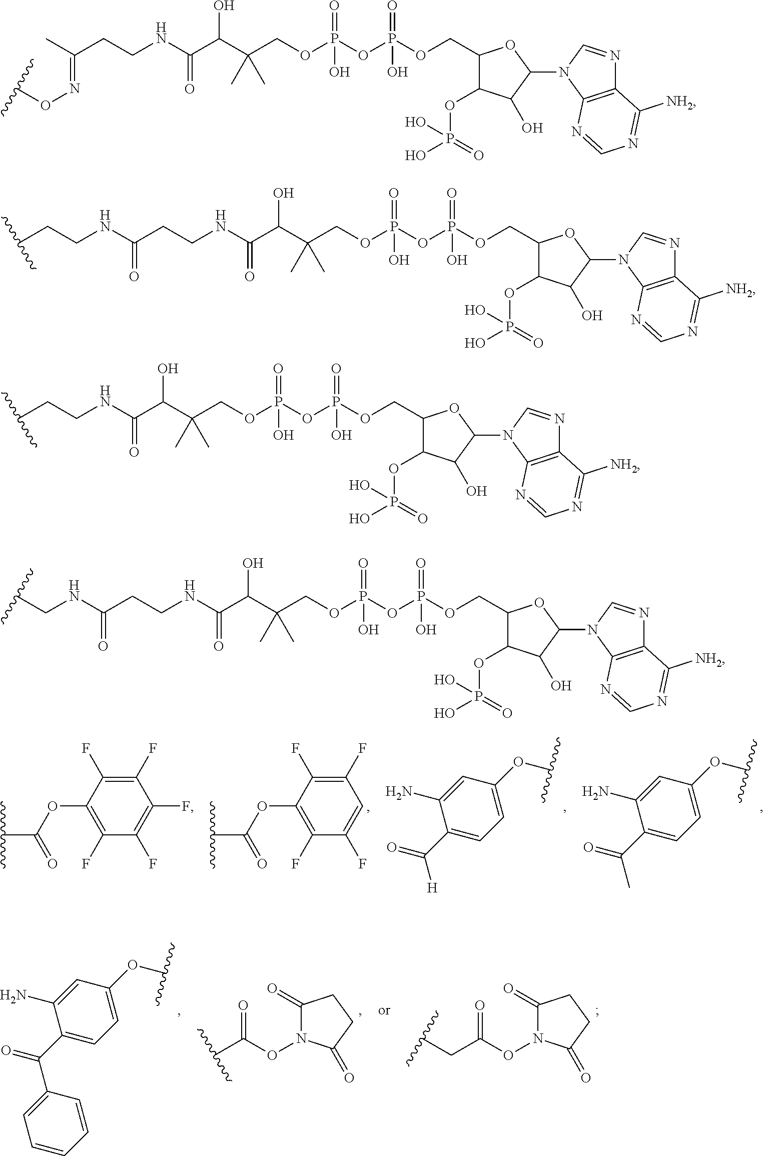

wherein
R¹ is independently selected for each occasion from H and C₁-C₆ alkyl;
R² is —S(CH₂)ₙCHR³NHC(═O)R¹;
R³ is R¹ or —C(═O)OR¹;
R⁴ is independently selected for each occasion from H, C₁-C₆ alkyl, F, Cl, and —OH;
R⁵ is independently selected for each occasion from H, C₁-C₆ alkyl, F, Cl, —NH₂, —OCH₃, —OCH₂CH₃, —N(CH₃)₂, —CN, —NO₂ and —OH; and
R⁶ is independently selected for each occasion from H, C₁-C₆ alkyl, F, benzyloxy substituted with —C(═O)OH, benzyl substituted with —C(═O)OH, C₁-C₄ alkoxy substituted with —C(═O)OH, and C₁-C₄ alkyl substituted with —C(═O)OH.

As depicted in Table 1, examples of suitably reactive substituents Z′ on the linker and reactive substituents on the antibody or antigen-binding fragment thereof include a nucleophile/electrophile pair (e.g., a thiol/haloalkyl pair, an amine/carbonyl pair, or a thiol/α,β-unsaturated carbonyl pair, and the like), a diene/dienophile pair (e.g., an azide/alkyne pair, or a diene/α,β-unsaturated carbonyl pair, among others), and the like. Coupling reactions between the reactive substitutents to form the chemical moiety Z include, without limitation, thiol alkylation, hydroxyl alkylation, amine alkylation, amine or hydroxylamine condensation, hydrazine formation, amidation, esterification, disulfide formation, cycloaddition (e.g., [4+2] Diels-Alder cycloaddition, [3+2] Huisgen cycloaddition, among others), nucleophilic aromatic substitution, electrophilic aromatic substitution, and other reactive modalities known in the art or described herein. In some embodiments, the reactive substituent Z′ is an electrophilic functional group suitable for reaction with a nucleophilic functional group on the antibody, or antigen-binding fragment thereof.

Reactive substituents that may be present within an antibody, or antigen-binding fragment thereof, as disclosed herein include, without limitation, nucleophilic groups such as (i) N-terminal amine groups, (ii) side chain amine groups, e.g. lysine, (iii) side chain thiol groups, e.g. cysteine, and (iv) sugar hydroxyl or amino groups where the antibody is glycosylated. Reactive substituents that may be present within an antibody, or antigen-binding fragment thereof, as disclosed herein include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. In some embodiments, the reactive substituents present within an antibody, or antigen-binding fragment thereof as disclosed herein include, are amine or thiol moieties. Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by introducing one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

In some embodiments, the reactive substituent Z′ attached to the linker is a nucleophilic group which is reactive with an electrophilic group present on an antibody. Useful electrophilic groups on an antibody include, but are not limited to, aldehyde and ketone carbonyl groups. A nucleophilic group (e.g., a) heteroatom of can react with an electrophilic group on an antibody and form a covalent bond to the antibody. Useful nucleophilic groups include, but are not limited to, hydrazide, oxime, amino, hydroxyl, hydrazine, thiosemicarbazone, hydrazine carboxylate, and arylhydrazide.

In some embodiments, chemical moiety Z is the product of a reaction between reactive nucleophilic substituents present within the antibodies, or antigen-binding fragments thereof, such as amine and thiol moieties, and a reactive electrophilic substituent Z′ attached to the linker. For instance, Z′ may be a Michael acceptor (e.g., maleimide), activated ester, electron-deficient carbonyl compound, or an aldehyde, among others.

Several representative and non-limiting examples of reactive substituents Z′ and the resulting chemical moieties Z are provided in Table 2.

TABLE 2
Complementary reactive substituents and chemical moieties

| | Functional Group | | |
|---|---|---|---|
| | on Antibody | Z′ group | Z group |
| Naturally Occurring | 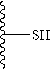 [SMILES: *S] | 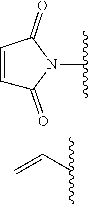 | 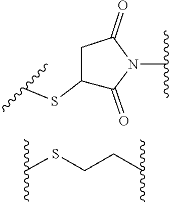 |
| | 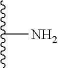 [SMILES: *N] | 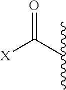 | 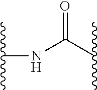 [SMILES: *NC(*)=O] |
| Synthetically Introduced | 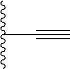 [SMILES: *C#C] | 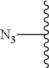 [SMILES: *N=[N+]=[N-]] | 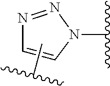 |
| | 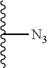 [SMILES: *N=[N+]=[N-]] | 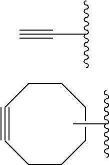 | 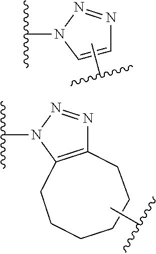 |
| | 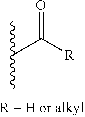 | 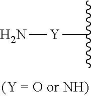 | 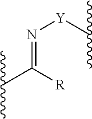 |

For instance, linkers suitable for the synthesis of linker-antibody conjugates and ADCs include, without limitation, reactive substituents Z′ attached to the linker, such as a maleimide or haloalkyl group. These may be attached to the linker by, for example, reagents such as succinimidyl 4-(N-maleimidomethyl)-cyclohexane-L-carboxylate (SMCC), N-succinimidyl iodoacetate (SIA), sulfo-SMCC, m-maleimidobenzoyl-N-hydroxysuccinimidyl ester (MBS), sulfo-MBS, and succinimidyl iodoacetate, among others described, in for instance, Liu et al., 18:690-697, 1979, the disclosure of which is incorporated herein by reference as it pertains to linkers for chemical conjugation.

In some embodiments, the reactive substituent Z′ attached to linker L is a maleimide, azide, or alkyne. An example of a maleimide-containing linker is the non-cleavable maleimidocaproyl-based linker, which is particularly useful for the conjugation of microtubule-disrupting agents such as auristatins. Such linkers are described by Doronina et al., Bioconjugate Chem. 17:14-24, 2006, the disclosure of which is incorporated herein by reference as it pertains to linkers for chemical conjugation.

In some embodiments, the reactive substituent Z′ is —(C═O)— or —NH(C═O)—, such that the linker may be joined to the antibody, or antigen-binding fragment thereof, by an amide or urea moiety, respectively, resulting from reaction of the —(C═O)— or —NH(C═O)— group with an amino group of the antibody or antigen-binding fragment thereof.

In some embodiments, the reactive substituent Z′ is an N-maleimidyl group, halogenated N-alkylamido group, sulfonyloxy N-alkylamido group, carbonate group, sulfonyl halide group, thiol group or derivative thereof, alkynyl group comprising an internal carbon-carbon triple bond, (hetero)cycloalkynyl group, bicyclo[6.1.0]non-4-yn-9-yl group, alkenyl group comprising an internal carbon-carbon double bond, cycloalkenyl group, tetrazinyl group, azido group, phosphine group, nitrile oxide group, nitrone group, nitrile imine group, diazo group, ketone group, (O-alkyl)hydroxylamino group, hydrazine group, halogenated N-maleimidyl group, 1,1-bis (sulfonylmethyl)methylcarbonyl group or elimination derivatives thereof, carbonyl halide group, or an allenamide group, each of which may be optionally substituted. In some embodiments, the reactive substituent comprises a cycloalkene group, a cycloalkyne group, or an optionally substituted (hetero)cycloalkynyl group.

In some embodiments, the chemical moiety Z is selected from Table 1. In some embodiments, the chemical moiety Z is:

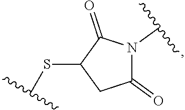

where S is a sulfur atom which represents the reactive substituent present within an antibody, or antigen-binding fragment thereof, that specifically binds to an antigen expressed on the cell surface of a human stem cell or a T cell (e.g., from the —SH group of a cysteine residue).

In some embodiments, wherein the linker is of formula (II), the linker-reactive substituent group, taken together as L-Z′, prior to conjugation with the antibody or antigen binding fragment thereof, has the structure:

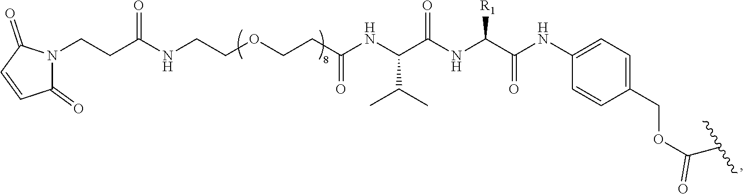

where the wavy line indicates the point of attachment to a substituent on the cytotoxin (e.g., a PBD or derivative thereof). The wavy line at the linker terminus indicates the point of attachment to the cytotoxin, e.g., a PBD. In some embodiments, the linker L and the chemical moiety Z, after conjugation to the antibody, taken together as L-Z-Ab, has the structure:

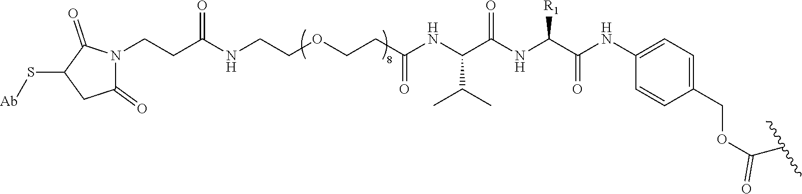

where S is a sulfur atom which represents the reactive substituent present within an antibody, or antigen-binding fragment thereof, that specifically binds to an antigen expressed on the cell surface of a human stem cell or a T cell (e.g., from the —SH group of a cysteine residue). The wavy line at the linker terminus indicates the point of attachment to the cytotoxin, e.g., a PBD or derivative thereof.

In some embodiments, the cytotoxin is a pyrrolobenzodiazepine dimer represented by the structural formula (I), and the linker is attached by a bond to the diazepine amino group. In such embodiments, the ADC may be represented by formula (III):

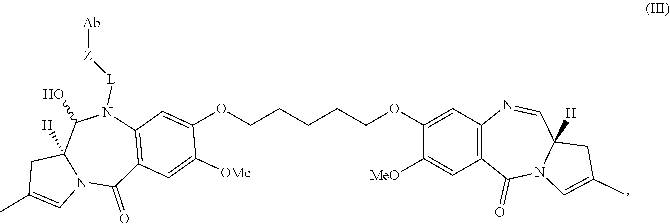

where each of L, Z, and Ab are as described herein.

In some embodiments, the linker L of the ADC of formula (III) is a cleavable linker. In some embodiments, the cleavable linker L comprises one or more of a hydrazine, a disulfide, a thioether, an amino acid, a peptide consisting of up to 10 amino acids, a p-aminobenzyl (PAB) group, a heterocyclic self-immolative group, C₁-C₁₂ alkyl, C₁-C₁₂ heteroalkyl, C₂-C₁₂ alkenyl, C₂-C₁₂ heteroalkenyl, C₂-C₁₂ alkynyl, C₂-C₁₂ heteroalkynyl, C₃-C₁₂ cycloalkyl, heterocycloalkyl, aryl, heteroaryl, a —(C═O)— group, a —C(O)NH— group, an —OC(O)NH— group, or a —(CH₂CH₂O)_{q}— group where p is an integer from 1-12;

wherein each C₁-C₁₂ alkyl, C₁-C₁₂ heteroalkyl, C₂-C₁₂ alkenyl, C₂-C₁₂ heteroalkenyl, C₂-C₁₂ alkynyl, C₂-C₁₂ heteroalkynyl, C₃-C₁₂ cycloalkyl, heterocycloalkyl, aryl, or heteroaryl group may be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro;

or each C₁-C₁₂alkyl, C₁-C₁₂ heteroalkyl, C₂-C₁₂ alkenyl, C₂-C₁₂ heteroalkenyl, C₂-C₁₂ alkynyl, C₂-C₁₂ heteroalkynyl, C₃-C₁₂cycloalkyl, heterocycloalkyl, aryl, or heteroaryl group may optionally be interrupted by one or more heteroatoms selected from O, S and N.

In some embodiments, the linker comprises a combination of one or more of a peptide, oligosaccharide, —(CH₂)ₚ—, —(CH₂CH₂O)_{q}—, —(C═O)(CH₂)ᵣ—, —(C═O)(CH₂CH₂O)ₜ—, —(NHCH₂CH₂)ᵤ—, -PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB, wherein each of p, q, r, t, and u are integers from 1-12, selected independently for each occurrence.

In some embodiments, the linker comprises PAB-Ala-Val- or PAB-Cit-Val-, a —(C═O)(CH₂)ᵣ— unit, a —(C═O)(CH₂CH₂O)ₜ— unit, and a —(NHCH₂CH₂)ᵤ— unit, wherein r=2, t=8, and u=1. In particular embodiments, the linker may be represented by formula (II):

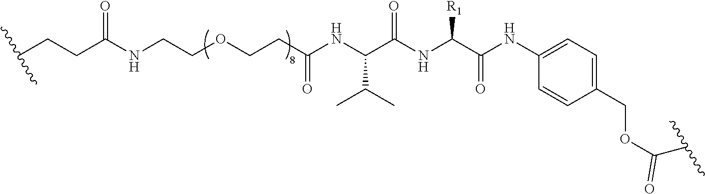

where R₁ is CH₃ (Ala) or (CH₂)₃NH(CO)NH₂ (Cit).

In specific embodiments, wherein the cytotoxin is of formula (I) and the linker is of formula (II) where R₁ is CH₃, the cytotoxin-linker-reactive moiety conjugate, taken together as Cy-L-Z′, may be represented by the formula (IV):

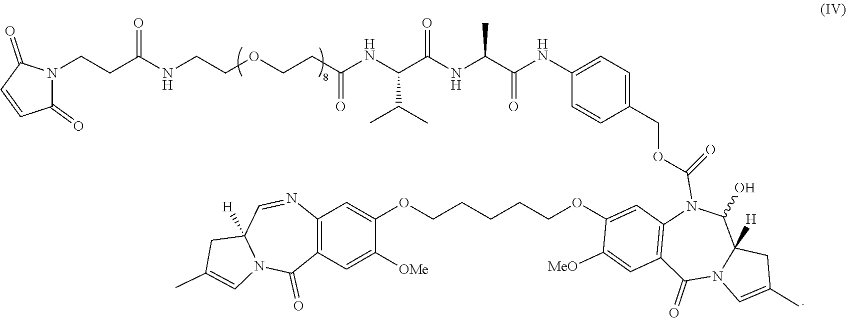

This particular cytotoxin-linker conjugate is known as tesirine (SG3249), and has been described in, for example, Howard et al., ACS Med. Chem. Lett. 2016, 7(11), 983-987, the disclosure of which is incorporated by reference herein in its entirety. A compound of formula (IV), when conjugated to an anti-CD117 antibody as disclosed herein, may be represented by formula (V):

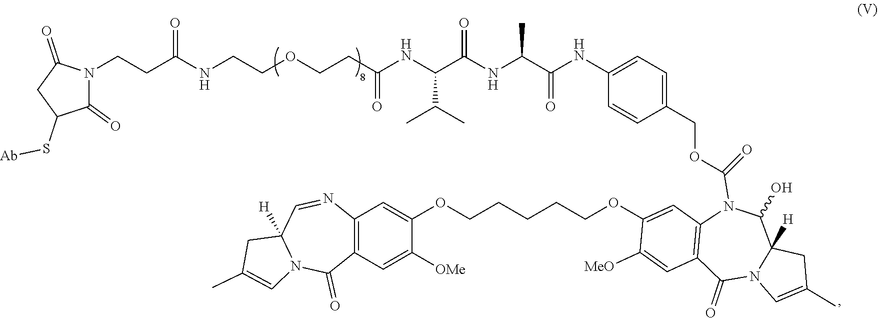

where Ab is the anti-CD117 antibody or antigen binding fragment thereof as disclosed herein, and S represents a sulfur atom (e.g., a cysteine residue thiol) present in or introduced into the antibody.

In some embodiments, the cytotoxin is a pyrrolobenzodiazepine dimer represented by the formula:

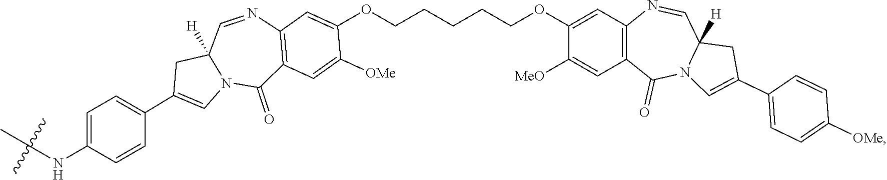 [SMILES: *Nc1ccc(C2=CN3C(=O)c4cc(OC)c(OCCCCCOc5cc6c(cc5OC)C(=O)N5C=C(c7ccc(OC)cc7)C[C@H]5C=N6)cc4N=C[C@@H]3C2)cc1]

wherein the wavy line indicates the attachment point of the linker.

In some embodiments, the cytotoxin-linker conjugate, prior to conjugation to the antibody and including the reactive substituent Z′, taken together as Cy-L-Z′, has the structure:

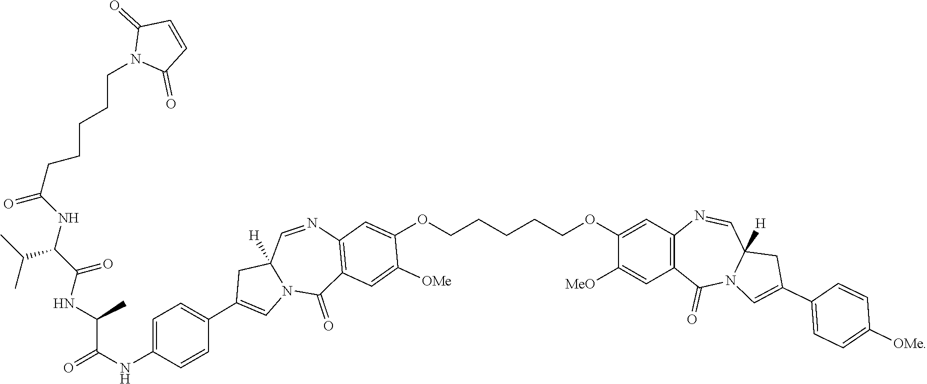 [SMILES: COc1ccc(C2=CN3C(=O)c4cc(OC)c(OCCCCCOc5cc6c(cc5OC)C(=O)N5C=C(c7ccc(NC(=O)[C@H](C)NC(=O)[C@@H](NC(=O)CCCCCN8C(=O)C=CC8=O)C(C)C)cc7)C[C@H]5C=N6)cc4N=C[C@@H]3C2)cc1]

This particular cytotoxin-linker conjugate is known as talirine, and has been described, for example, in connection with the ADC Vadastuximab talirine (SGN-CD33A), Mantaj et al., Angewandte Chemie International Edition English 2017, 56, 462-488, the disclosure of which is incorporated by reference herein in its entirety.

In some embodiments, the cytotoxin is an indolinobenzodiazepine pseudodimer having the structure of formula:

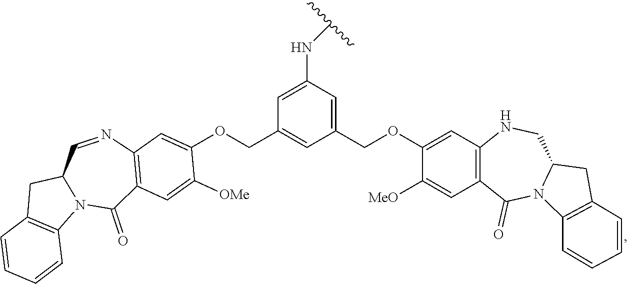 [SMILES: *Nc1cc(COc2cc3c(cc2OC)C(=O)N2c4ccccc4C[C@H]2C=N3)cc(COc2cc3c(cc2OC)C(=O)N2c4ccccc4C[C@H]2CN3)c1]

wherein the wavy line indicates the attachment point of the linker.

In some embodiments, the cytotoxin-linker conjugate, prior to conjugation to the antibody and including the reactive substituent Z′, taken together as Cy-L-Z′, has the structure:

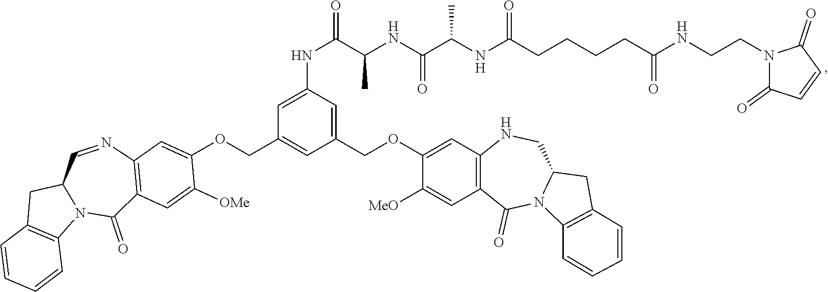 [SMILES: COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)NCCN3C(=O)C=CC3=O)c1)N=C[C@@H]1Cc3ccccc3N1C2=O]

which comprises the ADC IMGN632, disclosed in, for example, International Patent Application Publication No. WO2017004026, which is incorporated by reference herein.

### Preparation of Antibody-Drug Conjugates

In the ADCs of formula Ab-(Z-L-Cy)ₙ as disclosed herein, such as an ADC of formula (V), an anti-CD117 antibody or antigen binding fragment thereof (Ab) is conjugated to one or more cytotoxic drug moieties (Cy; e.g., a PBD), for example, from about 1 to about 20 cytotoxic moieties per antibody, through a linker L and a chemical moiety Z as disclosed herein. Any number of cytotoxins can be conjugated to the anti-CD117 antibody, e.g., 1, 2, 3, 4, 5, 6, 7, or 8. In some embodiments, n is from 1 to 4. In some embodiments, n is 2.

The ADCs of the present disclosure may be prepared by several routes, employing organic chemistry reactions, conditions, and reagents known to those skilled in the art, including: (1) reaction of a reactive substituent of an antibody or antigen binding fragment thereof with a bivalent linker reagent to form Ab-Z-L as described herein above, followed by reaction with a cytotoxic moiety Cy; or (2) reaction of a reactive substituent of a cytotoxic moiety with a bivalent linker reagent to form Cy-L-Z′, followed by reaction with a reactive substituent of an antibody or antigen binding fragment thereof as described herein above, to form an ADC of formula Ab-(Z-L-Cy)ₙ. Additional methods for preparing ADCs are described herein.

In one embodiment, the antibody or antigen binding fragment thereof can have one or more carbohydrate groups that can be chemically modified to have one or more sulfhydryl groups. The ADC is then formed by conjugation through the sulfhydryl group's sulfur atom as described herein above.

In another embodiment, the antibody can have one or more carbohydrate groups that can be oxidized to provide an aldehyde (—CHO) group (see, for e.g., Laguzza, et al., J. Med. Chem. 1989, 32(3), 548-55). The ADC is then formed by conjugation through the corresponding aldehyde as described herein above. Other protocols for the modification of proteins for the attachment or association of cytotoxins are described in Coligan et al., Current Protocols in Protein Science, vol. 2, John Wiley & Sons (2002), incorporated herein by reference.

Methods for the conjugation of linker-drug moieties to cell-targeted proteins such as antibodies, immunoglobulins or fragments thereof are found, for example, in U.S. Pat. Nos. 5,208,020; 6,441,163; WO2005037992; WO2005081711; and WO2006/034488, all of which are hereby expressly incorporated by reference in their entirety. Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

### Pharmaceutical Compositions

ADCs described herein can be administered to a patient (e.g., a human patient suffering from an immune disease or cancer) in a variety of dosage forms. For instance, ADCs described herein can be administered to a patient suffering from an immune disease or cancer in the form of an aqueous solution, such as an aqueous solution containing one or more pharmaceutically acceptable excipients. Suitable pharmaceutically acceptable excipients for use with the compositions and methods described herein include viscosity-modifying agents. The aqueous solution may be sterilized using techniques known in the art.

Pharmaceutical formulations comprising ADCs as described herein are prepared by mixing such ADC with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

### Administration

The ADCs described herein may be administered by a variety of routes, such as orally, transdermally, subcutaneously, intranasally, intravenously, intramuscularly, intraocularly, or parenterally. The most suitable route for administration in any given case will depend on the particular antibody, or antigen-binding fragment, administered, the patient, pharmaceutical formulation methods, administration methods (e.g., administration time and administration route), the patient's age, body weight, sex, severity of the diseases being treated, the patient's diet, and the patient's excretion rate.

The effective dose of an ADC described herein can range, for example from about 0.001 to about 100 mg/kg of body weight per single (e.g., bolus) administration, multiple administrations, or continuous administration, or to achieve an optimal serum concentration (e.g., a serum concentration of 0.0001-5000 μg/mL) of the antibody, antigen-binding fragment thereof. The dose may be administered one or more times (e.g., 2-10 times) per day, week, or month to a subject (e.g., a human) suffering from cancer, an autoimmune disease, or undergoing conditioning therapy in preparation for receipt of a hematopoietic stem cell transplant. In the case of a conditioning procedure prior to hematopoietic stem cell transplantation, the ADC, antibody, or antigen-binding fragment thereof, can be administered to the patient at a time that optimally promotes engraftment of the exogenous hematopoietic stem cells, for instance, from 1 hour to 1 week (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, or about 7 days) or more prior to administration of the exogenous hematopoietic stem cell transplant.

### Anti-CD117 Antibodies

The anti-CD117 ADC compositions and methods disclosed herein comprise an agent to facilitate the selective delivery of such ADCs to a population of cells in the target tissues (e.g., hematopoietic stem cells of the bone marrow stem cell niche).

In certain embodiments, an antibody, or antigen binding fragment thereof, in an ADC described herein has a certain dissociation rate for human CD117 which is particularly advantageous when used as a part of a conjugate. For example, an anti-CD117 antibody has, in certain embodiments, an off rate constant (Koff) for human CD117 and/or rhesus CD117 of 1×10⁻² to 1×10⁻³, 1×10⁻³ to 1×10⁻⁴, 1×10⁻⁵ to 1×10⁻⁶, 1×10⁻⁶ to 1×10⁻⁷ or 1×10⁻⁷ to 1×10⁻⁸, as measured by bio-layer interferometry (BLI). In some embodiments, the antibody or antigen-binding fragment thereof binds a cell surface antigen (e.g., human CD117 and/or rhesus CD117) with a K_{D} of about 100 nM or less, about 90 nM or less, about 80 nM or less, about 70 nM or less, about 60 nM or less, about 50 nM or less, about 40 nM or less, about 30 nM or less, about 20 nM or less, about 10 nM or less, about 8 nM or less, about 6 nM or less, about 4 nM or less, about 2 nM or less, about 1 nM or less as determined by a Bio-Layer Interferometry (BLI) assay.

In one embodiment, the present disclosure includes ADCs comprising antibodies, and antigen-binding fragments thereof, that specifically bind to CD117, such as GNNK+CD117. Such ADCs may be used as therapeutic agents to, for example, (i) treat cancers and autoimmune diseases characterized by CD117+ cells and (ii) promote the engraftment of transplanted hematopoietic stem cells in a patient in need of transplant therapy. These therapeutic activities can be caused, for instance, by the binding of isolated anti-CD117 antibodies, antigen-binding fragments thereof, that bind to CD117 (e.g., GNNK+CD117) expressed on the surface of a cell, such as a cancer cell, autoimmune cell, or hematopoietic stem cell and subsequently inducing cell death. The depletion of endogenous hematopoietic stem cells can provide a niche toward which transplanted hematopoietic stem cells can home, and subsequently establish productive hematopoiesis. In this way, transplanted hematopoietic stem cells may successfully engraft in a patient, such as human patient suffering from a stem cell disorder described herein.

Antibodies and antigen-binding fragments capable of binding human CD117 (also referred to as c-Kit, mRNA NCBI Reference Sequence: NM_000222.2, Protein NCBI Reference Sequence: NP_000213.1), including those capable of binding GNNK+CD117, can be used in conjunction with the compositions and methods described herein in order to condition a patient for hematopoietic stem cell transplant therapy. Polymorphisms affecting the coding region or extracellular domain of CD117 in a significant percentage of the population are not currently well-known in non-oncology indications. There are at least four isoforms of CD117 that have been identified, with the potential of additional isoforms expressed in tumor cells. Two of the CD117 isoforms are located on the intracellular domain of the protein, and two are present in the external juxtamembrane region. The two extracellular isoforms, GNNK+ and GNNK−, differ in the presence (GNNK+) or absence (GNNK−) of a 4 amino acid sequence. These isoforms are reported to have the same affinity for the ligand (SCF), but ligand binding to the GNNK− isoform was reported to increase internalization and degradation. The GNNK+ isoform can be used as an immunogen in order to generate antibodies capable of binding CD117, as antibodies generated against this isoform will be inclusive of the GNNK+ and GNNK− proteins. The amino acid sequences of human CD117 isoforms 1 and 2 are described in SEQ ID Nos: 145 and 146, respectively. In certain embodiments, anti-human CD117 (hCD117) antibodies disclosed herein are able to bind to both isoform 1 and isoform 2 of human CD117.

As described below, a yeast library screen of human antibodies was performed to identify novel anti-CD117 antibodies, and fragments thereof, having diagnostic and therapeutic use. Antibody 54 (Ab54), Antibody 55 (Ab55), Antibody 56 (Ab56), Antibody 57 (Ab57), Antibody 58 (Ab58), Antibody 61 (Ab61), Antibody 66 (Ab66), Antibody 67 (Ab67), Antibody 68 (Ab68), and Antibody 69 (Ab69) were human antibodies that were identified in this screen. These antibodies cross react with human CD117 and rhesus CD117. Further, these antibodies disclosed herein are able to bind to both isoforms of human CD117, i.e., isoform 1 (SEQ ID NO: 145) and isoform 2 (SEQ ID NO: 146).

The amino acid sequences for the various binding regions of anti-CD117 antibodies, including Ab54, Ab55, Ab56, Ab57, Ab58, Ab61, Ab66, Ab67, Ab68, and Ab69 are described in the Sequence Table below. Included in the present disclosure are human anti-CD117 antibodies comprising the CDRs as set forth in the Sequence Table, as well as human anti-CD117 antibodies comprising the variable regions set forth in the Sequence Table.

In one embodiment, the present disclosure provides an ADC comprising an anti-CD117 antibody, or antigen-binding fragment thereof, comprising binding regions, e.g., CDRs, variable regions, corresponding to those of Antibody 55. The heavy chain variable region (VH) amino acid sequence of Antibody 55 (i.e., Ab55) is set forth in SEQ ID NO: 19 (see Table 10). The VH CDR domain amino acid sequences of Antibody 55 are set forth in SEQ ID NO: 21 (VH CDR1); SEQ ID NO: 22 (VH CDR2), and SEQ ID NO: 23 (VH CDR3). The light chain variable region (VL) amino acid sequence of Antibody 55 is described in SEQ ID NO: 20 (see Table 10). The VL CDR domain amino acid sequences of Antibody 55 are set forth in SEQ ID NO: 24 (VL CDR1); SEQ ID NO: 25 (VL CDR2), and SEQ ID NO: 26 (VL CDR3). The heavy chain constant region of Antibody 55 is set forth in SEQ ID NO: 122. The light chain constant region of Antibody 55 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 21, 22, and 23, and a light chain variable region CDR set as set forth in SEQ ID Nos: 24, 25, and 26. In other embodiments, an anti-CD117 antibody, or antigen-binding portion thereof, comprises a variable light chain comprising the amino acid residues set forth in SEQ ID NO: 20, and a heavy chain variable region as set forth in SEQ ID NO: 19.

In one embodiment, the present disclosure provides an ADC comprising an anti-CD117 antibody, or antigen-binding fragment thereof, comprising binding regions, e.g., CDRs, variable regions, corresponding to those of Antibody 54. The heavy chain variable region (VH) amino acid sequence of Antibody 54 (i.e., Ab54) is set forth in SEQ ID NO: 29 (see Table 10). The VH CDR domain amino acid sequences of Antibody 54 are set forth in SEQ ID NO: 31 (VH CDR1); SEQ ID NO: 32 (VH CDR2), and SEQ ID NO: 33 (VH CDR3). The light chain variable region (VL) amino acid sequence of Antibody 54 is described in SEQ ID NO: 30 (see Table 10). The VL CDR domain amino acid sequences of Antibody 54 are set forth in SEQ ID NO: 34 (VL CDR1); SEQ ID NO: 35 (VL CDR2), and SEQ ID NO: 36 (VL CDR3). The heavy chain constant region of Antibody 54 is set forth in SEQ ID NO: 122. The light chain constant region of Antibody 54 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 31, 32, and 33, and a light chain variable region CDR set as set forth in SEQ ID Nos: 34, 35, and 36. In other embodiments, an anti-CD117 antibody, or antigen-binding portion thereof, comprises a variable light chain comprising the amino acid residues set forth in SEQ ID NO: 30, and a heavy chain variable region as set forth in SEQ ID NO: 29.

In one embodiment, the present disclosure provides an ADC comprising an anti-CD117 antibody, or antigen-binding fragment thereof, comprising binding regions, e.g., CDRs, variable regions, corresponding to those of Antibody 56. The heavy chain variable region (VH) amino acid sequence of Antibody 56 (i.e., Ab56) is set forth in SEQ ID NO: 39 (see Table 10). The VH CDR domain amino acid sequences of Antibody 56 are set forth in SEQ ID NO: 41 (VH CDR1); SEQ ID NO: 42 (VH CDR2), and SEQ ID NO: 43 (VH CDR3). The light chain variable region (VL) amino acid sequence of Antibody 56 is described in SEQ ID NO: 40 (see Table 10). The VL CDR domain amino acid sequences of Antibody 56 are set forth in SEQ ID NO: 44 (VL CDR1); SEQ ID NO: 45 (VL CDR2), and SEQ ID NO: 46 (VL CDR3). The heavy chain constant region of Antibody 56 is set forth in SEQ ID NO: 122. The light chain constant region of Antibody 56 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 41, 42, and 43, and a light chain variable region CDR set as set forth in SEQ ID Nos: 44, 45, and 46. In other embodiments, an anti-CD117 antibody, or antigen-binding portion thereof, comprises a variable light chain comprising the amino acid residues set forth in SEQ ID NO: 40, and a heavy chain variable region as set forth in SEQ ID NO: 39.

In one embodiment, the present disclosure provides an ADC comprising an anti-CD117 antibody, or antigen-binding fragment thereof, comprising binding regions, e.g., CDRs, variable regions, corresponding to those of Antibody 57. The heavy chain variable region (VH) amino acid sequence of Antibody 57 (i.e., Ab57) is set forth in SEQ ID NO: 49 (see Table 10). The VH CDR domain amino acid sequences of Antibody 57 are set forth in SEQ ID NO: 51 (VH CDR1); SEQ ID NO: 52 (VH CDR2), and SEQ ID NO: 53 (VH CDR3). The light chain variable region (VL) amino acid sequence of Antibody 57 is described in SEQ ID NO: 50 (see Table 10). The VL CDR domain amino acid sequences of Antibody 57 are set forth in SEQ ID NO: 54 (VL CDR1); SEQ ID NO: 55 (VL CDR2), and SEQ ID NO: 56 (VL CDR3). The heavy chain constant region of Antibody 57 is set forth in SEQ ID NO: 122. The light chain constant region of Antibody 57 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 51, 52, and 53, and a light chain variable region CDR set as set forth in SEQ ID Nos: 54, 55, and 56. In other embodiments, an anti-CD117 antibody, or antigen-binding portion thereof, comprises a variable light chain comprising the amino acid residues set forth in SEQ ID NO: 50, and a heavy chain variable region as set forth in SEQ ID NO: 49.

In one embodiment, the present disclosure provides an ADC comprising an anti-CD117 antibody, or antigen-binding fragment thereof, comprising binding regions, e.g., CDRs, variable regions, corresponding to those of Antibody 58. The heavy chain variable region (VH) amino acid sequence of Antibody 58 (i.e., Ab58) is set forth in SEQ ID NO: 59 (see Table 10). The VH CDR domain amino acid sequences of Antibody 58 are set forth in SEQ ID NO: 61 (VH CDR1); SEQ ID NO: 62 (VH CDR2), and SEQ ID NO: 63 (VH CDR3). The light chain variable region (VL) amino acid sequence of Antibody 58 is described in SEQ ID NO: 60 (see Table 10). The VL CDR domain amino acid sequences of Antibody 58 are set forth in SEQ ID NO: 64 (VL CDR1); SEQ ID NO: 65 (VL CDR2), and SEQ ID NO: 66 (VL CDR3). The heavy chain constant region of Antibody 58 is set forth in SEQ ID NO: 122. The light chain constant region of Antibody 58 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 61, 62, and 63, and a light chain variable region CDR set as set forth in SEQ ID Nos: 64, 65, and 66. In other embodiments, an anti-CD117 antibody, or antigen-binding portion thereof, comprises a variable light chain comprising the amino acid residues set forth in SEQ ID NO: 60, and a heavy chain variable region as set forth in SEQ ID NO: 59.

In one embodiment, the present disclosure provides an ADC comprising an anti-CD117 antibody, or antigen-binding fragment thereof, comprising binding regions, e.g., CDRs, variable regions, corresponding to those of Antibody 61. The heavy chain variable region (VH) amino acid sequence of Antibody 61 (i.e., Ab61) is set forth in SEQ ID NO: 69 (see Table 10). The VH CDR domain amino acid sequences of Antibody 61 are set forth in SEQ ID NO: 71 (VH CDR1); SEQ ID NO: 72 (VH CDR2), and SEQ ID NO: 73 (VH CDR3). The light chain variable region (VL) amino acid sequence of Antibody 61 is described in SEQ ID NO: 70 (see Table 10). The VL CDR domain amino acid sequences of Antibody 61 are set forth in SEQ ID NO: 74 (VL CDR1); SEQ ID NO: 75 (VL CDR2), and SEQ ID NO: 76 (VL CDR3). The heavy chain constant region of Antibody 61 is set forth in SEQ ID NO: 122. The light chain constant region of Antibody 61 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 71, 72, and 73, and a light chain variable region CDR set as set forth in SEQ ID Nos: 74, 75, and 76. In other embodiments, an anti-CD117 antibody, or antigen-binding portion thereof, comprises a variable light chain comprising the amino acid residues set forth in SEQ ID NO: 70, and a heavy chain variable region as set forth in SEQ ID NO: 69.

In one embodiment, the present disclosure provides an ADC comprising an anti-CD117 antibody, or antigen-binding fragment thereof, comprising binding regions, e.g., CDRs, variable regions, corresponding to those of Antibody 66. The heavy chain variable region (VH) amino acid sequence of Antibody 66 (i.e., Ab66) is set forth in SEQ ID NO: 79 (see Table 10). The VH CDR domain amino acid sequences of Antibody 66 are set forth in SEQ ID NO: 81 (VH CDR1); SEQ ID NO: 82 (VH CDR2), and SEQ ID NO: 83 (VH CDR3). The light chain variable region (VL) amino acid sequence of Antibody 66 is described in SEQ ID NO: 80 (see Table 10). The VL CDR domain amino acid sequences of Antibody 66 are set forth in SEQ ID NO: 84 (VL CDR1); SEQ ID NO: 85 (VL CDR2), and SEQ ID NO: 86 (VL CDR3). The heavy chain constant region of Antibody 66 is set forth in SEQ ID NO: 122. The light chain constant region of Antibody 66 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 81, 82, and 83, and a light chain variable region CDR set as set forth in SEQ ID Nos: 84, 85, and 86. In other embodiments, an anti-CD117 antibody, or antigen-binding portion thereof, comprises a variable light chain comprising the amino acid residues set forth in SEQ ID NO: 80, and a heavy chain variable region as set forth in SEQ ID NO: 79.

In one embodiment, the present disclosure provides an ADC comprising an anti-CD117 antibody, or antigen-binding fragment thereof, comprising binding regions, e.g., CDRs, variable regions, corresponding to those of Antibody 67. The heavy chain variable region (VH) amino acid sequence of Antibody 67 is set forth in SEQ ID NO: 9 (see Table 2). The VH CDR domain amino acid sequences of Antibody 67 are set forth in SEQ ID NO 11 (VH CDR1); SEQ ID NO: 12 (VH CDR2), and SEQ ID NO: 13 (VH CDR3). The light chain variable region (VL) amino acid sequence of Antibody 67 is described in SEQ ID NO: 10 (see Table 2). The VL CDR domain amino acid sequences of Antibody 67 are set forth in SEQ ID NO 14 (VL CDR1); SEQ ID NO: 15 (VL CDR2), and SEQ ID NO: 16 (VL CDR3). The full length heavy chain (HC) of Antibody 67 is set forth in SEQ ID NO: 110, and the full length heavy chain constant region of Antibody 67 is set forth in SEQ ID NO: 122. The light chain (LC) of Antibody 67 is set forth in SEQ ID NO: 109. The light chain constant region of Antibody 67 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 11, 12, and 13, and a light chain variable region CDR set as set forth in SEQ ID Nos: 14, 15, and 16. In other embodiments, an anti-CD117 antibody, or antigen-binding portion thereof, comprises a variable heavy chain comprising the amino acid residues set forth in SEQ ID NO: 9, and a heavy chain variable region as set forth in SEQ ID NO: 10. In further embodiments, an anti-CD117 antibody comprises a heavy chain comprising SEQ ID NO: 110 and a light chain comprising SEQ ID NO: 109.

In one embodiment, the present disclosure provides an ADC comprising an anti-CD117 antibody, or antigen-binding fragment thereof, comprising binding regions, e.g., CDRs, variable regions, corresponding to those of Antibody 68. The heavy chain variable region (VH) amino acid sequence of Antibody 68 (i.e., Ab68) is set forth in SEQ ID NO: 89 (see Table 10). The VH CDR domain amino acid sequences of Antibody 68 are set forth in SEQ ID NO: 91 (VH CDR1); SEQ ID NO: 92 (VH CDR2), and SEQ ID NO: 93 (VH CDR3). The light chain variable region (VL) amino acid sequence of Antibody 68 is described in SEQ ID NO: 90 (see Table 10). The VL CDR domain amino acid sequences of Antibody 68 are set forth in SEQ ID NO: 94 (VL CDR1); SEQ ID NO: 95 (VL CDR2), and SEQ ID NO: 96 (VL CDR3). The heavy chain constant region of Antibody 68 is set forth in SEQ ID NO: 122. The light chain constant region of Antibody 68 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 91, 92, and 93, and a light chain variable region CDR set as set forth in SEQ ID Nos: 94, 95, and 96. In other embodiments, an anti-CD117 antibody, or antigen-binding portion thereof, comprises a variable light chain comprising the amino acid residues set forth in SEQ ID NO: 90, and a heavy chain variable region as set forth in SEQ ID NO: 89.

In one embodiment, the present disclosure provides an ADC comprising an anti-CD117 antibody, or antigen-binding fragment thereof, comprising binding regions, e.g., CDRs, variable regions, corresponding to those of Antibody 69. The heavy chain variable region (VH) amino acid sequence of Antibody 69 (i.e., Ab69) is set forth in SEQ ID NO: 99 (see Table 10). The VH CDR domain amino acid sequences of Antibody 69 are set forth in SEQ ID NO: 101 (VH CDR1); SEQ ID NO: 102 (VH CDR2), and SEQ ID NO: 103 (VH CDR3). The light chain variable region (VL) amino acid sequence of Antibody 69 is described in SEQ ID NO: 100 (see Table 10). The VL CDR domain amino acid sequences of Antibody 69 are set forth in SEQ ID NO: 104 (VL CDR1); SEQ ID NO: 105 (VL CDR2), and SEQ ID NO: 106 (VL CDR3). The heavy chain constant region of Antibody 69 is set forth in SEQ ID NO: 122. The light chain constant region of Antibody 69 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 101, 102, and 103, and a light chain variable region CDR set as set forth in SEQ ID Nos: 104, 105, and 106. In other embodiments, an anti-CD117 antibody, or antigen-binding portion thereof, comprises a variable light chain comprising the amino acid residues set forth in SEQ ID NO: 100, and a heavy chain variable region as set forth in SEQ ID NO: 99.

Further, the amino acid sequences for the various binding regions of the anti-CD117 antibodies Ab77, Ab79, Ab81, Ab85, Ab86, Ab87, Ab88, and Ab89 are described in the Sequence Table below.

In one embodiment, the present disclosure provides an ADC comprising an anti-CD117 antibody, or antigen-binding fragment thereof, comprising binding regions, e.g., CDRs, variable regions, corresponding to those of Antibody 77. The heavy chain variable region (VH) amino acid sequence of Antibody 77 (i.e., Ab77) is set forth in SEQ ID NO: 147 (see Table 10). The VH CDR domain amino acid sequences of Antibody 77 are set forth in SEQ ID NO: 263 (VH CDR1); SEQ ID NO: 2 (VH CDR2), and SEQ ID NO: 3 (VH CDR3). The light chain variable region (VL) amino acid sequence of Antibody 77 is described in SEQ ID NO: 231 (see Table 10). The VL CDR domain amino acid sequences of Antibody 77 are set forth in SEQ ID NO: 264 (VL CDR1); SEQ ID NO: 265 (VL CDR2), and SEQ ID NO: 266 (VL CDR3). The heavy chain constant region of Antibody 77 is set forth in SEQ ID NO: 269. The light chain constant region of Antibody 77 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 263, 2, and 3, and a light chain variable region CDR set as set forth in SEQ ID Nos: 264, 265, and 266. In other embodiments, an anti-CD117 antibody, or antigen-binding portion thereof, comprises a variable light chain comprising the amino acid residues set forth in SEQ ID NO: 231, and a heavy chain variable region as set forth in SEQ ID NO: 147.

In one embodiment, the present disclosure provides an ADC comprising an anti-CD117 antibody, or antigen-binding fragment thereof, comprising binding regions, e.g., CDRs, variable regions, corresponding to those of Antibody 79. The heavy chain variable region (VH) amino acid sequence of Antibody 79 (i.e., Ab79) is set forth in SEQ ID NO: 147 (see Table 10). The VH CDR domain amino acid sequences of Antibody 79 are set forth in SEQ ID NO: 263 (VH CDR1); SEQ ID NO: 2 (VH CDR2), and SEQ ID NO: 3 (VH CDR3). The light chain variable region (VL) amino acid sequence of Antibody 79 is described in SEQ ID NO: 233 (see Table 10). The VL CDR domain amino acid sequences of Antibody 79 are set forth in SEQ ID NO: 267 (VL CDR1); SEQ ID NO: 265 (VL CDR2), and SEQ ID NO: 266 (VL CDR3). The heavy chain constant region of Antibody 79 is set forth in SEQ ID NO: 269. The light chain constant region of Antibody 79 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 263, 2, and 3, and a light chain variable region CDR set as set forth in SEQ ID Nos: 267, 265, and 266. In other embodiments, an anti-CD117 antibody, or antigen-binding portion thereof, comprises a variable light chain comprising the amino acid residues set forth in SEQ ID NO: 233, and a heavy chain variable region as set forth in SEQ ID NO: 147.

In one embodiment, the present disclosure provides an ADC comprising an anti-CD117 antibody, or antigen-binding fragment thereof, comprising binding regions, e.g., CDRs, variable regions, corresponding to those of Antibody 81. The heavy chain variable region (VH) amino acid sequence of Antibody 81 (i.e., Ab81) is set forth in SEQ ID NO: 147 (see Table 10). The VH CDR domain amino acid sequences of Antibody 81 are set forth in SEQ ID NO: 263 (VH CDR1); SEQ ID NO: 2 (VH CDR2), and SEQ ID NO: 3 (VH CDR3). The light chain variable region (VL) amino acid sequence of Antibody 81 is described in SEQ ID NO: 235 (see Table 10). The VL CDR domain amino acid sequences of Antibody 81 are set forth in SEQ ID NO: 264 (VL CDR1); SEQ ID NO: 268 (VL CDR2), and SEQ ID NO: 266 (VL CDR3). The heavy chain constant region of Antibody 81 is set forth in SEQ ID NO: 269. The light chain constant region of Antibody 81 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 263, 2, and 3, and a light chain variable region CDR set as set forth in SEQ ID Nos: 264, 268, and 266. In other embodiments, an anti-CD117 antibody, or antigen-binding portion thereof, comprises a variable light chain comprising the amino acid residues set forth in SEQ ID NO: 235, and a heavy chain variable region as set forth in SEQ ID NO: 147.

In one embodiment, the present disclosure provides an ADC comprising an anti-CD117 antibody, or antigen-binding fragment thereof, comprising binding regions, e.g., CDRs, variable regions, corresponding to those of Antibody 85. The heavy chain variable region (VH) amino acid sequence of Antibody 85 (i.e., Ab86) is set forth in SEQ ID NO: 243 (see Table 10). The VH CDR domain amino acid sequences of Antibody 85 are set forth in SEQ ID NO: 245 (VH CDR1); SEQ ID NO: 246 (VH CDR2), and SEQ ID NO: 247 (VH CDR3). The light chain variable region (VL) amino acid sequence of Antibody 85 is described in SEQ ID NO: 242 (see Table 10). The VL CDR domain amino acid sequences of Antibody 85 are set forth in SEQ ID NO: 248 (VL CDR1); SEQ ID NO: 249 (VL CDR2), and SEQ ID NO: 250 (VL CDR3). The heavy chain constant region of Antibody 85 is set forth in SEQ ID NO: 269. The light chain constant region of Antibody 85 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 245, 246, and 247, and a light chain variable region CDR set as set forth in SEQ ID Nos: 248, 249, and 250. In other embodiments, an anti-CD117 antibody, or antigen-binding portion thereof, comprises a variable light chain comprising the amino acid residues set forth in SEQ ID NO: 244, and a heavy chain variable region as set forth in SEQ ID NO: 243.

In one embodiment, the present disclosure provides an ADC comprising an anti-CD117 antibody, or antigen-binding fragment thereof, comprising binding regions, e.g., CDRs, variable regions, corresponding to those of Antibody 86. The heavy chain variable region (VH) amino acid sequence of Antibody 86 (i.e., Ab86) is set forth in SEQ ID NO: 251 (see Table 10). The VH CDR domain amino acid sequences of Antibody 86 are set forth in SEQ ID NO: 245 (VH CDR1); SEQ ID NO: 253 (VH CDR2), and SEQ ID NO: 3 (VH CDR3). The light chain variable region (VL) amino acid sequence of Antibody 86 is described in SEQ ID NO: 252 (see Table 10). The VL CDR domain amino acid sequences of Antibody 86 are set forth in SEQ ID NO: 254 (VL CDR1); SEQ ID NO: 249 (VL CDR2), and SEQ ID NO: 255 (VL CDR3). The heavy chain constant region of Antibody 86 is set forth in SEQ ID NO: 269. The light chain constant region of Antibody 86 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 245, 253, and 3, and a light chain variable region CDR set as set forth in SEQ ID Nos: 254, 249, and 255. In other embodiments, an anti-CD117 antibody, or antigen-binding portion thereof, comprises a variable light chain comprising the amino acid residues set forth in SEQ ID NO: 252, and a heavy chain variable region as set forth in SEQ ID NO: 251.

In one embodiment, the present disclosure provides an ADC comprising an anti-CD117 antibody, or antigen-binding fragment thereof, comprising binding regions, e.g., CDRs, variable regions, corresponding to those of Antibody 87. The heavy chain variable region (VH) amino acid sequence of Antibody 87 (i.e., Ab87) is set forth in SEQ ID NO: 243 (see Table 10). The VH CDR domain amino acid sequences of Antibody 87 are set forth in SEQ ID NO: 245 (VH CDR1); SEQ ID NO: 246 (VH CDR2), and SEQ ID NO: 247 (VH CDR3). The light chain variable region (VL) amino acid sequence of Antibody 87 is described in SEQ ID NO: 256 (see Table 10). The VL CDR domain amino acid sequences of Antibody 87 are set forth in SEQ ID NO: 257 (VL CDR1); SEQ ID NO: 5 (VL CDR2), and SEQ ID NO: 255 (VL CDR3). The heavy chain constant region of Antibody 87 is set forth in SEQ ID NO: 269. The light chain constant region of Antibody 87 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 245, 246, and 247, and a light chain variable region CDR set as set forth in SEQ ID Nos: 257, 5, and 255. In other embodiments, an anti-CD117 antibody, or antigen-binding portion thereof, comprises a variable light chain comprising the amino acid residues set forth in SEQ ID NO: 256, and a heavy chain variable region as set forth in SEQ ID NO: 243.

In one embodiment, the present disclosure provides an anti-CD117 antibody, or antigen-binding fragment thereof, comprising binding regions, e.g., CDRs, variable regions, corresponding to those of Antibody 88. The heavy chain variable region (VH) amino acid sequence of Antibody 88 (i.e., Ab88) is set forth in SEQ ID NO: 258 (see Table 10). The VH CDR domain amino acid sequences of Antibody 88 are set forth in SEQ ID NO: 245 (VH CDR1); SEQ ID NO: 259 (VH CDR2), and SEQ ID NO: 3 (VH CDR3). The light chain variable region (VL) amino acid sequence of Antibody 88 is described in SEQ ID NO: 256 (see Table 10). The VL CDR domain amino acid sequences of Antibody 88 are set forth in SEQ ID NO: 257 (VL CDR1); SEQ ID NO: 5 (VL CDR2), and SEQ ID NO: 255 (VL CDR3). The heavy chain constant region of Antibody 88 is set forth in SEQ ID NO: 269. The light chain constant region of Antibody 88 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 245, 259, and 3, and a light chain variable region CDR set as set forth in SEQ ID Nos: 257, 5, and 255. In other embodiments, an anti-CD117 antibody, or antigen-binding portion thereof, comprises a variable light chain comprising the amino acid residues set forth in SEQ ID NO: 256, and a heavy chain variable region as set forth in SEQ ID NO: 258.

In one embodiment, the present disclosure provides an ADC comprising an anti-CD117 antibody, or antigen-binding fragment thereof, comprising binding regions, e.g., CDRs, variable regions, corresponding to those of Antibody 89. The heavy chain variable region (VH) amino acid sequence of Antibody 89 (i.e., Ab89) is set forth in SEQ ID NO: 260 (see Table 10). The VH CDR domain amino acid sequences of Antibody 89 are set forth in SEQ ID NO: 245 (VH CDR1); SEQ ID NO: 2 (VH CDR2), and SEQ ID NO: 3 (VH CDR3). The light chain variable region (VL) amino acid sequence of Antibody 89 is described in SEQ ID NO: 252 (see Table 10). The VL CDR domain amino acid sequences of Antibody 89 are set forth in SEQ ID NO: 254 (VL CDR1); SEQ ID NO: 249 (VL CDR2), and SEQ ID NO: 255 (VL CDR3). The heavy chain constant region of Antibody 89 is set forth in SEQ ID NO: 269. The light chain constant region of Antibody 89 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 245, 2, and 3, and a light chain variable region CDR set as set forth in SEQ ID Nos: 254, 249, and 255. In other embodiments, an anti-CD117 antibody, or antigen-binding portion thereof, comprises a variable light chain comprising the amino acid residues set forth in SEQ ID NO: 252, and a heavy chain variable region as set forth in SEQ ID NO: 260.

In one embodiment, the present disclosure provides an ADC comprising an anti-CD117 antibody, or antigen-binding fragment thereof, comprising binding regions, e.g., CDRs, variable regions, corresponding to those of Antibody 249. The heavy chain variable region (VH) amino acid sequence of Antibody 249 (i.e., Ab249) is set forth in SEQ ID NO: 238 (see Table 10). The VH CDR domain amino acid sequences of Antibody 249 are set forth in SEQ ID NO: 286 (VH CDR1); SEQ ID NO: 2 (VH CDR2), and SEQ ID NO: 287 (VH CDR3). The light chain variable region (VL) amino acid sequence of Antibody 249 is described in SEQ ID NO: 242 (see Table 10). The VL CDR domain amino acid sequences of Antibody 249 are set forth in SEQ ID NO: 288 (VL CDR1); SEQ ID NO: 249 (VL CDR2), and SEQ ID NO: 289 (VL CDR3). The heavy chain constant region of Antibody 249 is set forth in SEQ ID NO: 269. The light chain constant region of Antibody 249 is set forth in SEQ ID NO: 283. Thus, in certain embodiments, an anti-CD117 antibody, or antigen-binding portion thereof, comprises a variable heavy chain CDR set (CDR1, CDR2, and CDR3) as set forth in SEQ ID Nos: 286, 2, and 287, and a light chain variable region CDR set as set forth in SEQ ID Nos: 288, 249, and 289. In other embodiments, an anti-CD117 antibody, or antigen-binding portion thereof, comprises a variable light chain comprising the amino acid residues set forth in SEQ ID NO: 242, and a heavy chain variable region as set forth in SEQ ID NO: 238.

Further, included in the disclosure is anti-CD117 antibody drug conjugates comprising binding regions (heavy and light chain CDRs or variable regions) as set forth in SEQ ID Nos: 147 to 168. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 148. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 149. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 150. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 151. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 152. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 153. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 154. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 155. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 156. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 157. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 158. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 159. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 160. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 161. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 162. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 163. In one embodiment, the anti-CD117 antibody, or antigen binding portion thereof, comprises a heavy chain variable region as set forth in the amino acid sequence of SEQ ID NO: 164, and a light chain variable region as set forth in the amino acid sequence of SEQ ID NO: 165. In one embodiment, the anti-CD117 antibody, or antigen binding portion thereof, comprises a heavy chain variable region as set forth in the amino acid sequence of SEQ ID NO: 166, and a light chain variable region as set forth in the amino acid sequence of SEQ ID NO: 167. In one embodiment, the anti-CD117 antibody, or antigen binding portion thereof, comprises a heavy chain variable region as set forth in the amino acid sequence of SEQ ID NO: 168, and a light chain variable region as set forth in the amino acid sequence of SEQ ID NO: 169. In one embodiment, the anti-CD117 antibody, or antigen binding portion thereof, comprises a heavy chain variable region as set forth in the amino acid sequence of SEQ ID NO: 170, and a light chain variable region as set forth in the amino acid sequence of SEQ ID NO: 171. In one embodiment, the anti-CD117 antibody, or antigen binding portion thereof, comprises a heavy chain variable region as set forth in the amino acid sequence of SEQ ID NO: 172, and a light chain variable region as set forth in the amino acid sequence of SEQ ID NO: 173. In one embodiment, the anti-CD117 antibody, or antigen binding portion thereof, comprises a heavy chain variable region as set forth in the amino acid sequence of SEQ ID NO: 174, and a light chain variable region as set forth in the amino acid sequence of SEQ ID NO: 175. In one embodiment, the anti-CD117 antibody, or antigen binding portion thereof, comprises a heavy chain variable region as set forth in the amino acid sequence of SEQ ID NO: 176, and a light chain variable region as set forth in the amino acid sequence of SEQ ID NO: 177. In one embodiment, the anti-CD117 antibody, or antigen binding portion thereof, comprises a heavy chain variable region as set forth in the amino acid sequence of SEQ ID NO: 178, and a light chain variable region as set forth in the amino acid sequence of SEQ ID NO: 179. In one embodiment, the anti-CD117 antibody, or antigen binding portion thereof, comprises a heavy chain variable region as set forth in the amino acid sequence of SEQ ID NO: 180, and a light chain variable region as set forth in the amino acid sequence of SEQ ID NO: 181. In one embodiment, the anti-CD117 antibody, or antigen binding portion thereof, comprises a heavy chain variable region as set forth in the amino acid sequence of SEQ ID NO: 172, and a light chain variable region as set forth in the amino acid sequence of SEQ ID NO: 182. In one embodiment, the anti-CD117 antibody, or antigen binding portion thereof, comprises a heavy chain variable region as set forth in the amino acid sequence of SEQ ID NO: 183, and a light chain variable region as set forth in the amino acid sequence of SEQ ID NO: 184. In one embodiment, the anti-CD117 antibody, or antigen binding portion thereof, comprises a heavy chain variable region as set forth in the amino acid sequence of SEQ ID NO: 185, and a light chain variable region as set forth in the amino acid sequence of SEQ ID NO: 186. In one embodiment, the anti-CD117 antibody, or antigen binding portion thereof, comprises a heavy chain variable region as set forth in the amino acid sequence of SEQ ID NO: 187, and a light chain variable region as set forth in the amino acid sequence of SEQ ID NO: 188. In one embodiment, the anti-CD117 antibody, or antigen binding portion thereof, comprises a heavy chain variable region as set forth in the amino acid sequence of SEQ ID NO: 189, and a light chain variable region as set forth in the amino acid sequence of SEQ ID NO: 190. In one embodiment, the anti-CD117 antibody, or antigen binding portion thereof, comprises a heavy chain variable region as set forth in the amino acid sequence of SEQ ID NO: 191, and a light chain variable region as set forth in the amino acid sequence of SEQ ID NO: 192. In one embodiment, the anti-CD117 antibody, or antigen binding portion thereof, comprises a heavy chain variable region as set forth in the amino acid sequence of SEQ ID NO: 193, and a light chain variable region as set forth in the amino acid sequence of SEQ ID NO: 194. In one embodiment, the anti-CD117 antibody, or antigen binding portion thereof, comprises a heavy chain variable region as set forth in the amino acid sequence of SEQ ID NO: 195, and a light chain variable region as set forth in the amino acid sequence of SEQ ID NO: 196. In one embodiment, the anti-CD117 antibody, or antigen binding portion thereof, comprises a heavy chain variable region as set forth in the amino acid sequence of SEQ ID NO: 197, and a light chain variable region as set forth in the amino acid sequence of SEQ ID NO: 198. In one embodiment, the anti-CD117 antibody, or antigen binding portion thereof, comprises a heavy chain variable region as set forth in the amino acid sequence of SEQ ID NO: 199, and a light chain variable region as set forth in the amino acid sequence of SEQ ID NO: 200. In one embodiment, the anti-CD117 antibody, or antigen binding portion thereof, comprises a heavy chain variable region as set forth in the amino acid sequence of SEQ ID NO: 201, and a light chain variable region as set forth in the amino acid sequence of SEQ ID NO: 190. In one embodiment, the anti-CD117 antibody, or antigen binding portion thereof, comprises a heavy chain variable region as set forth in the amino acid sequence of SEQ ID NO: 202, and a light chain variable region as set forth in the amino acid sequence of SEQ ID NO: 203. In one embodiment, the anti-CD117 antibody, or antigen binding portion thereof, comprises a heavy chain variable region as set forth in the amino acid sequence of SEQ ID NO: 204, and a light chain variable region as set forth in the amino acid sequence of SEQ ID NO: 205. In one embodiment, the anti-CD117 antibody, or antigen binding portion thereof, comprises a heavy chain variable region as set forth in the amino acid sequence of SEQ ID NO: 206, and a light chain variable region as set forth in the amino acid sequence of SEQ ID NO: 207. In one embodiment, the anti-CD117 antibody, or antigen binding portion thereof, comprises a heavy chain variable region as set forth in the amino acid sequence of SEQ ID NO: 208, and a light chain variable region as set forth in the amino acid sequence of SEQ ID NO: 209. In one embodiment, the anti-CD117 antibody, or antigen binding portion thereof, comprises a heavy chain variable region as set forth in the amino acid sequence of SEQ ID NO: 210, and a light chain variable region as set forth in the amino acid sequence of SEQ ID NO: 211. In one embodiment, the anti-CD117 antibody, or antigen binding portion thereof, comprises a heavy chain variable region as set forth in the amino acid sequence of SEQ ID NO: 212, and a light chain variable region as set forth in the amino acid sequence of SEQ ID NO: 213. In one embodiment, the anti-CD117 antibody, or antigen binding portion thereof, comprises a heavy chain variable region as set forth in the amino acid sequence of SEQ ID NO: 214, and a light chain variable region as set forth in the amino acid sequence of SEQ ID NO: 215. In one embodiment, the anti-CD117 antibody, or antigen binding portion thereof, comprises a heavy chain variable region as set forth in the amino acid sequence of SEQ ID NO: 216, and a light chain variable region as set forth in the amino acid sequence of SEQ ID NO: 217. In one embodiment, the anti-CD117 antibody, or antigen binding portion thereof, comprises a heavy chain variable region as set forth in the amino acid sequence of SEQ ID NO: 218, and a light chain variable region as set forth in the amino acid sequence of SEQ ID NO: 219. In one embodiment, the anti-CD117 antibody, or antigen binding portion thereof, comprises a heavy chain variable region as set forth in the amino acid sequence of SEQ ID NO: 220, and a light chain variable region as set forth in the amino acid sequence of SEQ ID NO: 221. In one embodiment, the anti-CD117 antibody, or antigen binding portion thereof, comprises a heavy chain variable region as set forth in the amino acid sequence of SEQ ID NO: 222, and a light chain variable region as set forth in the amino acid sequence of SEQ ID NO: 223. In one embodiment, the anti-CD117 antibody, or antigen binding portion thereof, comprises a heavy chain variable region as set forth in the amino acid sequence of SEQ ID NO: 224, and a light chain variable region as set forth in the amino acid sequence of SEQ ID NO: 225. In one embodiment, the anti-CD117 antibody, or antigen binding portion thereof, comprises a heavy chain variable region as set forth in the amino acid sequence of SEQ ID NO: 226, and a light chain variable region as set forth in the amino acid sequence of SEQ ID NO: 227. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 228. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 229. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 230. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 231. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 232. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 233. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 234. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 235. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 236.

In one embodiment, an ADC comprises an anti-CD117 antibody, or antigen binding portion thereof, comprising a heavy chain variable region as set forth in the amino acid sequence of SEQ ID NO: 147, and a light chain variable region as set forth in the amino acid sequence of SEQ ID NO: 237. In one embodiment, the anti-CD117 antibody, or antigen binding portion thereof, comprises a heavy chain variable region as set forth in the amino acid sequence of SEQ ID NO: 243, and a light chain variable region as set forth in the amino acid sequence of SEQ ID NO: 244. In one embodiment, the anti-CD117 antibody, or antigen binding portion thereof, comprises a heavy chain variable region as set forth in the amino acid sequence of SEQ ID NO: 251, and a light chain variable region as set forth in the amino acid sequence of SEQ ID NO: 252. In one embodiment, the anti-CD117 antibody, or antigen binding portion thereof, comprises a heavy chain variable region as set forth in the amino acid sequence of SEQ ID NO: 243, and a light chain variable region as set forth in the amino acid sequence of SEQ ID NO: 256. In one embodiment, the anti-CD117 antibody, or antigen binding portion thereof, comprises a heavy chain variable region as set forth in the amino acid sequence of SEQ ID NO: 258, and a light chain variable region as set forth in the amino acid sequence of SEQ ID NO: 256. In one embodiment, the anti-CD117 antibody, or antigen binding portion thereof, comprises a heavy chain variable region as set forth in the amino acid sequence of SEQ ID NO: 260, and a light chain variable region as set forth in the amino acid sequence of SEQ ID NO: 252. In one embodiment, the anti-CD117 antibody, or antigen binding portion thereof, comprises a heavy chain variable region as set forth in the amino acid sequence of SEQ ID NO: 238, and a light chain variable region as set forth in the amino acid sequence of SEQ ID NO: 239. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 239. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 240. In one embodiment, the anti-CD117 antibody, or antigen binding portion thereof, comprises a heavy chain variable region as set forth in the amino acid sequence of SEQ ID NO: 238, and a light chain variable region as set forth in the amino acid sequence of SEQ ID NO: 241. In one embodiment, the anti-CD117 antibody, or antigen binding portion thereof, comprises a heavy chain variable region as set forth in the amino acid sequence of SEQ ID NO: 238, and a light chain variable region as set forth in the amino acid sequence of SEQ ID NO: 242.

Certain of the anti-CD117 antibodies described herein are neutral antibodies, in that the antibodies do not substantially inhibit CD117 activity on a CD117 expressing cell. Neutral antibodies can be identified using, for example, an in in vitro stem cell factor (SCF)-dependent cell proliferation assay. In an SCF dependent cell proliferation assay, a neutral CD117 antibody will not kill CD34+ cells that are dependent on SCF to divide, as a neutral antibody will not block SCF from binding to CD117 such as to inhibit CD117 activity.

Neutral antibodies can be used for diagnostic purposes, given their ability to specifically bind to human CD117, but are also effective for killing CD117 expressing cells when conjugated to a cytotoxin, such as those described herein. Typically, antibodies used in conjugates have agonistic or antagonistic activity that is unique to the antibody. Described herein, however, is a unique approach to conjugates, especially in the context wherein the conjugate is being used as a conditioning agent prior to a stem cell transplantation. While antagonistic antibodies alone or in combination with a cytotoxin as a conjugate can be effective given the killing ability of the antibody alone in addition to the cytotoxin, conditioning with a conjugate comprising a neutral anti-CD117 antibody presents an alternative strategy where the activity of the antibody is secondary to the effect of the cytotoxin, but the internalizing and affinity characteristics, e.g., dissociation rate, of the antibody are important for effective delivery of the cytotoxin.

Examples of neutral anti-CD117 antibodies include Ab58, Ab61, Ab66, Ab67, Ab68, and Ab69. A comparison of the amino acid sequences of the CDRs of neutral, anti-CD117 antibody CDRs reveals consensus sequences among two groups of neutral antibodies identified. Ab58 and Ab61 share the same light chain CDRs and HC CDR3, with slight variations in the HC CDR1 and HC CDR2. Consensus sequences for the HC CDR1 and CDR2 are described in SEQ ID Nos: 133 and 134. Ab66, Ab67, Ab68, and Ab69 are also neutral antibodies. While Ab66, Ab67, Ab68, and Ab69 share the same light chain CDRs and the same HC CDR3, these antibodies have variability within their HC CDR1 and HC CDR2 regions. Consensus sequences for these antibodies in the HC CDR1 and HC CDR2 regions are provided in SEQ ID Nos: 139 and 140, respectively.

Antagonist antibodies are also provided herein, including Ab54, Ab55, Ab56, and Ab57. While Ab54, Ab55, Ab56, and Ab57 share the same light chain CDRs and the same HC CDR3, these antibodies have variability within their HC CDR1 and HC CDR2 regions. Consensus sequences for these antibodies in the HC CDR1 and HC CDR2 regions are provided in SEQ ID Nos: 127 and 128, respectively.

In one aspect, the present disclosure pertains to an antibody, or an antigen binding fragment thereof, capable of binding CD117, which binds to an epitope in CD117 comprising at least two, at least three, at least four, at least five, at least six, at least seven, or all eight of the amino acid residues of T67, K69, T71, S81, Y83, T114, T119, or K129 of SEQ ID NO:290. In another aspect, the present disclosure pertains to an antibody, or antigen binding fragment thereof, capable of binding CD117 that binds to an epitope having residues within at least amino acids 67-83 and 114-129 of SEQ ID NO:290.

In another aspect, the present disclosure pertains to an antibody, or antigen binding fragment thereof, capable of binding CD117 which binds to an epitope in CD117, comprising at least two, at least three, at least four, at least five, or all six of the amino acid residues S236, H238, Y244, S273, T277 or T279 of SEQ ID NO:290. In one aspect, the present disclosure provides an isolated anti-CD117 antibody, or antigen-binding fragment thereof, capable of binding CD117 that binds to an epitope having residues within at least amino acids 236-244 and 273-279 of SEQ ID NO: 290.

(SEQ ID NO: 290)
QPSVSPGEPSPPSIHPGKSDLIVRVGDEIRLLCTDPGFVKWTFEILDETN
ENKQNEWITEKAEATNTGKYTCTNKHGLSNSIYVFVRDPAKLFLVDRSLY
GKEDNDTLVRCPLTDPEVTNYSLKGCQGKPLPKDLRFIPDPKAGIMIKSV
KRAYHRLCLHCSVDQEGKSVLSEKFILKVRPAFKAVPVVSVSKASYLLRE
GEEFTVTCTIKDVSSSVYSTWKRENSQTKLQEKYNSWHHGDFNYERQATL
TISSARVNDSGVFMCYANNTFGSANVTTTLEVVDKGFINIFPMINTTVFV
NDGENVDLIVEYEAFPKPEHQQWIYMNRTFTDKWEDYPKSENESNIRYVS
ELHLTRLKGTEGGTYTFLVSNSDVNAAIAFNVYVNTKPEILTYDRLVNGM
LQCVAAGFPEPTIDWYFCPGTEQRCSASVLPVDVQTLNSSGPPFGKLVVQ
SSIDSSAFKHNGTVECKAYNDVGKTSAYFNFAFKGNNKEQIHPHTHHHHH
H

In one embodiment, the anti-CD117 antibody, or antigen binding fragment thereof, comprises variable regions having an amino acid sequence that is at least about 95%, about 96%, about 97%, about 98% or about 99% identical to the SEQ ID NOs disclosed herein. Alternatively, the anti-CD117 antibody, or antigen binding fragment thereof, comprises CDRs comprising the SEQ ID Nos disclosed herein with framework regions of the variable regions described herein having an amino acid sequence that is at least about 95%, about 96%, about 97%, about 98% or about 99% identical to the SEQ ID NOs disclosed herein.

The anti-CD117 antibodies described herein can be in the form of full-length antibodies, bispecific antibodies, dual variable domain antibodies, multiple chain or single chain antibodies, and/or binding fragments that specifically bind human CD117, including but not limited to Fab, Fab′, (Fab′)₂, Fv), scFv (single chain Fv), surrobodies (including surrogate light chain construct), single domain antibodies, camelized antibodies and the like. They also can be of, or derived from, any isotype, including, for example, IgA (e.g., IgA1 or IgA2), IgD, IgE, IgG (e.g. IgG1, IgG2, IgG3 or IgG4), or IgM. In some embodiments, the anti-CD117 antibody is an IgG (e.g. IgG1, IgG2, IgG3 or IgG4).

Antibodies for use in conjunction with the methods described herein include variants of those antibodies described above, such as antibody fragments that contain or lack an Fc domain, as well as humanized variants of non-human antibodies described herein and antibody-like protein scaffolds (e.g., ¹⁰Fn3 domains) containing one or more, or all, of the CDRs or equivalent regions thereof of an antibody, or antibody fragment, described herein. Exemplary antigen-binding fragments of the foregoing antibodies include a dual-variable immunoglobulin domain, a single-chain Fv molecule (scFv), a diabody, a triabody, a nanobody, an antibody-like protein scaffold, a Fv fragment, a Fab fragment, a F(ab′)₂ molecule, and a tandem di-scFv, among others.

In one embodiment, anti-CD117 antibodies comprising one or more radiolabeled amino acids are provided. A radiolabeled anti-CD117 antibody may be used for both diagnostic and therapeutic purposes (conjugation to radiolabeled molecules is another possible feature). Nonlimiting examples of labels for polypeptides include, but are not limited to 3H, 14C, 15N, 35S, 90Y, 99Tc, and 125I, 131I, and 186Re. Methods for preparing radiolabeled amino acids and related peptide derivatives are known in the art (see for instance Junghans et al., in Cancer Chemotherapy and Biotherapy 655-686 (2d edition, Chafner and Longo, eds., Lippincott Raven (1996)) and U.S. Pat. Nos. 4,681,581, 4,735,210, 5,101,827, 5,102,990 (RE35,500), 5,648,471 and 5,697,902. For example, a radioisotope may be conjugated by a chloramine T method.

Further, in certain embodiments the anti-CD117 antibodies, as described herein, have a serum half-life in a human subject of about 3 days or less. In certain embodiments, the anti-CD117 antibodies, as described herein, have a half-life (e.g., in humans) equal to or less than about 24 hours, equal to or less than about 23 hours, equal to or less than about 22 hours, equal to or less than about 21 hours, equal to or less than about 20 hours, equal to or less than about 19 hours, equal to or less than about 18 hours, equal to or less than about 17 hours, equal to or less than about 16 hours, equal to or less than about 15 hours, equal to or less than about 14 hours, equal to or less than about 13 hours, equal to or less than about 12 hours, or equal to or less than about 11 hours.

In one embodiment, the anti-CD117 antibodies, as described herein, have a half-life (e.g., in humans) about 1-5 hours, about 5-10 hours, about 10-15 hours, about 15-20 hours, or about 20 to 25 hours.

### Fc Modified Antibodies

The present invention disclosure is based in part on the discovery that antibodies, or antigen-binding fragments thereof, having Fc modifications that allow Fc silencing capable of binding an antigen expressed by, e.g., a hematopoietic stem cell of a bone marrow stem cell niche, or a CD117+ leukemic cell or a CD117+ autoimmune lymphocyte), such as CD117, can be used as therapeutic agents alone or as ADCs to (i) facilitate the engraftment of transplanted hematopoietic stem cells in a patient in need of transplant therapy and (ii) treat cancers and autoimmune diseases. These therapeutic activities can be caused, for instance, by the binding of an anti-CD117 antibody, or antigen-binding fragment thereof, which binds to CD117 expressed by a cell,

The anti-CD117 antibodies or binding fragments described herein may also include modifications and/or mutations that alter the properties of the antibodies and/or fragments, such as those that increase half-life, increase or decrease ADCC, etc., as is known in the art.

In one embodiment, the anti-CD117 antibody, or binding fragment thereof, comprises a variant (or modified) Fc region, wherein said variant Fc region comprises at least one amino acid modification relative to a wild-type Fc region, such that said molecule has an altered affinity for an FcgammaR. Certain amino acid positions within the Fc region are known through crystallography studies to make a direct contact with FcγR. Specifically amino acids 234-239 (hinge region), amino acids 265-269 (B/C loop), amino acids 297-299 (C′/E loop), and amino acids 327-332 (F/G) loop. (see Sondermann et al., 2000 Nature, 406: 267-273). In some embodiments, the anti-CD117 antibodies described herein may comprise variant Fc regions comprising modification of at least one residue that makes a direct contact with an Fcγ R based on structural and crystallographic analysis. In one embodiment, the Fc region of the anti-CD117 antibody (or fragment thereof) comprises an amino acid substitution at amino acid 265 according to the EU index as in Kabat et al., Sequences of Proteins of Immunological Interest, 5th Ed. Public Health Service, NH₁, MD (1991), expressly incorporated herein by references. The “EU index as in Kabat” refers to the numbering of the human IgG1 EU antibody. The EU index or EU index as in Kabat or EU numbering scheme refers to the numbering of the EU antibody (Edelman et al., 1969, Proc Natl Acad Sci USA 63:78-85, hereby entirely incorporated by reference.) In one embodiment, the Fc region comprises a D265A mutation. In one embodiment, the Fc region comprises a D265C mutation. In some embodiments, the Fc region of the anti-CD117 antibody (or fragment thereof) comprises an amino acid substitution at amino acid 234 according to the EU index as in Kabat. In one embodiment, the Fc region comprises a L234A mutation. In some embodiments, the Fc region of the anti-CD117 antibody (or fragment thereof) comprises an amino acid substitution at amino acid 235 according to the EU index as in Kabat. In one embodiment, the Fc region comprises a L235A mutation. In yet another embodiment, the Fc region comprises a L234A and L235A mutation. In a further embodiment, the Fc region of the antibody of an ADC described herein comprises a D265C, L234A, and L235A mutation.

In one embodiment, the Fc region comprises a mutation at an amino acid position of D265, V205, H435, I253, and/or H310. For example, specific mutations at these positions include D265C, V205C, H435A, I253A, and/or H310A.

In one embodiment, the Fc region comprises a L234A mutation. In some embodiments, the Fc region of the anti-CD117 antibody (or fragment thereof) comprises an amino acid substitution at amino acid 235 according to the EU index as in Kabat. In one embodiment, the Fc region comprises a L235A mutation. In yet another embodiment, the Fc region comprises a L234A and L235A mutation. In a further embodiment, the Fc region comprises a D265C, L234A, and L235A mutation. In yet a further embodiment, the Fc region comprises a D265C, L234A, L235A, and H435A mutation. In a further embodiment, the Fc region comprises a D265C and H435A mutation.

In yet another embodiment, the Fc region comprises a L234A and L235A mutation (also referred to herein as “L234A.L235A” or as “LALA”). In another embodiment, the Fc region comprises a L234A and L235A mutation, wherein the Fc region does not include a P329G mutation. In a further embodiment, the Fc region comprises a D265C, L234A, and L235A mutation (also referred to herein as “D265C.L234A.L235A”). In another embodiment, the Fc region comprises a D265C, L234A, and L235A mutation, wherein the Fc region does not include a P329G mutation. In yet a further embodiment, the Fc region comprises a D265C, L234A, L235A, and H435A mutation (also referred to herein as “D265C.L234A.L235A.H435A”). In another embodiment, the Fc region comprises a D265C, L234A, L235A, and H435A mutation, wherein the Fc region does not include a P329G mutation. In a further embodiment, the Fc region comprises a D265C and H435A mutation (also referred to herein as “D265C.H435A”). In yet another embodiment, the Fc region comprises a D265A, S239C, L234A, and L235A mutation (also referred to herein as “D265A.S239C.L234A.L235A”). In yet another embodiment, the Fc region comprises a D265A, S239C, L234A, and L235A mutation, wherein the Fc region does not include a P329G mutation. In another embodiment, the Fc region comprises a D265C, N297G, and H435A mutation (also referred to herein as “D265C.N297G.H435A”). In another embodiment, the Fc region comprises a D265C, N297Q, and H435A mutation (also referred to herein as “D265C.N297Q.H435A”). In another embodiment, the Fc region comprises a E233P, L234V, L235A and delG236 (deletion of 236) mutation (also referred to herein as “E233P.L234V.L235A.delG236” or as “EPLVLAdelG”). In another embodiment, the Fc region comprises a E233P, L234V, L235A and delG236 (deletion of 236) mutation, wherein the Fc region does not include a P329G mutation. In another embodiment, the Fc region comprises a E233P, L234V, L235A, delG236 (deletion of 236) and H435A mutation (also referred to herein as “E233P.L234V.L235A.delG236.H435A” or as “EPLVLAdeIG.H435A”). In another embodiment, the Fc region comprises a E233P, L234V, L235A, delG236 (deletion of 236) and H435A mutation, wherein the Fc region does not include a P329G mutation. In another embodiment, the Fc region comprises a L234A, L235A, S239C and D265A mutation. In another embodiment, the Fc region comprises a L234A, L235A, S239C and D265A mutation, wherein the Fc region does not include a P329G mutation. In another embodiment, the Fc region comprises a H435A, L234A, L235A, and D265C mutation. In another embodiment, the Fc region comprises a H435A, L234A, L235A, and D265C mutation, wherein the Fc region does not include a P329G mutation.

In some embodiments, the antibody has a modified Fc region such that, the anti-CD117 antibody decreases an effector function in an in vitro effector function assay with a decrease in binding to an Fc receptor (Fc R) relative to binding of an identical antibody comprising an unmodified Fc region to the FcR. In some embodiments, the antibody has a modified Fc region such that, the anti-CD117 antibody decreases an effector function in an in vitro effector function assay with a decrease in binding to an Fc gamma receptor (FcγR) relative to binding of an identical antibody comprising an unmodified Fc region to the FcγR. In some embodiments, the FcγR is FcγR₁. In some embodiments, the FcγR is FcγR₂A. In some embodiments, the FcγR is FcγR₂B. In other embodiments, the FcγR is FcγR₂C. In some embodiments, the FcγR is FcγR₃A. In some embodiments, the FcγR is FcγR₃B. In other embodiments, the decrease in binding is at least a 70% decrease, at least a 80% decrease, at least a 90% decrease, at least a 95% decrease, at least a 98% decrease, at least a 99% decrease, or a 100% decrease in antibody binding to a FcγR relative to binding of the identical antibody comprising an unmodified Fc region to the FcγR. In other embodiments, the decrease in binding is at least a 70% to a 100% decrease, at least a 80% to a 100% decrease, at least a 90% to a 100% decrease, at least a 95% to a 100% decrease, or at least a 98% to a 100% decrease, in antibody binding to a FcγR relative to binding of the identical antibody comprising an unmodified Fc region to the FcγR

In some embodiments, the anti-CD117 antibody has a modified Fc region such that, the antibody decreases cytokine release in an in vitro cytokine release assay with a decrease in cytokine release of at least 50% relative to cytokine release of an identical antibody comprising an unmodified Fc region. In some embodiments, the decrease in cytokine release is at least a 70% decrease, at least a 80% decrease, at least a 90% decrease, at least a 95% decrease, at least a 98% decrease, at least a 99% decrease, or a 100% decrease in cytokine release relative to cytokine release of the identical antibody comprising an unmodified Fc region. In some embodiments, the decrease in cytokine release is at least a 70% to a 100% decrease, at least an 80% to a 100% decrease, at least a 90% to a 100% decrease, at least a 95% to a 100% decrease in cytokine release relative to cytokine release of the identical antibody comprising an unmodified Fc region. In certain embodiments, cytokine release is by immune cells.

In some embodiments, the anti-CD117 antibody has a modified Fc region such that, the antibody decreases mast cell degranulation in an in vitro mast cell degranulation assay with a decrease in mast cell degranulation of at least 50% relative to mast cell degranulation of an identical antibody comprising an unmodified Fc region. In some embodiments, the decrease in mast cell degranulation is at least a 70% decrease, at least a 80% decrease, at least a 90% decrease, at least a 95% decrease, at least a 98% decrease, at least a 99% decrease, or a 100% decrease in mast cell degranulation relative to mast cell degranulation of the identical antibody comprising an unmodified Fc region. In some embodiments, the decrease in mast cell degranulation is at least a 70% to a 100% decrease, at least a 80% to a 100% decrease, at least a 90% to a 100% decrease, or at least a 95% to a 100% decrease, in mast cell degranulation relative to mast cell degranulation of the identical antibody comprising an unmodified Fc region.

In some embodiments, the anti-CD117 antibody has a modified Fc region such that, the antibody decreases or prevents antibody dependent cell phagocytosis (ADCP) in an in vitro antibody dependent cell phagocytosis assay, with a decrease in ADCP of at least 50% relative to ADCP of an identical antibody comprising an unmodified Fc region. In some embodiments, the decrease in ADCP is at least a 70% decrease, at least a 80% decrease, at least a 90% decrease, at least a 95% decrease, at least a 98% decrease, at least a 99% decrease, or a 100% decrease in cytokine release relative to cytokine release of the identical antibody comprising an unmodified Fc region.

In some embodiments, the anti-CD117 antibody, as described herein, comprises an Fc region comprising one of the following modifications or combinations of modifications: D265A, D265C, D265C/H435A, D265C/LALA, D265C/LALA/H435A, D265A/S239C/L234A/L235A/H435A, D265A/S239C/L234A/L235A, D265C/N297G, D265C/N297G/H435A, D265C (EPLVLAdeIG *), D265C (EPLVLAdeIG)/H435A, D265C/N297Q/H435A, D265C/N297Q, EPLVLAdeIG/H435A, EPLVLAdeIG/D265C, EPLVLAdeIG/D265A, N297A, N297G, or N297Q. In some embodiments, the anti-CD117 antibody herein comprises an Fc region comprising one of the following modifications or combinations of modifications: D265A, D265C, D265C/H435A, D265C/LALA, D265C/LALA/H435A, D265C/N297G, D265C/N297G/H435A, D265C (IgG2*), D265C (IgG2)/H435A, D265C/N297Q/H435A, D265C/N297Q, EPLVLAdeIG/H435A, N297A, N297G, or N2970.

Binding or affinity between a modified Fc region and a Fc gamma receptor can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE® analysis or Octet™ analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmuno assay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

In one embodiment, an antibody having the Fc modifications described herein (e.g., D265C, L234A, L235A, and/or H435A) has at least a 70% decrease, at least a 80% decrease, at least a 90% decrease, at least a 95% decrease, at least a 98% decrease, at least a 99% decrease, or a 100% decrease in binding to a Fc gamma receptor relative to binding of the identical antibody comprising an unmodified Fc region to the Fc gamma receptor (e.g., as assessed by biolayer interferometry (BLI)).

Without wishing to be bound by any theory, it is believed that Fc region binding interactions with a Fc gamma receptor are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a modified Fc region (e.g., comprising a L234A, L235A, and/or a D265C mutation) has substantially reduced or abolished effector functions. Effector functions can be assayed using a variety of methods known in the art, e.g., by measuring cellular responses (e.g., mast cell degranulation or cytokine release) in response to the antibody of interest. For example, using standard methods in the art, the Fc-modified antibodies can be assayed for their ability to trigger mast cell degranulation in or for their ability to trigger cytokine release, e.g. by human peripheral blood mononuclear cells.

In certain aspects a variant IgG Fc domain comprises one or more amino acid substitutions resulting in decreased or ablated binding affinity for an Fc.gamma.R and/or C1q as compared to the wild type Fc domain not comprising the one or more amino acid substitutions. Fc binding interactions are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a modified Fc region (e.g., comprising a L234A, L235A, and a D265C mutation) has substantially reduced or abolished effector functions.

Affinity to an Fc region can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE™. analysis or Octet™ analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

Antibodies may be further engineered to further modulate antibody half-life by (e.g., relative to an antibody having an unmodified Fc region) introducing additional Fc mutations, such as those described for example in (Dall'Acqua et al. (2006) J Biol Chem 281: 23514-24), (Zalevsky et al. (2010) Nat Biotechnol 28: 157-9), (Hinton et al. (2004) J Biol Chem 279: 6213-6), (Hinton et al. (2006) J Immunol 176: 346-56), (Shields et al. (2001) J Biol Chem 276: 6591-604), (Petkova et al. (2006) Int Immunol 18: 1759-69), (Datta-Mannan et al. (2007) Drug Metab Dispos 35: 86-94), (Vaccaro et al. (2005) Nat Biotechnol 23: 1283-8), (Yeung et al. (2010) Cancer Res 70: 3269-77) and (Kim et al. (1999) Eur J Immunol 29: 2819-25), and include positions 250, 252, 253, 254, 256, 257, 307, 376, 380, 428, 434 and 435. Exemplary mutations that may be made singularly or in combination are T250Q, M252Y, I253A, S254T, T256E, P2571, T307A, D376V, E380A, M428L, H₄₃₃K, N434S, N434A, N434H, N434F, H435A and H435R mutations.

Thus, in one embodiment, the Fc region comprises a mutation resulting in a decrease in half life. An antibody having a short half life may be advantageous in certain instances where the antibody is expected to function as a short-lived therapeutic, e.g., the conditioning step described herein where the antibody is administered followed by HSCs. Ideally, the antibody would be substantially cleared prior to delivery of the HSCs, which also generally express an antigen targeted by an ADC described herein, e.g., CD117, but are not the target of the ADC, unlike the endogenous stem cells. In one embodiment, the Fc regions comprise a mutation at position 435 (EU index according to Kabat). In one embodiment, the mutation is an H435A mutation.

In one embodiment, the antibody described herein has a half life of equal to or less than about 24 hours, a half life of equal to or less than about 22 hours, a half life of equal to or less than about 20 hours, a half life of equal to or less than about 18 hours, a half life of equal to or less than about 16 hours, a half life of equal to or less than about 14 hours, equal to or less than about 13 hours, equal to or less than about 12 hours, or equal to or less than about 11 hours. In one embodiment, the half life of the antibody is about 11 hours to about 24 hours; about 12 hours to about 22 hours; about 10 hours to about 20 hours; about 8 hours to about 18 hours; or about 14 hours to about 24 hours.

In some aspects, the Fc region comprises two or more mutations that confer reduced half-life and greatly diminish or completely abolish an effector function of the antibody. In some embodiments, the Fc region comprises a mutation resulting in a decrease in half-life and a mutation of at least one residue that can make direct contact with an FcγR (e.g., as based on structural and crystallographic analysis). In one embodiment, the Fc region comprises a H435A mutation, a L234A mutation, and a L235A mutation. In one embodiment, the Fc region comprises a H435A mutation and a D265C mutation. In one embodiment, the Fc region comprises a H435A mutation, a L234A mutation, a L235A mutation, and a D265C mutation.

In some embodiments, the anti-CD117 antibody or antigen-binding fragment thereof is conjugated to a cytotoxin (e.g., pyrrolobenzodiazepine) by way of a cysteine residue in the Fc domain of the antibody or antigen-binding fragment thereof. In some embodiments, the cysteine residue is introduced by way of a mutation in the Fc domain of the antibody or antigen-binding fragment thereof. For instance, the cysteine residue may be selected from the group consisting of Cys118, Cys239, and Cys265. In one embodiment, the Fc region of the anti-CD117 antibody (or fragment thereof) comprises an amino acid substitution at amino acid 265 according to the EU index as in Kabat. In one embodiment, the Fc region comprises a D265C mutation. In one embodiment, the Fc region comprises a D265C and H435A mutation. In one embodiment, the Fc region comprises a D265C, a L234A, and a L235A mutation. In one embodiment, the Fc region comprises a D265C, a L234A, a L235A, and a H435A mutation. In one embodiment, the Fc region of the anti-CD117 antibody, or antigen-binding fragment thereof, comprises an amino acid substitution at amino acid 239 according to the EU index as in Kabat. In one embodiment, the Fc region comprises a S239C mutation. In one embodiment, the Fc region comprises a L234A mutation, a L235A mutation, a S239C mutation and a D265A mutation. In another embodiment, the Fc region comprises a S239C and H435A mutation. In another embodiment, the Fc region comprises a L234A mutation, a L235A mutation, and S239C mutation. In yet another embodiment, the Fc region comprises a H435A mutation, a L234A mutation, a L235A mutation, and S239C mutation. In yet another embodiment, the Fc region comprises a H435A mutation, a L234A mutation, a L235A mutation, a S239C mutation and D265A mutation.

Notably, Fc amino acid positions are in reference to the EU numbering index unless otherwise indicated.

In some embodiments of these aspects, the cysteine residue is naturally occurring in the Fc domain of the anti-CD117 antibody or antigen-binding fragment thereof. For instance, the Fc domain may be an IgG Fc domain, such as a human IgG1 Fc domain, and the cysteine residue may be selected from the group consisting of Cys261, Csy321, Cys367, and Cys425.

For example, in one embodiment, the Fc region of Antibody 67 is modified to comprise a D265C mutation (e.g., SEQ ID NO: 111). In another embodiment, the Fc region of Antibody 67 is modified to comprise a D265C, L234A, and L235A mutation (e.g., SEQ ID NO: 112). In yet another embodiment, the Fc region of Antibody 67 is modified to comprise a D265C and H435A mutation (e.g., SEQ ID NO: 113). In a further embodiment, the Fc region of Antibody 67 is modified to comprise a D265C, L234A, L235A, and H435A mutation (e.g., SEQ ID NO: 114).

In regard to Antibody 55, in one embodiment, the Fc region of Antibody 55 is modified to comprise a D265C mutation (e.g., SEQ ID NO: 117). In another embodiment, the Fc region of Antibody 55 is modified to comprise a D265C, L234A, and L235A mutation (e.g., SEQ ID NO: 118). In yet another embodiment, the Fc region of Antibody 55 is modified to comprise a D265C and H435A mutation (e.g., SEQ ID NO: 119). In a further embodiment, the Fc region of Antibody 55 is modified to comprise a D265C, L234A, L235A, and H435A mutation (e.g., SEQ ID NO: 120).

The Fc regions of any one of Antibody 54, Antibody 55, Antibody 56, Antibody 57, Antibody 58, Antibody 61, Antibody 66, Antibody 67, Antibody 68, or Antibody 69 can be modified to comprise a D265C mutation (e.g., as in SEQ ID NO: 123); a D265C, L234A, and L235A mutation (e.g., as in SEQ ID NO: 124); a D265C and H435A mutation (e.g., as in SEQ ID NO: 125); or a D265C, L234A, L235A, and H435A mutation (e.g., as in SEQ ID NO: 126).

The variant Fc domains described herein are defined according to the amino acid modifications that compose them. For all amino acid substitutions discussed herein in regard to the Fc region, numbering is always according to the EU index. Thus, for example, D265C is an Fc variant with the aspartic acid (D) at EU position 265 substituted with cysteine (C) relative to the parent Fc domain. Likewise, e.g., D265C/L234A/L235A defines a variant Fc variant with substitutions at EU positions 265 (D to C), 234 (L to A), and 235 (L to A) relative to the parent Fc domain. A variant can also be designated according to its final amino acid composition in the mutated EU amino acid positions. For example, the L234A/L235A mutant can be referred to as LALA. It is noted that the order in which substitutions are provided is arbitrary.

In one embodiment, the anti-CD117 antibody, or antigen binding fragment thereof, comprises variable regions having an amino acid sequence that is at least about 95%, about 96%, about 97%, about 98% or about 99% identical to the SEQ ID Nos disclosed herein. Alternatively, the anti-CD117 antibody, or antigen binding fragment thereof, comprises CDRs comprising the SEQ ID Nos disclosed herein with framework regions of the variable regions described herein having an amino acid sequence that is at least about 95%, about 96%, about 97%, about 98% or about 99% identical to the SEQ ID Nos disclosed herein.

In one embodiment, the anti-CD117 antibody, or antigen binding fragment thereof, comprises a heavy chain variable region and a heavy chain constant region having an amino acid sequence that is disclosed herein. In another embodiment, the anti-CD117 antibody, or antigen binding fragment thereof, comprises a light chain variable region and a light chain constant region having an amino acid sequence that is disclosed herein. In yet another embodiment, the anti-CD117 antibody, or antigen binding fragment thereof, comprises a heavy chain variable region, a light chain variable region, a heavy chain constant region and a light chain constant region having an amino acid sequence that is disclosed herein.

### Methods of Identifying Antibodies

Provided herein are novel ADCs that may be used, for example, in conditioning methods for stem cell transplantation. In view of the disclosure herein, other antibodies, e.g., anti-CD117 antibodies, can be identified that can be used in the ADCs and methods of the present disclosure.

Methods for high throughput screening of antibody, or antibody fragment libraries for molecules capable of binding CD117 can be used to identify and affinity mature antibodies useful for treating cancers, autoimmune diseases, and conditioning a patient (e.g., a human patient) in need of hematopoietic stem cell therapy as described herein. Such methods include in vitro display techniques known in the art, such as phage display, bacterial display, yeast display, mammalian cell display, ribosome display, mRNA display, and cDNA display, among others. The use of phage display to isolate ligands that bind biologically relevant molecules has been reviewed, for example, in Felici et al., Biotechnol. Annual Rev. 1:149-183, 1995; Katz, Annual Rev. Biophys. Biomol. Struct. 26:27-45, 1997; and Hoogenboom et al., Immunotechnology 4:1-20, 1998, the disclosures of each of which are incorporated herein by reference as they pertain to in vitro display techniques. Randomized combinatorial peptide libraries have been constructed to select for polypeptides that bind cell surface antigens as described in Kay, Perspect. Drug Discovery Des. 2:251-268, 1995 and Kay et al., Mol. Divers. 1:139-140, 1996, the disclosures of each of which are incorporated herein by reference as they pertain to the discovery of antigen-binding molecules. Proteins, such as multimeric proteins, have been successfully phage-displayed as functional molecules (see, for example, EP 0349578; EP 4527839; and EP 0589877, as well as Chiswell and McCafferty, Trends Biotechnol. 10:80-84 1992, the disclosures of each of which are incorporated herein by reference as they pertain to the use of in vitro display techniques for the discovery of antigen-binding molecules). In addition, functional antibody fragments, such as Fab and scFv fragments, have been expressed in in vitro display formats (see, for example, McCafferty et al., Nature 348:552-554, 1990; Barbas et al., Proc. Natl. Acad. Sci. USA 88:7978-7982, 1991; and Clackson et al., Nature 352:624-628, 1991, the disclosures of each of which are incorporated herein by reference as they pertain to in vitro display platforms for the discovery of antigen-binding molecules). These techniques, among others, can be used to identify and improve the affinity of antibodies that bind CD117 (e.g., GNNK+CD117) that can in turn be used to deplete endogenous hematopoietic stem cells in a patient (e.g., a human patient) in need of hematopoietic stem cell transplant therapy.

In addition to in vitro display techniques, computational modeling techniques can be used to design and identify antibodies, and antibody fragments, in silico that bind a cell surface antigen (e.g., CD117). For example, using computational modeling techniques, one of skill in the art can screen libraries of antibodies, and antibody fragments, in silico for molecules capable of binding specific epitopes, such as extracellular epitopes of this antigen. The antibodies, and antigen-binding fragments thereof, identified by these computational techniques can be used in conjunction with the therapeutic methods described herein, such as the cancer and autoimmune disease treatment methods described herein and the patient conditioning procedures described herein.

Additional techniques can be used to identify antibodies, and antigen-binding fragments thereof, that bind a cell surface antigen (e.g., CD117) on the surface of a cell (e.g., a cancer cell, autoimmune cell, or hematopoietic stem cell) and that are internalized by the cell, for instance, by receptor-mediated endocytosis. For example, the in vitro display techniques described above can be adapted to screen for antibodies, and antigen-binding fragments thereof, that bind a cell surface antigen (e.g., CD117) on the surface of a cancer cell, autoimmune cell, or hematopoietic stem cell and that are subsequently internalized. Phage display represents one such technique that can be used in conjunction with this screening paradigm. To identify antibodies, and fragments thereof, that bind a cell surface antigen (e.g., CD117) and are subsequently internalized by cancer cells, autoimmune cells, or hematopoietic stem cells, one of skill in the art can adapt the phage display techniques described, for example, in Williams et al., Leukemia 19:1432-1438, 2005, the disclosure of which is incorporated herein by reference in its entirety. For example, using mutagenesis methods known in the art, recombinant phage libraries can be produced that encode antibodies, antibody fragments, such as scFv fragments, Fab fragments, diabodies, triabodies, and ¹⁰Fn3 domains, among others, or ligands that contain randomized amino acid cassettes (e.g., in one or more, or all, of the CDRs or equivalent regions thereof or an antibody or antibody fragment). The framework regions, hinge, Fc domain, and other regions of the antibodies or antibody fragments may be designed such that they are non-immunogenic in humans, for instance, by virtue of having human germline antibody sequences or sequences that exhibit only minor variations relative to human germline antibodies.

Using phage display techniques described herein or known in the art, phage libraries containing randomized antibodies, or antibody fragments, covalently bound to the phage particles can be incubated with a cell surface target antigen (e.g., CD117) antigen, for instance, by first incubating the phage library with blocking agents (such as, for instance, milk protein, bovine serum albumin, and/or IgG so as to remove phage encoding antibodies, or fragments thereof, that exhibit non-specific protein binding and phage that encode antibodies or fragments thereof that bind Fc domains, and then incubating the phage library with a population of hematopoietic stem cells. The phage library can be incubated with the target cells, such as cancer cells, autoimmune cells, or hematopoietic stem cells for a time sufficient to allow cell surface antigen specific antibodies, or antigen-binding fragments thereof, (e.g., CD117-specific antibodies, or antigen-binding fragments thereof) to bind cell-surface antigen (e.g., sell-surface CD117) antigen and to subsequently be internalized by the cancer cells, autoimmune cells, or hematopoietic stem cells (e.g., from 30 minutes to 6 hours at 4° C., such as 1 hour at 4° C.). Phage containing antibodies, or fragments thereof, that do not exhibit sufficient affinity for one or more of these antigens so as to permit binding to, and internalization by, cancer cells, autoimmune cells, or hematopoietic stem cells can subsequently be removed by washing the cells, for instance, with cold (4° C.) 0.1 M glycine buffer at pH 2.8. Phage bound to antibodies, or fragments thereof, that have been internalized by the cancer cells, autoimmune cells, or hematopoietic stem cells can be identified, for instance, by lysing the cells and recovering internalized phage from the cell culture medium. The phage can then be amplified in bacterial cells, for example, by incubating bacterial cells with recovered phage in 2×YT medium using methods known in the art. Phage recovered from this medium can then be characterized, for instance, by determining the nucleic acid sequence of the gene(s) encoding the antibodies, or fragments thereof, inserted within the phage genome. The encoded antibodies, or fragments thereof, can subsequently be prepared de novo by chemical synthesis (for instance, of antibody fragments, such as scFv fragments) or by recombinant expression (for instance, of full-length antibodies).

An exemplary method for in vitro evolution of a cell surface antigen antibody (e.g., anti-CD117) antibodies for use with the compositions and methods described herein is phage display. Phage display libraries can be created by making a designed series of mutations or variations within a coding sequence for the CDRs of an antibody or the analogous regions of an antibody-like scaffold (e.g., the BC, CD, and DE loops of ¹⁰Fn3 domains). The template antibody-encoding sequence into which these mutations are introduced may be, for example, a naive human germline sequence. These mutations can be performed using standard mutagenesis techniques known in the art. Each mutant sequence thus encodes an antibody corresponding to the template save for one or more amino acid variations. Retroviral and phage display vectors can be engineered using standard vector construction techniques known in the art. P3 phage display vectors along with compatible protein expression vectors can be used to generate phage display vectors for antibody diversification.

The mutated DNA provides sequence diversity, and each transformant phage displays one variant of the initial template amino acid sequence encoded by the DNA, leading to a phage population (library) displaying a vast number of different but structurally related amino acid sequences. Due to the well-defined structure of antibody hypervariable regions, the amino acid variations introduced in a phage display screen are expected to alter the binding properties of the binding peptide or domain without significantly altering its overall molecular structure.

In a typical screen, a phage library may be contacted with and allowed to bind one of the foregoing antigens or an epitope thereof. To facilitate separation of binders and non-binders, it is convenient to immobilize the target on a solid support. Phage bearing a cell surface-binding moiety can form a complex with the target on the solid support, whereas non-binding phage remain in solution and can be washed away with excess buffer. Bound phage can then liberated from the target by changing the buffer to an extreme pH (pH 2 or pH 10), changing the ionic strength of the buffer, adding denaturants, or other known means.

The recovered phage can then be amplified through infection of bacterial cells, and the screening process can be repeated with the new pool that is now depleted in non-binding antibodies and enriched for antibodies that bind a target antigen (e.g., CD117). The recovery of even a few binding phage is sufficient to amplify the phage for a subsequent iteration of screening. After a few rounds of selection, the gene sequences encoding the antibodies or antigen-binding fragments thereof derived from selected phage clones in the binding pool are determined by conventional methods, thus revealing the peptide sequence that imparts binding affinity of the phage to the target. During the panning process, the sequence diversity of the population diminishes with each round of selection until desirable peptide-binding antibodies remain. The sequences may converge on a small number of related antibodies or antigen-binding fragments thereof. An increase in the number of phage recovered at each round of selection is an indication that convergence of the library has occurred in a screen.

Another method for identifying antibodies includes using humanizing non-human antibodies that bind a cell surface target antigen (e.g., CD117), for instance, according to the following procedure. Consensus human antibody heavy chain and light chain sequences are known in the art (see e.g., the “VBASE” human germline sequence database; Kabat et al. Sequences of Proteins of Immunological Interest, Fifth Edition, U.S. Department of Health and Human Services, NIH Publication No. 91-3242, 1991; Tomlinson et al., J. Mol. Biol. 227:776-798, 1992; and Cox et al. Eur. J. Immunol. 24:827-836, 1994, the disclosures of each of which are incorporated herein by reference as they pertain to consensus human antibody heavy chain and light chain sequences. Using established procedures, one of skill in the art can identify the variable domain framework residues and CDRs of a consensus antibody sequence (e.g., by sequence alignment). One can substitute one or more CDRs of the heavy chain and/or light chain variable domains of consensus human antibody with one or more corresponding CDRs of a non-human antibody that binds a cell surface antigen (e.g., CD117) as described herein in order to produce a humanized antibody. This CDR exchange can be performed using gene editing techniques described herein or known in the art.

To produce humanized antibodies, one can recombinantly express a polynucleotide encoding the above consensus sequence in which one or more variable region CDRs have been replaced with one or more variable region CDR sequences of a non-human antibody that binds a cell surface target antigen (e.g., CD117). As the affinity of the antibody for the hematopoietic stem cell antigen is determined primarily by the CDR sequences, the resulting humanized antibody is expected to exhibit an affinity for the hematopoietic stem cell antigen that is about the same as that of the non-human antibody from which the humanized antibody was derived. Methods of determining the affinity of an antibody for a target antigen include, for instance, ELISA-based techniques described herein and known in the art, as well as surface plasmon resonance, fluorescence anisotropy, and isothermal titration calorimetry, among others.

The internalizing capacity of an antibody, or fragment thereof, can be assessed, for instance, using radionuclide internalization assays known in the art. For example, antibodies, or fragments thereof, identified using in vitro display techniques described herein or known in the art can be functionalized by incorporation of a radioactive isotope, such as ¹⁸F, ⁷⁵Br, ⁷⁷Br, ¹²²I, ¹²³I, ¹²⁴I, ¹²⁵I, ¹²⁹I, ¹³¹I, ²¹¹At, ⁶⁷Ga, ¹¹¹In, ⁹⁹TC, ¹⁶⁹Yb, ¹⁸⁶Re, ⁶⁴Cu, ⁶⁷Cu, ¹⁷⁷Lu, ⁷⁷As, ⁷²As, ⁸⁶Y, ⁹⁰Y, ⁸⁹Zr, ²¹²Bi, ²¹³Bi, or ²²⁵Ac. For instance, radioactive halogens, such as ¹⁸F, ⁷⁵Br, ⁷⁷Br, ¹²²I, ¹²³I, ¹²⁴I, ¹²⁵I, ¹²⁹I, ¹³¹I, ²¹¹At, can be incorporated into antibodies, or fragments thereof, using beads, such as polystyrene beads, containing electrophilic halogen reagents (e.g., Iodination Beads, Thermo Fisher Scientific, Inc., Cambridge, Mass.). Radiolabeled antibodies, or fragments thereof, can be incubated with cancer cells, autoimmune cells, or hematopoietic stem cells for a time sufficient to permit internalization (e.g., from 30 minutes to 6 hours at 4° C., such as 1 hour at 4° C.). The cells can then be washed to remove non-internalized antibodies, or fragments thereof, (e.g., using cold (4° C.) 0.1 M glycine buffer at pH 2.8). Internalized antibodies, or fragments thereof, can be identified by detecting the emitted radiation (e.g., γ-radiation) of the resulting cancer cells, autoimmune cells, or hematopoietic stem cells in comparison with the emitted radiation (e.g., γ-radiation) of the recovered wash buffer.

Antibodies may be produced using recombinant methods and compositions, e.g., as described in U.S. Pat. No. 4,816,567. In one embodiment, isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-CLL-1 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### Methods of Use

Anti-CD117 ADCs described herein may be administered by a variety of routes, such as orally, transdermally, subcutaneously, intranasally, intravenously, intramuscularly, intraocularly, or parenterally. The most suitable route for administration in any given case will depend on the particular antibody, or antigen-binding fragment, administered, the patient, pharmaceutical formulation methods, administration methods (e.g., administration time and administration route), the patient's age, body weight, sex, severity of the diseases being treated, the patient's diet, and the patient's excretion rate.

The effective dose of an anti-CD117 ADC, antibody, or antigen-binding fragment thereof, described herein can range, for example from about 0.001 to about 100 mg/kg of body weight per single (e.g., bolus) administration, multiple administrations, or continuous administration, or to achieve an optimal serum concentration (e.g., a serum concentration of 0.0001-5000 μg/mL) of the antibody, antigen-binding fragment thereof. The dose may be administered one or more times (e.g., 2-10 times) per day, week, or month to a subject (e.g., a human) suffering from cancer, an autoimmune disease, or undergoing conditioning therapy in preparation for receipt of a hematopoietic stem cell transplant. In the case of a conditioning procedure prior to hematopoietic stem cell transplantation, the ADC, antibody, or antigen-binding fragment thereof, can be administered to the patient at a time that optimally promotes engraftment of the exogenous hematopoietic stem cells, for instance, from 1 hour to 1 week (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, or about 7 days) or more prior to administration of the exogenous hematopoietic stem cell transplant.

As described herein, hematopoietic stem cell transplant therapy described herein can be administered to a subject in need of treatment so as to populate or re-populate one or more blood cell types. Hematopoietic stem cells generally exhibit multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Hematopoietic stem cells are additionally capable of self-renewal, and can thus give rise to daughter cells that have equivalent potential as the mother cell, and also feature the capacity to be reintroduced into a transplant recipient whereupon they home to the hematopoietic stem cell niche and re-establish productive and sustained hematopoiesis.

ADCs described herein may be used as a conditioning agent for HSC transplanation. Hematopoietic stem cells can then be administered to a patient defective or deficient in one or more cell types of the hematopoietic lineage in order to re-constitute the defective or deficient population of cells in vivo, thereby treating the pathology associated with the defect or depletion in the endogenous blood cell population.

The compositions and methods described herein can thus be used to treat a non-malignant hemoglobinopathy (e.g., a hemoglobinopathy selected from the group consisting of sickle cell anemia, thalassemia, Fanconi anemia, aplastic anemia, and Wiskott-Aldrich syndrome).

Additionally or alternatively, the compositions and methods described herein can be used to treat an immunodeficiency, such as a congenital immunodeficiency. Additionally or alternatively, the compositions and methods described herein can be used to treat an acquired immunodeficiency (e.g., an acquired immunodeficiency selected from the group consisting of HIV and AIDS).

The compositions and methods described herein can be used to treat a metabolic disorder (e.g., a metabolic disorder selected from the group consisting of glycogen storage diseases, mucopolysaccharidoses, Gaucher's Disease, Hurlers Disease, sphingolipidoses, and metachromatic leukodystrophy).

Additionally or alternatively, the compositions and methods described herein can be used to treat a malignancy or proliferative disorder, such as a hematologic cancer, myeloproliferative disease. In the case of cancer treatment, the compositions and methods described herein may be administered to a patient so as to deplete a population of endogenous hematopoietic stem cells prior to hematopoietic stem cell transplantation therapy, in which case the transplanted cells can home to a niche created by the endogenous cell depletion step and establish productive hematopoiesis. This, in turn, can re-constitute a population of cells depleted during cancer cell eradication, such as during systemic chemotherapy. Exemplary hematological cancers that can be treated using the compositions and methods described heein include, without limitation, acute myeloid leukemia, acute lymphoid leukemia, chronic myeloid leukemia, chronic lymphoid leukemia, multiple myeloma, diffuse large B-cell lymphoma, and non-Hodgkin's lymphoma, as well as other cancerous conditions, including neuroblastoma.

Additional diseases that can be treated with the compositions and methods described herein include, without limitation, adenosine deaminase deficiency and severe combined immunodeficiency, hyper immunoglobulin M syndrome, Chediak-Higashi disease, hereditary lymphohistiocytosis, osteopetrosis, osteogenesis imperfecta, storage diseases, thalassemia major, systemic sclerosis, systemic lupus erythematosus, multiple sclerosis, and juvenile rheumatoid arthritis.

The ADCs described herein may be used to induce solid organ transplant tolerance. For instance, the compositions and methods described herein may be used to deplete or ablate a population of cells from a target tissue (e.g., to deplete hematopoietic stem cells from the bone marrow stem cell niche). Following such depletion of cells from the target tissues, a population of stem or progenitor cells from an organ donor (e.g., hematopoietic stem cells from the organ donor) may be administered to the transplant recipient, and following the engraftment of such stem or progenitor cells, a temporary or stable mixed chimerism may be achieved, thereby enabling long-term transplant organ tolerance without the need for further immunosuppressive agents. For example, the compositions and methods described herein may be used to induce transplant tolerance in a solid organ transplant recipient (e.g., a kidney transplant, lung transplant, liver transplant, and heart transplant, among others). The compositions and methods described herein are well-suited for use in connection the induction of solid organ transplant tolerance, for instance, because a low percentage temporary or stable donor engraftment is sufficient to induce long-term tolerance of the transplanted organ.

In addition, the compositions and methods described herein can be used to treat cancers directly, such as cancers characterized by cells that are CD117+. For instance, the compositions and methods described herein can be used to treat leukemia, particularly in patients that exhibit CD117+ leukemic cells. By depleting CD117+ cancerous cells, such as leukemic cells, the compositions and methods described herein can be used to treat various cancers directly. Exemplary cancers that may be treated in this fashion include hematological cancers, such as acute myeloid leukemia, acute lymphoid leukemia, chronic myeloid leukemia, chronic lymphoid leukemia, multiple myeloma, diffuse large B-cell lymphoma, and non-Hodgkin's lymphoma.

Acute myeloid leukemia (AML) is a cancer of the myeloid line of blood cells, characterized by the rapid growth of abnormal white blood cells that build up in the bone marrow and interfere with the production of normal blood cells. AML is the most common acute leukemia affecting adults, and its incidence increases with age. The symptoms of AML are caused by replacement of normal bone marrow with leukemic cells, which causes a drop in red blood cells, platelets, and normal white blood cells. As an acute leukemia, AML progresses rapidly and may be fatal within weeks or months if left untreated. In one embodiment, the anti-CD117 ADCs described herein are used to treat AML in a human patient in need thereof. In certain embodiments the anti-CD117 ADC treatment depletes AML cells in the treated subjects. In some embodiments 50% or more of the AML cells are depleted. In other embodiments, 60% or more of the AML cells are depleted, or 70% or more of the AML cells are depleted, or 80% of more or 90% or more, or 95% or more of the AML cells are depleted. In certain embodiments the anti-CD117 ADC treatments is a single dose treatment. In certain embodiments the single dose anti-CD117 ADC treatment depletes 60%, 70%, 80%, 90% or 95% or more of the AML cells.

In addition, the compositions and methods described herein can be used to treat autoimmune disorders. For instance, an antibody, or antigen-binding fragment thereof, can be administered to a subject, such as a human patient suffering from an autoimmune disorder, so as to kill a CD117+ immune cell. The CD117+ immune cell may be an autoreactive lymphocyte, such as a T-cell that expresses a T-cell receptor that specifically binds, and mounts an immune response against, a self antigen. By depleting self-reactive, CD117+ cells, the compositions and methods described herein can be used to treat autoimmune pathologies, such as those described below. Additionally or alternatively, the compositions and methods described herein can be used to treat an autoimmune disease by depleting a population of endogenous hematopoietic stem cells prior to hematopoietic stem cell transplantation therapy, in which case the transplanted cells can home to a niche created by the endogenous cell depletion step and establish productive hematopoiesis. This, in turn, can re-constitute a population of cells depleted during autoimmune cell eradication.

Autoimmune diseases that can be treated using the compositions and methods described herein include, without limitation, psoriasis, psoriatic arthritis, Type 1 diabetes mellitus (Type 1 diabetes), rheumatoid arthritis (RA), human systemic lupus (SLE), multiple sclerosis (MS), inflammatory bowel disease (IBD), lymphocytic colitis, acute disseminated encephalomyelitis (ADEM), Addison's disease, alopecia universalis, ankylosing spondylitisis, antiphospholipid antibody syndrome (APS), aplastic anemia, autoimmune hemolytic anemia, autoimmune hepatitis, autoimmune inner ear disease (AIED), autoimmune lymphoproliferative syndrome (ALPS), autoimmune oophoritis, Balo disease, Behcet's disease, bullous pemphigoid, cardiomyopathy, Chagas' disease, chronic fatigue immune dysfunction syndrome (CFIDS), chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome (GBS), Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease (MCTD), myasthenia gravis, neuromyotonia, opsoclonus myoclonus syndrome (OMS), optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis (also known as “giant cell arteritis”), ulcerative colitis, collagenous colitis, uveitis, vasculitis, vitiligo, vulvodynia (“vulvar vestibulitis”), and Wegener's granulomatosis.

Other methods in which ADCS described herein can be used are described in US 2019-0153114 and US 2019-0144558, both of which are incorporated by reference herein.

### EXAMPLES

The following examples are put forth so as to provide those of ordinary skill in the art with a description of how the compositions and methods described herein may be used, made, and evaluated, and are intended to be purely exemplary of the present disclosure and are not intended to limit the scope of what the inventors regard as their invention.

### Example 1. Baboon CD34+ Bone Marrow Killing Assay

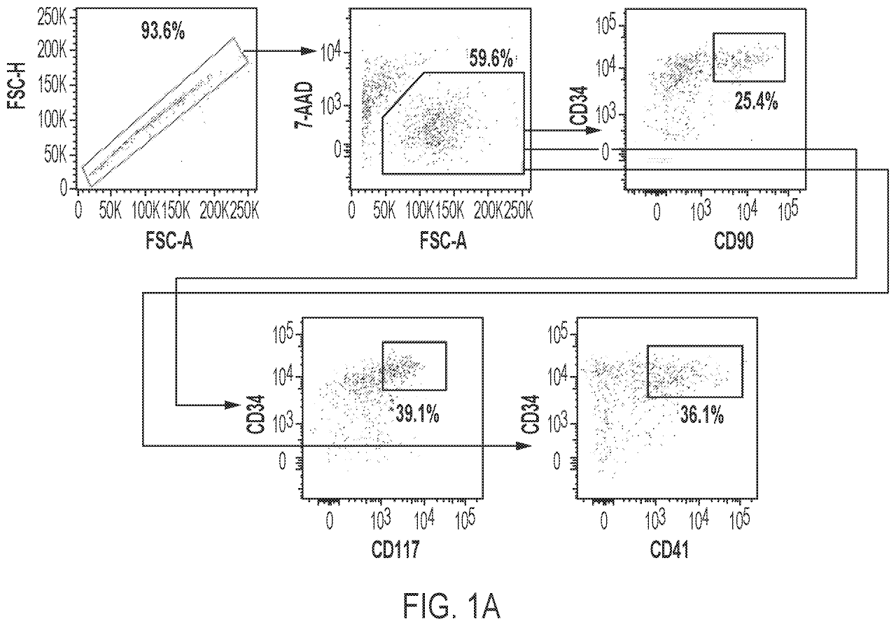

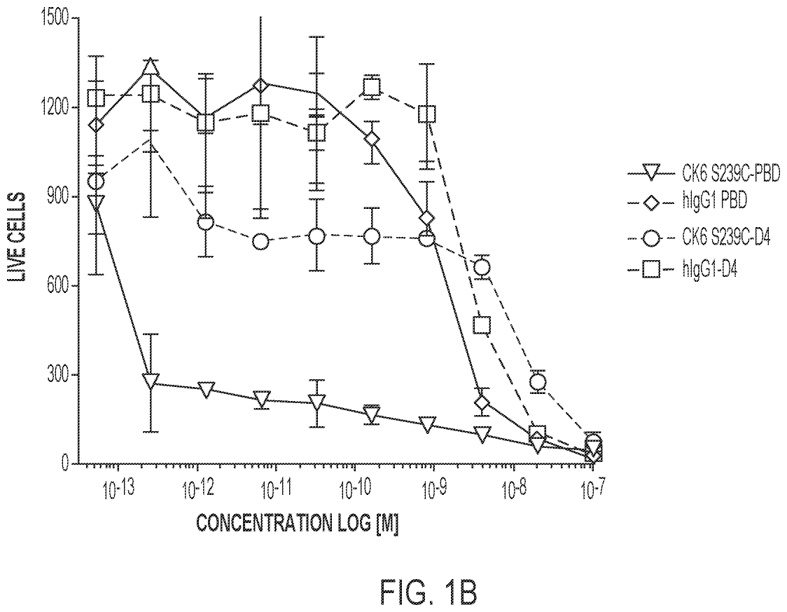

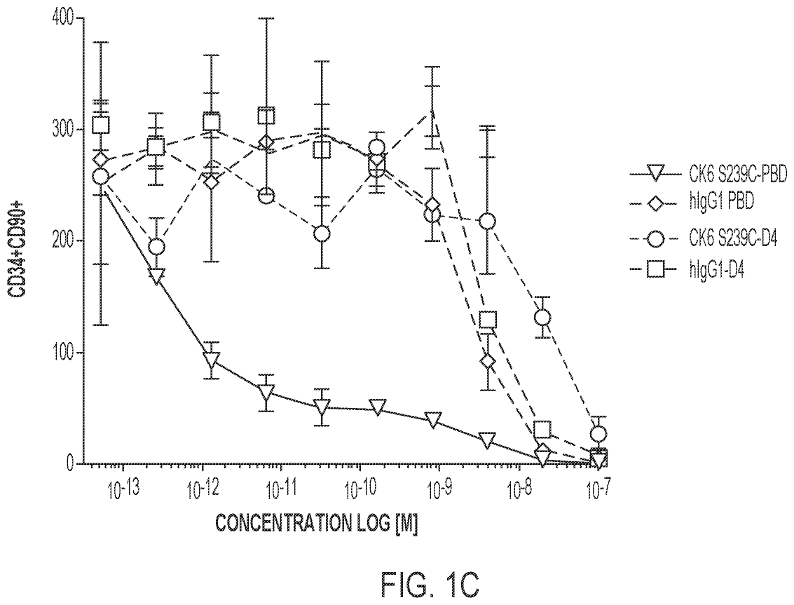

The killing of CD34+ bone marrow cells was investigated in vivo with an anti-CD117 antibody conjugated to PBD, or calicheamicin. Bone marrow cell were isolated from baboons treated with anti-CD117-PBD, or anti-CD117-calicheamicin (D4) ADC. CK6 was used as the anti-CD117 antibody in the ADCs. A non-specific IgG was used as a control. Bone marrow cells were resuspended in SFEM media containing 100 ng/mL of recombinant human Tpo, Flt3L, and IL-6 (no SCF). An Aldefluor assay was run. Then, cells were plated and antibodies and reagents were titrated and added to cells. On day 6, cells were stained with CD34, CD90, CD117 (104D2), CD41, and 7-AAD. Data was collected by flow cytometry on Celesta. An example of the flow cytometry gating strategy used is depicted in FIG. 1A. Live cell counts were determined for all cells or CD34+CD90+ gated cells by flow cytometry.

The results are described in FIGS. 1A-1C and indicate that baboon CD34 cells are sensitive to anti-CD117-PBD in vivo.

### Table:

| | CON- | | | | |
|---|---|---|---|---|---|
| ANTIBODY | JUGATE | DAR | IC50 | | EFFICACY @ [ ] |
| CK6 S239C | PBD | 1.70 | 0.66 | pM | 70.4% @ 1-6 pM |
| CK6 S239C | D4 | 1.72 | 14.6 | nM | NC |

### Example 2. In Vitro Cell Killing Assay

An anti-CD117 antibody was conjugated to PNU, PBD, D4 (calicheamicin), or DM1 (duocarmycin). Each ADC was assessed in a cell killing assay in Kasumi-1 cells or primary human stem cells.

For in vitro killing assays using Kasumi-1 cells, Kasumi-1 cells were grown according to ATCC guidelines. More specifically, Kasumi-1 cells were cultured in the presence of the indicated CD117-ADC or the controls (Isotype ADC). Cell viability of all cells or CD117(−) cells was measured by CellTiter-Glo. For in vitro killing assays using human HSCs (i.e., isolated primary human CD34+ selected Bone Marrow Cells (BMCs)), human CD34+ BMCs were cultured with the indicated CD117-ADCs or the controls (Isotype-ADC). Live cell counts were determined for CD34+CD90+ gated cells by flow cytometry.

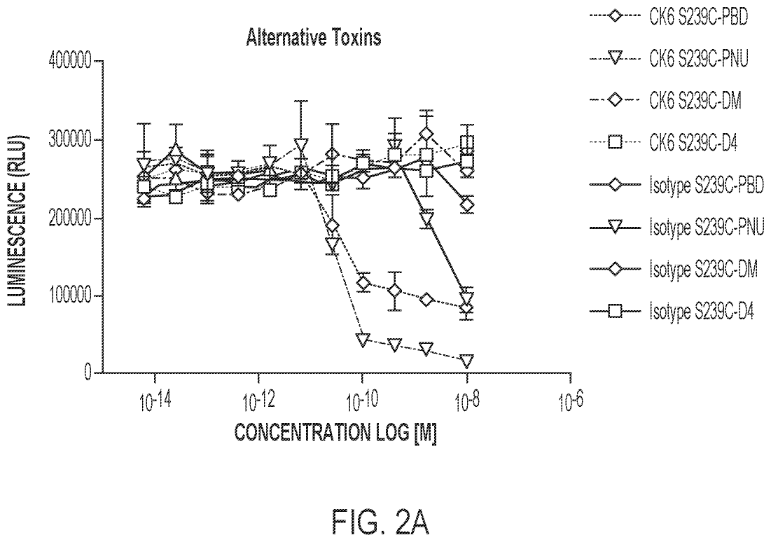

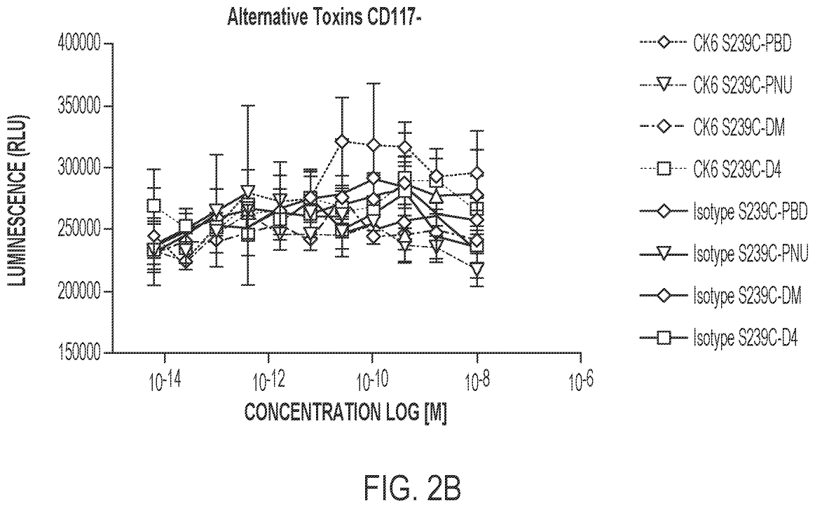

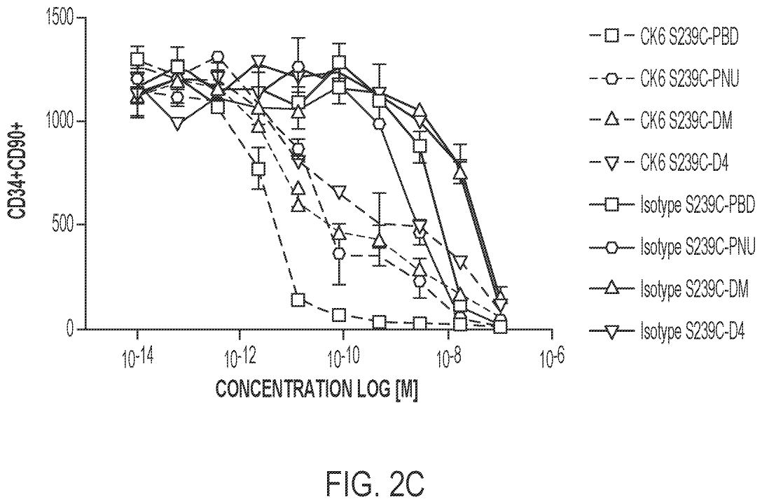

The results for the Kasumi cell killing assay are shown in the below table and in FIGS. 2A and 2B. As shown in FIG. 2A, the anti-CD117 ADCs displayed different degrees of killing potency on Kasumi cells (order of potency from greatest to least: PNU>PBD>>>D4, DM). These results indicate that PNU demonstrated potent killing. As shown in FIG. 2B, no activity was observed in CD117(−) cells.

TABLE
In vitro Kasumi cell killing assay

| Fc | | | | | % Effi- |
|---|---|---|---|---|---|
| modification | Label | | DAR | IC50 | cacy⁺ |
| (S239C) | CK6-PBD | DNA cross | 1.7 | 3.5E−11 | 60.71 |
| | | linker | | | |
| (S239C) | CK6-PNU | DNA | 1.4 | 3.7E−11 | 85.74 |
| | | Topoisomerase | | | |
| | | I/II inhibitor | | | |
| (S239C) | CK6-DM | Duocarmycin | 1.65 | 8.4E−12 | 0 |
| (S239C) | CK6-D4 | Calicheamicin | 1.72 | 9E−11 | 0 |
| (S239C) | Isotype-PBD | Isotype | 1.57 | — | 0 |
| (S239C) | Isotype-PNU | Isotype | 1.3 | — | 0 |
| (S239C) | Isotype-DM | Isotype | 1.44 | — | 0 |
| (S239C) | Isotype-D4 | Isotype | 1.57 | — | 0 |
| D265C | CK6-SET0207 | Non-cleavable | 2.0 | 1.1E−12 | 0 |
| H435A | | natural | | | |

The results for the human CD34+ cell killing assay are shown in the below table and in FIG. 2C. The anti-CD117 ADCs displayed different degrees of killing potency in hCD34 cells (order of potency from greatest to least: PBD>PNU=Duocarmycin>Calicheamicin).

TABLE
In vitro hCD34 cell killing assay

| Fc modi- | | | | | % Effi- |
|---|---|---|---|---|---|
| fication | Label | | DAR | IC50⁺ | cacy⁺⁺ |
| (S239C) | CK6-PBD | DNA cross linker | 1.7 | 2.75E−12 | 97.29 |
| (S239C) | CK6-PNU | DNA Topoisomerase | 1.4 | 2.65E−11 | 74.38 |
| | | I/II inhibitor | | | |
| (S239C) | CK6-DM | Duocarmycin | 1.65 | 8.54E−12 | 66.19 |
| (S239C) | CK6-D4 | Calicheamicin | 1.72 | 2.45E−11 | 52.32 |
| (S239C) | Isotype-PBD | Isotype | 1.57 | 6.16E−09 | 0 |
| (S239C) | Isotype-PNU | Isotype | 1.3 | 1.96E−09 | 24.58 |
| (S239C) | Isotype-DM | Isotype | 1.44 | 5.52E−08 | 0 |
| (S239C) | Isotype-D4 | Isotype | 1.57 | 5.42E−08 | 0 |
| D265C | CK6 | Non-cleavable | 2.0 | 1.11E−08 | 9.18 |
| H435A | SET0207 | natural | | | |

### Example 3. In Vivo HSC Depletion in hNSG Mice with Anti-CD117 ADCs

To identify toxins with potent activity against hematopoietic stem cells, in vivo HSC depletion by an anti-CD117 conjugated to different toxins (PNU, PBD, D4 and DM1) was assessed in hNSG mice. Anti CD117-3100 was conjugated (DAR 2 ss) to PNU (DNA Topoisomeriase I/II inhibitor), PBD (DNA cross-linker), D4 (calicheamicin), or DM1 (duocarmycin). Standard humanized NSG female mice (Jackson Laboratories) were administered an anti-CD117 ADC intravenously (n=3 mice/group) at one of the dosages outlined in the below table. Blood and bone marrow was collected on day 7, day 14, or day 21 post-administration and assessed by flow cytometry (Blood FC: mCD45, hCD45, CD33, CD19, CD3; Bone Marrow FC: mCD45, hCD45, CD33, CD19, CD3, CD38, CD34, CD117, CD90, CD45RA).

TABLE
Study Design

| Antibody - toxin | Doses (mg/kg) |
|---|---|
| CK6 S239C-PNU | 0.01, 0.03, 0.1, 0.3 |
| ISO hlgG S239C-PNU | 0.3 |
| CK6-S239C-PBD | 0.01, 0.03, 0.1, 0.3 |
| ISO hlgG S239C-PBD | 0.3 |
| CK6 S239C-D4 | 0.01, 0.03, 0.1, 0.3 |
| ISO hlgG S239C-D4 | 0.3 |
| CK6-S239C-DM1 | 0.01, 0.03, 0.1, 0.3 |
| ISO hlgG S239C-DM1 | 0.3 |

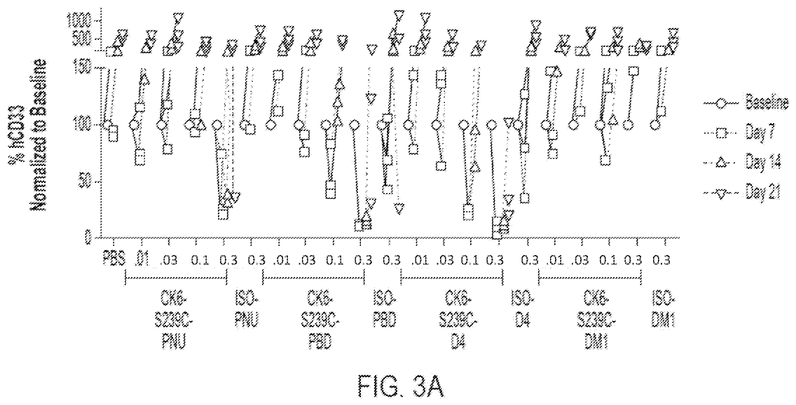

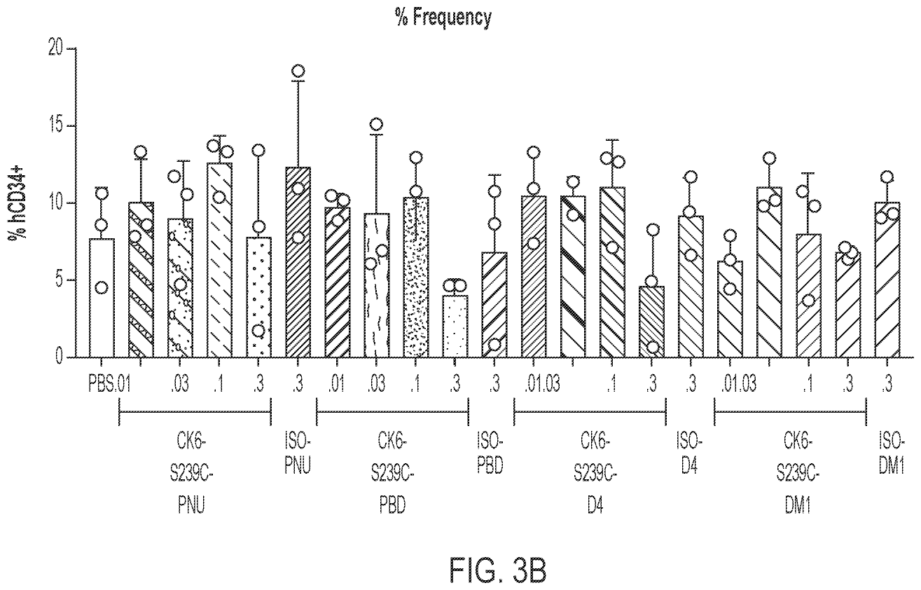

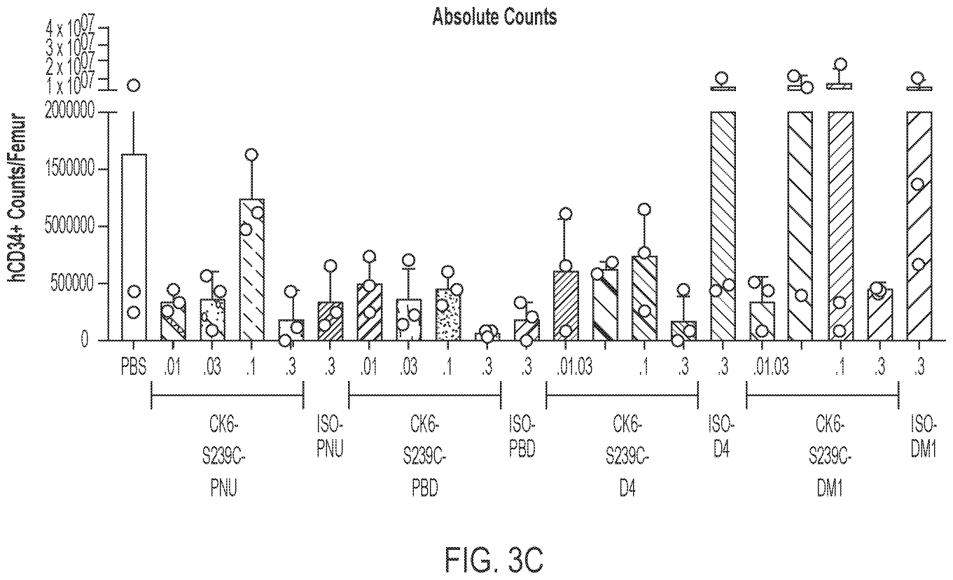

FIG. 3A depicts the percentage of hCD33 cells normalized to baseline in mice treated with the indicated ADC 1 week, 2 weeks, or 3 weeks post-administration. These results show that anti-CD117-PNU, Anti-CD117-PBD, and Anti-CD117-D4 showed good myeloid depletion at 0.3 mg/kg.

The percentage of hCD34+ cells and the hCD34+ count per femur in mice treated with the indicated ADC and dosage 1 week, 2 weeks, or 3 weeks post-administration are shown in FIGS. 3B and 3C. These results indicate that anti-CD117-PNU, anti-CD117-PBD, and anti-CD117-D4 showed good CD34+ cell depletion at 0.3 mg/kg.

### Example 4. In Vivo Efficacy of a Higher Dose Response with Anti-CD117 ADCs

The efficacy of a higher dose response to an anti-CD117-PBD ADC or anti-CD117-calicheamicin ADC was evaluated in hNSG mice. Standard humanized NSG female mice (Jackson Laboratories) were administered an anti-CD117 ADC intravenously (n=5 mice/group) at one of the dosages outlined in the below table. Peripheral blood and bone marrow was collected on day 7, day 14, or day 21 post-administration and assessed by flow cytometry.

TABLE
Study Design

| | Groups | | Request |
|---|---|---|---|
| ADC Description | (mg/kg) | DAR | Total mg |
| CK6-S239C-PBD | 0.3, 1, 3 | 1.7 | 0.516 |
| CK6-S239C-D4 (Calicheamicin) | 0.3, 1, 3 | 1.72 | 0.516 |
| Isotype-S239C-PBD | 1 | 1.57 | 0.12 |
| Isotype-S239C-D4 (Calicheamicin) | 1 | 1.57 | 0.12 |
| PBS | | | |

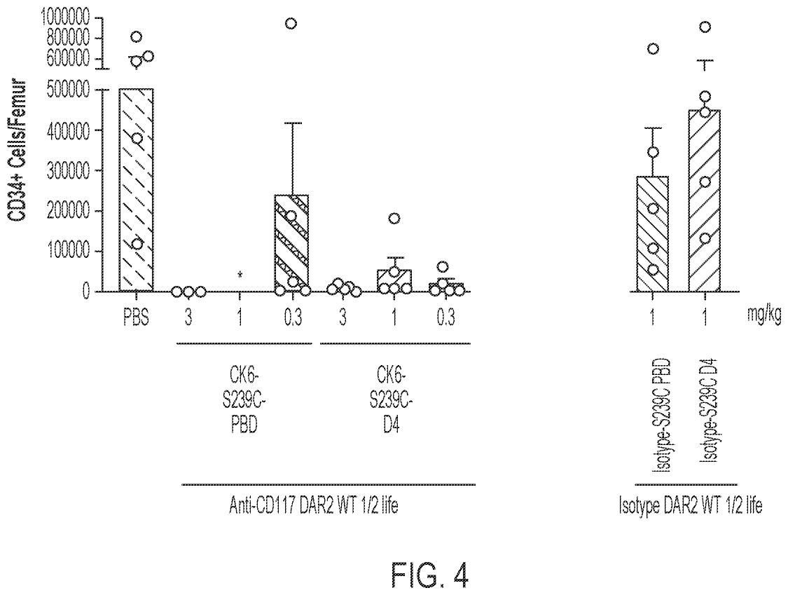

The hCD34+ count per femur 21 days post-administration in mice treated with the indicated ADC and dosage is shown in FIG. 4. These results indicate that anti-CD117-calicheamicin demonstrates depletion of HSCs in the bone marrow of a NSG mouse model.

### Example 5. Maximum Tolerated Dose of PBD and Calicheamicin in C₅₇BL/6 Mice

The maximum tolerated dose (MTD) of a single dose IV administration of an anti-CD117-antibody conjugate to PBD or calicheamicin was determined in C₅₇BL/6 mice. C₅₇BL/6 mice were intravenously administered an anti-CD117-PBD or anti-CD117-calichemicin ADC at 15 mg/kg, as outlined in the table below. Body weight was assessed after Day 0, Day 3, Day 4, or Day 7 after administration along with survival.

TABLE
Study Design

| | Groups | |
|---|---|---|
| ADC Description | (mg/kg) | DAR |
| CK6-S239C-PBD | 15 | 1.7 |
| CK6-S239C-D4 (Calicheamicin) | 15 | 1.72 |

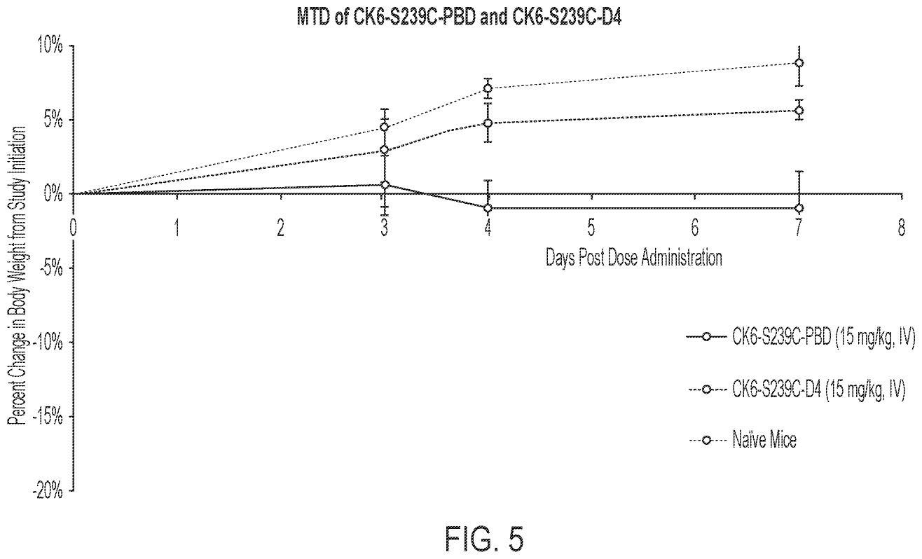

The maximum tolerated dose of the anti-CD117-PBD ADC and anti-CD117-calicheamicin ADC was >15 mg/kg with a single IV dose administration. No significant change in body weight over the course of 7 days (≤−5% body weight loss for individual animals) was observed, as shown in FIG. 5. These results indicate that the anti-CD117-PBD ADC and anti-CD117-calicheamicin ADC are tolerated at 15 mg/kg in C₅₇BL/6 mice.

SEQUENCE TABLE

| Sequence | | |
|---|---|---|
| Identifier | Description | Sequence |
| SEQ ID NO: 1 | CK6 CDR-H1 | SYWIG |
| SEQ ID NO: 2 | CK6 CDR-H2 | IIYPGDSDTRYSPSFQG |
| SEQ ID NO: 3 | CK6 CDR-H3 | HGRGYNGYEGAFDI |
| SEQ ID NO: 4 | CK6 CDR-L1 | RASQGISSALA |
| SEQ ID NO: 5 | CK6 CDR-L2 | DASSLES |
| SEQ ID NO: 6 | CK6 CDR-L3 | CQQFNSYPLT |
| SEQ ID NO: 7 | Consensus human | EVQLVESGGGLVQPGGSLRLSCA |
| | Ab | ASGFTFSDYAMSWVRQAPGKGLE |
| | Heavy chain variable | WVAVISENGSDTYYADSVKGRFTI |
| | domain | SRDDSKNTLYLQMNSLRAEDTAV |
| | | YYCARDRGGAVSYFDVWGQGTL |
| | | VTVSS |
| SEQ ID NO: 8 | Consensus human | DIQMTQSPSSLSASVGDRVTITCR |
| | Ab | ASQDVSSYLAWYQQKPGKAPKLL |
| | Light chain variable | IYAASSLESGVPSRFSGSGSGTDF |
| | domain | TLTISSLQPEDFATYYCQQYNSLP |
| | | YTFGQGTKVEIKRT |
| SEQ ID NO: 9 | Ab67 Heavy chain | EVQLVESGGGLVQPGGSLRLSCA |
| | variable region (e.g., | ASGFTFSDADMDWVRQAPGKGL |
| | as found in HC-67) | EWVGRTRNKAGSYTTEYAASVK |
| | (CDRs in bold) | GRFTISRDDSKNSLYLQMNSLKTE |
| | | DTAVYYCAREPKYWIDFDLWGRG |
| | | TLVTVSS |
| SEQ ID NO: 10 | Ab67 Light chain | DIQMTQSPSSLSASVGDRVTITCR |
| | variable region (e.g., | ASQSISSYLNWYQQKPGKAPKLLI |
| | as found in LC-67) | YAASSLQSGVPSRFSGSGSGTDF |
| | (CDRs in bold) | TLTISSLQPEDFATYYCQQSYIAPY |
| | | TFGGGTKVEIK |
| SEQ ID NO: 11 | Ab67 CDR-H1 | FTFSDADMD |
| SEQ ID NO: 12 | Ab67 CDR-H2 | RTRNKAGSYTTEYAASVKG |
| SEQ ID NO: 13 | Ab67 CDR-H3 | AREPKYWIDFDL |
| SEQ ID NO: 14 | Ab67 CDR-L1 | RASQSISSYLN |
| SEQ ID NO: 15 | Ab67 CDR-L2 | AASSLQS |
| SEQ ID NO: 16 | Ab67 CDR-L3 | QQSYIAPYT |
| SEQ ID NO: 17 | Ab67 Heavy chain | GAGGTGCAGCTGGTGGAGTCTG |
| | variable region | GGGGAGGCTTGGTCCAGCCTGG |
| | (nucl) | AGGGTCCCTGAGACTCTCCTGT |
| | | GCAGCCTCTGGATTCACCTTCAG |
| | | TGACGCCGACATGGACTGGGTC |
| | | CGCCAGGCTCCAGGGAAGGGGC |
| | | TGGAGTGGGTTGGCCGTACTAG |
| | | AAACAAAGCAGGAAGTTACACCA |
| | | CAGAATACGCCGCGTCTGTGAAA |
| | | GGCAGATTCACCATCTCAAGAGA |
| | | TGATTCAAAGAACTCACTGTATC |
| | | TGCAAATGAACAGCCTGAAAACC |
| | | GAGGACACGGCGGTGTACTACT |
| | | GCGCCAGAGAGCCTAAATACTG |
| | | GATCGACTTCGACCTATGGGGG |
| | | AGAGGTACCTTGGTCACCGTCTC |
| | | CTCA |
| SEQ ID NO: 18 | Ab67 Light chain | GACATCCAGATGACCCAGTCTCC |
| | variable region | ATCCTCCCTGTCTGCATCTGTAG |
| | (nucl) | GAGACAGAGTCACCATCACTTGC |
| | | CGGGCAAGTCAGAGCATTAGCA |
| | | GCTATTTAAATTGGTATCAGCAG |
| | | AAACCAGGGAAAGCCCCTAAGC |
| | | TCCTGATCTATGCTGCATCCAGT |
| | | TTGCAAAGTGGGGTCCCATCAAG |
| | | GTTCAGTGGCAGTGGATCTGGG |
| | | ACAGATTTCACTCTCACCATCAG |
| | | CAGTCTGCAACCTGAAGATTTTG |
| | | CAACTTACTACTGTCAGCAAAGC |
| | | TACATCGCCCCTTACACTTTTGG |
| | | CGGAGGGACCAAGGTTGAGATC |
| | | AAA |
| SEQ ID NO: 19 | Ab55 Heavy chain | QVQLVQSGAEVKKPGSSVKVSCK |
| | variable region (e.g., | ASGGTFRIYAISWVRQAPGQGLE |
| | as found in HC-55) | WMGGIIPDFGVANYAQKFQGRVTI |
| | (CDRs in bold) | TADESTSTAYMELSSLRSEDTAVY |
| | | YCARGGLDTDEFDLWGRGTLVTV |
| | | SS |
| SEQ ID NO: 20 | Ab55 Light chain | DIQMTQSPSSLSASVGDRVTITCR |
| | variable region (e.g., | ASQSINSYLNWYQQKPGKAPKLLI |
| | as found in LC-55) | YAASSLQSGVPSRFSGSGSGTDF |
| | (CDRs in bold) | TLTISSLQPEDFATYYCQQGVSDIT |
| | | FGGGTKVEIK |
| SEQ ID NO: 21 | Ab55 CDR-H1 | GTFRIYAIS |
| SEQ ID NO: 22 | Ab55 CDR-H2 | GIIPDFGVANYAQKFQG |
| SEQ ID NO: 23 | Ab55 CDR-H3 | ARGGLDTDEFDL |
| SEQ ID NO: 24 | Ab55 CDR-L1 | RASQSINSYLN |
| SEQ ID NO: 25 | Ab55 CDR-L2 | AASSLQS |
| SEQ ID NO: 26 | Ab55 CDR-L3 | QQGVSDIT |
| SEQ ID NO: 27 | Ab55 Heavy chain | CAGGTGCAGCTGGTGCAGTCTG |
| | variable region | GGGCTGAGGTGAAGAAGCCTGG |
| | (nucl) | GTCCTCGGTGAAGGTCTCCTGC |
| | | AAGGCTTCTGGAGGCACCTTCC |
| | | GAATCTATGCTATCAGCTGGGTG |
| | | CGACAGGCCCCTGGACAAGGGC |
| | | TTGAGTGGATGGGAGGGATCAT |
| | | CCCTGACTTCGGTGTAGCAAACT |
| | | ACGCACAGAAGTTCCAGGGCAG |
| | | AGTCACGATTACCGCGGACGAAT |
| | | CCACGAGCACAGCCTACATGGA |
| | | GCTGAGCAGCCTGAGATCTGAG |
| | | GACACGGCGGTGTACTACTGCG |
| | | CCAGAGGTGGATTGGACACAGA |
| | | CGAGTTCGACCTATGGGGGAGA |
| | | GGTACCTTGGTCACCGTCTCCTC |
| | | A |
| SEQ ID NO: 28 | Ab55 Light chain | GACATCCAGATGACCCAGTCTCC |
| | variable region | ATCCTCCCTGTCTGCATCTGTAG |
| | (nucl) | GAGACAGAGTCACCATCACTTGC |
| | | CGGGCAAGTCAGAGCATTAACA |
| | | GCTATTTAAATTGGTATCAGCAG |
| | | AAACCAGGGAAAGCCCCTAAGC |
| | | TCCTGATCTATGCTGCATCCAGT |
| | | TTGCAAAGTGGGGTCCCATCAAG |
| | | GTTCAGTGGCAGTGGATCTGGG |
| | | ACAGATTTCACTCTCACCATCAG |
| | | CAGTCTGCAACCTGAAGATTTTG |
| | | CAACTTACTACTGTCAGCAAGGA |
| | | GTCAGTGACATCACTTTTGGCGG |
| | | AGGGACCAAGGTTGAGATCAAA |
| SEQ ID NO: 29 | Ab54 Heavy chain | QVQLVQSGAEVKKPGSSVKVSCK |
| | variable region (e.g., | ASGGTFSSYAISWVRQAPGQGLE |
| | as found in HC-54) | WMGGIIPIFGTANYAQKFQGRVTI |
| | hIgG1 backbone | TADESTSTAYMELSSLRSEDTAVY |
| | (CDRs in bold) | YCARGGLDTDEFDLWGRGTLVTV |
| | | SS |
| SEQ ID NO: 30 | Ab54 Light chain | DIQMTQSPSSLSASVGDRVTITCR |
| | variable region (e.g., | ASQSINSYLNWYQQKPGKAPKLLI |
| | as found in LC-54) | YAASSLQSGVPSRFSGSGSGTDF |
| | (CDRs in bold) | TLTISSLQPEDFATYYCQQGVSDIT |
| | | FGGGTKVEIK |
| SEQ ID NO: 31 | Ab54 CDR-H1 | GTFSSYAIS |
| SEQ ID NO: 32 | Ab54 CDR-H2 | GIIPIFGTANYAQKFQG |
| SEQ ID NO: 33 | Ab54 CDR-H3 | ARGGLDTDEFDL |
| SEQ ID NO: 34 | Ab54 CDR-L1 | RASQSINSYLN |
| SEQ ID NO: 35 | Ab54 CDR-L2 | AASSLQS |
| SEQ ID NO: 36 | Ab54 CDR-L3 | QQGVSDIT |
| SEQ ID NO: 37 | Ab54 Heavy chain | CAGGTGCAGCTGGTGCAGTCTG |
| | variable region | GGGCTGAGGTGAAGAAGCCTGG |
| | (nucl) | GTCCTCGGTGAAGGTCTCCTGC |
| | | AAGGCTTCTGGAGGCACCTTCA |
| | | GCAGCTATGCTATCAGCTGGGT |
| | | GCGACAGGCCCCTGGACAAGGG |
| | | CTTGAGTGGATGGGAGGGATCA |
| | | TCCCTATCTTTGGTACAGCAAAC |
| | | TACGCACAGAAGTTCCAGGGCA |
| | | GAGTCACGATTACCGCGGACGA |
| | | ATCCACGAGCACAGCCTACATG |
| | | GAGCTGAGCAGCCTGAGATCTG |
| | | AGGACACGGCGGTGTACTACTG |
| | | CGCCAGAGGTGGATTGGACACA |
| | | GACGAGTTCGACCTATGGGGGA |
| | | GAGGTACCTTGGTCACCGTCTCC |
| | | TCA |
| SEQ ID NO: 38 | Ab54 Light chain | GACATCCAGATGACCCAGTCTCC |
| | variable region | ATCCTCCCTGTCTGCATCTGTAG |
| | (nucl) | GAGACAGAGTCACCATCACTTGC |
| | | CGGGCAAGTCAGAGCATTAACA |
| | | GCTATTTAAATTGGTATCAGCAG |
| | | AAACCAGGGAAAGCCCCTAAGC |
| | | TCCTGATCTATGCTGCATCCAGT |
| | | TTGCAAAGTGGGGTCCCATCAAG |
| | | GTTCAGTGGCAGTGGATCTGGG |
| | | ACAGATTTCACTCTCACCATCAG |
| | | CAGTCTGCAACCTGAAGATTTTG |
| | | CAACTTACTACTGTCAGCAAGGA |
| | | GTCAGTGACATCACTTTTGGCGG |
| | | AGGGACCAAGGTTGAGATCAAA |
| SEQ ID NO: 39 | Ab56 Heavy chain | QVQLVQSGAEVKKPGSSVKVSCK |
| | variable region | ASGGTFSLYAISWVRQAPGQGLE |
| | (e.g., as found in | WMGGIIPAFGTANYAQKFQGRVTI |
| | HC-56) | TADESTSTAYMELSSLRSEDTAVY |
| | (CDRs in bold) | YCARGGLDTDEFDLWGRGTLVTV |
| | | SS |
| SEQ ID NO: 40 | Ab56 Light chain | DIQMTQSPSSLSASVGDRVTITCR |
| | variable region (e.g., | ASQSINSYLNWYQQKPGKAPKLLI |
| | as found in LC-56) | YAASSLQSGVPSRFSGSGSGTDF |
| | | TLTISSLQPEDFATYYCQQGVSDIT |
| | (CDRs in bold) | FGGGTKVEIK |
| SEQ ID NO: 41 | Ab56 CDR-H1 | GTFSLYAIS |
| SEQ ID NO: 42 | Ab56 CDR-H2 | GIIPAFGTANYAQKFQG |
| SEQ ID NO: 43 | Ab56 CDR-H3 | ARGGLDTDEFDL |
| SEQ ID NO: 44 | Ab56 CDR-L1 | RASQSINSYLN |
| SEQ ID NO: 45 | Ab56 CDR-L2 | AASSLQS |
| SEQ ID NO: 46 | Ab56 CDR-L3 | QQGVSDIT |
| SEQ ID NO: 47 | Ab56 Heavy chain | CAGGTGCAGCTGGTGCAGTCTG |
| | variable region | GGGCTGAGGTGAAGAAGCCTGG |
| | (nucl) | GTCCTCGGTGAAGGTCTCCTGC |
| | | AAGGCTTCTGGAGGCACCTTCA |
| | | GCCTCTATGCTATCTCCTGGGTG |
| | | CGACAGGCCCCTGGACAAGGGC |
| | | TTGAGTGGATGGGAGGGATCAT |
| | | CCCTGCCTTCGGTACCGCAAACT |
| | | ACGCACAGAAGTTCCAGGGCAG |
| | | AGTCACGATTACCGCGGACGAAT |
| | | CCACGAGCACAGCCTACATGGA |
| | | GCTGAGCAGCCTGAGATCTGAG |
| | | GACACGGCGGTGTACTACTGCG |
| | | CCAGAGGTGGATTGGACACAGA |
| | | CGAGTTCGACCTATGGGGGAGA |
| | | GGTACCTTGGTCACCGTCTCCTC |
| | | A |
| SEQ ID NO: 48 | Ab56 Light chain | GACATCCAGATGACCCAGTCTCC |
| | variable region | ATCCTCCCTGTCTGCATCTGTAG |
| | (nucl) | GAGACAGAGTCACCATCACTTGC |
| | | CGGGCAAGTCAGAGCATTAACA |
| | | GCTATTTAAATTGGTATCAGCAG |
| | | AAACCAGGGAAAGCCCCTAAGC |
| | | TCCTGATCTATGCTGCATCCAGT |
| | | TTGCAAAGTGGGGTCCCATCAAG |
| | | GTTCAGTGGCAGTGGATCTGGG |
| | | ACAGATTTCACTCTCACCATCAG |
| | | CAGTCTGCAACCTGAAGATTTTG |
| | | CAACTTACTACTGTCAGCAAGGA |
| | | GTCAGTGACATCACTTTTGGCGG |
| | | AGGGACCAAGGTTGAGATCAAA |
| SEQ ID NO: 49 | Ab57 Heavy chain | QVQLVQSGAEVKKPGSSVKVSCK |
| | variable region (e.g., | ASGGTFSLYAISWVRQAPGQGLE |
| | as found in HC-57) | WMGGIIPHFGLANYAQKFQGRVTI |
| | (CDRs in bold) | TADESTSTAYMELSSLRSEDTAVY |
| | | YCARGGLDTDEFDLWGRGTLVTV |
| | | SS |
| SEQ ID NO: 50 | Ab57 Light chain | DIQMTQSPSSLSASVGDRVTITCR |
| | variable region (e.g., | ASQSINSYLNWYQQKPGKAPKW |
| | as found in LC-57) | YAASSLQSGVPSRFSGSGSGTDF |
| | (CDRs in bold) | TLTISSLQPEDFATYYCQQGVSDIT |
| | | FGGGTKVEIK |
| SEQ ID NO: 51 | Ab57 CDR-H1 | GTFSLYAIS |
| SEQ ID NO: 52 | Ab57 CDR-H2 | GIIPHFGLANYAQKFQG |
| SEQ ID NO: 53 | Ab57 CDR-H3 | ARGGLDTDEFDL |
| SEQ ID NO: 54 | Ab57 CDR-L1 | RASQSINSYLN |
| SEQ ID NO: 55 | Ab57 CDR-L2 | AASSLQS |
| SEQ ID NO: 56 | Ab57 CDR-L3 | QQGVSDIT |
| SEQ ID NO: 57 | Ab57 Heavy chain | CAGGTGCAGCTGGTGCAGTCTG |
| | variable region | GGGCTGAGGTGAAGAAGCCTGG |
| | (nucl) | GTCCTCGGTGAAGGTCTCCTGC |
| | | AAGGCTTCTGGAGGCACCTTCTC |
| | | CCTCTATGCTATCAGCTGGGTGC |
| | | GACAGGCCCCTGGACAAGGGCT |
| | | TGAGTGGATGGGAGGGATCATC |
| | | CCTCACTTCGGTCTCGCAAACTA |
| | | CGCACAGAAGTTCCAGGGCAGA |
| | | GTCACGATTACCGCGGACGAAT |
| | | CCACGAGCACAGCCTACATGGA |
| | | GCTGAGCAGCCTGAGATCTGAG |
| | | GACACGGCGGTGTACTACTGCG |
| | | CCAGAGGTGGATTGGACACAGA |
| | | CGAGTTCGACCTATGGGGGAGA |
| | | GGTACCTTGGTCACCGTCTCCTC |
| | | A |
| SEQ ID NO: 58 | Ab57 Light chain | GACATCCAGATGACCCAGTCTCC |
| | variable region | ATCCTCCCTGTCTGCATCTGTAG |
| | (nucl) | GAGACAGAGTCACCATCACTTGC |
| | | CGGGCAAGTCAGAGCATTAACA |
| | | GCTATTTAAATTGGTATCAGCAG |
| | | AAACCAGGGAAAGCCCCTAAGC |
| | | TCCTGATCTATGCTGCATCCAGT |
| | | TTGCAAAGTGGGGTCCCATCAAG |
| | | GTTCAGTGGCAGTGGATCTGGG |
| | | ACAGATTTCACTCTCACCATCAG |
| | | CAGTCTGCAACCTGAAGATTTTG |
| | | CAACTTACTACTGTCAGCAAGGA |
| | | GTCAGTGACATCACTTTTGGCGG |
| | | AGGGACCAAGGTTGAGATCAAA |
| SEQ ID NO: 59 | Ab58 Heavy chain | EVQLLESGGGLVQPGGSLRLSCA |
| | variable region (e.g., | ASGFTFSNYAMSWVRQAPGKGLE |
| | as found in HC-58) | WVSAISGSGGSTYYADSVKGRFTI |
| | (CDRs in bold) | SRDNSKNTLYLQMNSLRAEDTAV |
| | | YYCAKGPPTYHTNYYYMDVWGK |
| | | GTTVTVSS |
| SEQ ID NO: 60 | Ab58 Light chain | DIQMTQSPSSVSASVGDRVTITCR |
| | variable region (e.g., | ASQGISSWLAWYQQKPGKAPKLL |
| | as found in LC-58) | IYAASSLQSGVPSRFSGSGSGTD |
| | (CDRs in bold) | FTLTISSLQPEDFATYYCQQTNSF |
| | | PYTFGGGTKVEIK |
| SEQ ID NO: 61 | Ab58 CDR-H1 | FTFSNYAMS |
| SEQ ID NO: 62 | Ab58 CDR-H2 | AISGSGGSTYYADSVKG |
| SEQ ID NO: 63 | Ab58 CDR-H3 | AKGPPTYHTNYYYMDV |
| SEQ ID NO: 64 | Ab58 CDR-L1 | RASQGISSWLA |
| SEQ ID NO: 65 | Ab58 CDR-L2 | AASSLQS |
| SEQ ID NO: 66 | Ab58 CDR-L3 | QQTNSFPYT |
| SEQ ID NO: 67 | Ab58 Heavy chain | GAGGTGCAGCTGTTGGAGTCTG |
| | variable region | GGGGAGGCTTGGTACAGCCTGG |
| | (nucl) | GGGGTCCCTGAGACTCTCCTGT |
| | | GCAGCCTCTGGATTCACCTTTAG |
| | | CAATTATGCCATGAGCTGGGTCC |
| | | GCCAGGCTCCAGGGAAGGGGCT |
| | | GGAGTGGGTCTCAGCTATTAGTG |
| | | GTAGTGGTGGTAGCACATACTAC |
| | | GCAGACTCCGTGAAGGGCCGGT |
| | | TCACCATCTCCAGAGACAATTCC |
| | | AAGAACACGCTGTATCTGCAAAT |
| | | GAACAGCCTGAGAGCCGAGGAC |
| | | ACGGCGGTGTACTACTGCGCCA |
| | | AGGGCCCTCCTACATACCACACA |
| | | AACTACTACTACATGGACGTATG |
| | | GGGCAAGGGTACAACTGTCACC |
| | | GTCTCCTCA |
| SEQ ID NO: 68 | Ab58 Light chain | GACATCCAGATGACCCAGTCTCC |
| | variable region | ATCTTCCGTGTCTGCATCTGTAG |
| | (nucl) | GAGACAGAGTCACCATCACTTGT |
| | | CGGGCGAGTCAGGGTATTAGCA |
| | | GCTGGTTAGCCTGGTATCAGCA |
| | | GAAACCAGGGAAAGCCCCTAAG |
| | | CTCCTGATCTATGCTGCATCCAG |
| | | TTTGCAAAGTGGGGTCCCATCAA |
| | | GGTTCAGCGGCAGTGGATCTGG |
| | | GACAGATTTCACTCTCACCATCA |
| | | GCAGCCTGCAGCCTGAAGATTTT |
| | | GCAACTTATTACTGTCAGCAAAC |
| | | AAATAGTTTCCCTTACACTTTTGG |
| | | CGGAGGGACCAAGGTTGAGATC |
| | | AAA |
| SEQ ID NO: 69 | Ab61 Heavy chain | EVQLLESGGGLVQPGGSLRLSCA |
| | variable region (e.g., | ASGFTFSSYVMIWVRQAPGKGLE |
| | as found in HC-61) | WVSSISGDSVTTYYADSVKGRFTI |
| | (CDRs in bold) | SRDNSKNTLYLQMNSLRAEDTAV |
| | | YYCAKGPPTYHTNYYYMDVWGK |
| | | GTTVTVSS |
| SEQ ID NO: 70 | Ab61 Light chain | DIQMTQSPSSVSASVGDRVTITCR |
| | variable region (e.g., | ASQGISSWLAWYQQKPGKAPKLL |
| | as found in LC-61) | IYAASSLQSGVPSRFSGSGSGTD |
| | (CDRs in bold) | FTLTISSLQPEDFATYYCQQTNSF |
| | | PYTFGGGTKVEIK |
| SEQ ID NO: 71 | Ab61 CDR-H1 | FTFSSYVMI |
| SEQ ID NO: 72 | Ab61 CDR-H2 | SISGDSVTTYYADSVKG |
| SEQ ID NO: 73 | Ab61 CDR-H3 | AKGPPTYHTNYYYMDV |
| SEQ ID NO: 74 | Ab61 CDR-L1 | RASQGISSWLA |
| SEQ ID NO: 75 | Ab61 CDR-L2 | AASSLQS |
| SEQ ID NO: 76 | Ab61 CDR-L3 | QQTNSFPYT |
| SEQ ID NO: 77 | Ab61 Heavy chain | GAGGTGCAGCTGTTGGAGTCTG |
| | variable region | GGGGAGGCTTGGTACAGCCTGG |
| | (nucl) | GGGGTCCCTGAGACTCTCCTGT |
| | | GCAGCCTCTGGATTCACCTTTAG |
| | | CAGCTATGTCATGATCTGGGTCC |
| | | GCCAGGCTCCAGGGAAGGGGCT |
| | | GGAGTGGGTCTCAAGCATTAGT |
| | | GGTGACAGCGTAACAACATACTA |
| | | CGCAGACTCCGTGAAGGGCCGG |
| | | TTCACCATCTCCAGAGACAATTC |
| | | CAAGAACACGCTGTATCTGCAAA |
| | | TGAACAGCCTGAGAGCCGAGGA |
| | | CACGGCGGTGTACTACTGCGCC |
| | | AAGGGCCCTCCTACATACCACAC |
| | | AAACTACTACTACATGGACGTAT |
| | | GGGGCAAGGGTACAACTGTCAC |
| | | CGTCTCCTCA |
| SEQ ID NO: 78 | Ab61 Light chain | GACATCCAGATGACCCAGTCTCC |
| | variable region | ATCTTCCGTGTCTGCATCTGTAG |
| | (nuc) | GAGACAGAGTCACCATCACTTGT |
| | | CGGGCGAGTCAGGGTATTAGCA |
| | | GCTGGTTAGCCTGGTATCAGCA |
| | | GAAACCAGGGAAAGCCCCTAAG |
| | | CTCCTGATCTATGCTGCATCCAG |
| | | TTTGCAAAGTGGGGTCCCATCAA |
| | | GGTTCAGCGGCAGTGGATCTGG |
| | | GACAGATTTCACTCTCACCATCA |
| | | GCAGCCTGCAGCCTGAAGATTTT |
| | | GCAACTTATTACTGTCAGCAAAC |
| | | AAATAGTTTCCCTTACACTTTTGG |
| | | CGGAGGGACCAAGGTTGAGATC |
| | | AAA |
| SEQ ID NO: 79 | Ab66 Heavy chain | EVQLVESGGGLVQPGGSLRLSCA |
| | variable region (e.g., | ASGFTFSDHYMDWVRQAPGKGL |
| | as found in HC-66) | EWVGRTRNKASSYTTEYAASVKG |
| | (CDRs in bold) | RFTISRDDSKNSLYLQMNSLKTED |
| | | TAVYYCAREPKYWIDFDLWGRGT |
| | | LVTVSS |
| SEQ ID NO: 80 | Ab66 Light chain | DIQMTQSPSSLSASVGDRVTITCR |
| | variable region (e.g., | ASQSISSYLNWYQQKPGKAPKLLI |
| | as found in LC-66) | YAASSLQSGVPSRFSGSGSGTDF |
| | (CDRs in bold) | TLTISSLQPEDFATYYCQQSYIAPY |
| | | TFGGGTKVEIK |
| SEQ ID NO: 81 | Ab66 CDR-H1 | FTFSDHYMD |
| SEQ ID NO: 82 | Ab66 CDR-H2 | RTRNKASSYTTEYAASVKG |
| SEQ ID NO: 83 | Ab66 CDR-H3 | AREPKYWIDFDL |
| SEQ ID NO: 84 | Ab66 CDR-L1 | RASQSISSYLN |
| SEQ ID NO: 85 | Ab66 CDR-L2 | AASSLQS |
| SEQ ID NO: 86 | Ab66 CDR-L3 | QQSYIAPYT |
| SEQ ID NO: 87 | Ab66 Heavy chain | GAGGTGCAGCTGGTGGAGTCTG |
| | variable region | GGGGAGGCTTGGTCCAGCCTGG |
| | (nucl) | AGGGTCCCTGAGACTCTCCTGT |
| | | GCAGCCTCTGGATTCACCTTCAG |
| | | TGACCACTACATGGACTGGGTCC |
| | | GCCAGGCTCCAGGGAAGGGGCT |
| | | GGAGTGGGTTGGCCGTACTAGA |
| | | AACAAAGCTAGTAGTTACACCAC |
| | | AGAATACGCCGCGTCTGTGAAA |
| | | GGCAGATTCACCATCTCAAGAGA |
| | | TGATTCAAAGAACTCACTGTATC |
| | | TGCAAATGAACAGCCTGAAAACC |
| | | GAGGACACGGCGGTGTACTACT |
| | | GCGCCAGAGAGCCTAAATACTG |
| | | GATCGACTTCGACCTATGGGGG |
| | | AGAGGTACCTTGGTCACCGTCTC |
| | | CTCA |
| SEQ ID NO: 88 | Ab66 Light chain | GACATCCAGATGACCCAGTCTCC |
| | variable region | ATCCTCCCTGTCTGCATCTGTAG |
| | (nucl) | GAGACAGAGTCACCATCACTTGC |
| | | CGGGCAAGTCAGAGCATTAGCA |
| | | GCTATTTAAATTGGTATCAGCAG |
| | | AAACCAGGGAAAGCCCCTAAGC |
| | | TCCTGATCTATGCTGCATCCAGT |
| | | TTGCAAAGTGGGGTCCCATCAAG |
| | | GTTCAGTGGCAGTGGATCTGGG |
| | | ACAGATTTCACTCTCACCATCAG |
| | | CAGTCTGCAACCTGAAGATTTTG |
| | | CAACTTACTACTGTCAGCAAAGC |
| | | TACATCGCCCCTTACACTTTTGG |
| | | CGGAGGGACCAAGGTTGAGATC |
| | | AAA |
| SEQ ID NO: 89 | Ab68 Heavy chain | EVQLVESGGGLVQPGRSLRLSCT |
| | variable region (e.g., | ASGFTFSDHDMNWVRQAPGKGL |
| | as found in HC-68) | EWVGRTRNAAGSYTTEYAASVK |
| | (CDRs in bold) | GRFTISRDDSKNSLYLQMNSLKTE |
| | | DTAVYYCAREPKYWIDFDLWGRG |
| | | TLVTVSS |
| SEQ ID NO: 90 | Ab68 Light chain | DIQMTQSPSSLSASVGDRVTITCR |
| | variable region (e.g., | ASQSISSYLNWYQQKPGKAPKLLI |
| | as found in LC-68) | YAASSLQSGVPSRFSGSGSGTDF |
| | (CDRs in bold) | TLTISSLQPEDFATYYCQQSYIAPY |
| | | TFGGGTKVEIK |
| SEQ ID NO: 91 | Ab68 CDR-H1 | FTFSDHDMN |
| SEQ ID NO: 92 | Ab68 CDR-H2 | RTRNAAGSYTTEYAASVKG |
| SEQ ID NO: 93 | Ab68 CDR-H3 | AREPKYWIDFDL |
| SEQ ID NO: 94 | Ab68 CDR-L1 | RASQSISSYLN |
| SEQ ID NO: 95 | Ab68 CDR-L2 | AASSLQS |
| SEQ ID NO: 96 | Ab68 CDR-L3 | QQSYIAPYT |
| SEQ ID NO: 97 | Ab68 Heavy chain | GAGGTGCAGCTGGTGGAGTCTG |
| | variable region | GGGGAGGCTTGGTACAGCCAGG |
| | (nucl) | GCGGTCCCTGAGACTCTCCTGTA |
| | | CAGCTTCTGGATTCACCTTCAGT |
| | | GACCACGACATGAACTGGGTCC |
| | | GCCAGGCTCCAGGGAAGGGGCT |
| | | GGAGTGGGTTGGCCGTACTAGA |
| | | AACGCCGCTGGAAGTTACACCA |
| | | CAGAATACGCCGCGTCTGTGAAA |
| | | GGCAGATTCACCATCTCAAGAGA |
| | | TGATTCAAAGAACTCACTGTATC |
| | | TGCAAATGAACAGCCTGAAAACC |
| | | GAGGACACGGCGGTGTACTACT |
| | | GCGCCAGAGAGCCTAAATACTG |
| | | GATCGACTTCGACCTATGGGGG |
| | | AGAGGTACCTTGGTCACCGTCTC |
| | | CTCA |
| SEQ ID NO: 98 | Ab68 Light chain | GACATCCAGATGACCCAGTCTCC |
| | variable region | ATCCTCCCTGTCTGCATCTGTAG |
| | (nucl) | GAGACAGAGTCACCATCACTTGC |
| | | CGGGCAAGTCAGAGCATTAGCA |
| | | GCTATTTAAATTGGTATCAGCAG |
| | | AAACCAGGGAAAGCCCCTAAGC |
| | | TCCTGATCTATGCTGCATCCAGT |
| | | TTGCAAAGTGGGGTCCCATCAAG |
| | | GTTCAGTGGCAGTGGATCTGGG |
| | | ACAGATTTCACTCTCACCATCAG |
| | | CAGTCTGCAACCTGAAGATTTTG |
| | | CAACTTACTACTGTCAGCAAAGC |
| | | TACATCGCCCCTTACACTTTTGG |
| | | CGGAGGGACCAAGGTTGAGATC |
| | | AAA |
| SEQ ID NO: 99 | Ab69 Heavy chain | EVQLVESGGGLVQPGGSLRLSCA |
| | variable region (e.g., | ASGFTFVDHDMDWVRQAPGKGL |
| | as found in HC-69) | EWVGRTRNKLGSYTTEYAASVKG |
| | (CDRs in bold) | RFTISRDDSKNSLYLQMNSLKTED |
| | | TAVYYCAREPKYWIDFDLWGRGT |
| | | LVTVSS |
| SEQ ID NO: 100 | Ab69 Light chain | DIQMTQSPSSLSASVGDRVTITCR |
| | variable region (e.g., | ASQSISSYLNWYQQKPGKAPKLLI |
| | as found in LC-69) | YAASSLQSGVPSRFSGSGSGTDF |
| | (CDRs in bold) | TLTISSLQPEDFATYYCQQSYIAPY |
| | | TFGGGTKVEIK |
| SEQ ID NO: 101 | Ab69 CDR-H1 | FTFVDHDMD |
| SEQ ID NO: 102 | Ab69 CDR-H2 | RTRNKLGSYTTEYAASVKG |
| SEQ ID NO: 103 | Ab69 CDR-H3 | AREPKYWIDFDL |
| SEQ ID NO: 104 | Ab69 CDR-L1 | RASQSISSYLN |
| SEQ ID NO: 105 | Ab69 CDR-L2 | AASSLQS |
| SEQ ID NO: 106 | Ab69 CDR-L3 | QQSYIAPYT |
| SEQ ID NO: 107 | Ab69 Heavy chain | GAGGTGCAGCTGGTGGAGTCTG |
| | variable region | GGGGAGGCTTGGTCCAGCCTGG |
| | (nucl) | AGGGTCCCTGAGACTCTCCTGT |
| | | GCAGCCTCTGGATTCACCTTCGT |
| | | AGACCACGACATGGACTGGGTC |
| | | CGCCAGGCTCCAGGGAAGGGGC |
| | | TGGAGTGGGTTGGCCGTACTAG |
| | | AAACAAACTAGGAAGTTACACCA |
| | | CAGAATACGCCGCGTCTGTGAAA |
| | | GGCAGATTCACCATCTCAAGAGA |
| | | TGATTCAAAGAACTCACTGTATC |
| | | TGCAAATGAACAGCCTGAAAACC |
| | | GAGGACACGGCGGTGTACTACT |
| | | GCGCCAGAGAGCCTAAATACTG |
| | | GATCGACTTCGACCTATGGGGG |
| | | AGAGGTACCTTGGTCACCGTCTC |
| | | CTCA |
| SEQ ID NO: 108 | Ab69 Light chain | GACATCCAGATGACCCAGTCTCC |
| | variable region | ATCCTCCCTGTCTGCATCTGTAG |
| | (nucl) | GAGACAGAGTCACCATCACTTGC |
| | | CGGGCAAGTCAGAGCATTAGCA |
| | | GCTATTTAAATTGGTATCAGCAG |
| | | AAACCAGGGAAAGCCCCTAAGC |
| | | TCCTGATCTATGCTGCATCCAGT |
| | | TTGCAAAGTGGGGTCCCATCAAG |
| | | GTTCAGTGGCAGTGGATCTGGG |
| | | ACAGATTTCACTCTCACCATCAG |
| | | CAGTCTGCAACCTGAAGATTTTG |
| | | CAACTTACTACTGTCAGCAAAGC |
| | | TACATCGCCCCTTACACTTTTGG |
| | | CGGAGGGACCAAGGTTGAGATC |
| | | AAA |
| SEQ ID NO: 109 | Ab67 Light chain | DIQMTQSPSSLSASVGDRVTIT |
| | LC constant region | CRASQSISSYLNWYQQKPGKA |
| | underlined | PKLLIYAASSLQSGVPSRFSGS |
| | | GSGTDFTLTISSLQPEDFATYY |
| | | CQQSYIAPYTFGGGTKVEIKRT |
| | | VAAPSVFIFPPSDEQLKSGTAS |
| | | VVCLLNNFYPREAKVQWKVDN |
| | | ALQSGNSQESVTEQDSKDSTY |
| | | SLSSTLTLSKADYEKHKVYACE |
| | | VTHQGLSSPVTKSFNRGEC |
| SEQ ID NO: 110 | Ab67 Heavy chain | EVQLVESGGGLVQPGGSLRLS |
| | HC constant region | CAASGFTFSDADMDWVRQAP |
| | underlined | GKGLEWVGRTRNKAGSYTTEY |
| | | AASVKGRFTISRDDSKNSLYLQ |
| | | MNSLKTEDTAVYYCAREPKYW |
| | | IDFDLWGRGTLVTVSSASTKGP |
| | | SVFPLAPSSKSTSGGTAALGCL |
| | | VKDYFPEPVTVSWNSGALTSG |
| | | VHTFPAVLQSSGLYSLSSVVTV |
| | | PSSSLGTQTYICNVNHKPSNTK |
| | | VDKKVEPKSCDKTHTCPPCPA |
| | | PELLGGPSVFLFPPKPKDTLMI |
| | | SRTPEVTCVVVDVSHEDPEVK |
| | | FNWYVDGVEVHNAKTKPREEQ |
| | | YNSTYRVVSVLTVLHQDWLNG |
| | | KEYKCKVSNKALPAPIEKTISKA |
| | | KGQPREPQVYTLPPSRDELTK |
| | | NQVSLTCLVKGFYPSDIAVEWE |
| | | SNGQPENNYKTTPPVLDSDGS |
| | | FFLYSKLTVDKSRWQQGNVFS |
| | | CSVMHEALHNHYTQKSLSLSP |
| | | GK |
| SEQ ID NO: 111 | Ab67 Heavy chain | EVQLVESGGGLVQPGGSLRLS |
| | (D265C)* | CAASGFTFSDADMDWVRQAP |
| | HC constant region | GKGLEWVGRTRNKAGSYTTEY |
| | underlined | AASVKGRFTISRDDSKNSLYLQ |
| | | MNSLKTEDTAVYYCAREPKYW |
| | | IDFDLWGRGTLVTVSSASTKGP |
| | | SVFPLAPSSKSTSGGTAALGCL |
| | | VKDYFPEPVTVSWNSGALTSG |
| | | VHTFPAVLQSSGLYSLSSVVTV |
| | | PSSSLGTQTYICNVNHKPSNTK |
| | | VDKKVEPKSCDKTHTCPPCPA |
| | | PELLGGPSVFLFPPKPKDTLMI |
| | | SRTPEVTCVVVCVSHEDPEVK |
| | | FNWYVDGVEVHNAKTKPREEQ |
| | | YNSTYRVVSVLTVLHQDWLNG |
| | | KEYKCKVSNKALPAPIEKTISKA |
| | | KGQPREPQVYTLPPSRDELTK |
| | | NQVSLTCLVKGFYPSDIAVEWE |
| | | SNGQPENNYKTTPPVLDSDGS |
| | | FFLYSKLTVDKSRWQQGNVFS |
| | | CSVMHEALHNHYTQKSLSLSP |
| | | GK |
| SEQ ID NO: 112 | Ab67 Heavy chain | EVQLVESGGGLVQPGGSLRLS |
| | (L234A/L235A/ | CAASGFTFSDADMDWVRQAP |
| | D265C)* | GKGLEWVGRTRNKAGSYTTEY |
| | HC constant region | AASVKGRFTISRDDSKNSLYLQ |
| | underlined | MNSLKTEDTAVYYCAREPKYW |
| | | IDFDLWGRGTLVTVSSASTKGP |
| | | SVFPLAPSSKSTSGGTAALGCL |
| | | VKDYFPEPVTVSWNSGALTSG |
| | | VHTFPAVLQSSGLYSLSSVVTV |
| | | PSSSLGTQTYICNVNHKPSNTK |
| | | VDKKVEPKSCDKTHTCPPCPA |
| | | PEAAGGPSVFLFPPKPKDTLMI |
| | | SRTPEVTCVVVCVSHEDPEVK |
| | | FNWYVDGVEVHNAKTKPREEQ |
| | | YNSTYRVVSVLTVLHQDWLNG |
| | | KEYKCKVSNKALPAPIEKTISKA |
| | | KGQPREPQVYTLPPSRDELTK |
| | | NQVSLTCLVKGFYPSDIAVEWE |
| | | SNGQPENNYKTTPPVLDSDGS |
| | | FFLYSKLTVDKSRWQQGNVFS |
| | | CSVMHEALHNHYTQKSLSLSP |
| | | GK |
| SEQ ID NO: 113 | Ab67 Heavy chain | EVQLVESGGGLVQPGGSLRLS |
| | (D265C/H435A)* | CAASGFTFSDADMDWVRQAP |
| | HC constant region | GKGLEWVGRTRNKAGSYTTEY |
| | underlined | AASVKGRFTISRDDSKNSLYLQ |
| | | MNSLKTEDTAVYYCAREPKYW |
| | | IDFDLWGRGTLVTVSSASTKGP |
| | | SVFPLAPSSKSTSGGTAALGCL |
| | | VKDYFPEPVTVSWNSGALTSG |
| | | VHTFPAVLQSSGLYSLSSVVTV |
| | | PSSSLGTQTYICNVNHKPSNTK |
| | | VDKKVEPKSCDKTHTCPPCPA |
| | | PELLGGPSVFLFPPKPKDTLMI |
| | | SRTPEVTCVVVCVSHEDPEVK |
| | | FNWYVDGVEVHNAKTKPREEQ |
| | | YNSTYRVVSVLTVLHQDWLNG |
| | | KEYKCKVSNKALPAPIEKTISKA |
| | | KGQPREPQVYTLPPSRDELTK |
| | | NQVSLTCLVKGFYPSDIAVEWE |
| | | SNGQPENNYKTTPPVLDSDGS |
| | | FFLYSKLTVDKSRWQQGNVFS |
| | | CSVMHEALHNAYTQKSLSLSP |
| | | GK |
| SEQ ID NO: 114 | Ab67 Heavy chain | EVQLVESGGGLVQPGGSLRLS |
| | (L234A/L235A/ | CAASGFTFSDADMDWVRQAP |
| | D265C/H435A)* | GKGLEWVGRTRNKAGSYTTEY |
| | HC constant region | AASVKGRFTISRDDSKNSLYLQ |
| | underlined | MNSLKTEDTAVYYCAREPKYW |
| | | IDFDLWGRGTLVTVSSASTKGP |
| | | SVFPLAPSSKSTSGGTAALGCL |
| | | VKDYFPEPVTVSWNSGALTSG |
| | | VHTFPAVLQSSGLYSLSSVVTV |
| | | PSSSLGTQTYICNVNHKPSNTK |
| | | VDKKVEPKSCDKTHTCPPCPA |
| | | PEAAGGPSVFLFPPKPKDTLMI |
| | | SRTPEVTCVVVCVSHEDPEVK |
| | | FNWYVDGVEVHNAKTKPREEQ |
| | | YNSTYRVVSVLTVLHQDWLNG |
| | | KEYKCKVSNKALPAPIEKTISKA |
| | | KGQPREPQVYTLPPSRDELTK |
| | | NQVSLTCLVKGFYPSDIAVEWE |
| | | SNGQPENNYKTTPPVLDSDGS |
| | | FFLYSKLTVDKSRWQQGNVFS |
| | | CSVMHEALHNAYTQKSLSLSP |
| | | GK |
| SEQ ID NO: 115 | Ab55 Light chain | DIQMTQSPSSLSASVGDRVTITCR |
| | LC constant region | ASQSINSYLNWYQQKPGKAPKLLI |
| | underlined | YAASSLQSGVPSRFSGSGSGTDF |
| | | TLTISSLQPEDFATYYCQQGVSDIT |
| | | FGGGTKVEIKRTVAAPSVFIFPP |
| | | SDEQLKSGTASVVCLLNNFYP |
| | | REAKVQWKVDNALQSGNSQE |
| | | SVTEQDSKDSTYSLSSTLTLSK |
| | | ADYEKHKVYACEVTHQGLSSP |
| | | VTKSFNRGEC |
| SEQ ID NO: 116 | Ab55 Heavy chain | QVQLVQSGAEVKKPGSSVKVSCK |
| | HC constant region | ASGGTFRIYAISWVRQAPGQGLE |
| | underlined | WMGGIIPDFGVANYAQKFQGRVTI |
| | | TADESTSTAYMELSSLRSEDTAVY |
| | | YCARGGLDTDEFDLWGRGTLVTV |
| | | SSASTKGPSVFPLAPSSKSTSG |
| | | GTAALGCLVKDYFPEPVTVSW |
| | | NSGALTSGVHTFPAVLQSSGL |
| | | YSLSSVVTVPSSSLGTQTYICN |
| | | VNHKPSNTKVDKKVEPKSCDK |
| | | THTCPPCPAPELLGGPSVFLFP |
| | | PKPKDTLMISRTPEVTCVVVDV |
| | | SHEDPEVKFNWYVDGVEVHNA |
| | | KTKPREEQYNSTYRVVSVLTVL |
| | | HQDWLNGKEYKCKVSNKALPA |
| | | PIEKTISKAKGQPREPQVYTLP |
| | | PSRDELTKNQVSLTCLVKGFYP |
| | | SDIAVEWESNGQPENNYKTTP |
| | | PVLDSDGSFFLYSKLTVDKSR |
| | | WQQGNVFSCSVMHEALHNHY |
| | | TQKSLSLSPGK |
| SEQ ID NO: 117 | Ab55 Heavy chain | QVQLVQSGAEVKKPGSSVKVSCK |
| | (D265C)* | ASGGTFRIYAISWVRQAPGQGLE |
| | HC constant region | WMGGIIPDFGVANYAQKFQGRVTI |
| | underlined | TADESTSTAYMELSSLRSEDTAVY |
| | | YCARGGLDTDEFDLWGRGTLVTV |
| | | SSASTKGPSVFPLAPSSKSTSG |
| | | GTAALGCLVKDYFPEPVTVSW |
| | | NSGALTSGVHTFPAVLQSSGL |
| | | YSLSSVVTVPSSSLGTQTYICN |
| | | VNHKPSNTKVDKKVEPKSCDK |
| | | THTCPPCPAPELLGGPSVFLFP |
| | | PKPKDTLMISRTPEVTCVVVCV |
| | | SHEDPEVKFNWYVDGVEVHNA |
| | | KTKPREEQYNSTYRVVSVLTVL |
| | | HQDWLNGKEYKCKVSNKALPA |
| | | PIEKTISKAKGQPREPQVYTLP |
| | | PSRDELTKNQVSLTCLVKGFYP |
| | | SDIAVEWESNGQPENNYKTTP |
| | | PVLDSDGSFFLYSKLTVDKSR |
| | | WQQGNVFSCSVMHEALHNHY |
| | | TQKSLSLSPGK |
| SEQ ID NO: 118 | Ab55 Heavy chain | QVQLVQSGAEVKKPGSSVKVSCK |
| | (L234A/ L235A/ | ASGGTFRIYAISWVRQAPGQGLE |
| | D265C)* | WMGGIIPDFGVANYAQKFQGRVTI |
| | HC constant region | TADESTSTAYMELSSLRSEDTAVY |
| | underlined | YCARGGLDTDEFDLWGRGTLVTV |
| | | SSASTKGPSVFPLAPSSKSTSG |
| | | GTAALGCLVKDYFPEPVTVSW |
| | | NSGALTSGVHTFPAVLQSSGL |
| | | YSLSSVVTVPSSSLGTQTYICN |
| | | VNHKPSNTKVDKKVEPKSCDK |
| | | THTCPPCPAPEAAGGPSVFLF |
| | | PPKPKDTLMISRTPEVTCVVVC |
| | | VSHEDPEVKFNWYVDGVEVHN |
| | | AKTKPREEQYNSTYRVVSVLT |
| | | VLHQDWLNGKEYKCKVSNKAL |
| | | PAPIEKTISKAKGQPREPQVYT |
| | | LPPSRDELTKNQVSLTCLVKGF |
| | | YPSDIAVEWESNGQPENNYKT |
| | | TPPVLDSDGSFFLYSKLTVDKS |
| | | RWQQGNVFSCSVMHEALHNH |
| | | YTQKSLSLSPGK |
| SEQ ID NO: 119 | Ab55 Heavy chain | QVQLVQSGAEVKKPGSSVKVSCK |
| | (D265C/H435A)* | ASGGTFRIYAISWVRQAPGQGLE |
| | HC constant region | WMGGIIPDFGVANYAQKFQGRVTI |
| | underlined | TADESTSTAYMELSSLRSEDTAVY |
| | | YCARGGLDTDEFDLWGRGTLVTV |
| | | SSASTKGPSVFPLAPSSKSTSG |
| | | GTAALGCLVKDYFPEPVTVSW |
| | | NSGALTSGVHTFPAVLQSSGL |
| | | YSLSSVVTVPSSSLGTQTYICN |
| | | VNHKPSNTKVDKKVEPKSCDK |
| | | THTCPPCPAPELLGGPSVFLFP |
| | | PKPKDTLMISRTPEVTCVVVCV |
| | | SHEDPEVKFNWYVDGVEVHNA |
| | | KTKPREEQYNSTYRVVSVLTVL |
| | | HQDWLNGKEYKCKVSNKALPA |
| | | PIEKTISKAKGQPREPQVYTLP |
| | | PSRDELTKNQVSLTCLVKGFYP |
| | | SDIAVEWESNGQPENNYKTTP |
| | | PVLDSDGSFFLYSKLTVDKSR |
| | | WQQGNVFSCSVMHEALHNAY |
| | | TQKSLSLSPGK |
| SEQ ID NO: 120 | Ab55 Heavy chain | QVQLVQSGAEVKKPGSSVKVSCK |
| | (L234A/L235A/ | ASGGTFRIYAISWVRQAPGQGLE |
| | D265C/H435A)* | WMGGIIPDFGVANYAQKFQGRVTI |
| | HC constant region | TADESTSTAYMELSSLRSEDTAVY |
| | underlined | YCARGGLDTDEFDLWGRGTLVTV |
| | | SSASTKGPSVFPLAPSSKSTSG |
| | | GTAALGCLVKDYFPEPVTVSW |
| | | NSGALTSGVHTFPAVLQSSGL |
| | | YSLSSVVTVPSSSLGTQTYICN |
| | | VNHKPSNTKVDKKVEPKSCDK |
| | | THTCPPCPAPEAAGGPSVFLF |
| | | PPKPKDTLMISRTPEVTCVVVC |
| | | VSHEDPEVKFNWYVDGVEVHN |
| | | AKTKPREEQYNSTYRVVSVLT |
| | | VLHQDWLNGKEYKCKVSNKAL |
| | | PAPIEKTISKAKGQPREPQVYT |
| | | LPPSRDELTKNQVSLTCLVKGF |
| | | YPSDIAVEWESNGQPENNYKT |
| | | TPPVLDSDGSFFLYSKLTVDKS |
| | | RWQQGNVFSCSVMHEALHNA |
| | | YTQKSLSLSPGK |
| SEQ ID NO: 121 | Light chain constant | RTVAAPSVFIFPPSDEQLKSGT |
| | region of LC-54, | ASVVCLLNNFYPREAKVQWKV |
| | LC-55, LC-56, LC-57, | DNALQSGNSQESVTEQDSKDS |
| | LC-58, LC-61, | TYSLSSTLTLSKADYEKHKVYA |
| | LC-66, LC-67, LC-68, | CEVTHQGLSSPVTKSFNRGEC |
| | LC-69 | |
| SEQ ID NO: 122 | Heavy chain | ASTKGPSVFPLAPSSKSTSGG |
| | constant region of | TAALGCLVKDYFPEPVTVSWN |
| | WT | SGALTSGVHTFPAVLQSSGLY |
| | | SLSSVVTVPSSSLGTQTYICNV |
| | | NHKPSNTKVDKKVEPKSCDKT |
| | | HTCPPCPAPELLGGPSVFLFP |
| | | PKPKDTLMISRTPEVTCVVVDV |
| | | SHEDPEVKFNWYVDGVEVHNA |
| | | KTKPREEQYNSTYRVVSVLTVL |
| | | HQDWLNGKEYKCKVSNKALPA |
| | | PIEKTISKAKGQPREPQVYTLP |
| | | PSRDELTKNQVSLTCLVKGFYP |
| | | SDIAVEWESNGQPENNYKTTP |
| | | PVLDSDGSFFLYSKLTVDKSR |
| | | WQQGNVFSCSVMHEALHNHY |
| | | TQKSLSLSPGK |
| SEQ ID NO: 123 | Heavy chain | ASTKGPSVFPLAPSSKSTSGG |
| | constant region | TAALGCLVKDYFPEPVTVSWN |
| | (D265C)* | SGALTSGVHTFPAVLQSSGLY |
| | | SLSSVVTVPSSSLGTQTYICNV |
| | | NHKPSNTKVDKKVEPKSCDKT |
| | | HTCPPCPAPELLGGPSVFLFPP |
| | | KPKDTLMISRTPEVTCVVVCVS |
| | | HEDPEVKFNWYVDGVEVHNAK |
| | | TKPREEQYNSTYRVVSVLTVLH |
| | | QDWLNGKEYKCKVSNKALPAP |
| | | IEKTISKAKGQPREPQVYTLPP |
| | | SRDELTKNQVSLTCLVKGFYPS |
| | | DIAVEWESNGQPENNYKTTPP |
| | | VLDSDGSFFLYSKLTVDKSRW |
| | | QQGNVFSCSVMHEALHNHYT |
| | | QKSLSLSPGK |
| SEQ ID NO: 124 | Heavy chain | ASTKGPSVFPLAPSSKSTSGG |
| | constant region | TAALGCLVKDYFPEPVTVSWN |
| | (L234A/L235A/ | SGALTSGVHTFPAVLQSSGLY |
| | D265C)* | SLSSVVTVPSSSLGTQTYICNV |
| | | NHKPSNTKVDKKVEPKSCDKT |
| | | HTCPPCPAPEAAGGPSVFLFP |
| | | PKPKDTLMISRTPEVTCVVVCV |
| | | SHEDPEVKFNWYVDGVEVHNA |
| | | KTKPREEQYNSTYRVVSVLTVL |
| | | HQDWLNGKEYKCKVSNKALPA |
| | | PIEKTISKAKGQPREPQVYTLP |
| | | PSRDELTKNQVSLTCLVKGFYP |
| | | SDIAVEWESNGQPENNYKTTP |
| | | PVLDSDGSFFLYSKLTVDKSR |
| | | WQQGNVFSCSVMHEALHNHY |
| | | TQKSLSLSPGK |
| SEQ ID NO: 125 | Heavy chain | ASTKGPSVFPLAPSSKSTSGG |
| | constant region | TAALGCLVKDYFPEPVTVSWN |
| | (H435A/D265C)* | SGALTSGVHTFPAVLQSSGLY |
| | | SLSSVVTVPSSSLGTQTYICNV |
| | | NHKPSNTKVDKKVEPKSCDKT |
| | | HTCPPCPAPELLGGPSVFLFPP |
| | | KPKDTLMISRTPEVTCVVVCVS |
| | | HEDPEVKFNWYVDGVEVHNAK |
| | | TKPREEQYNSTYRVVSVLTVLH |
| | | QDWLNGKEYKCKVSNKALPAP |
| | | IEKTISKAKGQPREPQVYTLPP |
| | | SRDELTKNQVSLTCLVKGFYPS |
| | | DIAVEWESNGQPENNYKTTPP |
| | | VLDSDGSFFLYSKLTVDKSRW |
| | | QQGNVFSCSVMHEALHNAYT |
| | | QKSLSLSPGK |
| SEQ ID NO: 126 | Heavy chain | ASTKGPSVFPLAPSSKSTSGG |
| | constant region | TAALGCLVKDYFPEPVTVSWN |
| | (L234A/L235A/ | SGALTSGVHTFPAVLQSSGLY |
| | H435A/D265C)* | SLSSVVTVPSSSLGTQTYICNV |
| | | NHKPSNTKVDKKVEPKSCDKT |
| | | HTCPPCPAPEAAGGPSVFLFP |
| | | PKPKDTLMISRTPEVTCVVVCV |
| | | SHEDPEVKFNWYVDGVEVHNA |
| | | KTKPREEQYNSTYRVVSVLTVL |
| | | HQDWLNGKEYKCKVSNKALPA |
| | | PIEKTISKAKGQPREPQVYTLP |
| | | PSRDELTKNQVSLTCLVKGFYP |
| | | SDIAVEWESNGQPENNYKTTP |
| | | PVLDSDGSFFLYSKLTVDKSR |
| | | WQQGNVFSCSVMHEALHNAY |
| | | TQKSLSLSPGK |
| SEQ ID NO: 127 | Consensus | GTF(S/R)(S/I/L)YAIS |
| | sequence of variable | |
| | heavy chain CDR1 | |
| | (Abs 54-57) | |
| SEQ ID NO: 128 | Consensus | GIIP(I/D/A/H)FG(T/V/L)A |
| | sequence of variable | NYAQKFQG |
| | heavy chain CDR2 | |
| | (Abs 54-57) | |
| SEQ ID NO: 129 | Variable heavy chain | ARGGLDTDEFDL |
| | CDR3 (Abs 54-57) | |
| SEQ ID NO: 130 | Variable light chain | RASQSINSYLN |
| | CDR1 (Abs 54-57) | |
| SEQ ID NO: 131 | Variable light chain | AASSLQS |
| | CDR2 (Abs 54-57) | |
| SEQ ID NO: 132 | Variable light chain | QQGVSDIT |
| | CDR3 (Abs 54-57) | |
| SEQ ID NO: 133 | Consensus | FTFS(N/S)Y(A/V)M(S/1) |
| | sequence of variable | |
| | heavy chain CDR1 | |
| | (Abs 58, 61) | |
| SEQ ID NO: 134 | Consensus | (A/S)ISG(S/D)(G/S)(G/V) |
| | sequence of variable | (S/T)TYYADSVKG |
| | heavy chain CDR2 | |
| | (Abs 58, 61) | |
| SEQ ID NO: 135 | Variable heavy chain | AKGPPTYHTNYYYMDV |
| | CDR3 (Abs 58, 61) | |
| SEQ ID NO: 136 | Variable light chain | RASQGISSWLA |
| | CDR1 (Abs 58, 61) | |
| SEQ ID NO: 137 | Variable light chain | AASSLQS |
| | CDR2 (Abs 58, 61) | |
| SEQ ID NO: 138 | Variable light chain | QQINSFPYT |
| | CDR3 (Abs 58, 61) | |
| SEQ ID NO: 139 | Consensus | FTF(S/V)D(H/A)(Y/D) |
| | sequence of variable | M(D/N) |
| | heavy chain CDR1 | |
| | (Abs 66-69) | |
| SEQ ID NO: 140 | Consensus | RTRN(K/A)(A/L)(S/G) |
| | sequence of variable | SYTTEYAASVKG |
| | heavy chain CDR2 | |
| | (Abs 66-69) | |
| SEQ ID NO: 141 | Variable heavy chain | AREPKYWIDFDL |
| | CDR3 (Abs 66-69) | |
| SEQ ID NO: 142 | Variable light chain | RASQSISSYLN |
| | CDR1 (Abs 66-69) | |
| SEQ ID NO: 143 | Variable light chain | AASSLQS |
| | CDR2 (Abs 66-69) | |
| SEQ ID NO: 144 | Variable light chain | QQSYIAPYT |
| | CDR3 (Abs 66-69) | |
| SEQ ID NO: 145 | Human CD117 | MRGARGAWDFLCVLLLLLRVQ |
| | (mast/stem cell | TGSSQPSVSPGEPSPPSIHPG |
| | growth factor | KSDLIVRVGDEIRLLCTDPGFV |
| | receptor Kit isoform | KWTFEILDETNENKQNEWITEK |
| | 1 precursor) | AEATNTGKYTCTNKHGLSNSIY |
| | Protein NCBI | VFVRDPAKLFLVDRSLYGKED |
| | Reference | NDTLVRCPLTDPEVTNYSLKG |
| | Sequence: | CQGKPLPKDLRFIPDPKAGIMI |
| | NP_000213.1 | KSVKRAYHRLCLHCSVDQEGK |
| | | SVLSEKFILKVRPAFKAVPVVS |
| | | VSKASYLLREGEEFTVTCTIKD |
| | | VSSSVYSTWKRENSQTKLQEK |
| | | YNSWHHGDFNYERQATLTISS |
| | | ARVNDSGVFMCYANNTFGSAN |
| | | VTTTLEVVDKGFINIFPMINTTV |
| | | FVNDGENVDLIVEYEAFPKPEH |
| | | QQWIYMNRTFTDKWEDYPKSE |
| | | NESNIRYVSELHLTRLKGTEGG |
| | | TYTFLVSNSDVNAAIAFNVYVN |
| | | TKPEILTYDRLVNGMLQCVAAG |
| | | FPEPTIDWYFCPGTEQRCSAS |
| | | VLPVDVQTLNSSGPPFGKLVV |
| | | QSSIDSSAFKHNGTVECKAYN |
| | | DVGKTSAYFNFAFKGNNKEQI |
| | | HPHTLFTPLLIGFVIVAGMMCII |
| | | VMILTYKYLQKPMYEVQWKVV |
| | | EEINGNNYVYIDPTQLPYDHKW |
| | | EFPRNRLSFGKTLGAGAFGKV |
| | | VEATAYGLIKSDAAMTVAVKML |
| | | KPSAHLTEREALMSELKVLSYL |
| | | GNHMNIVNLLGACTIGGPTLVI |
| | | TEYCCYGDLLNFLRRKRDSFIC |
| | | SKQEDHAEAALYKNLLHSKES |
| | | SCSDSTNEYMDMKPGVSYVVP |
| | | TKADKRRSVRIGSYIERDVTPAI |
| | | MEDDELALDLEDLLSFSYQVAK |
| | | GMAFLASKNCIHRDLAARNILL |
| | | THGRITKICDFGLARDIKNDSN |
| | | YVVKGNARLPVKWMAPESIFN |
| | | CVYTFESDVWSYGIFLWELFSL |
| | | GSSPYPGMPVDSKFYKMIKEG |
| | | FRMLSPEHAPAEMYDIMKTCW |
| | | DADPLKRPTFKQIVQLIEKQISE |
| | | STNHIYSNLANCSPNRQKPVV |
| | | DHSVRINSVGSTASSSQPLLVH |
| | | DDV |
| SEQ ID NO: 146 | Human CD117 | MRGARGAWDFLCVLLLLLRVQ |
| | (mast/stem cell | TGSSQPSVSPGEPSPPSIHPG |
| | growth factor | KSDLIVRVGDEIRLLCTDPGFV |
| | receptor Kit isoform | KWTFEILDETNENKQNEWITEK |
| | 2 precursor) | AEATNTGKYTCTNKHGLSNSIY |
| | Protein NCBI | VFVRDPAKLFLVDRSLYGKED |
| | Reference | NDTLVRCPLTDPEVTNYSLKG |
| | Sequence: | CQGKPLPKDLRFIPDPKAGIMI |
| | NP_001087241.1 | KSVKRAYHRLCLHCSVDQEGK |
| | | SVLSEKFILKVRPAFKAVPVVS |
| | | VSKASYLLREGEEFTVTCTIKD |
| | | VSSSVYSTWKRENSQTKLQEK |
| | | YNSWHHGDFNYERQATLTISS |
| | | ARVNDSGVFMCYANNTFGSAN |
| | | VTTTLEVVDKGFINIFPMINTTV |
| | | FVNDGENVDLIVEYEAFPKPEH |
| | | QQWIYMNRTFTDKWEDYPKSE |
| | | NESNIRYVSELHLTRLKGTEGG |
| | | TYTFLVSNSDVNAAIAFNVYVN |
| | | TKPEILTYDRLVNGMLQCVAAG |
| | | FPEPTIDWYFCPGTEQRCSAS |
| | | VLPVDVQTLNSSGPPFGKLVV |
| | | QSSIDSSAFKHNGTVECKAYN |
| | | DVGKTSAYFNFAFKEQIHPHTL |
| | | FTPLLIGFVIVAGMMCIIVMILTY |
| | | KYLQKPMYEVQWKVVEEINGN |
| | | NYVYIDPTQLPYDHKWEFPRN |
| | | RLSFGKTLGAGAFGKVVEATA |
| | | YGLIKSDAAMTVAVKMLKPSAH |
| | | LTEREALMSELKVLSYLGNHM |
| | | NIVNLLGACTIGGPTLVITEYCC |
| | | YGDLLNFLRRKRDSFICSKQED |
| | | HAEAALYKNLLHSKESSCSDST |
| | | NEYMDMKPGVSYVVPTKADKR |
| | | RSVRIGSYIERDVTPAIMEDDE |
| | | LALDLEDLLSFSYQVAKGMAFL |
| | | ASKNCIHRDLAARNILLTHGRIT |
| | | KICDFGLARDIKNDSNYVVKGN |
| | | ARLPVKWMAPESIFNCVYTFES |
| | | DVWSYGIFLWELFSLGSSPYP |
| | | GMPVDSKFYKMIKEGFRMLSP |
| | | EHAPAEMYDIMKTCWDADPLK |
| | | RPTFKQIVQLIEKQISESTNHIY |
| | | SNLANCSPNRQKPVVDHSVRI |
| | | NSVGSTASSSQPLLVHDDV |
| SEQ ID NO: 147 | Heavy chain variable | QVQLVQSGAAVKKPGESLKISCKG |
| | region of HC-1 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 148 | Light chain variable | AIQLTQSPSSLSASVGDRVTITCRA |
| | region of LC-1 | SQGVSSALAWYQQKPGKAPKLLIY |
| | | DASSLESGVPSRFSGSGSGTDFT |
| | | LTISSLQPEDFATYYCQQFNSYPL |
| | | TFGGGTKVEIK |
| SEQ ID NO: 147 | Heavy chain variable | QVQLVQSGAAVKKPGESLKISCKG |
| | region of HC-2 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 149 | Light chain variable | DIQLTQSPSSLSASVGDRVTITCRA |
| | region of LC-2 | SQGIRTDLGWYQQKPGKAPKLLIY |
| | | DASSLESGVPSRFSGSGSGTDFT |
| | | LTISSLQPEDFATYYCQQFNSYPL |
| | | TFGGGTKVEIK |
| SEQ ID NO: 147 | Heavy chain variable | QVQLVQSGAAVKKPGESLKISCKG |
| | region of HC-3 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 150 | Light chain variable | AIRMTQSPSSLSASVGDRVTITCR |
| | region of LC-3 | ASQGIRNDLAWYQQKPGKTPKLLI |
| | | YDASSLESGVPSRFSGSGSGTDF |
| | | TLTISSLQPEDFATYYCQQFNSYP |
| | | LTFGGGTKVEIK |
| SEQ ID NO: 147 | Heavy chain variable | QVQLVQSGAAVKKPGESLKISCKG |
| | region of HC-4 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 151 | Light chain variable | AIQMTQSPSSLSASVGDRVTITCR |
| | region of LC-4 | ASQGIRNDLGWYQQKPGKAPKLLI |
| | | YDASSLESGVPSRFSGSGSGTDF |
| | | TLTISSLQPEDFATYYCQQFNSYP |
| | | LTFGGGTKVDIK |
| SEQ ID NO: 147 | Heavy chain variable | QVQLVQSGAAVKKPGESLKISCKG |
| | region of HC-5 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 152 | Light chain variable | NIQMTQSPSSLSASVGDRVTITCR |
| | region of LC-5 | ASQAISDYLAWFQQKPGKAPKLLI |
| | | YDASNLETGVPSRFSGSGSGTDF |
| | | TLTISSLQPEDFATYYCQQLNSYPL |
| | | TFGGGTKVEIK |
| SEQ ID NO: 147 | Heavy chain variable | QVQLVQSGAAVKKPGESLKISCKG |
| | region of HC-6 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 153 | Light chain variable | AIRMTQSPSSLSASVGDRVIIACRA |
| | region of LC-6 | SQGIGGALAWYQQKPGNAPKVLV |
| | | YDASTLESGVPSRFSGGGSGTDF |
| | | TLTISSLQPEDFATYYCQQFNSYP |
| | | LTFGGGTKLEIK |
| SEQ ID NO: 147 | Heavy chain variable | QVQLVQSGAAVKKPGESLKISCKG |
| | region of HC-7 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 154 | Light chain variable | DIAMTQSPPSLSAFVGDRVTITCR |
| | region of LC-7 | ASQGIISSLAWYQQKPGKAPKLLIY |
| | | DASSLESGVPSRFSGSGSGTDFT |
| | | LTIRSLQPEDFATYYCQQFNSYPL |
| | | TFGGGTKLEIK |
| SEQ ID NO 147: | Heavy chain variable | QVQLVQSGAAVKKPGESLKISCKG |
| | region of HC-8 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 155 | Light chain variable | DIQMTQSPSSLSASVGDRVTITCR |
| | region of LC-8 | ASQGISSALAWYQQKAGKAPKVLI |
| | | SDASSLESGVPSRFSGSGSGTDF |
| | | TLSISSLQPEDFATYYCQQFNGYP |
| | | LTFGGGTKVDIK |
| SEQ ID NO: 147 | Heavy chain variable | QVQLVQSGAAVKKPGESLKISCKG |
| | region of HC-9 | SGYRFTTYWIGWVRQMPGKGLE |
| | amino acid | WMGIIYPGDSDTRYSPSFQGQVTI |
| | sequence | SAGKSISTAYLQWSSLKASDTAMY |
| | | YCARHGRGYNGYEGAFDIWGQG |
| | | TMVTVSS |
| SEQ ID NO: 156 | Light chain variable | AIRMTQSPSSLSASVGDRVTITCQ |
| | region of LC-9 | ASQGIRNDLGWYQQKPGKAPKLLI |
| | | YDASNLETGVPSRFSGSGSGTDF |
| | | TFTISSLQPEDIATYYCQQFNSYPL |
| | | TFGGGTKLEIK |
| SEQ ID NO: 147 | Heavy chain variable | QVQLVQSGAAVKKPGESLKISCKG |
| | region of HC-10 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 157 | Light chain variable | NIQMTQSPSSLSTSVGDRVTITCR |
| | region of LC-10 | ASQGIGTSLAWYQQKPGKPPKLLI |
| | | YDASSLESGVPSRLSGSGSGTDF |
| | | TLTISSLQPEDFATYYCQQSNSYPI |
| | | TFGQGTRLEIK |
| SEQ ID NO: 147 | Heavy chain variable | QVQLVQSGAAVKKPGESLKISCKG |
| | region of HC-11 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 158 | Light chain variable | AIQLTQSPSSLSASVGDRVTITCRA |
| | region of LC-11 | SQSIGDYLTWYQQKPGKAPKVLIY |
| | | GASSLQSGVPPRFSGSGSGTDFT |
| | | LTVSSLQPEDFATYYCQQLNSYPL |
| | | TFGGGTKLEIK |
| SEQ ID NO: 147 | Heavy chain variable | QVQLVQSGAAVKKPGESLKISCKG |
| | region of HC-12 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 159 | Light chain variable | DIQLTQSPSSLSASVGDRVTITCRA |
| | region of LC-12 | SQGVRSTLAWYQQKPGKAPKLLIY |
| | | DASILESGVPSRFSGSGSGTDFTL |
| | | TISSLQPEDFATYYCQQFNGYPLT |
| | | FGQGTRLEIK |
| SEQ ID NO: 147 | Heavy chain variable | QVQLVQSGAAVKKPGESLKISCKG |
| | region of HC-13 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 160 | Light chain variable | DIVMTQSPSSLSASVGDRVTITCR |
| | region of LC-13 | ASQGIRNDLGWYQQKPGKAPKLLI |
| | | YDASSLESGVPSRFSGSGSGTDF |
| | | TLTISSLQPEDFATYYCQQFNSYP |
| | | LTFGGGTKLEIK |
| SEQ ID NO: 147 | Heavy chain variable | QVQLVQSGAAVKKPGESLKISCKG |
| | region of HC-14 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 161 | Light chain variable | DIQLTQSPSSLSASVGDRVTITCRA |
| | region of LC-14 | SQGISSFLAWYQQKPGKAPKLLIY |
| | | DASTLQSGVPSRFSGSASGTDFTL |
| | | TISSLQPEDFATYYCQQLNGYPLT |
| | | FGGGTKVEIK |
| SEQ ID NO: 147 | Heavy chain variable | QVQLVQSGAAVKKPGESLKISCKG |
| | region of HC-15 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 162 | Light chain variable | AIQLTQSPSSLSASVGDRVTITCRA |
| | region of LC-15 | SQGIGSALAWYQQKPGIGPKLLIY |
| | | DASTLESGVPARFSGSGSRTDFTL |
| | | TITSLQPEDFATYYCQQFNGYPLT |
| | | FGGGTKLEIK |
| SEQ ID NO: 147 | Heavy chain variable | QVQLVQSGAAVKKPGESLKISCKG |
| | region of HC-16 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 163 | Light chain variable | AIQLTQSPSSLSASVGDRVTITCRA |
| | region of LC-16 | SQGITSALAWYQEKPGKAPNLLIY |
| | | DASSLESGVPSRFSGSGYGTDFT |
| | | LTISSLQPEDFATYYCQQLNSYPLT |
| | | FGGGTKVDIK |
| SEQ ID NO: 164 | Heavy chain variable | QIQLVQSGPELRKPGESVKISCKA |
| | region of HC-17 | SGYTFTDYAMYWVKQAPGKGLK |
| | | WMGWINTYTGKPTYADDFKGRFV |
| | | FSLEASANTANLQISNLKNEDTATY |
| | | FCARARGLVDDYVMDAWGQGTS |
| | | VTVSS |
| SEQ ID NO: 165 | Light chain variable | SYELIQPPSASVTLGNTVSLTCVG |
| | region of LC-17 | DELSKRYAQWYQQKPDKTIVSVIY |
| | | KDSERPSGISDRFSGSSSGTTATL |
| | | TIHGTLAEDEADYYCLSTYSDDNL |
| | | PVFGGGTKLTVL |
| SEQ ID NO: 166 | Heavy chain variable | EVQLQQYGAELGKPGTSVRLSCK |
| | region of HC-18 | VSGYNIRNTYIHWVNQRPGEGLE |
| | | WIGRIDPTNGNTISAEKFKTKATLT |
| | | ADTSSHTAYLQFSQLKSDDTAIYF |
| | | CALNYEGYADYWGQGVMVTGSS |
| SEQ ID NO: 167 | Light chain variable | DIQMTQSPSFLSASVGDRVTINCK |
| | region of LC-18 | ASQNINKYLNWYQQKVGEAPKRLI |
| | | FKTNSLQTGIPSRFSGSGSGTDYT |
| | | LTISSLQTEDVATYFCFQYNIGYTF |
| | | GAGTKVELK |
| SEQ ID NO: 168 | Heavy chain variable | EVQLQESGPGLVKPSQSLSLTCSV |
| | region of HC-19 | TGYSISSNYRWNWIRKFPGNKVE |
| | | WMGYINSAGSTNYNPSLKSRISMT |
| | | RDTSKNQFFLQVNSVTTEDTATYY |
| | | CARSLRGYITDYSGFFDYWGQGV |
| | | MVTVSS |
| SEQ ID NO: 169 | Light chain variable | DIRMTQSPASLSASLGETVNIECLA |
| | region of LC-19 | SEDIFSDLAWYQQKPGKSPQLLIY |
| | | NANSLQNGVPSRFSGSGSGTRYS |
| | | LKINSLQSEDVATYFCQQYKNYPL |
| | | TFGSGTKLEIK |
| SEQ ID NO: 170 | Heavy chain variable | EVQLQQYGAELGKPGTSVRLSCK |
| | region of HC-20 | LSGYKIRNTYIHWVNQRPGKGLE |
| | | WIGRIDPANGNTIYAEKFKSKVTLT |
| | | ADTSSNTAYMQLSQLKSDDTALYF |
| | | CAMNYEGYEDYWGQGVMVTVSS |
| SEQ ID NO: 171 | Light chain variable | DIQMTQSPSFLSASVGDSVTINCK |
| | region of LC-20 | ASQNINKYLNWYQQKLGEAPKRLI |
| | | HKTDSLQTGIPSRFSGSGSGTDYT |
| | | LTISSLQPEDVATYFCFQYKSGFM |
| | | FGAGTKLELK |
| SEQ ID NO: 172 | Heavy chain variable | QIQLVQSGPELKKPGESVKISCKA |
| | region of HC-21 | SGYTFTDYAVYWVIQAPGKGLKW |
| | | MGWINTYTGKPTYADDFKGRFVF |
| | | SLETSASTANLQISNLKNEDTATYF |
| | | CARGAGMTKDYVMDAWGRGVLV |
| | | TVS |
| SEQ ID NO: 173 | Light chain variable | SYELIQPPSASVTLGNTVSLTCVG |
| | region of LC-21 | DELSKRYAQWYQQKPDKTIVSVIY |
| | | KDSERPSDISDRFSGSSSGTTATL |
| | | TIHGTLAEDEADYYCLSTYSDDNL |
| | | PVFGGGTKLTVL |
| SEQ ID NO: 174 | Heavy chain variable | QVQLKESGPGLVQPSQTLSLTCTV |
| | region of HC-22 | SGFSLTSYLVHWVRQPPGKTLEW |
| | | VGLMWNDGDTSYNSALKSRLSIS |
| | | RDTSKSQVFLKMHSLQAEDTATY |
| | | YCARESNLGFTYWGHGTLVTVSS |
| SEQ ID NO: 175 | Light chain variable | DIQMTQSPASLSASLEEIVTITCKA |
| | region of LC-22 | SQGIDDDLSWYQQKPGKSPQLLIY |
| | | DVTRLADGVPSRFSGSRSGTQYS |
| | | LKISRPQVADSGIYYCLQSYSTPYT |
| | | FGAGTKLELK |
| SEQ ID NO: 176 | Heavy chain variable | EVQLQQYGAELGKPGTSVRLSCK |
| | region of HC-23 | VSGYNIRNTYIHWVHQRPGEGLE |
| | | WIGRIDPTNGNTISAEKFKSKATLT |
| | | ADTSSNTAYMQFSQLKSDDTAIYF |
| | | CAMNYEGYADYWGQGVMVTVSS |
| SEQ ID NO: 177 | Light chain variable | DIQMTQSPSFLSASVGDRLTINCK |
| | region of LC-23 | ASQNINKYLNWYQQKLGEAPKRLI |
| | | FKTNSLQTGIPSRFSGSGSGTDYT |
| | | LTISSLQPEDVATYFCFQYNIGFTF |
| | | GAGTKLELK |
| SEQ ID NO: 178 | Heavy chain variable | EVQLVESGGGLVQSGRSLKLSCA |
| | region of HC-24 | ASGFTVSDYYMAWVRQAPTKGLE |
| | | WVATINYDGSTTYHRDSVKGRFTI |
| | | SRDNAKSTLYLQMDSLRSEDTATY |
| | | YCARHGDYGYHYGAYYFDYWGQ |
| | | GVMVTVSS |
| SEQ ID NO: 179 | Light chain variable | DIVLTQSPALAVSLGQRATISCRAS |
| | region of LC-24 | QTVSLSGYNLIHWYQQRTGQQPK |
| | | LLIYRASNLAPGIPARFSGSGSGTD |
| | | FTLTISPVQSDDIATYYCQQSRES |
| | | WTFGGGTNLEMK |
| SEQ ID NO: 180 | Heavy chain variable | QIQLVQSGPELKKPGESVKISCKA |
| | region of HC-25 | SGYTFTDYAIHWVKQAPGQGLRW |
| | | MAWINTETGKPTYADDFKGRFVF |
| | | SLEASASTAHLQISNLKNEDTATFF |
| | | CAGGSHWFAYWGQGTLVTVSS |
| SEQ ID NO: 181 | Light chain variable | SYELIQPPSASVTLENTVSITCSGD |
| | region of LC-25 | ELSNKYAHWYQQKPDKTILEVIYN |
| | | DSERPSGISDRFSGSSSGTTAILTI |
| | | RDAQAEDEADYYCLSTFSDDDLPI |
| | | FGGGTKLTVL |
| SEQ ID NO: 172 | Heavy chain variable | QIQLVQSGPELKKPGESVKISCKA |
| | region of HC-26 | SGYTFTDYAVYWVIQAPGKGLKW |
| | | MGWINTYTGKPTYADDFKGRFVF |
| | | SLETSASTANLQISNLKNEDTATYF |
| | | CARGAGMTKDYVMDAWGRGVLV |
| | | TVS |
| SEQ ID NO: 182 | Light chain variable | SYELIQPPSTSVTLGNTVSLTCVG |
| | region of LC-26 | NELPKRYAYWFQQKPDQSIVRLIY |
| | | DDDRRPSGISDRFSGSSSGTTATL |
| | | TIRDAQAEDEAYYYCHSTYTDDKV |
| | | PIFGGGTKLTVL |
| SEQ ID NO: 183 | Heavy chain variable | EVQLVESGGGLVQPGRSMKLSCK |
| | region of HC-27 | ASGFTFSNYDMAWVRQAPTRGLE |
| | | WVASISYDGITAYYRDSVKGRFTIS |
| | | RENAKSTLYLQLVSLRSEDTATYY |
| | | CTTEGGYVYSGPHYFDYWGQGV |
| | | MVTVSS |
| SEQ ID NO: 184 | Light chain variable | DIQMTQSPSSMSVSLGDTVTITCR |
| | region of LC-27 | ASQDVGIFVNWFQQKPGRSPRRM |
| | | IYRATNLADGVPSRFSGSRSGSDY |
| | | SLTISSLESEDVADYHCLQYDEFP |
| | | RTFGGGTKLELK |
| SEQ ID NO: 185 | Heavy chain variable | EVQLQQYGAELGKPGTSVRLSCK |
| | region of HC-28 | VSGYKIRNTYIHWVNQRPGKGLE |
| | | WIGRIDPANGNTIYAEKFKSKVTLT |
| | | ADTSSNTAYMQLSQLKSDDTALYF |
| | | CAMNYEGYEDYWGQGVMVTVSS |
| SEQ ID NO: 186 | Light chain variable | DIQMTQSPSFLSASVGDSVTINCK |
| | region of LC-28 | ASQNINKYLNWYQQKLGEAPKRLI |
| | | HKTNSLQPGFPSRFSGSGSGTDY |
| | | TLTISSLQPEDVAAYFCFQYNSGF |
| | | TFGAGTKLELK |
| SEQ ID NO: 187 | Heavy chain variable | QVQLVQSGAEVKKPGASVKVSCK |
| | region of HC-29 | ASGYTFTDYYIHWVRQAPGQGLE |
| | | WMGWMNPHSGDTGYAQKFQGR |
| | | VTMTRDTSTSTVYMELSSLRSEDT |
| | | AVYYCARHGRGYNGYEGAFDIWG |
| | | QGTLVTVSSAS |
| SEQ ID NO: 188 | Light chain variable | DIQMTQSPSSLSASVGDRVTITCR |
| | region of LC-29 | ASQGIGNELGWYQQKPGKAPKLLI |
| | | YAASNLQSGVPSRFSGSGSGTDF |
| | | TLTISSLQPEDFATYYCQQYDNLP |
| | | LTFGQGTKVEIK |
| SEQ ID NO: 189 | Heavy chain variable | QVQLVQSGAEVKKPGASVKVSCK |
| | region of HC-30 | ASGYTFTGYYLHWVRQAPGQGLE |
| | | WMGWINPNSGDTNYAQNFQGRV |
| | | TMTRDTSTSTVYMELSSLRSEDTA |
| | | VYYCARHGRGYNGYEGAFDIWG |
| | | QGTLVTVSSAS |
| SEQ ID NO: 190 | Light chain variable | DIQMTQSPSSLSASVGDRVTITCR |
| | region of LC-30 | ASQGIRNDLGWYQQKPGKAPKLLI |
| | | YDASSLESGVPSRFSGSGSGTDF |
| | | TLTISSLQPEDFATYYCQQLNGYP |
| | | LTFGGGTKVEIK |
| SEQ ID NO: 191 | Heavy chain variable | QVQLVQSGAEVKKPGASVKVSCK |
| | region of HC-31 | ASGYTFTGYYLHWVRQAPGQGLE |
| | | WMGWINPNSGGTNYAQKFQGRV |
| | | TMTRDTSTSTVYMELSSLRSEDTA |
| | | VYYCARHGRGYEGYEGAFDIWGQ |
| | | GTLVTVSSAS |
| SEQ ID NO: 192 | Light chain variable | DIQMTQSPSSLSASVGDRVTITCR |
| | region of LC-31 | ASQGIRNDLGWYQQKPGKAPKLLI |
| | | YDASELETGVPSRFSGSGSGTDF |
| | | TLTISSLQPEDFATYYCQQLNGYPI |
| | | TFGQGTKVEIK |
| SEQ ID NO: 193 | Heavy chain variable | QVQLVQSGAEVKKPGASVKVSCK |
| | region of HC-32 | ASGYTFTSYYIHWVRQAPGQGLE |
| | | WMGWLNPSGGGTSYAQKFQGRV |
| | | TMTRDTSTSTVYMELSSLRSEDTA |
| | | VYYCARHGRGYDGYEGAFDIWG |
| | | QGTLVTVSSAS |
| SEQ ID NO: 194 | Light chain variable | DIQMTQSPSSLSASVGDRVTITCR |
| | region of LC-32 | ASQGIRNDLGWYQQKPGKAPKLLI |
| | | YDASNLETGVPSRFSGSGSGTDF |
| | | TLTISSLQPEDFATYYCQQLNGYP |
| | | LTFGGGTKVEIK |
| SEQ ID NO: 195 | Heavy chain variable | QVQLVQSGAEVKKPGASVKVSCK |
| | region of HC-33 | ASGYTFSTYYMHWVRQAPGQGL |
| | | EWMGIINPSGGSTSYAQKFQGRV |
| | | TMTRDTSTSTVYMKLSSLRSEDTA |
| | | VYYCARHGRGYEGYEGAFDIWGQ |
| | | GTLVTVSSAS |
| SEQ ID NO: 196 | Light chain variable | DIQMTQSPSSLSASVGDRVTITCR |
| | region of LC-33 | ASQGIRDDLGWYQQKPGKAPKLLI |
| | | YDASNLETGVPSRFSGSGSGTDF |
| | | TLTISSLQPEDFATYYCQQANGFP |
| | | LTFGGGTKVEIK |
| SEQ ID NO: 197 | Heavy chain variable | QVQLVQSGAEVKKPGASVKVSCK |
| | region of HC-34 | ASGYTFTGYYIHWVRQAPGQGLE |
| | | WMGIINPSGGNTNYAQNFQGRVT |
| | | MTRDTSTSTVYMELSSLRSEDTAV |
| | | YYCARHGRGYNAYEGAFDIWGQ |
| | | GTLVTVSSAS |
| SEQ ID NO: 198 | Light chain variable | DIQMTQSPSSLSASVGDRVTITCR |
| | region of LC-34 | ASQGIRNDLGWYQQKPGKAPKLLI |
| | | YDASNLETGVPSRFSGSGSGTDF |
| | | TLTISSLQPEDFATYYCQQVNGYP |
| | | LTFGGGTKVEIK |
| SEQ ID NO: 199 | Heavy chain variable | QVQLVQSGAEVKKPGASVKVSCK |
| | region of HC-35 | ASGGTFSSYAISWVRQAPGQGLE |
| | | WMGVINPTVGGANYAQKFQGRVT |
| | | MTRDTSTSTVYMELSSLRSEDTAV |
| | | YYCARHGRGYNEYEGAFDIWGQ |
| | | GTLVTVSSAS |
| SEQ ID NO: 200 | Light chain variable | DIQMTQSPSSLSASVGDRVTITCQ |
| | region of LC-35 | ASQDISDYLNWYQQKPGKAPKLLI |
| | | YDASNLETGVPSRFSGSGSGTDF |
| | | TLTISSLQPEDFATYYCQQGNSFP |
| | | LTFGGGTKLEIK |
| SEQ ID NO: 201 | Heavy chain variable | |
| | | QVQLVQSGAEVKKLGASVKVSCK |
| | region of HC-36 | ASGYTFSSYYMHWVRQAPGQGL |
| | | EWMGVINPNGAGTNFAQKFQGRV |
| | | TMTRDTSTSTVYMELSSLRSEDTA |
| | | VYYCARHGRGYEGYEGAFDIWGQ |
| | | GTLVTVSSAS |
| SEQ ID NO: 190 | Light chain variable | DIQMTQSPSSLSASVGDRVTITCR |
| | region of LC-36 | ASQGIRNDLGWYQQKPGKAPKLLI |
| | | YDASSLESGVPSRFSGSGSGTDF |
| | | TLTISSLQPEDFATYYCQQLNGYP |
| | | LTFGGGTKVEIK |
| SEQ ID NO: 202 | Heavy chain variable | QVQLVQSGAEVKKPGASVKVSCK |
| | region of HC-37 | ASGYTFTTYYMHWVRQAPGQGLE |
| | | WMGWINPTGGGTNYAQNFQGRV |
| | | TMTRDTSTSTVYMELSSLRSEDTA |
| | | VYYCARHGRGYEGYEGAFDIWGQ |
| | | GTLVTVSSAS |
| SEQ ID NO: 203 | Light chain variable | DIQMTQSPSSLSASVGDRVTITCR |
| | region of LC-37 | ASQGIRNDVSWYQQKPGKAPKLLI |
| | | YDASNLETGVPSRFSGSGSGTDF |
| | | TLTISSLQPEDFATYYCQQLSGYPI |
| | | TFGQGTKLEIK |
| SEQ ID NO: 204 | Heavy chain variable | QVQLVQSGAEVKKPGASVKVSCK |
| | region of HC-38 | ASGYTFTSYYIHWVRQAPGQGLE |
| | | WMGMINPSGGSTNYAQKFQGRV |
| | | TMTRDTSTSTVYMELSSLRSEDTA |
| | | VYYCARHGRGYNDYEGAFDIWGQ |
| | | GTLVTVSSAS |
| SEQ ID NO: 205 | Light chain variable | DIQMTQSPSSLSASVGDRVTITCR |
| | region of LC-38 | ASQSISDWLAWYQQKPGKAPKLLI |
| | | YEASNLEGGVPSRFSGSGSGTDF |
| | | TLTISSLQPEDFATYYCQQANSFP |
| | | YTFGQGTKVEIK |
| SEQ ID NO: 206 | Heavy chain variable | QVQLVQSGAEVKKPGASVKVSCK |
| | region of HC-39 | ASGYIFSAYYIHWVRQAPGQGLE |
| | | WMGIINPSGGSTRYAQKFQGRVT |
| | | MTRDTSTSTVYMELSSLRSEDTAV |
| | | YYCARHGRGYGGYEGAFDIWDQ |
| | | GTLVTVSSAS |
| SEQ ID NO: 207 | Light chain variable | DIQMTQSPSSLSASVGDRVTITCR |
| | region of LC-39 | ASQGIGDYVAWYQQKPGKAPKLLI |
| | | YDASNLETGVPSRFSGSGSGTDF |
| | | TLTISSLQPEDFATYYCQQLNGYPI |
| | | TFGQGTRLEIK |
| SEQ ID NO: 208 | Heavy chain variable | EVQLVQSGAEVKKPGESLKISCKG |
| | region of HC-40 | SGYRFTSYWIGWVRQMPGKGLE |
| | | WMGIIYPDDSDTRYSPSFQGQVTI |
| | | SVDKSNSTAYLQWSSLKASDTAM |
| | | YYCARHGRGYNGYEGAFDIWGQ |
| | | GTLVTVSSAS |
| SEQ ID NO: 209 | Light chain variable | DIQMTQSPSSLSASVGDRVTITCR |
| | region of LC-40 | ASQGISSYLAWYQQKPGKAPKLLI |
| | | YDASNLETGVPSRFSGSGSGTYF |
| | | TLTISSLQPEDFATYYCQQGASFPI |
| | | TFGQGTKVEIK |
| SEQ ID NO: 210 | Heavy chain variable | EVQLVQSGAEVKKPGESLKISCKG |
| | region of HC-41 | SGSSFPNSWIAWVRQMPGKGLE |
| | | WMGIIYPSDSDTRYSPSFQGQVTI |
| | | SADKSISTAYLQWSSLEASDTAMY |
| | | YCARHGRGYNGYEGAFDIWGQG |
| | | TLVTVSSAS |
| SEQ ID NO: 211 | Light chain variable | DIQMTQSPSSLSASVGDRVTITCR |
| | region of LC-41 | ASQGIRNYLAWYQQKPGKAPKLLI |
| | | YDASSLQSGVPSRFSGSGSGTDF |
| | | TLTISSLQPEDFATYYCQQLNSYPL |
| | | TFGGGTKVEIK |
| SEQ ID NO: 212 | Heavy chain variable | EVQLVQSGAEVKKPGESLKISCKG |
| | region of HC-42 | SGYSFDSYWIGWVRQMPGKGLE |
| | | WMGIMYPGDSDTRYSPSFQGQVT |
| | | ISADKSISTAYLQWSSLKASDTAM |
| | | YYCARHGRGYNAYEGAFDIWGQ |
| | | GTLVTVSSAS |
| SEQ ID NO: 213 | Light chain variable | DIQMTQSPSSLSASVGDRVTITCR |
| | region of LC-42 | ASQSINNWLAWYQQKPGKAPKLLI |
| | | YDAFILQSGVPSRFSGSGSGTDFT |
| | | LTISSLQPEDFATYYCLQLNSYPLT |
| | | FGPGTKVDIK |
| SEQ ID NO: 214 | Heavy chain variable | EVQLVQSGAEVKKPGESLKISCKG |
| | region of HC-43 | SGYSFTNWIAWVRQMPGKGLEW |
| | | MGIIYPGDSETRYSPSFQGQVTIS |
| | | ADKSISTAYLQWSSLKASDTAMYY |
| | | CARHGRGYYGYEGAFDIWGQGTL |
| | | VTVSSAS |
| SEQ ID NO: 215 | Light chain variable | DIQMTQSPSSLSASVGDRVTITCR |
| | region of LC-43 | ASQGISDNLNWYQQKPGKAPKLLI |
| | | YDASNLETGVPSRFSGSGSGTDF |
| | | TLTISSLQPEDFATYYCQQAISFPL |
| | | TFGQGTKVEIK |
| SEQ ID NO: 216 | Heavy chain variable | EVQLVQSGAEVKKPGESLKISCKG |
| | region of HC-44 | SGYNFTSYWIGWVRQMPGKGLE |
| | | WMGVIYPDDSETRYSPSFQGQVTI |
| | | SADKSISTAYLQWSSLKASDTAMY |
| | | YCARHGRGYNGYEGAFDIWGQG |
| | | TLVTVSSAS |
| SEQ ID NO: 217 | Light chain variable | DIQMTQSPSSLSASVGDRVTITCR |
| | region of LC-44 | ASRDIRDDLGWYQQKPGKAPKLLI |
| | | YDASNLETGVPSRFSGSGSGTDF |
| | | TLTISSLQPEDFATYYCQQANSFP |
| | | LTFGGGTKVEIK |
| SEQ ID NO: 218 | Heavy chain variable | EVQLVQSGAEVKKPGESLKISCKG |
| | region of HC-45 | SGYTFNTYIGWVRQMPGKGLEW |
| | | MGIIYPGDSGTRYSPSFQGQVTIS |
| | | ADKAISTAYLQWSSLKASDTAMYY |
| | | CARHSRGYNGYEGAFDIWGQGTL |
| | | VTVSSAS |
| SEQ ID NO: 219 | Light chain variable | DIQMTQSPSSLSASVGDRVTITCR |
| | region of LC-45 | ASQGISNYLAWYQQKPGKAPKLLI |
| | | YDASNLETGVPSRFSGSGSGTDF |
| | | TLTISSLQPEDFATYYCQQANSFP |
| | | VTFGQGTKVEIK |
| SEQ ID NO: 220 | Heavy chain variable | EVQLVQSGAEVKKPGESLKISCKG |
| | region of HC-46 | SGYNFTTYWIGWVRQMPGKGLE |
| | | WMGIIHPADSDTRYNPSFQGQVTI |
| | | SADKSISTAYLQWSSLKASDTAMY |
| | | YCARHGRGYNGYEGAFDIWGQG |
| | | TLVTVSSAS |
| SEQ ID NO: 221 | Light chain variable | DIQMTQSPSSLSASVGDRVTITCR |
| | region of LC-46 | VSQGISSYLAWYQQKPGKAPKLLI |
| | | YDASNLETGVPSRFSGSGSGTDF |
| | | TLTISSLQPEDFATYYCQQANSFP |
| | | LTFGGGTKVEIK |
| SEQ ID NO: 222 | Heavy chain variable | EVQLVQSGAEVKKPGESLKISCKG |
| | region of HC-47 | SGYRFSNYWIAWVRQMPGKGLE |
| | | WMGIIYPDNSDTRYSPSFQGQVTI |
| | | SADKSISTAYLQWSSLKASDTAMY |
| | | YCARHGRGYDGYEGAFDIWGQG |
| | | TLVTVSSAS |
| SEQ ID NO: 223 | Light chain variable | DIQMTQSPSSLSASVGDRVTITCR |
| | region of LC-47 | ASQGIRSDLAWYQQKPGKAPKLLI |
| | | YGASSLQSGVPSRFSGSGSGTDF |
| | | TLTISSLQPEDFATYYCQQANSFP |
| | | LSFGQGTKVEIK |
| SEQ ID NO: 224 | Heavy chain variable | EVQLVQSGAEVKKPGESLKISCKG |
| | region of HC-48 | SGYRFASYWIGWVRQMPGKGLE |
| | | WMGITYPGDSETRYNPSQGQVTI |
| | | SADKSISTAYLQWSSLKASDTAMY |
| | | YCARHGRGYGGYEGAFDIWGQG |
| | | TLVTVSSAS |
| SEQ ID NO: 225 | Light chain variable | DIQMTQSPSSLSASVGDRVTITCR |
| | region of LC-48 | ASQGIRNDLGWYQQKPGKAPKLLI |
| | | YDASNLETGVPSRFSGSGSGTDF |
| | | TLTISSLQPEDFATYYCQQANSFP |
| | | LTFGGGTKVEIK |
| SEQ ID NO: 226 | Heavy chain variable | EVQLVQSGAEVKKPGESLKISCKG |
| | region of HC-49 | SGYSFTSYWIGWVRQMPGKGLE |
| | | WMGIIYPGDSDTRYSPSFQGQVTI |
| | | SADKSISTAYLQWSSLKASDTAMY |
| | | YCARHGRGYNGYEGAFDIWGQG |
| | | TLVTVSSAS |
| SEQ ID NO: 227 | Light chain variable | DIQMTQSPSSLSASVGDRVTITCR |
| | region of LC-49 | ASQSISNWLAWYQQKPGKAPKLLI |
| | | YDASNLETGVPSRFSGSGSGTDF |
| | | TLTISSLQPEDFATYYCQQTNSFPL |
| | | TFGQGTRLEIK |
| SEQ ID NO: 147 | Heavy chain variable | QVQLVQSGAAVKKPGESLKISCKG |
| | region of HC-74 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 228 | Light chain variable | DIQLTQSPSSLSASVGDRVTITCRA |
| | region of LC-74 | SQGVISALAWYQQKPGKAPKLLIY |
| | | DASSLESGVPSRFSGSGSGTDFT |
| | | LTISSLQPEDFATYYCQQFNSYPL |
| | | TFGGGTKVEIK |
| SEQ ID NO: 147 | Heavy chain variable | QVQLVQSGAAVKKPGESLKISCKG |
| | region of HC-75 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 229 | Light chain variable | DIQLTQSPSSLSASVGDRVTITCRA |
| | region of LC-75 | SQGIRSALAWYQQKPGKAPKLLIY |
| | | DASSLESGVPSRFSGSGSGTDFT |
| | | LTISSLQPEDFATYYCQQFNSYPL |
| | | TFGGGTKVEIK |
| SEQ ID NO: 147 | Heavy chain variable | QVQLVQSGAAVKKPGESLKISCKG |
| | region of HC-76 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 230 | Light chain variable | DIQLTQSPSSLSASVGDRVTITCRA |
| | region of LC-76 | SQGVGSALAWYQQKPGKAPKLLI |
| | | YDASSLESGVPSRFSGSGSGTDF |
| | | TLTISSLQPEDFATYYCQQFNSYP |
| | | LTFGGGTKVEIK |
| SEQ ID NO: 147 | Heavy chain variable | QVQLVQSGAAVKKPGESLKISCKG |
| | region of HC-77 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 231 | Light chain variable | DIQLTQSPSSLSASVGDRVTITCRA |
| | region of LC-77 | SQGVISALAWYQQKPGKAPKLLIY |
| | | DASILESGVPSRFSGSGSGTDFTL |
| | | TISSLQPEDFATYYCQQFNSYPLT |
| | | FGGGTKVEIK |
| SEQ ID NO: 147 | Heavy chain variable | QVQLVQSGAAVKKPGESLKISCKG |
| | region of HC-78 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 232 | Light chain variable | DIQLTQSPSSLSASVGDRVTITCRA |
| | region of LC-78 | SQGIRSALAWYQQKPGKAPKLLIY |
| | | DASILESGVPSRFSGSGSGTDFTL |
| | | TISSLQPEDFATYYCQQFNSYPLT |
| | | FGGGTKVEIK |
| SEQ ID NO: 147 | Heavy chain variable | QVQLVQSGAAVKKPGESLKISCKG |
| | region of HC-79 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 233 | Light chain variable | DIQLTQSPSSLSASVGDRVTITCRA |
| | region of LC-79 | SQGVGSALAWYQQKPGKAPKLLI |
| | | YDASILESGVPSRFSGSGSGTDFT |
| | | LTISSLQPEDFATYYCQQFNSYPL |
| | | TFGGGTKVEIK |
| SEQ ID NO: 147 | Heavy chain variable | QVQLVQSGAAVKKPGESLKISCKG |
| | region of HC-80 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 234 | Light chain variable | DIQLTQSPSSLSASVGDRVTITCRA |
| | region of LC-80 | SQGISSALAWYQQKPGKAPKLLIY |
| | | DASILESGVPSRFSGSGSGTDFTL |
| | | TISSLQPEDFATYYCQQFNSYPLT |
| | | FGGGTKVEIK |
| SEQ ID NO: 147 | Heavy chain variable | QVQLVQSGAAVKKPGESLKISCKG |
| | region of HC-81 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 235 | Light chain variable | DIQLTQSPSSLSASVGDRVTITCRA |
| | region of LC-81 | SQGVISALAWYQQKPGKAPKLLIY |
| | | DASTLESGVPSRFSGSGSGTDFTL |
| | | TISSLQPEDFATYYCQQFNSYPLT |
| | | FGGGTKVEIK |
| SEQ ID NO: 147 | Heavy chain variable | QVQLVQSGAAVKKPGESLKISCKG |
| | region of HC-82 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 236 | Light chain variable | DIQLTQSPSSLSASVGDRVTITCRA |
| | region of LC-82 | SQGIRSALAWYQQKPGKAPKLLIY |
| | | DASTLESGVPSRFSGSGSGTDFTL |
| | | TISSLQPEDFATYYCQQFNSYPLT |
| | | FGGGTKVEIK |
| SEQ ID NO: 147 | Heavy chain variable | QVQLVQSGAAVKKPGESLKISCKG |
| | region of HC-83 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 237 | Light chain variable | DIQLTQSPSSLSASVGDRVTITCRA |
| | region of LC-83 | SQGVGSALAWYQQKPGKAPKLLI |
| | | YDASTLESGVPSRFSGSGSGTDF |
| | | TLTISSLQPEDFATYYCQQFNSYP |
| | | LTFGGGTKVEIK |
| SEQ ID NO: 147 | Heavy chain variable | QVQLVQSGAAVKKPGESLKISCKG |
| | region of HC-84 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 237 | Light chain variable | DIQLTQSPSSLSASVGDRVTITCRA |
| | region of LC-84 | SQGVGSALAWYQQKPGKAPKLLI |
| | | YDASTLESGVPSRFSGSGSGTDF |
| | | TLTISSLQPEDFATYYCQQFNSYP |
| | | LTFGGGTKVEIK |
| SEQ ID NO: 238 | Heavy chain variable | EVQLVQSGAEVKKPGESLKISCKG |
| | region of HC-245 | SGYRFTTSWIGWVRQMPGKGLE |
| | | WMGIIYPGDSDTRYSPSFQGQVTI |
| | | SADKSISTAYLQWSSLKASDTAMY |
| | | YCARHGLGYNGYEGAFDIWGQGT |
| | | LVTVSS |
| SEQ ID NO: 239 | Light chain variable | DIQMTQSPSSLSASVGDRVTITCR |
| | region of LC-245 | ASQGIGSALAWYQQKPGKAPKLLI |
| | | YDASTLESGVPSRFSGSGSGTDF |
| | | TLTISSLQPEDFATYYCQQFNGYP |
| | | LTFGQGTRLEIK |
| SEQ ID NO: 147 | Heavy chain variable | QVQLVQSGAAVKKPGESLKISCKG |
| | region of HC-246 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 239 | Light chain variable | DIQMTQSPSSLSASVGDRVTITCR |
| | region of LC-246 | ASQGIGSALAWYQQKPGKAPKLLI |
| | | YDASTLESGVPSRFSGSGSGTDF |
| | | TLTISSLQPEDFATYYCQQFNGYP |
| | | LTFGQGTRLEIK |
| SEQ ID NO: 147 | Heavy chain variable | QVQLVQSGAAVKKPGESLKISCKG |
| | region of HC-247 | SGYRFTTYWIGWVRQMPGKGLE |
| | | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 240 | Light chain variable | DIQMTQSPSSLSASVGDRVTITCR |
| | region of LC-247 | ASRGISDYLAWYQQKPGKAPKLLI |
| | | YDASNLETGVPSRFSGSGSGTDF |
| | | TLTISSLQPEDFATYYCQQANSFPI |
| | | TFGQGTRLEIK |
| SEQ ID NO: 238 | Heavy chain variable | EVQLVQSGAEVKKPGESLKISCKG |
| | region of HC-248 | SGYRFTTSWIGWVRQMPGKGLE |
| | | WMGIIYPGDSDTRYSPSFQGQVTI |
| | | SADKSISTAYLQWSSLKASDTAMY |
| | | YCARHGLGYNGYEGAFDIWGQGT |
| | | LVTVSS |
| SEQ ID NO: 241 | Light chain variable | DIQMTQSPSSLSASVGDRVTITCR |
| | region of LC-248 | ASQGIGSALAWYQQKPGKAPKLLI |
| | | YDASTLESGVPSRFSGSGSGTDF |
| | | TLTISSLQPEDFATYYCQQLNGYP |
| | | LTFGQGTRLEIK |
| SEQ ID NO: 238 | Heavy chain variable | EVQLVQSGAEVKKPGESLKISCKG |
| | region of HC-249 | SGYRFTTSWIGWVRQMPGKGLE |
| | | WMGIIYPGDSDTRYSPSFQGQVTI |
| | | SADKSISTAYLQWSSLKASDTAMY |
| | | YCARHGLGYNGYEGAFDIWGQGT |
| | | LVTVSS |
| SEQ ID NO: 242 | Light chain variable | DIQMTQSPSSLSASVGDRVTITCR |
| | region of LC-249 | ASQGIGSALAWYQQKPGKAPKLLI |
| | | YDASNLETGVPSRFSGSGSGTDF |
| | | TLTISSLQPEDFATYYCQQLNGYP |
| | | LTFGQGTRLEIK |
| SEQ ID NO: 243 | Heavy chain variable | EVQLVQSGAEVKKPGESLKISCKG |
| | region of Ab85 | SGYSFTNYWIGWVRQMPGKGLE |
| | | WMAIINPRDSDTRYRPSFQGQVTI |
| | | SADKSISTAYLQWSSLKASDTAMY |
| | | YCARHGRGYEGYEGAFDIWGQG |
| | | TLVTVSS |
| SEQ ID NO: 244 | Light chain variable | DIQMTQSPSSLSASVGDRVTITCR |
| | region of Ab 85 | SSQGIRSDLGWYQQKPGKAPKLLI |
| | | YDASNLETGVPSRFSGSGSGTDF |
| | | TLTISSLQPEDFATYYCQQANGFP |
| | | LTFGGGTKVEIK |
| SEQ ID NO: 245 | Ab85 CDR-H1 | NYWIG |
| SEQ ID NO: 246 | Ab85 CDR-H2 | IINPRDSDTRYRPSFQG |
| SEQ ID NO: 247 | Ab85 CDR-H3 | HGRGYEGYEGAFDI |
| SEQ ID NO: 248 | Ab85 CDR-L1 | RSSQGIRSDLG |
| SEQ ID NO: 249 | Ab85 CDR-L2 | DASNLET |
| | Ab249 CDR-L2 | |
| SEQ ID NO: 250 | Ab85 CDR-L3 | QQANGFPLT |
| SEQ ID NO: 251 | Heavy chain variable | EVQLVQSGAEVKKPGESLKISCKG |
| | region of Ab86 | SGYSFTNYWIGWVRQMPGKGLE |
| | | WMGIIYPGDSDIRYSPSLQGQVTIS |
| | | VDTSTSTAYLQWNSLKPSDTAMY |
| | | YCARHGRGYNGYEGAFDIWGQG |
| | | TLVTVSS |
| SEQ ID NO: 252 | Light chain variable | DIQMTQSPSSLSASVGDRVTITCR |
| | region of Ab86 | ASQGIGDSLAWYQQKPGKAPKLLI |
| | | YDASNLETGVPSRFSGSGSGTDF |
| | | TLTISSLQPEDFATYYCQQLNGYPI |
| | | TFGQGTKVEIK |
| SEQ ID NO: 245 | Ab86 CDR-H1 | NYWIG |
| SEQ ID NO: 253 | Ab86 CDR-H2 | IIYPGDSDIRYSPSLQG |
| SEQ ID NO: 3 | Ab86 CDR-H3 | HGRGYNGYEGAFDI |
| SEQ ID NO: 254 | Ab86 CDR-L1 | RASQGIGDSLA |
| SEQ ID NO: 249 | Ab86 CDR-L2 | DASNLET |
| SEQ ID NO: 255 | Ab86 CDR-L3 | QQLNGYPIT |
| SEQ ID NO: 243 | Heavy chain variable | EVQLVQSGAEVKKPGESLKISCKG |
| | region of Ab87 | SGYSFTNYWIGWVRQMPGKGLE |
| | | WMAIINPRDSDTRYRPSFQGQVTI |
| | | SADKSISTAYLQWSSLKASDTAMY |
| | | YCARHGRGYEGYEGAFDIWGQG |
| | | TLVTVSS |
| SEQ ID NO: 256 | Light chain variable | DIQMTQSPSSLSASVGDRVTITCR |
| | region of Ab87 | ASQGIRNDLGWYQQKPGKAPKLLI |
| | | YDASSLESGVPSRFSGSGSGTDF |
| | | TLTISSLQPEDFATYYCQQLNGYPI |
| | | TFGQGTKVEIK |
| SEQ ID NO: 245 | Ab87 CDR-H1 | NYWIG |
| SEQ ID NO: 246 | Ab87 CDR-H2 | IINPRDSDTRYRPSFQG |
| SEQ ID NO: 247 | Ab87 CDR-H3 | HGRGYEGYEGAFDI |
| SEQ ID NO: 257 | Ab87 CDR-L1 | RASQGIRNDLG |
| SEQ ID NO: 5 | Ab87 CDR-L2 | DASSLES |
| SEQ ID NO: 255 | Ab87 CDR-L3 | QQLNGYPIT |
| SEQ ID NO: 258 | Heavy chain variable | EVQLVQSGAEVKKPGESLKISCKG |
| | region of Ab88 | SGYSFTNYWIGWVRQMPGKGLE |
| | | WMGIIYPGDSLTRYSPSFQGQVTI |
| | | SADKSISTAYLQWSSLKASDTAMY |
| | | YCARHGRGYNGYEGAFDIWGQG |
| | | TLVTVSS |
| SEQ ID NO: 256 | Light chain variable | DIQMTQSPSSLSASVGDRVTITCR |
| | region of Ab88 | ASQGIRNDLGWYQQKPGKAPKLLI |
| | | YDASSLESGVPSRFSGSGSGTDF |
| | | TLTISSLQPEDFATYYCQQLNGYPI |
| | | TFGQGTKVEIK |
| SEQ ID NO: 245 | Ab88 CDR-H1 | NYWIG |
| SEQ ID NO: 259 | Ab88 CDR-H2 | IIYPGDSLTRYSPSFQG |
| SEQ ID NO: 3 | Ab88 CDR-H3 | HGRGYNGYEGAFDI |
| SEQ ID NO: 257 | Ab88 CDR-L1 | RASQGIRNDLG |
| SEQ ID NO: 5 | Ab88 CDR-L2 | DASSLES |
| SEQ ID NO: 255 | Ab88 CDR-L3 | QQLNGYPIT |
| SEQ ID NO: 260 | Heavy chain variable | EVQLVQSGAEVKKPGESLKISCKG |
| | region of Ab89 | SGYSFTNYWIGWVRQMPGKGLE |
| | | WMGIIYPGDSDTRYSPSFQGQVTI |
| | | SADKSISTAYLQWSSLKASDTAMY |
| | | YCARHGRGYNGYEGAFDIWGQG |
| | | TLVTVSS |
| SEQ ID NO: 252 | Light chain variable | DIQMTQSPSSLSASVGDRVTITCR |
| | region of Ab89 | ASQGIGDSLAWYQQKPGKAPKLLI |
| | | YDASNLETGVPSRFSGSGSGTDF |
| | | TLTISSLQPEDFATYYCQQLNGYPI |
| | | TFGQGTKVEIK |
| SEQ ID NO: 245 | Ab89 CDR-H1 | NYWIG |
| SEQ ID NO: 2 | Ab89 CDR-H2 | IIYPGDSDTRYSPSFQG |
| SEQ ID NO: 3 | Ab89 CDR-H3 | HGRGYNGYEGAFDI |
| SEQ ID NO: 254 | Ab89 CDR-L1 | RASQGIGDSLA |
| SEQ ID NO: 249 | Ab89 CDR-L2 | DASNLET |
| SEQ ID NO: 255 | Ab89 CDR-L3 | QQLNGYPIT |
| SEQ ID NO: 261 | Heavy chain variable | QVQLVQSGAAVKKPGESLKISCKG |
| | region amino acid | SGYRFTSYWIGWVRQMPGKGLE |
| | sequence of CK6 | WMGIIYPGDSDTRYSPSFQGQVTI |
| | | SAGKSISTAYLQWSSLKASDTAMY |
| | | YCARHGRGYNGYEGAFDIWGQG |
| | | TMVTVSS |
| SEQ ID NO: 262 | Light chain variable | AIQLTQSPSSLSASVGDRVTITCRA |
| | region amino acid | SQGISSALAWYQQKPGKAPKLLIY |
| | sequence of CK6 | DASSLESGVPSRFSGSGSGTDFT |
| | | LTISSLQPEDFATYYCQQFNSYPL |
| | | TFGGGTKVEIK |
| SEQ ID NO: 263 | Ab77 CDR-H1 | TYWIG |
| SEQ ID NO: 2 | Ab77 CDR-H2 | IIYPGDSDTRYSPSFQG |
| SEQ ID NO: 3 | Ab77 CDR-H3 | HGRGYNGYEGAFDI |
| SEQ ID NO: 264 | Ab77 CDR-L1 | RASQGVISALA |
| SEQ ID NO: 265 | Ab77 CDR-L2 | DASILES |
| SEQ ID NO: 266 | Ab77 CDR-L3 | QQFNSYPLT |
| SEQ ID NO: 263 | Ab79 CDR-H1 | TYWIG |
| SEQ ID NO: 2 | Ab79 CDR-H2 | IIYPGDSDTRYSPSFQG |
| SEQ ID NO: 3 | Ab79 CDR-H3 | HGRGYNGYEGAFDI |
| SEQ ID NO: 267 | Ab79 CDR-L1 | RASQGVGSALA |
| SEQ ID NO: 265 | Ab79 CDR-L2 | DASILES |
| SEQ ID NO: 266 | Ab79 CDR-L3 | QQFNSYPLT |
| SEQ ID NO: 263 | Ab81 CDR-H1 | TYWIG |
| SEQ ID NO: 2 | Ab81 CDR-H2 | IIYPGDSDTRYSPSFQG |
| SEQ ID NO: 3 | Ab81 CDR-H3 | HGRGYNGYEGAFDI |
| SEQ ID NO: 264 | Ab81 CDR-L1 | RASQGVISALA |
| SEQ ID NO: 268 | Ab81 CDR-L2 | DASTLES |
| SEQ ID NO: 266 | Ab81 CDR-L3 | QQFNSYPLT |
| SEQ ID NO: 269 | Heavy chain | ASTKGPSVFPLAPSSKSTSGGTAA |
| | constant region | LGCLVKDYFPEPVTVSWNSGALT |
| | (Wild type (WT)) | SGVHTFPAVLQSSGLYSLSSVVTV |
| | | PSSSLGTQTYICNVNHKPSNTKVD |
| | | KKVEPKSCDKTHTCPPCPAPELL |
| | | GGPSVFLFPPKPKDTLMISRTPEV |
| | | TCVVVDVSHEDPEVKFNWYVDGV |
| | | EVHNAKTKPREEQYNSTYRVVSV |
| | | LTVLHQDWLNGKEYKCKVSNKAL |
| | | PAPIEKTISKAKGQPREPQVYTLPP |
| | | SRDELTKNQVSLTCLVKGFYPSDI |
| | | AVEWESNGQPENNYKTTPPVLDS |
| | | DGSFFLYSKLTVDKSRWQQGNVF |
| | | SCSVMHEALHNHYTQKSLSLSPG |
| | | K |
| SEQ ID NO: 270 | Heavy chain | ASTKGPSVFPLAPSSKSTSGGTAA |
| | constant region with | LGCLVKDYFPEPVTVSWNSGALT |
| | L234A, L235A | SGVHTFPAVLQSSGLYSLSSVVTV |
| | (LALA) mutations | PSSSLGTQTYICNVNHKPSNTKVD |
| | (mutations in bold)* | KKVEPKSCDKTHTCPPCPAPEAA |
| | | GGPSVFLFPPKPKDTLMISRTPEV |
| | | TCVVVDVSHEDPEVKFNWYVDGV |
| | | EVHNAKTKPREEQYNSTYRVVSV |
| | | LTVLHQDWLNGKEYKCKVSNKAL |
| | | PAPIEKTISKAKGQPREPQVYTLPP |
| | | SRDELTKNQVSLTCLVKGFYPSDI |
| | | AVEWESNGQPENNYKTTPPVLDS |
| | | DGSFFLYSKLTVDKSRWQQGNVF |
| | | SCSVMHEALHNHYTQKSLSLSPG |
| | | K |
| SEQ ID NO: 271 | Heavy chain constant | ASTKGPSVFPLAPSSKSTSGGTAA |
| | region with D265C | LGCLVKDYFPEPVTVSWNSGALT |
| | mutation | SGVHTFPAVLQSSGLYSLSSVVTV |
| | (mutation in bold)* | PSSSLGTQTYICNVNHKPSNTKVD |
| | | KKVEPKSCDKTHTCPPCPAPELLG |
| | | GPSVFLFPPKPKDTLMISRTPEVT |
| | | CVVVCVSHEDPEVKFNWYVDGVE |
| | | VHNAKTKPREEQYNSTYRVVSVLT |
| | | VLHQDWLNGKEYKCKVSNKALPA |
| | | PIEKTISKAKGQPREPQVYTLPPSR |
| | | DELTKNQVSLTCLVKGFYPSDIAV |
| | | EWESNGQPENNYKTTPPVLDSDG |
| | | SFFLYSKLTVDKSRWQQGNVFSC |
| | | SVMHEALHNHYTQKSLSLSPGK |
| SEQ ID NO: 272 | Heavy chain constant | ASTKGPSVFPLAPSSKSTSGGTAA |
| | region with H435A | LGCLVKDYFPEPVTVSWNSGALT |
| | mutation | SGVHTFPAVLQSSGLYSLSSVVTV |
| | (mutation in bold)* | PSSSLGTQTYICNVNHKPSNTKVD |
| | | KKVEPKSCDKTHTCPPCPAPELLG |
| | | GPSVFLFPPKPKDTLMISRTPEVT |
| | | CVVVDVSHEDPEVKFNWYVDGVE |
| | | VHNAKTKPREEQYNSTYRVVSVLT |
| | | VLHQDWLNGKEYKCKVSNKALPA |
| | | PIEKTISKAKGQPREPQVYTLPPSR |
| | | DELTKNQVSLTCLVKGFYPSDIAV |
| | | EWESNGQPENNYKTTPPVLDSDG |
| | | SFFLYSKLTVDKSRWQQGNVFSC |
| | | SVMHEALHNAYTQKSLSLSPGK |
| SEQ ID NO: 273 | Heavy chain | ASTKGPSVFPLAPSSKSTSGGTAA |
| | constant region: | LGCLVKDYFPEPVTVSWNSGALT |
| | modified Fc region | SGVHTFPAVLQSSGLYSLSSVVTV |
| | with L234A, L235A, | PSSSLGTQTYICNVNHKPSNTKVD |
| | D265C mutations | KKVEPKSCDKTHTCPPCPAPEAA |
| | (mutations in bold)* | GGPSVFLFPPKPKDTLMISRTPEV |
| | | TCVVVCVSHEDPEVKFNWYVDGV |
| | | EVHNAKTKPREEQYNSTYRVVSV |
| | | LTVLHQDWLNGKEYKCKVSNKAL |
| | | PAPIEKTISKAKGQPREPQVYTLPP |
| | | SRDELTKNQVSLTCLVKGFYPSDI |
| | | AVEWESNGQPENNYKTTPPVLDS |
| | | DGSFFLYSKLTVDKSRWQQGNVF |
| | | SCSVMHEALHNHYTQKSLSLSPG |
| | | K |
| SEQ ID NO: 274 | Heavy chain | ASTKGPSVFPLAPSSKSTSGGTAA |
| | constant region: | LGCLVKDYFPEPVTVSWNSGALT |
| | modified Fc region | SGVHTFPAVLQSSGLYSLSSVVTV |
| | with L234A, L235A, | PSSSLGTQTYICNVNHKPSNTKVD |
| | D265C, H435A | KKVEPKSCDKTHTCPPCPAPEAA |
| | mutations (mutations | GGPSVFLFPPKPKDTLMISRTPEV |
| | in bold)* | TCVVVCVSHEDPEVKFNWYVDGV |
| | | EVHNAKTKPREEQYNSTYRVVSV |
| | | LTVLHQDWLNGKEYKCKVSNKAL |
| | | PAPIEKTISKAKGQPREPQVYTLPP |
| | | SRDELTKNQVSLTCLVKGFYPSDI |
| | | AVEWESNGQPENNYKTTPPVLDS |
| | | DGSFFLYSKLTVDKSRWQQGNVF |
| | | SCSVMHEALHNAYTQKSLSLSPG |
| | | K |
| SEQ ID NO: 275 | Ab85 full length | EVQLVQSGAEVKKPGESLKISCKG |
| | heavy chain | SGYSFTNYWIGWVRQMPGKGLE |
| | sequence; constant | WMAIINPRDSDTRYRPSFQGQVTI |
| | region underlined | SADKSISTAYLQWSSLKASDTAMY |
| | | YCARHGRGYEGYEGAFDIWGQG |
| | | TLVTVSSASTKGPSVFPLAPSSKS |
| | | TSGGTAALGCLVKDYFPEPVTVS |
| | | WNSGALTSGVHTFPAVLQSSGLY |
| | | SLSSVVTVPSSSLGTQTYICNVNH |
| | | KPSNTKVDKKVEPKSCDKTHTCP |
| | | PCPAPELLGGPSVFLFPPKPKDTL |
| | | MISRTPEVTCVVVDVSHEDPEVKF |
| | | NWYVDGVEVHNAKTKPREEQYNS |
| | | TYRVVSVLTVLHQDWLNGKEYKC |
| | | KVSNKALPAPIEKTISKAKGQPREP |
| | | QVYTLPPSRDELTKNQVSLTCLVK |
| | | GFYPSDIAVEWESNGQPENNYKT |
| | | TPPVLDSDGSFFLYSKLTVDKSRW |
| | | QQGNVFSCSVMHEALHNHYTQKS |
| | | LSLSPGK |
| SEQ ID NO: 276 | Ab85 full length | EVQLVQSGAEVKKPGESLKISCKG |
| | heavy chain | SGYSFTNYWIGWVRQMPGKGLE |
| | sequence; constant | WMAIINPRDSDTRYRPSFQGQVTI |
| | region underlined; | SADKSISTAYLQWSSLKASDTAMY |
| | modified Fc region | YCARHGRGYEGYEGAFDIWGQG |
| | with L234A, L235A | TLVTVSSASTKGPSVFPLAPSSKS |
| | mutations (mutations | TSGGTAALGCLVKDYFPEPVTVS |
| | in bold)* | WNSGALTSGVHTFPAVLQSSGLY |
| | | SLSSVVTVPSSSLGTQTYICNVNH |
| | | KPSNTKVDKKVEPKSCDKTHTCP |
| | | PCPAPEAAGGPSVFLFPPKPKDTL |
| | | MISRTPEVTCVVVDVSHEDPEVKF |
| | | NWYVDGVEVHNAKTKPREEQYNS |
| | | TYRVVSVLTVLHQDWLNGKEYKC |
| | | KVSNKALPAPIEKTISKAKGQPREP |
| | | QVYTLPPSRDELTKNQVSLTCLVK |
| | | GFYPSDIAVEWESNGQPENNYKT |
| | | TPPVLDSDGSFFLYSKLTVDKSRW |
| | | QQGNVFSCSVMHEALHNHYTQKS |
| | | LSLSPGK |
| SEQ ID NO: 277 | Ab85 full length | EVQLVQSGAEVKKPGESLKISCKG |
| | heavy chain | SGYSFTNYWIGWVRQMPGKGLE |
| | sequence: constant | WMAIINPRDSDTRYRPSFQGQVTI |
| | region underlined; | SADKSISTAYLQWSSLKASDTAMY |
| | modified Fc region | YCARHGRGYEGYEGAFDIWGQG |
| | with L234A, L235A, | TLVTVSSASTKGPSVFPLAPSSKS |
| | D265C mutations | TSGGTAALGCLVKDYFPEPVTVS |
| | (mutations in bold)* | WNSGALTSGVHTFPAVLQSSGLY |
| | | SLSSVVTVPSSSLGTQTYICNVNH |
| | | KPSNTKVDKKVEPKSCDKTHTCP |
| | | PCPAPEAAGGPSVFLFPPKPKDTL |
| | | MISRTPEVTCVVVCVSHEDPEVKF |
| | | NWYVDGVEVHNAKTKPREEQYNS |
| | | TYRVVSVLTVLHQDWLNGKEYKC |
| | | KVSNKALPAPIEKTISKAKGQPREP |
| | | QVYTLPPSRDELTKNQVSLTCLVK |
| | | GFYPSDIAVEWESNGQPENNYKT |
| | | TPPVLDSDGSFFLYSKLTVDKSRW |
| | | QQGNVFSCSVMHEALHNHYTQKS |
| | | LSLSPGK |
| SEQ ID NO: 278 | Ab85 full length | EVQLVQSGAEVKKPGESLKISCKG |
| | heavy chain | SGYSFTNYWIGWVRQMPGKGLE |
| | sequence (LALA- | WMAIINPRDSDTRYRPSFQGQVTI |
| | D265C-H435A | SADKSISTAYLQWSSLKASDTAMY |
| | mutant); constant | YCARHGRGYEGYEGAFDIWGQG |
| | region underlined | TLVTVSSASTKGPSVFPLAPSSKS |
| | | TSGGTAALGCLVKDYFPEPVTVS |
| | | WNSGALTSGVHTFPAVLQSSGLY |
| | | SLSSVVTVPSSSLGTQTYICNVNH |
| | | KPSNTKVDKKVEPKSCDKTHTCP |
| | | PCPAPEAAGGPSVFLFPPKPKDTL |
| | | MISRTPEVTCVVVCVSHEDPEVKF |
| | | NWYVDGVEVHNAKTKPREEQYNS |
| | | TYRVVSVLTVLHQDWLNGKEYKC |
| | | KVSNKALPAPIEKTISKAKGQPREP |
| | | QVYTLPPSRDELTKNQVSLTCLVK |
| | | GFYPSDIAVEWESNGQPENNYKT |
| | | TPPVLDSDGSFFLYSKLTVDKSRW |
| | | QQGNVFSCSVMHEALHNAYTQKS |
| | | LSLSPGK |
| SEQ ID NO: 279 | Ab249 full length | EVQLVQSGAEVKKPGESLKISCKG |
| | heavy chain | SGYRFTTSWIGWVRQMPGKGLE |
| | sequence; constant | WMGIIYPGDSDTRYSPSFQGQVTI |
| | region underlined | SADKSISTAYLQWSSLKASDTAMY |
| | | YCARHGLGYNGYEGAFDIWGQGT |
| | | LVTVSSASTKGPSVFPLAPSSKST |
| | | SGGTAALGCLVKDYFPEPVTVSW |
| | | NSGALTSGVHTFPAVLQSSGLYSL |
| | | SSVVTVPSSSLGTQTYICNVNHKP |
| | | SNTKVDKKVEPKSCDKTHTCPPC |
| | | PAPELLGGPSVFLFPPKPKDTLMIS |
| | | RTPEVTCVVVDVSHEDPEVKFNW |
| | | YVDGVEVHNAKTKPREEQYNSTY |
| | | RVVSVLTVLHQDWLNGKEYKCKV |
| | | SNKALPAPIEKTISKAKGQPREPQ |
| | | VYTLPPSRDELTKNQVSLTCLVKG |
| | | FYPSDIAVEWESNGQPENNYKTT |
| | | PPVLDSDGSFFLYSKLTVDKSRW |
| | | QQGNVFSCSVMHEALHNHYTQKS |
| | | LSLSPGK |
| SEQ ID NO: 280 | Ab249 full length | EVQLVQSGAEVKKPGESLKISCKG |
| | heavy chain | SGYRFTTSWIGWVRQMPGKGLE |
| | sequence; constant | WMGIIYPGDSDTRYSPSFQGQVTI |
| | region underlined | SADKSISTAYLQWSSLKASDTAMY |
| | (LALA mutations)* | YCARHGLGYNGYEGAFDIWGQGT |
| | | LVTVSSASTKGPSVFPLAPSSKST |
| | | SGGTAALGCLVKDYFPEPVTVSW |
| | | NSGALTSGVHTFPAVLQSSGLYSL |
| | | SSVVTVPSSSLGTQTYICNVNHKP |
| | | SNTKVDKKVEPKSCDKTHTCPPC |
| | | PAPEAAGGPSVFLFPPKPKDTLMI |
| | | SRTPEVTCVVVDVSHEDPEVKFN |
| | | WYVDGVEVHNAKTKPREEQYNST |
| | | YRVVSVLTVLHQDWLNGKEYKCK |
| | | VSNKALPAPIEKTISKAKGQPREP |
| | | QVYTLPPSRDELTKNQVSLTCLVK |
| | | GFYPSDIAVEWESNGQPENNYKT |
| | | TPPVLDSDGSFFLYSKLTVDKSRW |
| | | QQGNVFSCSVMHEALHNHYTQKS |
| | | LSLSPGK |
| SEQ ID NO: 281 | Ab249 full length | EVQLVQSGAEVKKPGESLKISCKG |
| | heavy chain | SGYRFTTSWIGWVRQMPGKGLE |
| | sequence; constant | WMGIIYPGDSDTRYSPSFQGQVTI |
| | region underlined | SADKSISTAYLQWSSLKASDTAMY |
| | (LALA-D265C | YCARHGLGYNGYEGAFDIWGQGT |
| | mutations)* | LVTVSSASTKGPSVFPLAPSSKST |
| | | SGGTAALGCLVKDYFPEPVTVSW |
| | | NSGALTSGVHTFPAVLQSSGLYSL |
| | | SSVVTVPSSSLGTQTYICNVNHKP |
| | | SNTKVDKKVEPKSCDKTHTCPPC |
| | | PAPEAAGGPSVFLFPPKPKDTLMI |
| | | SRTPEVTCVVVCVSHEDPEVKFN |
| | | WYVDGVEVHNAKTKPREEQYNST |
| | | YRVVSVLTVLHQDWLNGKEYKCK |
| | | VSNKALPAPIEKTISKAKGQPREP |
| | | QVYTLPPSRDELTKNQVSLTCLVK |
| | | GFYPSDIAVEWESNGQPENNYKT |
| | | TPPVLDSDGSFFLYSKLTVDKSRW |
| | | QQGNVFSCSVMHEALHNHYTQKS |
| | | LSLSPGK |
| SEQ ID NO: 282 | Ab249 full length | EVQLVQSGAEVKKPGESLKISCKG |
| | heavy chain | SGYRFTTSWIGWVRQMPGKGLE |
| | sequence; constant | WMGIIYPGDSDTRYSPSFQGQVTI |
| | region underlined; | SADKSISTAYLQWSSLKASDTAMY |
| | (LALA-D265C- | YCARHGLGYNGYEGAFDIWGQGT |
| | H435A mutations)* | LVTVSSASTKGPSVFPLAPSSKST |
| | | SGGTAALGCLVKDYFPEPVTVSW |
| | | NSGALTSGVHTFPAVLQSSGLYSL |
| | | SSVVTVPSSSLGTQTYICNVNHKP |
| | | SNTKVDKKVEPKSCDKTHTCPPC |
| | | PAPEAAGGPSVFLFPPKPKDTLMI |
| | | SRTPEVTCVVVCVSHEDPEVKFN |
| | | WYVDGVEVHNAKTKPREEQYNST |
| | | YRVVSVLTVLHQDWLNGKEYKCK |
| | | VSNKALPAPIEKTISKAKGQPREP |
| | | QVYTLPPSRDELTKNQVSLTCLVK |
| | | GFYPSDIAVEWESNGQPENNYKT |
| | | TPPVLDSDGSFFLYSKLTVDKSRW |
| | | QQGNVFSCSVMHEALHNAYTQKS |
| | | LSLSPGK |
| SEQ ID NO: 283 | Light chain constant | RTVAAPSVFIFPPSDEQLKSGTAS |
| | region | VVCLLNNFYPREAKVQWKVDNAL |
| | | QSGNSQESVTEQDSKDSTYSLSS |
| | | TLTLSKADYEKHKVYACEVTHQGL |
| | | SSPVTKSFNRGEC |
| SEQ ID NO: 284 | Ab85 full length light | DIQMTQSPSSLSASVGDRVTITCR |
| | chain; constant | SSQGIRSDLGWYQQKPGKAPKLLI |
| | region underlined | YDASNLETGVPSRFSGSGSGTDF |
| | | TLTISSLQPEDFATYYCQQANGFP |
| | | LTFGGGTKVEIKRTVAAPSVFIFPP |
| | | SDEQLKSGTASVVCLLNNFYPREA |
| | | KVQWKVDNALQSGNSQESVTEQ |
| | | DSKDSTYSLSSTLTLSKADYEKHK |
| | | VYACEVTHQGLSSPVTKSFNRGE |
| | | C |
| SEQ ID NO: 285 | Ab249 light chain; | DIQMTQSPSSLSASVGDRVTITCR |
| | constant region | ASQGIGSALAWYQQKPGKAPKLLI |
| | underlined | YDASNLETGVPSRFSGSGSGTDF |
| | | TLTISSLQPEDFATYYCQQLNGYP |
| | | LTFGQGTRLEIKRTVAAPSVFIFPP |
| | | SDEQLKSGTASVVCLLNNFYPREA |
| | | KVQWKVDNALQSGNSQESVTEQ |
| | | DSKDSTYSLSSTLTLSKADYEKHK |
| | | VYACEVTHQGLSSPVTKSFNRGE |
| | | C |
| SEQ ID NO: 286 | Ab249 HC-CDR1 | TSWIG |
| SEQ ID NO: 287 | Ab249 HC-CDR3 | HGLGYNGYEGAFDI |
| SEQ ID NO: 288 | Ab249 LC-CDR1 | RASQGIGSALA |
| SEQ ID NO: 289 | Ab249 LC-CDR3 | CQQLNGYPLT |

### OTHER EMBODIMENTS

All publications, patents, and patent applications mentioned in this specification are incorporated herein by reference to the same extent as if each independent publication or patent application was specifically and individually indicated to be incorporated by reference.

While the present disclosure has been described in connection with specific embodiments thereof, it will be understood that it is capable of further modifications and this application is intended to cover any variations, uses, or adaptations of the present disclosure following, in general, the principles of the present disclosure and including such departures from the present disclosure that come within known or customary practice within the art to which the present disclosure pertains and may be applied to the essential features hereinbefore set forth, and follows in the scope of the claims.

Other embodiments are within the claims.

1. An antibody-drug conjugate (ADC) comprising an anti-CD117 antibody, or antigen binding portion thereof (Ab), conjugated to a cytotoxin (Cy) via a linker (L), wherein the cytotoxin comprises a pyrrolobenzodiaze pine (PBD), wherein the antibody, or the antigen binding fragment thereof, wherein anti-CD117 antibody, or antigen binding portion thereof, comprises
a heavy chain comprising a heavy chain (HC)-CDR1, HC-CDR2, and HC-CDR3 comprising an amino acid sequence as set forth in SEQ ID No: 11, 12, and 13, respectively, and a light chain comprising a light chain (LC)-CDR1, LC-CDR2, and LC-CDR3 comprising an amino acid sequence as set forth in SEQ ID Nos: 14, 15, and 16, respectively;
a heavy chain comprising a heavy chain (HC)-CDR1, HC-CDR2, and HC-CDR3 comprising an amino acid sequence as set forth in SEQ ID Nos: 245, 246, and 247, respectively, and a light chain comprising a light chain (LC)-CDR1, LC-CDR2, and LC-CDR3 comprising an amino acid sequence as set forth in SEQ ID Nos: 248, 249, and 250, respectively; or
a heavy chain comprising an HC-CDR1, an HC-CDR2, and an HC-CDR3 or a variable region from the heavy chain variable region amino acid sequence of SEQ ID NO: 147, 164, 166, 168, 170, 172, 174, 176, 178, 180, 183, 185, 187, 189, 191, 193, 195, 197, 199, 201, 202, 204, 206, 208, 210, 212, 214, 216, 218, 220, 222, 224, 226, or 238, and a light chain comprising an LC-CDR1, an LC-CDR2, and an LC-CDR3 or a variable region from the light chain variable region amino acid sequence of SEQ ID NO: 148, 149, 150, 151, 152, 153, 154, 155, 156, 157, 158, 159, 160, 161, 162, 163, 165, 167, 169, 171, 173, 175, 177, 179, 181, 182, 184, 186, 188, 190, 192, 194, 196, 198, 200, 203, 205, 207, 209, 211, 213, 215, 217, 219, 221, 223, 225, 227, 228, 229, 230, 231, 232, 233, 234, 235, 236, 237, 239, 240, 241, or 242.

2. The ADC of claim 1, wherein the cytotoxin is a PBD dimer.

3. The ADC of claim 1 or 2, wherein the PBD is represented by Formula (I): 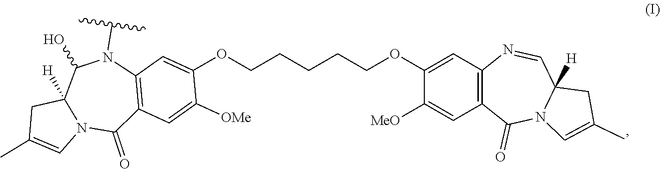
wherein the wavy line indicates the point of covalent attachment to the linker of the ADC.

4. The ADC of claim 1, wherein the linker comprises one or more of a peptide, oligosaccharide, —(CH₂)ₚ—, —(CH₂CH₂O)_{q}—, —(C═O)(CH₂)ᵣ—, —(C═O)(CH₂CH₂O)ₜ, —(NHCH₂CH₂)ᵤ—, -PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB, wherein each of p, q, r, t, and u are integers from 1-12, selected independently for each occurrence.

5. The ADC of claim 1, wherein the linker has the structure of formula (II): 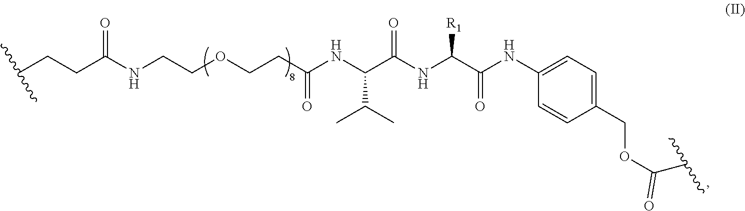
wherein R₁ is CH₃ (Ala) or (CH₂)₃NH(CO)NH₂ (Cit).

6. The ADC of claim 1, wherein the linker, prior to conjugation to the antibody and including the reactive substituent Z′, taken together as L-Z′, has the structure:

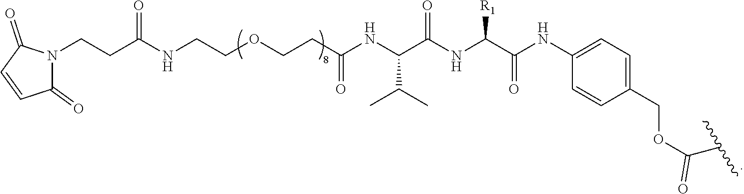

7. The ADC of claim 6, wherein R₁ is CH₃.

8. The ADC of claim 1, wherein the cytotoxin-linker conjugate, prior to conjugation to the antibody and including the reactive substituent Z′, taken together as Cy-L-Z′, is tesirine, having the structure of formula (IV):

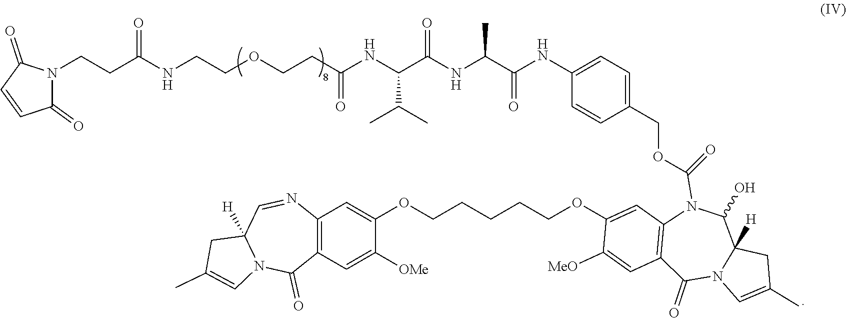

9. The ADC of claim 1, having the structure of formula (V): 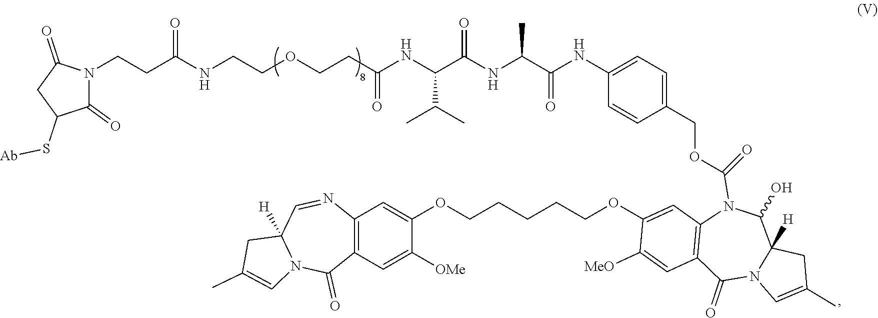
wherein Ab is the anti-CD117 antibody or antigen binding fragment thereof, and S represents a sulfur atom present in or introduced into the antibody or antigen binding fragment thereof.

10. The ADC of claim 1, wherein the antibody, or antigen binding portion thereof, comprises an Fc domain, and wherein the antibody, or antigen binding portion thereof, is conjugated to the PBD by way of a cysteine residue in the Fc domain.

11. The ADC of claim 10, wherein the cysteine residue is introduced by way of an amino acid substitution in the Fc domain, wherein the amino acid substitution is D265C and/or V205C (EU numbering).

12.-14. (canceled)

15. The ADC of claim 1, wherein the antibody, or antigen binding portion thereof, is an IgG.

16.-17. (canceled)

18. The ADC of claim 1, wherein the antibody, or the antigen binding fragment thereof, wherein anti-CD117 antibody, or antigen binding portion thereof, comprises
a heavy chain comprising a variable region comprising an amino acid sequence as set forth in SEQ ID NO: 9 and a light chain comprising a variable region comprising an amino acid sequence as set forth in SEQ ID NO: 10; or
a heavy chain comprising a variable region comprising an amino acid sequence as set forth in SEQ ID NO: 243, and a light chain comprising a variable region comprising an amino acid sequence as set forth in SEQ ID NO: 244.

19. (canceled)

20. The ADC of claim 1, wherein the antibody, or the antigen binding fragment thereof, comprises an Fc region comprising at least one mutation selected from the group consisting of D265C, H435A, L234A, or L235A (according to EU index).

21. The ADC of claim 1, wherein the antibody or antigen binding fragment thereof comprises an Fc region comprising D265C, H435A, L234A, or L235A (according to EU index) mutations.

22. A pharmaceutical composition comprising the ADC of claim 1, and a pharmaceutically acceptable carrier.

23. (canceled)

24. (canceled)

25. (canceled)

26. (canceled)

27. A method comprising administering to a human patient a transplant comprising hematopoietic stem cells, wherein the patient has previously been administered the ADC of claim 1, in an amount sufficient to deplete a population of hematopoietic stem cells from the patient.

28. The method of claim 27, wherein the patient has a blood disease, a metabolic disorder, a cancer, an autoimmune disease, or severe combined immunodeficiency disease (SCID).

29. The method of claim 28, wherein the patient has a hematological cancer.

30. The method of claim 29, wherein the hematological cancer is leukemia or lymphoma.

31. The method of claim 28, wherein the autoimmune disease is multiple sclerosis or scleroderma.

32. (canceled)

33. A method of depleting a population of CD117+ cells in a human patient in need of a hematopoietic stem cell transplant, the method comprising administering to the human patient an effective amount of an antibody-drug conjugate (ADC) comprising a pyrrolobenzodiazepine (PBD) conjugated to an antibody or antigen binding portion thereof capable of specifically binding human CD117, wherein the antibody or antigen binding portion thereof comprises an Fc domain and is internalized by a CD117+ cell, and wherein the antibody comprises
a heavy chain comprising an HC-CDR1, an HC-CDR2, and an HC-CDR3 or a variable region from the heavy chain variable region amino acid sequence of SEQ ID NO: 147, 164, 166, 168, 170, 172, 174, 176, 178, 180, 183, 185, 187, 189, 191, 193, 195, 197, 199, 201, 202, 204, 206, 208, 210, 212, 214, 216, 218, 220, 222, 224, 226, 238, or 243, and a light chain comprising an LC-CDR1, an LC-CDR2, and an LC-CDR3 or a variable region from the light chain variable region amino acid sequence of SEQ ID NO: 148, 149, 150, 151, 152, 153, 154, 155, 156, 157, 158, 159, 160, 161, 162, 163, 165, 167, 169, 171, 173, 175, 177, 179, 181, 182, 184, 186, 188, 190, 192, 194, 196, 198, 200, 203, 205, 207, 209, 211, 213, 215, 217, 219, 221, 223, 225, 227, 228, 229, 230, 231, 232, 233, 234, 235, 236, 237, 239, 240, 241, 242, or 244;
a heavy chain comprising a variable region comprising an amino acid sequence as set forth in SEQ ID NO: 9 and a light chain comprising a variable region comprising an amino acid sequence as set forth in SEQ ID NO: 10; or
a heavy chain comprising a variable region comprising an amino acid sequence as set forth in SEQ ID NO: 243, and a light chain comprising a variable region comprising an amino acid sequence as set forth in SEQ ID NO: 244.

34. The method of claim 33, wherein the patient has a hematological cancer.

35. The method of claim 34, wherein the hematological cancer is leukemia or lymphoma.

36. A method of conditioning a human patient for receiving a hematopoietic stem cell (HSC) transplant, the method comprising administering to the human patient an effective amount of an antibody-drug conjugate (ADC) comprising a pyrrolobenzodiazepine (PBD) conjugated to an antibody or antigen binding portion thereof capable of specifically binding human CD117, wherein the antibody or antigen binding portion thereof, comprises an Fc domain and is internalized by a CD117+ cell, and wherein the human patient has a stem cell disorder, and wherein the antibody comprises
a heavy chain comprising an HC-CDR1, an HC-CDR2, and an HC-CDR3 or a variable region from the heavy chain variable region amino acid sequence of SEQ ID NO: 147, 164, 166, 168, 170, 172, 174, 176, 178, 180, 183, 185, 187, 189, 191, 193, 195, 197, 199, 201, 202, 204, 206, 208, 210, 212, 214, 216, 218, 220, 222, 224, 226, 238, or 243, and a light chain comprising an LC-CDR1, an LC-CDR2, and an LC-CDR3 or a variable region from the light chain variable region amino acid sequence of SEQ ID NO: 148, 149, 150, 151, 152, 153, 154, 155, 156, 157, 158, 159, 160, 161, 162, 163, 165, 167, 169, 171, 173, 175, 177, 179, 181, 182, 184, 186, 188, 190, 192, 194, 196, 198, 200, 203, 205, 207, 209, 211, 213, 215, 217, 219, 221, 223, 225, 227, 228, 229, 230, 231, 232, 233, 234, 235, 236, 237, 239, 240, 241, 242, or 244;
a heavy chain comprising a variable region comprising an amino acid sequence as set forth in SEQ ID NO: 9 and a light chain comprising a variable region comprising an amino acid sequence as set forth in SEQ ID NO: 10; or
a heavy chain comprising a variable region comprising an amino acid sequence as set forth in SEQ ID NO: 243, and a light chain comprising a variable region comprising an amino acid sequence as set forth in SEQ ID NO: 244.

37. The method of claim 36, wherein the stem cell disorder is a hematological cancer or an autoimmune disease.

38. The method of claim 36 or 37, further comprising administering a hematopoietic stem cell transplant to the subject.

39. The method of claim 38, wherein the transplant is administered to the human patient after the ADC has substantially cleared from the blood of the human patient.

40. The method of claim 38, wherein the hematopoietic stem cell transplant comprises allogeneic cells.

41. The method of claim 38, wherein the hematopoietic stem cell transplant comprises autologous cells.
